(19) United States
(12) Patent Application Publication
(10) Pub. No.: US 2025/0109119 A1
(43) Pub. Date: Apr. 3, 2025

(54) AMPK AGONISTS AND METHODS OF USE THEREOF

(71) Applicant: Evvia Therapeutics, Inc., Belmont, CA (US)

(72) Inventors: Michael J. Green, Half Moon Bay, CA (US); Amna T. Adam, Mountain View, CA (US); Alam Jahangir, San Jose, CA (US)

(21) Appl. No.: 18/832,088

(22) PCT Filed: Jan. 24, 2023
(86) PCT No.: PCT/US2023/061185

§ 371 Date: Jul. 22, 2024

(51) Int. Cl. C07D 401/04; A61K 31/404; A61K 31/4439; A61K 31/444; A61K 31/4545; A61K 31/496; A61K 31/506; A61K 31/5377; A61K 31/553; C07D 209/42; C07D 401/14; C07D 403/04; C07D 405/10; C07D 405/14; C07D 409/04; C07D 471/04; C07D 493/04

(52) U.S. Cl. CPC C07D 401/04; A61K 31/404; A61K 31/4439; A61K 31/444; A61K 31/4545; A61K 31/496; A61K 31/506; A61K 31/5377; A61K 31/553; C07D 209/42; C07D 401/14; C07D 403/04; C07D 405/10; C07D 405/14; C07D 409/04; C07D 471/04; C07D 493/04

(57) ABSTRACT

Compounds and methods of using the same for treating conditions alleviated by AMPK activation are provided.

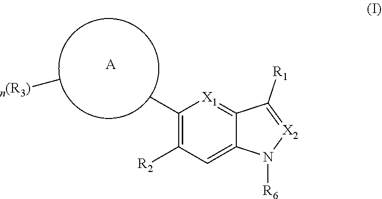

### CROSS REFERENCE TO RELATED APPLICATIONS

The present application is an International Application which claims priority to U.S. Provisional Application No. 63/302,942, filed Jan. 25, 2022, which is herein incorporated by reference in its entirety.

### FIELD

The disclosure relates generally to compounds and methods of using the same for treating conditions alleviated by AMPK activation.

### BACKGROUND

Previously studied drug candidates were conventional AMP-dependent AMPK activators, with the mechanism of action requiring elevations of AMP caused either by RC inhibition (e.g., metformin, resveratrol) or conversion into AMP mimetics (e.g., AICAR). The indirect mechanism involving RC inhibition is not suitable for cases with underlying mitochondrial dysfunction, and AMP-dependent activation of AMPK results in the activation of other AMP-regulated enzymes, thereby compounding pleiotropic effects. Additionally, previous studies have identified direct, AMP-independent AMPK agonists for the purpose of treating diabetes, obesity, and metabolic syndrome.

Primary mitochondrial diseases are a clinically heterogeneous group of disorders that are usually progressive, multi-systemic, and are associated with a high mortality rate in children. They are caused by inherited deficiencies in the mitochondrial respiratory chain (RC), leading to an increased production of reactive oxygen and nitrogen species (ROS and RNS) as well as a deficiency in overall energy production. These resulting metabolic imbalances lead to cellular damage and ultimately to cell death.

There is currently no curative treatment for primary mitochondrial disease. Only supportive treatment is available and involves treating specific symptoms (e.g., Diabetes, cardiac disease, and ptosis) and a “mitochondrial cocktail” consisting of vitamin cofactors and antioxidants. Unfortunately, meta-analyses have shown that the available supportive interventions lacks efficacy, highlighting the need for a novel treatment.

Secondary mitochondrial diseases also demonstrate mitochondrial dysfunction but, unlike primary mitochondrial diseases, are not caused by genes related to the mitochondrial respiratory chain. Secondary mitochondrial diseases, such as Parkinson's disease or Alzheimer's disease, are due to acquired mitochondrial abnormalities caused by other diseases, conditions, or environmental factors that indirectly damage the mitochondria. Consequently, any treatment identified for primary mitochondrial disease, would be expected to also benefit disorders and conditions associated with secondary mitochondrial dysfunction, including neurodegenerative, neuromuscular, and muscle wasting disorders.

There is a need in the art for novel treatments for primary and secondary mitochondrial diseases. This disclosure addresses this need in the art.

### SUMMARY

In one aspect, the disclosure provides a compound of formula (I), or a pharmaceutically acceptable salt, solvate, hydrate, cocrystal, or prodrug thereof:

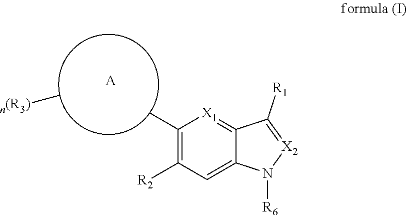

wherein in formula (I):
X₁ is CR₄ₐ or N;
X₂ is CR_{4b} or N;
R₁ is selected from

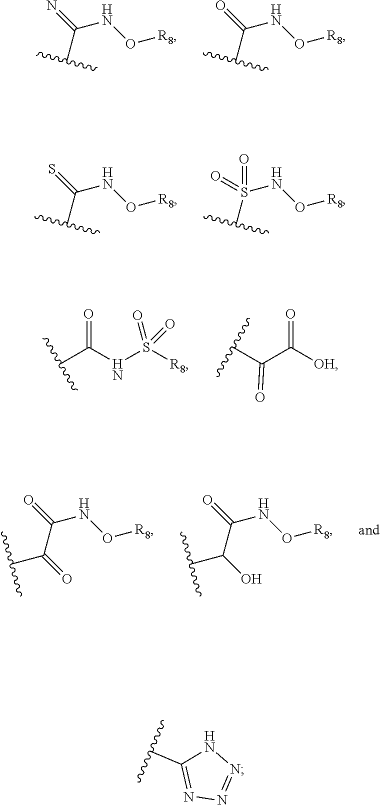

R₂ is selected from H, C₁₋₃ alkyl, —CF₃, and halo;
R³ is at each occurrence independently selected from halo, —CN,

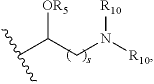

—N(R₁₀)₂, C₁₋₁₀ alkyl, C₂₋₁₀ alkynyl, C₁₋₁₀ alkoxy, C₃₋₇ cycloalkyl, C₃₋₇ cycloalkyloxy, C₃₋₁₀ heterocyclyl, C₄₋₁₂ alkylcycloalkyl, C₄₋₁₀ cycloalkylalkyl, C₃₋₇ heterocycloalkenyl, C₄₋₁₂ alkylheterocycloalkenyl, C₄₋₁₀ heterocycloalkenylalkyl, aryl and heteroaryl, wherein the C₁₋₁₀ alkyl, C₂₋₁₀ alkynyl, C₁₋₁₀ alkoxy, C₃₋₇ cycloalkyl, C₃₋₁₀ heterocyclyl, C₄₋₁₂ alkylcycloalkyl, C₄₋₁₀ cycloalkylalkyl, C₃₋₇ heterocycloalkenyl, C₄₋₁₂ alkylheterocycloalkenyl, C₄₋₁₀ heterocycloalkenylalkyl, aryl, or heteroaryl is unsubstituted or substituted with one to three substituents selected from deuterium, halo, C₁₋₆ alkyl, C₃₋₆ cycloalkyl, —OR₅ and —OCOR₇;
R₄ₐ is at each occurrence independently selected from H, halo, C₁₋₁₀ alkyl, C₃₋₇ cycloalkyl, C₃₋₁₀ heterocyclyl, C₄₋₁₂ alkylcycloalkyl, and C₄₋₁₀ cycloalkylalkyl, wherein the C₁₋₁₀ alkyl, C₃₋₇ cycloalkyl, C₃₋₁₀ heterocyclyl, C₄₋₁₂ alkylcycloalkyl, or C₄₋₁₀ cycloalkylalkyl is unsubstituted or substituted with one to three halogen substituents;
R_{4b} is at each occurrence independently selected from H, C₁₋₁₀ alkyl, C₃₋₇ cycloalkyl, C₃₋₁₀ heterocyclyl, C₄₋₁₂ alkylcycloalkyl, and C₄₋₁₀ cycloalkylalkyl, wherein the C₁₋₁₀ alkyl, C₃₋₇ cycloalkyl, C₃₋₁₀ heterocyclyl, C₄₋₁₂ alkylcycloalkyl, or C₄₋₁₀ cycloalkylalkyl is unsubstituted or substituted with one to three halogen substituents;
R₅ is at each occurrence independently selected from H, C₁₋₁₀ alkyl, C₁₋₁₀ alkoxy, C₃₋₇ cycloalkyl, C₄₋₁₂ alkylcycloalkyl, and C₄₋₁₀ cycloalkylalkyl, wherein the C₁₋₁₀ alkyl, C₁₋₁₀ alkoxy, C₃₋₇ cycloalkyl, C₄₋₁₂ alkylcycloalkyl, or C₄₋₁₀ cycloalkylalkyl is unsubstituted or substituted with one to three halogen substituents;
R₆ is at each occurrence independently selected from H, C₁₋₆ alkyl, and C₃₋₆ cycloalkyl;
R₇ is at each occurrence independently selected from C₁₋₁₀ alkyl, C₃₋₇ cycloalkyl, and C₄₋₁₂ alkylcycloalkyl, wherein the C₁₋₁₀ alkyl, C₃₋₇ cycloalkyl, or C₄₋₁₂ alkylcycloalkyl is unsubstituted or substituted with one to three substituents selected from halo, C₁₋₁₀ alkyl, and —NR₁₂, R₁₂;
R₈ is selected from H, C₁₋₁₀ alkyl, C₁₋₁₀ alkoxy, C₃₋₇ cycloalkyl, C₄₋₁₂ alkylcycloalkyl, C₄₋₁₀ cycloalkylalkyl, C₃₋₁₀ heterocyclyl, C₄₋₁₂ alkylheterocyclyl, C₃₋₇ heterocycloalkenyl, C₄₋₁₂ alkylheterocycloalkenyl, C₄₋₁₀ heterocycloalkenylalkyl, aryl, heteroaryl,

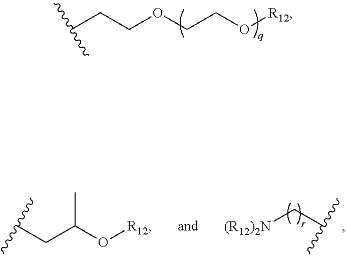

wherein the C₁₋₁₀ alkyl, C₁₋₁₀ alkoxy, C₃₋₇ cycloalkyl, C₄₋₁₂ alkylcycloalkyl, C₄₋₁₀ cycloalkylalkyl, C₃₋₁₀ heterocyclyl, C₄₋₁₂ alkylheterocyclyl, C₃₋₇ heterocycloalkenyl, C₄₋₁₂ alkylheterocycloalkenyl, C₄₋₁₀ heterocycloalkenylalkyl, aryl, or heteroaryl is unsubstituted or substituted with one to three substituents selected from deuterium, halo and —OR₅;
R₁₀ is at each occurrence independently selected from H, C₁₋₆ alkyl, C₃₋₆ cycloalkyl, C₄₋₁₂ alkylcycloalkyl,

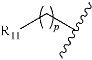

and C₃₋₁₀ heterocyclyl, wherein the C₁₋₆ alkyl, C₃₋₆ cycloalkyl, C₄₋₁₂ alkylcycloalkyl, or C₃₋₁₀ heterocyclyl is unsubstituted or substituted with one to three substituents selected from deuterium, halo, C₁₋₆ alkyl, and C₃₋₆ cycloalkyl substituents;
R₁₁ is at each occurrence independently selected from —OR₁₂ and —N(R₁₂)₂;
R₁₂ is at each occurrence independently selected from H, C₁₋₆ alkyl, C₁₋₁₀ haloalkoxy, and C₃₋₆ cycloalkyl;
n is an integer from 0-3;
p is an integer from 1-5;
q is an integer from 0-5;
r is an integer from 1-5;
s is an integer from 1-5;

A is selected from phenyl, pyridyl, pyrimidyl, pyridazyl and the following 5-membered ring heterocycles.

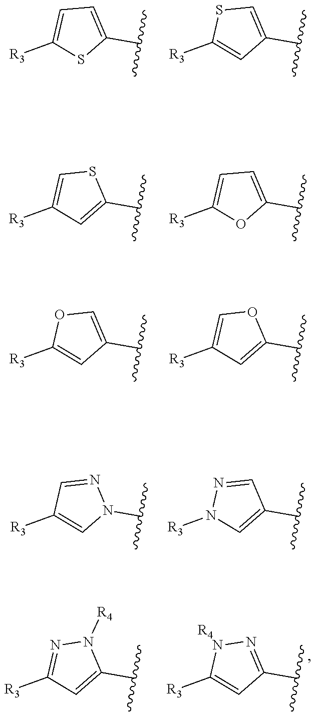

provided that the compound of formula (I) is not

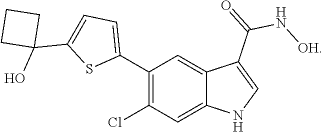 [SMILES: O=C(NO)c1c[nH]c2cc(Cl)c(-c3ccc(C4(O)CCC4)s3)cc12]

In some embodiments, X₁ is CR₄ₐ. In some embodiments, R₄ₐ is H, Cl, or F. In some embodiments, X₂ is N or CR₄, wherein R_{4b} is H. In some embodiments, R₂ is halo. In some embodiments, R₂ is Cl or F. In some embodiments, In some embodiments, R₆ is H.

In some embodiments, A is selected from:

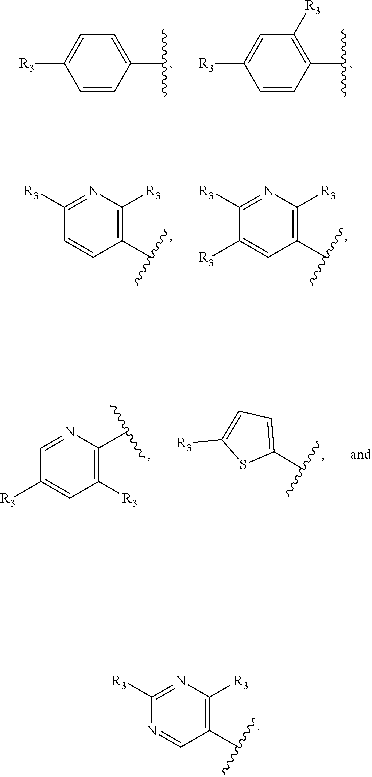

In some embodiments, R₆ is H.

In some embodiments, the compound of formula (I) is a compound of formula (10), formula (11), formula (12), formula (13), formula (14), formula (15), formula (16), or formula (17), or a pharmaceutically acceptable salt, solvate, hydrate, cocrystal, or prodrug thereof. In some embodiments, R₁ is

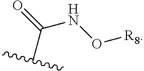

In some embodiments, the compound of formula (I) is a compound of formula (100), formula (110), formula (120), formula (130), formula (140), formula (150), formula (160), or formula (170), or a pharmaceutically acceptable salt, solvate, hydrate, cocrystal, or prodrug thereof.

In some embodiments, R₁ is

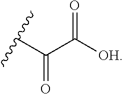 [SMILES: *C(=O)C(=O)O]

In some embodiments, the compound of formula (I) is a compound of formula (200), formula (210), formula (220), formula (230), formula (240), formula (250), formula (260), or formula (270) or a pharmaceutically acceptable salt, solvate, hydrate, cocrystal, or prodrug thereof.
In some embodiments, R₁ is

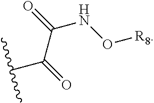

In some embodiments, the compound of formula (I) is a compound of formula (300), formula (310), formula (320), formula (330), formula (340), formula (350), formula (360), or formula (370) or a pharmaceutically acceptable salt, solvate, hydrate, cocrystal, or prodrug thereof. In some embodiments, the compound is selected from:

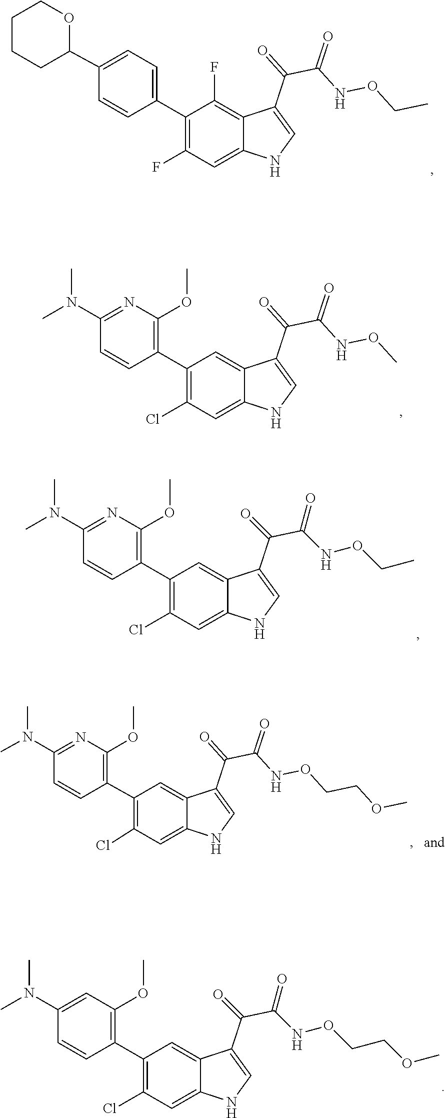

In some embodiments, R₁ is

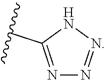 [SMILES: *c1nnn[nH]1]

In some embodiments, the compound of formula (I) is a compound of formula (400), formula (410), formula (420), formula (430), formula (440), formula (450), formula (460), or formula (470) or a pharmaceutically acceptable salt, solvate, hydrate, cocrystal, or prodrug thereof.

In some embodiments, R₁ is

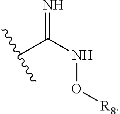

In some embodiments, the compound of formula (I) is a compound of formula (500), formula (510), formula (520), formula (530), formula (540), formula (550), formula (560), or formula (570), or a pharmaceutically acceptable salt, solvate, hydrate, cocrystal, or prodrug thereof.

In some embodiments, R₁ is

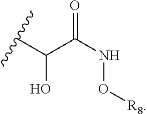

In some embodiments, the compound of formula (I) is a compound of formula (600), formula (610), formula (620), formula (630), formula (640), formula (650), formula (660), or formula (670), or a pharmaceutically acceptable salt, solvate, hydrate, cocrystal, or prodrug thereof.

In some embodiments, R₁ is

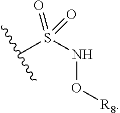

In some embodiments, the compound of formula (I) is a compound of formula (700), formula (710), formula (720), formula (730), formula (740), formula (750), formula (760), or formula (770), or a pharmaceutically acceptable salt, solvate, hydrate, cocrystal, or prodrug thereof.

In some embodiments, R₁ is

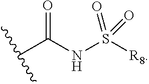

In some embodiments, the compound of formula (I) is a compound of formula (800), formula (810), formula (820), formula (830), formula (840), formula (850), formula (860), or formula (870), or a pharmaceutically acceptable salt, solvate, hydrate, cocrystal, or prodrug thereof.

In some embodiments, R₁ is

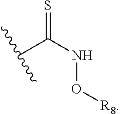

In some embodiments, the compound of formula (I) is a compound of formula (900), formula (910), formula (920), formula (930), formula (940), formula (950), formula (960), or formula (970), or a pharmaceutically acceptable salt, solvate, hydrate, cocrystal, or prodrug thereof.

In some embodiments, R₁₀ is selected from H, methyl, ethyl, —CD₃, n-butyl,

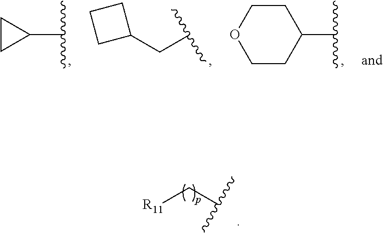

In some embodiments, R₁ is selected from

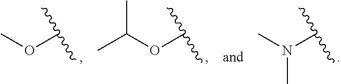

In some embodiments, p is 2 or 3. In some embodiments, R₃ is selected from —F, —CN, ethyl,

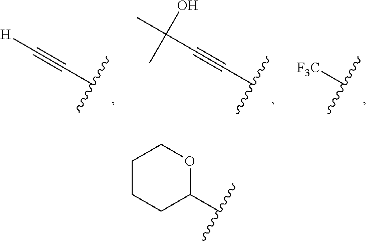

optionally

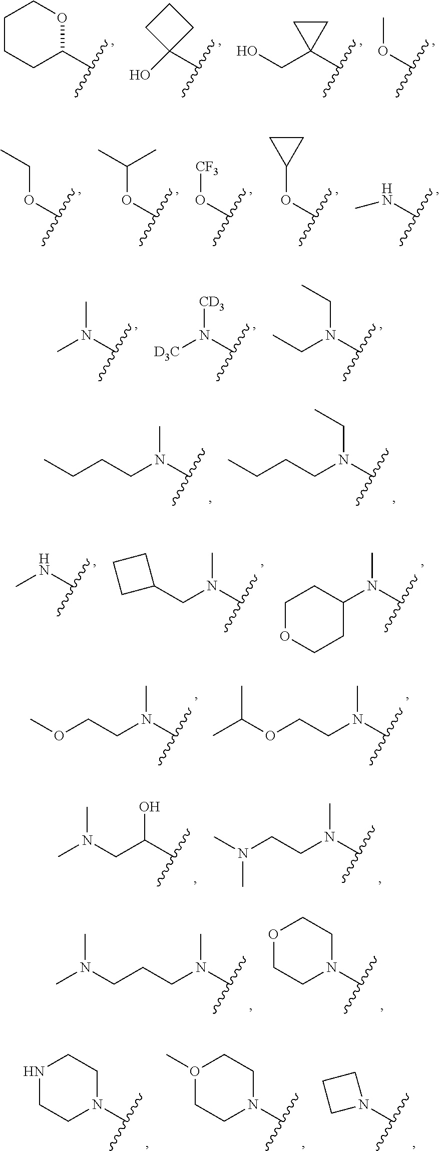

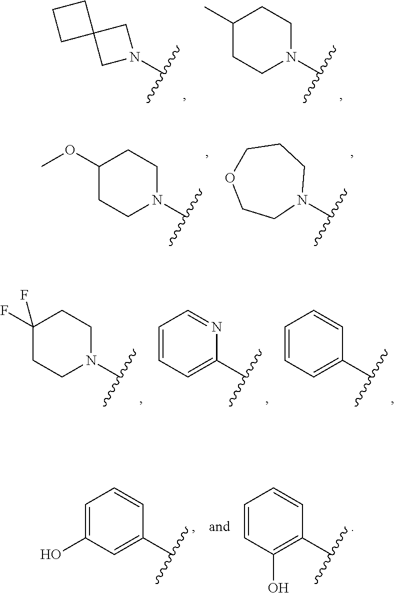

In some embodiments, R₃ is selected from

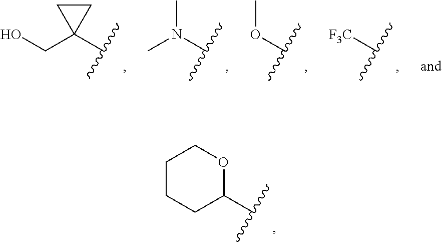

optionally

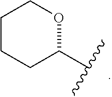 [SMILES: *[C@@H]1CCCCO1]

In some embodiments, q is 0 or 1. In some embodiments, r is 2 or 3. In some embodiments, s is 1. In some embodiments, R₁₂ is selected from methyl, ethyl, —OCF₃, and

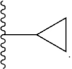 [SMILES: *C1CC1]

In some embodiments, R₈ is selected from H, methyl, ethyl, isopropyl, n-butyl, t-butyl,

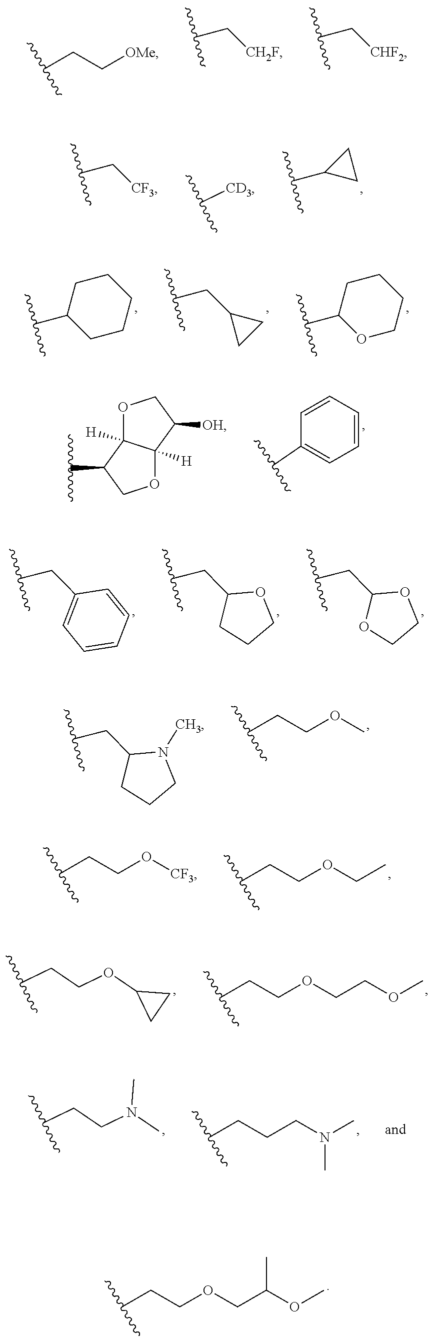

In some embodiments, R₈ is selected from H, methyl, ethyl, n-butyl,

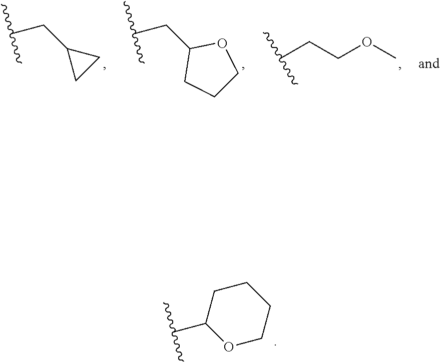

In some embodiments, R₈ is selected from H, methyl, and ethyl.

In some embodiments, the compound of formula (I) is selected from a compound having a formula selected from formula 1001-1114, or a pharmaceutically acceptable salt, solvate, hydrate, cocrystal, or prodrug thereof.

In some embodiments, the compound of formula (I) is selected from a compound having a formula selected from formula 1003, 1007, 1024, 1044, 1077, 1078, 1081, 1084, 1088, 1112, 1113, or a pharmaceutically acceptable salt, solvate, hydrate, cocrystal, or prodrug thereof.

In some embodiments, the compound of formula (I) is selected from a compound having a formula selected from formula 1001, 1006, 1008, 1010, 1013, 1014, 1016, 1017, 1018, 1022, or a pharmaceutically acceptable salt, solvate, hydrate, cocrystal, or prodrug thereof.

In some embodiments, the compound of formula (I) is selected from a compound having a formula selected from formula 1003, 1009, 1011, 1015, or a pharmaceutically acceptable salt, solvate, hydrate, cocrystal, or prodrug thereof.

In some embodiments, A is a 5-membered ring heterocycle and R₁ is

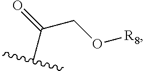

### R₈ is not H.

In some embodiments, the compound of formula (I) is selected from a compound having a formula selected from formula 1001-1114, or a pharmaceutically acceptable salt, solvate, hydrate, cocrystal, or prodrug thereof. In some embodiments, the compound of formula (I) is selected from a compound having a formula selected from formula 1003, 1007, 1009, 1011, 1015, 1019, 1023, 1024, 1025, 1026, and 1078, or a pharmaceutically acceptable salt, solvate, hydrate, cocrystal, or prodrug thereof. In some embodiments, the compound of formula (I) is selected from a compound having a formula selected from formula 1001, 1006, 1008, 1010, 1013, 1014, 1016, 1024, 1017, 1018, and 1022, or a pharmaceutically acceptable salt, solvate, hydrate, cocrystal, or prodrug thereof. In some embodiments, the compound of formula (I) is selected from a compound having a formula selected from formula 1003, 1009, 1011, and 1015, or a pharmaceutically acceptable salt, solvate, hydrate, cocrystal, or prodrug thereof.

In some embodiments, in formula (I), when A is a 5-membered ring heterocycle and R₁ is

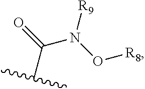

### R₈ is not H.

In another aspect, the disclosure provides a pharmaceutical formulation including a compound having a formula of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114.

In another aspect, the disclosure provides a method of treating a patient with a mitochondrial dysfunction. In some embodiments, the method includes identifying a mitochondrial dysfunction in an individual; and administering a compound having a formula of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114 to the patient.

In another aspect, the disclosure provides a method of treating a patient with a mitochondrial dysfunction. In some embodiment, the method includes administering a therapeutically effective amount of a compound having a formula of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114 to the patient.

In some embodiments, the mitochondrial dysfunction is a primary mitochondrial dysfunction. In some embodiments, the primary mitochondrial dysfunction is selected from the group consisting of Autosomal Dominant Optic Atrophy (ADOA), Alpers-Huttenlocher syndrome (nDNA defect), Ataxia neuropathy syndrome, (nDNA defect), Barth syndrome/Lethal Infantile Cardiomyopathy (LIC), Co-enzyme Q deficiency, Complex I, complex II, complex III, complex IV and complex V deficiencies (either single deficiencies or any combination of deficiency), Chronic progressive external ophthalmoplegia (CPEO), Diabetes mellitus and deafness, Kearns-Sayre syndrome (mtDNA defect), Leukoencephalopathy with Brainstem and Spinal Cord Involvement and Lactate Elevation (LBSL-leukodystrophy), Leigh syndrome (mtDNA and nDNA defects), Leber's hereditary optic neuropathy (LHON), Luft Disease, Mitochondrial myopathy, encephalopathy, lactic acidosis, and stroke syndrome (MELAS) (mtDNA defect), Mitochondrial Enoyl CoA Reductase Protein-Associated Neurodegeneration (MEPAN), Myoclonic epilepsy with ragged red fibers (MERRF), mitochondrial recessive ataxia syndrome (MIRAS), mtDNA deletion syndrome, mtDNA Depletion syndrome, mtDNA maintenance disorders, mtDNA/RNA translation defects, Mitochondrial tRNA synthetase deficiencies, Mitochondrial Myopathy, Mitochondrial neurogastrointestinal encephalopathy syndrome (MNGIE), Neurogenic muscle weakness, ataxia, and retinitis pigmentosa (NARP), Pearson syndrome, Pyruvate dehydrogenase complex deficiency (PDCD/PDH), DNA polymerase gamma deficiency (POLG), Pyruvate carboxylase deficiency, and Thymidine kinase 2 deficiency (TK2).

In some embodiments, the mitochondrial dysfunction is a secondary mitochondrial dysfunction. In some embodiments, the secondary mitochondrial dysfunction is selected from the group consisting of age-related macular degeneration (AMD), Amyotrophic Lateral Sclerosis (ALS), Alzheimer's disease (AD) and other dementias, Friedreich's ataxia (FA), Huntington's disease (HD), Motor neuron diseases (MND), N-glycanase deficiency (NGLY1), Organic acidemias, Parkinson's disease (PD) and PD-related disorders, Prion disease, Spinal muscular atrophy (SMA), Spinocerebellar ataxia (SCA), Becker muscular dystrophy, Congenital muscular dystrophies, Duchenne muscular dystrophy, Emery-Dreifuss muscular dystrophy, Facioscapulohumeral muscular dystrophy, Myotonic dystrophy, Oculopharyngeal muscular dystrophy, Charcot-Marie-Tooth disease, Congenital myopathies, Distal myopathies, Endocrine myopathies (hyperthyroid myopathy, hypothyroid myopathy), Giant axonal neuropathy, Hereditary spastic paraplegia, Inflammatory myopathies (dermatomyositis, inclusion-body myositis, polymyositis), Metabolic myopathies, Neuromuscular junction diseases, Autism, Cancer, Diabetes, Metabolic syndrome, Chronic fatigue syndrome, an inflammatory disorder, arthritis, aging, and mitochondrial epilepsy (epilepsy secondary to primary mitochondrial disease).

In some embodiments, the compound having a formula of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114 is administered in a pharmaceutical formulation. In some embodiments, the pharmaceutical formulation comprises the compound and at least one selected from a binding agent, a lubricating agent, a buffer, and a coating. In some embodiments, the compound is administered orally. In some embodiments, the compound is administered daily for at least one week.

In some embodiments, the method further includes assessing the efficacy of the compound in the individual.

In some embodiments, the compound having a formula of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114 is administered by oral administration, subcutaneous administration, intravenous administration, intraperitoneal administration, intranasal administration, dermal administration, intravitreal injection, or inhalation.

### BRIEF DESCRIPTION OF THE FIGURES

FIG. 1 illustrates the structures of non-limiting examples of compounds of the disclosure.

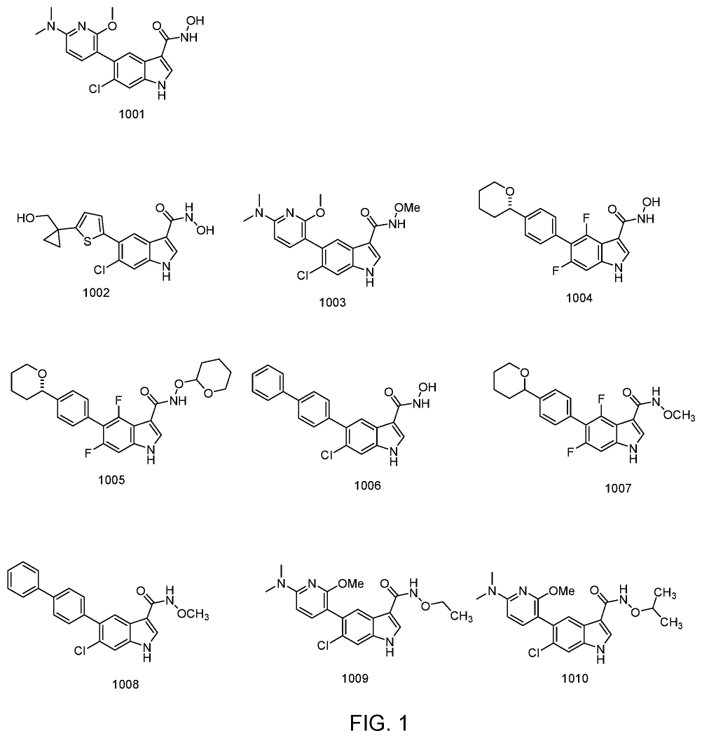

FIG. 2 illustrates the structures of non-limiting examples of compounds of the disclosure.

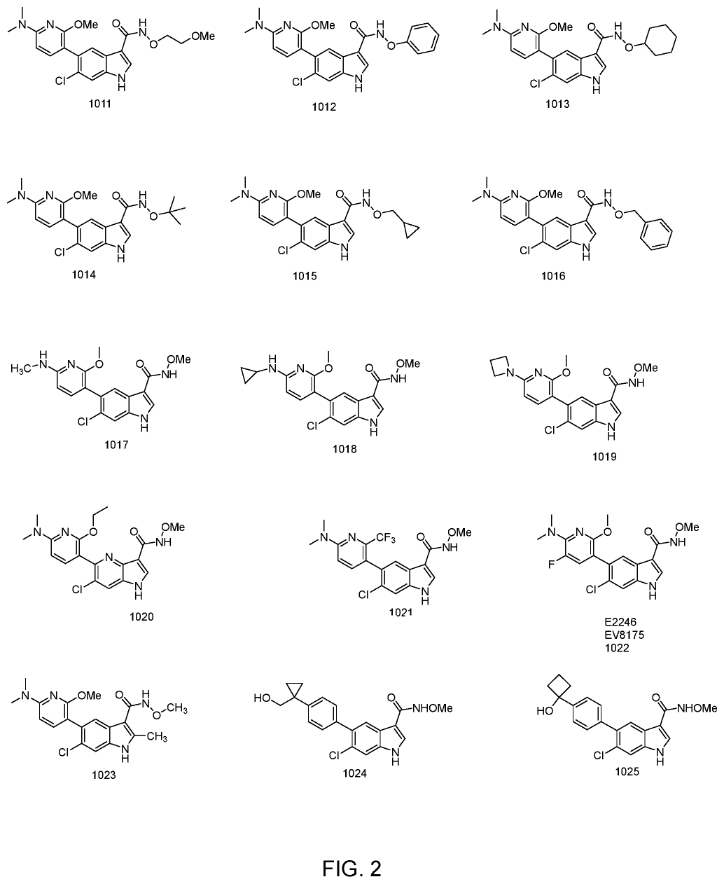

FIG. 3 illustrates the structures of non-limiting examples of compounds of the disclosure.

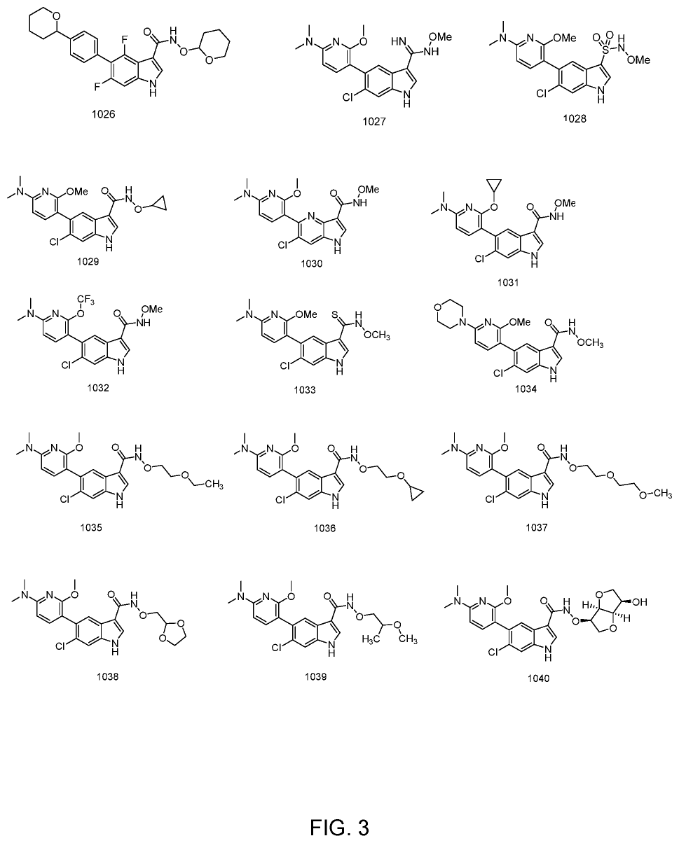

FIG. 4 illustrates the structures of non-limiting examples of compounds of the disclosure.

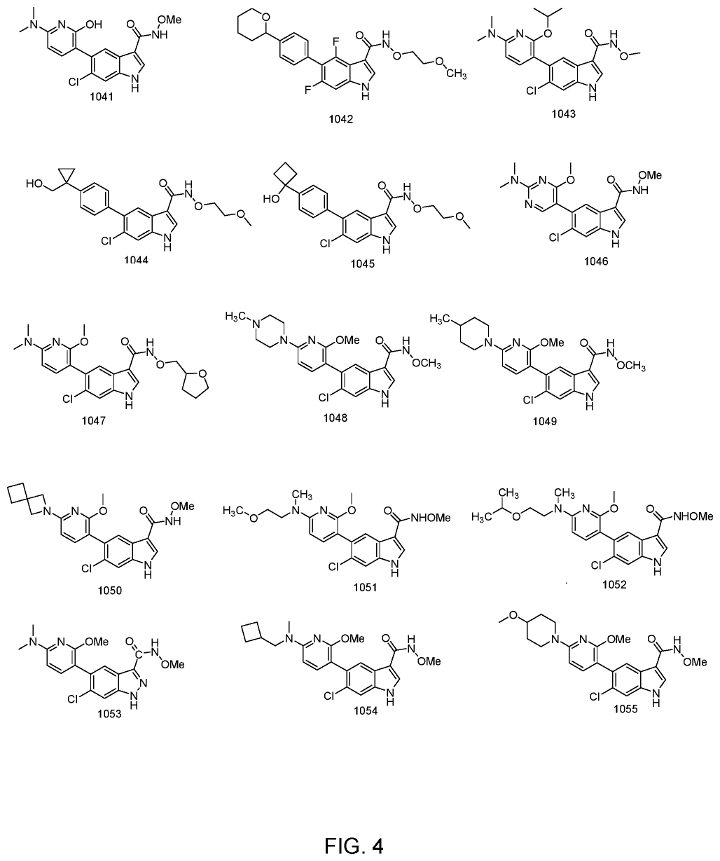

FIG. 5 illustrates the structures of non-limiting examples of compounds of the disclosure.

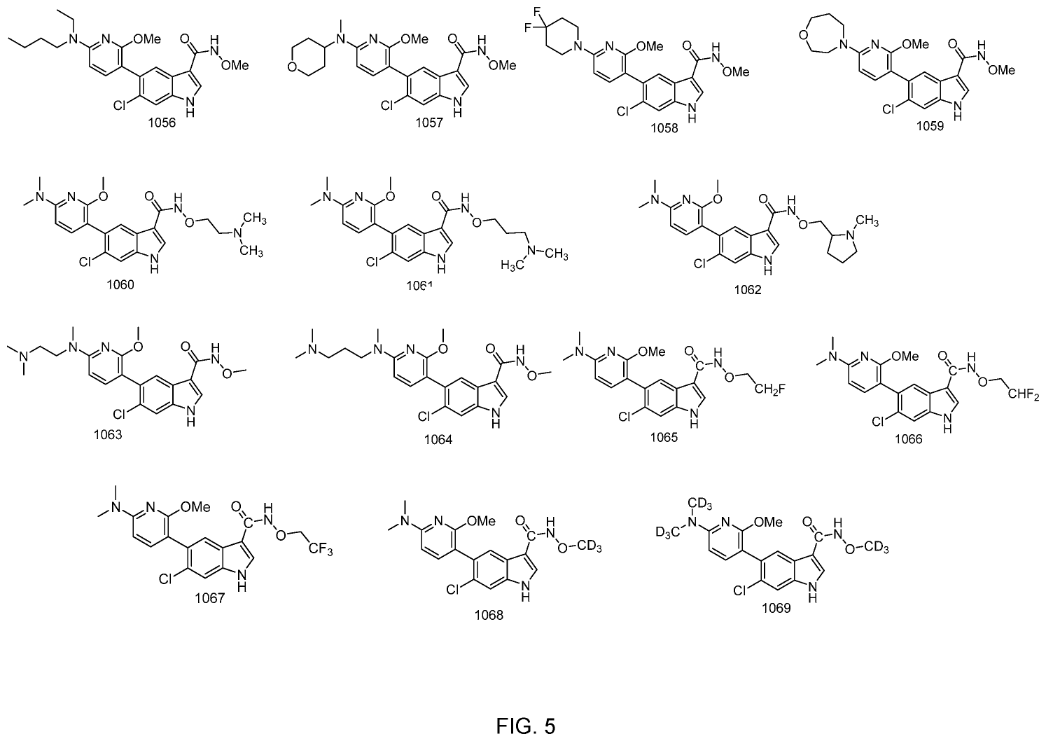

FIG. 6 illustrates the structures of non-limiting examples of compounds of the disclosure.

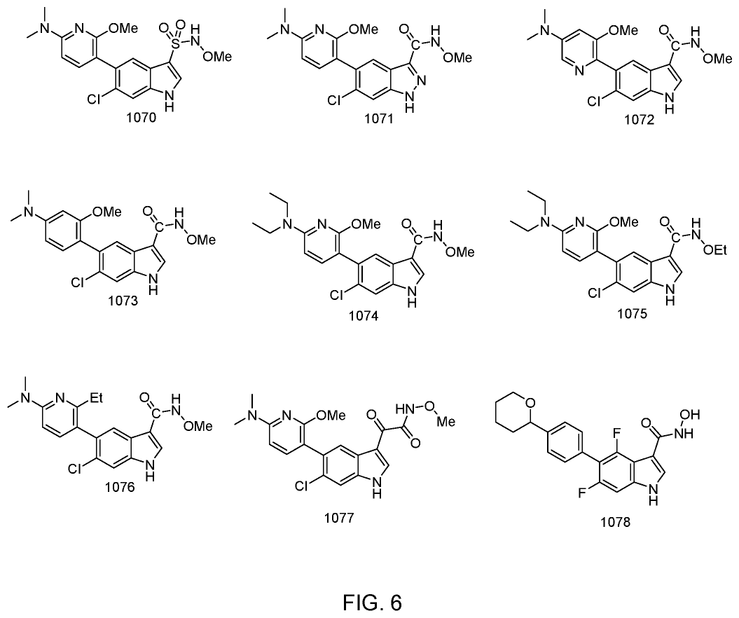

FIG. 7 illustrates the structures of non-limiting examples of compounds of the disclosure.

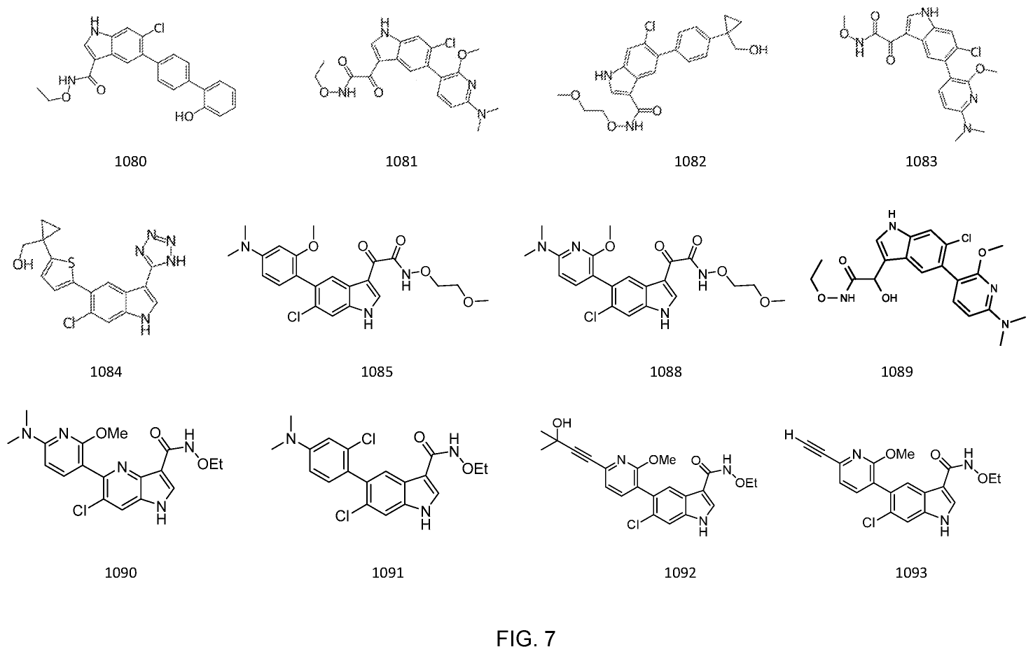

FIG. 8 illustrates the structures of non-limiting examples of compounds of the disclosure.

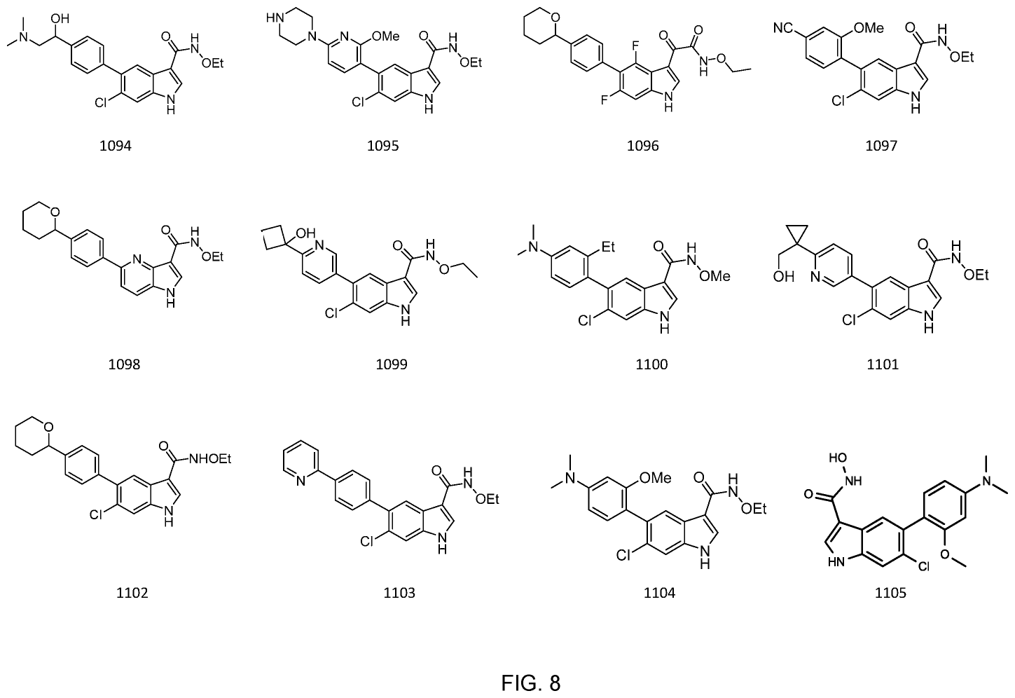

FIG. 9 illustrates the structures of non-limiting examples of compounds of the disclosure.

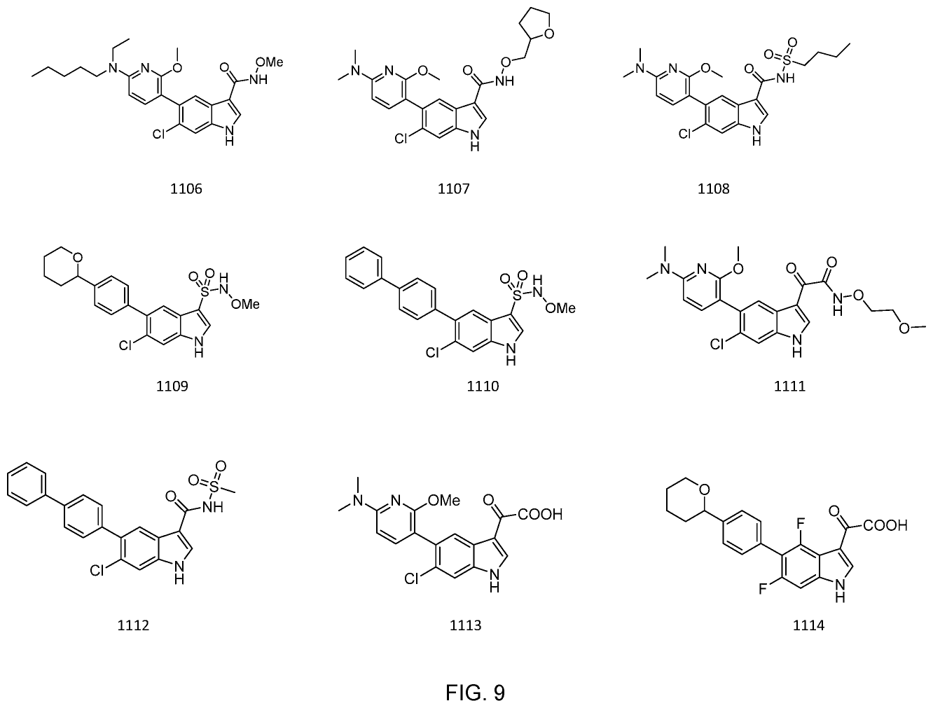

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include, but are not limited to, the compounds described herein and, more specifically, compounds of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, and their features and limitations as described herein.

As used herein, the terms “patient,” “subject,” and “individual” are used interchangeably.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., increased sensitivity to apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs disclosed herein, can also be incorporated into the described compositions and methods.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule. In some embodiments, the biological molecules modulated by the methods and compounds of the disclosure to effect treatment may include the Mcl-1 oncoprotein and Bcl-2 oncoprotein.

As used herein, the term “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of a compound of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone.

Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114) is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(Rₐ)₂ (where t is 1 or 2), or PO₃(Rₐ)₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” or “alkylheterocyclyl” refers to an -(alkyl) heterocycloalkyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(Rₐ)₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(═O)H radical.

“Carboxyl” refers to a —(═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 10 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “cycloalkyloxy” represents a cycloalkyl group having the indicated number of carbon atoms attached through an oxygen atom (e.g., cyclopropyloxy and cyclohexyloxy).

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acylsulfonamide” refers a —S(O)₂—N(R^{a})—C(═O)— radical, where R^{a} is hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Unless stated otherwise specifically in the specification, an acylsulfonamide group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl

“Alkylcycloalkyl” refers to an optionally substituted ring system comprising a cycloalkyl group having one or more alkyl substituents, wherein cycloalkyl and alkyl are each as previously defined. Exemplary alkylcycloalkyl groups include, for example, 2-methylcyclohexyl, 3,3-dimethylcyclopentyl, trans-2,3-dimethylcyclooctyl, and 4-methyldecahydronaphthalenyl.

“Alkylheterocycloalkenyl” refers to a heterocycloalkyl or heterocyclyl as defined herein and further including 1 or 2 double bonds and having one or more alkyl substituents.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{a}, and NR^{a}R^{a} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “aryloxy” refers to the group —O-aryl.

The term “substituted aryloxy” refers to aryloxy wherein the aryl substituent is substituted (i.e., —O-(substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Cycloalkylalkyl” refers to an alkyl group in which one of the hydrogen atoms is replaced by a cycloalkyl group. In some embodiments, the hydrogen atom on the terminal carbon atom of the alkyl group is substituted with a cycloalkyl group. In some embodiments, the cycloalkyl group is a C₃₋₆ cycloalkyl group, in some embodiments a C₅₋₆ cycloalkyl group, and in some embodiments, a cyclopropyl, a cyclobutyl, a cyclopentyl, or a cyclohexyl group. In some embodiments, the alkanediyl portion of a cycloalkylalkyl group may be, for example, C₁₋₁₀ alkanediyl, C₁₋₆ alkanediyl, C₁₋₄ alkanediyl, C₁₋₃ alkanediyl, propane-1,3-diyl, ethane-1,2-diyl, or methane-diyl. In some embodiments, the cycloalkylalkyl group is C₄₋₁₆ cycloalkylalkyl, C₄₋₁₂ cycloalkylalkyl, C₄₋₁₀ cycloalkylalkyl, C₆₋₁₂ cycloalkylalkyl, or C₆₋₉ cycloalkylalkyl. For example, C₆₋₉ cycloalkylalkyl includes a C₃ alkyl group bonded to a cyclopentyl or a cyclohexyl group.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” or “Het” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” or “heterocyclyl” refer to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Heterocycloalkenyl” refers to a heterocycloalkyl or heterocyclyl as defined above and further including 1 or 2 double bonds. Non-limiting examples of heterocycloalkenyls include (C₄-C₉)heterocycloalkenyl.

“Heterocycloalkenylalkyl” refers to an alkyl group in which one of the hydrogen atoms is replaced by a heterocycloalkenyl group. In some embodiments, the hydrogen atom on the terminal carbon atom of the alkyl group is substituted with a heterocycloalkenyl group. Non-limiting examples include a dihydrofurylmethyl group (e.g., 2,5-dihydrofuran-3-ylmethyl group), a dihydropyranylmethyl group (e.g., a 5,6-dihydro-2H-pyran-ylmethyl group), a dihydropyrrolylmethyl group (a 3-pyrrolin-3-ylmethyl group), a tetrahydropyridylmethyl group (e.g., a 1,2,3,6-tetrahydropyridin-4-ylmethyl group), a tetrahydropyridylethyl group (e.g., a 1,2,3,6-tetrahydropyridin-4-yl-2-ethyl group), a dihydrothienylmethyl group (e.g., a 2,5-dihydrothiophen-3-ylmethyl group), a dihydrothiopyranylmethyl group (e.g., a 5,6-dihydro-2H-thiopyran-4-ylmethyl group), a dehydrohomopiperidinylmethyl group (e.g., a 4,5-dehydrohomopiperidin-4-ylmethyl group) and the like.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)— (optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the disclosure. All embodiments of the disclosure can, in the alternative, be more specifically defined by any of the transitional terms “comprising,” “consisting essentially of,” and “consisting of.”

### AMPK Modulators

In one aspect, the disclosure provides novel compounds that modulate AMPK. In some embodiments, the compounds of the disclosure are AMPK agonists. In some embodiments, the compounds of the disclosure are AMPK inhibitors.

In one aspect, the disclosure provides a compound of formula (I), or a pharmaceutically acceptable salt, solvate, hydrate, cocrystal, or prodrug thereof:

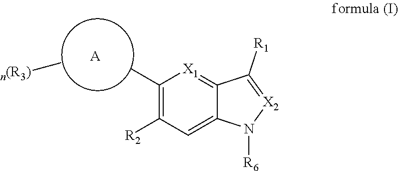

wherein in formula (I):
X₁ is CR₄ₐ or N;
X₂ is CR_{4b} or N;
R₁ is selected from

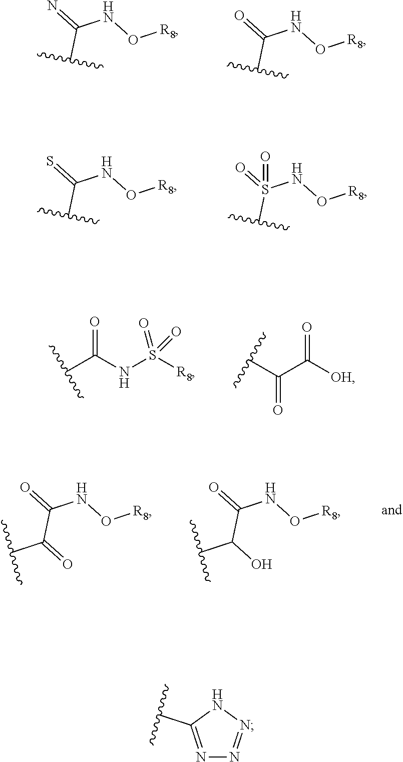

R₂ is H, —CF₃ or halo;
R₃ is at each occurrence independently selected from halo, —CN,

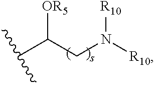

—N(R₁₀)₂, C₁₋₁₀ alkyl, C₂₋₁₀ alkynyl, C₁₋₁₀ alkoxy, C₃₋₇ cycloalkyl, C₃₋₇ cycloalkyloxy, C₃₋₁₀ heterocyclyl, C₄₋₁₂ alkylcycloalkyl, C₄₋₁₀ cycloalkylalkyl, C₃₋₇ heterocycloalkenyl, C₄₋₁₂ alkylheterocycloalkenyl, C₄₋₁₀ heterocycloalkenylalkyl, aryl and heteroaryl, wherein the C₁₋₁₀ alkyl, C₂₋₁₀ alkynyl, C₁₋₁₀ alkoxy, C₃₋₇ cycloalkyl, C₃₋₁₀ heterocyclyl, C₄₋₁₂ alkylcycloalkyl, C₄₋₁₀ cycloalkylalkyl, C₃₋₇ heterocycloalkenyl, C₄₋₁₂ alkylheterocycloalkenyl, C₄₋₁₀ heterocycloalkenylalkyl, aryl, or heteroaryl is unsubstituted or substituted with one to three substituents selected from deuterium, halo, C₁₋₆ alkyl, C₃₋₆ cycloalkyl, —OR₅ and —OCOR₇;
R₄ₐ is at each occurrence independently selected from H, halo, C₁₋₁₀ alkyl, C₃₋₇ cycloalkyl, C₃₋₁₀ heterocyclyl, C₄₋₁₂ alkylcycloalkyl, and C₄₋₁₀ cycloalkylalkyl, wherein the C₁₋₁₀ alkyl, C₃₋₇ cycloalkyl, C₃₋₁₀ heterocyclyl, C₄₋₁₂ alkylcycloalkyl, or C₄₋₁₀ cycloalkylalkyl is unsubstituted or substituted with one to three halogen substituents;
R_{4b} is at each occurrence independently selected from H, C₁₋₁₀ alkyl, C₃₋₇ cycloalkyl, C₃₋₁₀ heterocyclyl, C₄₋₁₂ alkylcycloalkyl, and C₄₋₁₀ cycloalkylalkyl, wherein the C₁₋₁₀ alkyl, C₃₋₇ cycloalkyl, C₃₋₁₀ heterocyclyl, C₄₋₁₂ alkylcycloalkyl, or C₄₋₁₀ cycloalkylalkyl is unsubstituted or substituted with one to three halogen substituents;
R₅ is at each occurrence independently selected from H, C₁₋₁₀ alkyl, C₁₋₁₀ alkoxy, C₃₋₇ cycloalkyl, C₄₋₁₂ alkylcycloalkyl, and C₄₋₁₀ cycloalkylalkyl, wherein the C₁₋₁₀ alkyl, C₁₋₁₀ alkoxy, C₃₋₇ cycloalkyl, C₄₋₁₂ alkylcycloalkyl, or C₄₋₁₀ cycloalkylalkyl is unsubstituted or substituted with one to three halogen substituents;
R₆ is at each occurrence independently selected from H, C₁₋₆ alkyl, and C₃₋₆ cycloalkyl;
R₇ is at each occurrence independently selected from C₁₋₁₀ alkyl, C₃₋₇ cycloalkyl, and C₄₋₁₂ alkylcycloalkyl, wherein the C₁₋₁₀ alkyl, C₃₋₇ cycloalkyl, or C₄₋₁₂ alkylcycloalkyl is unsubstituted or substituted with one to three substituents selected from halo, C₁₋₁₀ alkyl, and —NR₁₂,R₁₂; R₈ is selected from H, C₁₋₁₀ alkyl, C₁₋₁₀ alkoxy, C₃₋₇ cycloalkyl, C₄₋₁₂ alkylcycloalkyl, C₄₋₁₀ cycloalkylalkyl, C₃₋₁₀ heterocyclyl, C₄₋₁₂ alkylheterocyclyl, C₃₋₇ heterocycloalkenyl, C₄₋₁₂ alkylheterocycloalkenyl, C₄₋₁₀ heterocycloalkenylalkyl, aryl, heteroaryl,

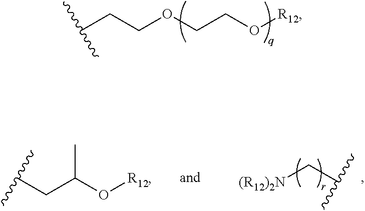

wherein the C₁₋₁₀ alkyl, C₁₋₁₀ alkoxy, C₃₋₇ cycloalkyl, C₄₋₁₂ alkylcycloalkyl, C₄₋₁₀ cycloalkylalkyl, C₃₋₁₀ heterocyclyl, C₄₋₁₂ alkylheterocyclyl, C₃₋₇ heterocycloalkenyl, C₄₋₁₂ alkylheterocycloalkenyl, C₄₋₁₀ heterocycloalkenylalkyl, aryl, or heteroaryl is unsubstituted or substituted with one to three substituents selected from deuterium, halo and —OR₅;
R₁₀ is at each occurrence independently selected from H, C₁₋₆ alkyl, C₃₋₆ cycloalkyl, C₄₋₁₂ alkylcycloalkyl,

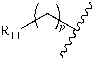

and C₃₋₁₀ heterocyclyl, wherein the C₁₋₆ alkyl, C₃₋₆ cycloalkyl, C₄₋₁₂ alkylcycloalkyl, or C₃₋₁₀ heterocyclyl is unsubstituted or substituted with one to three substituents selected from deuterium, halo, C₁₋₆ alkyl, and C₃₋₆ cycloalkyl substituents;
R₁₁ is at each occurrence independently selected from —OR₁₂ and —N(R₁₂)₂;
R₁₂ is at each occurrence independently selected from H, C₁₋₆ alkyl, C₁₋₁₀ haloalkoxy, and C₃₋₆ cycloalkyl;
n is an integer from 0-3;
p is an integer from 1-5;
q is an integer from 0-5;
r is an integer from 1-5;
s is an integer from 1-5;

A is selected from phenyl, pyridyl, pyrimidyl, pyridazyl and the following 5-membered ring heterocycles:

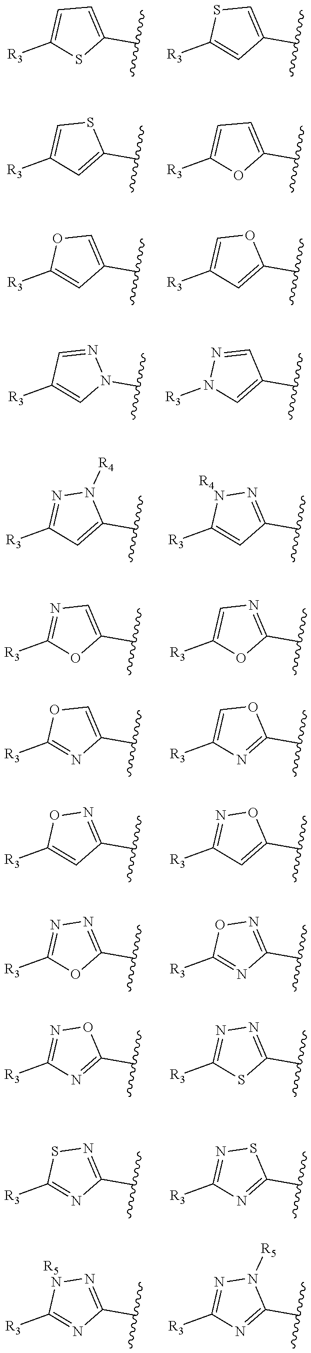

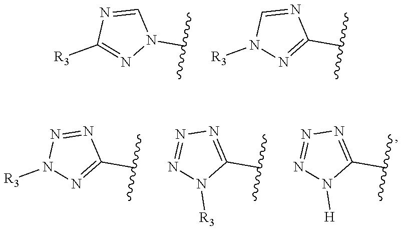

provided that the compound of formula (I) is not

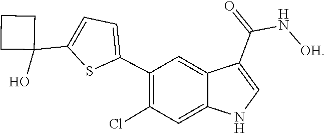 [SMILES: O=C(NO)c1c[nH]c2cc(Cl)c(-c3ccc(C4(O)CCC4)s3)cc12]

In some embodiments, X₁ is CR₄ₐ. In some embodiments, R₄ₐ is H, Cl, or F.

In some embodiments, X₂ is CR_{4b}. In some embodiments, X₂ is N or CR_{4b}, wherein R_{4b} is H. In some embodiments, R_{4b} is C₁₋₁₀ alkyl. In one embodiment, R_{4b} is CH₃.

In one embodiment, A is selected from phenyl, pyridyl, pyrimidyl, pyridazyl and the following 5-membered ring heterocycles:

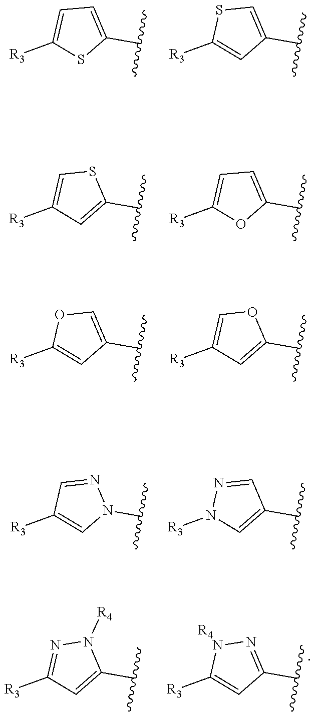

In some embodiments, A and/or the 5-membered ring heterocycle is selected from

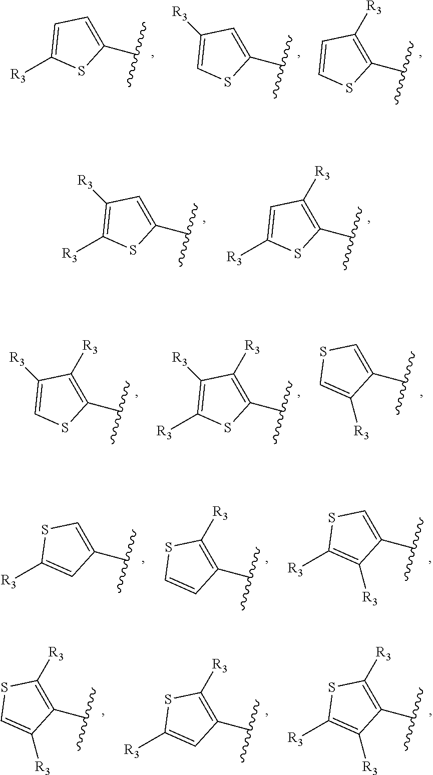

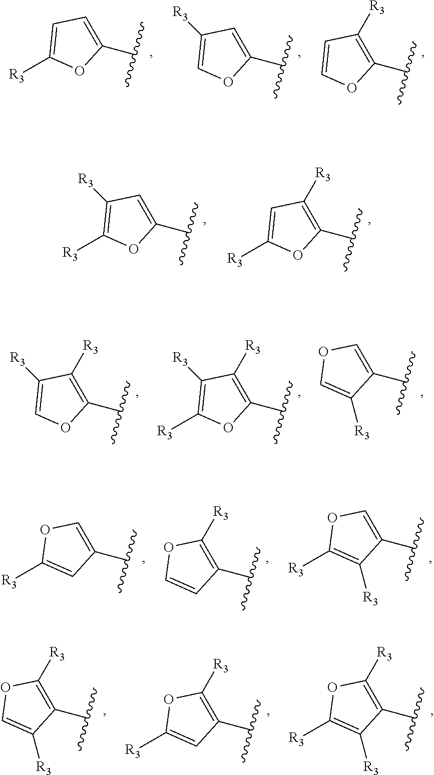

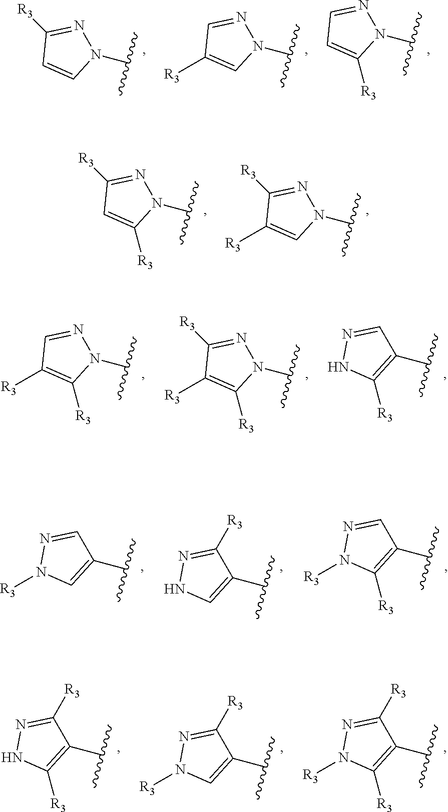

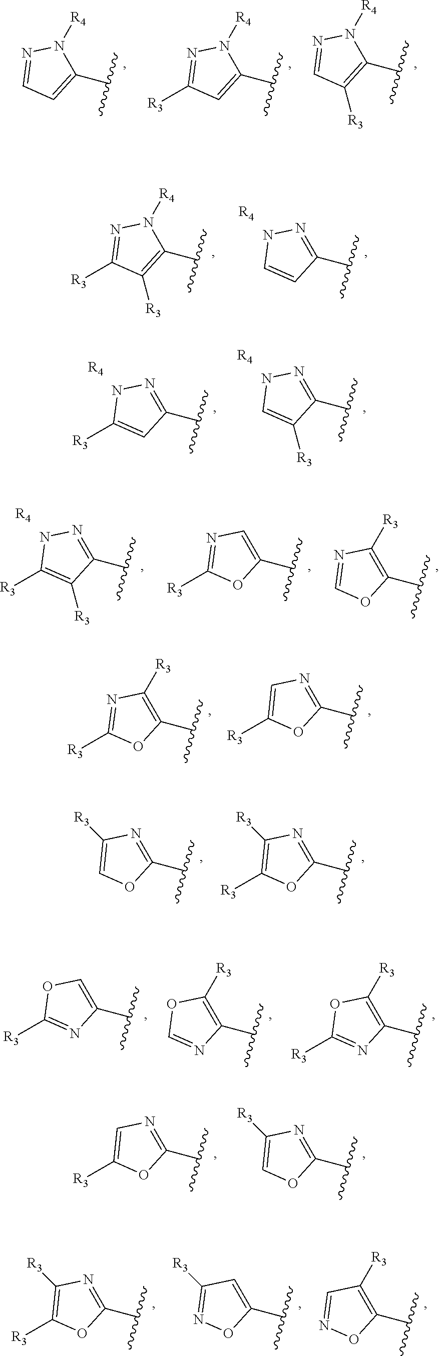

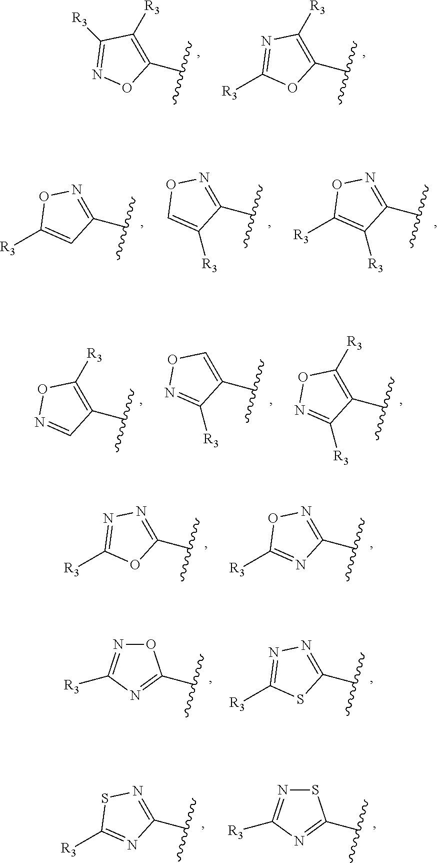

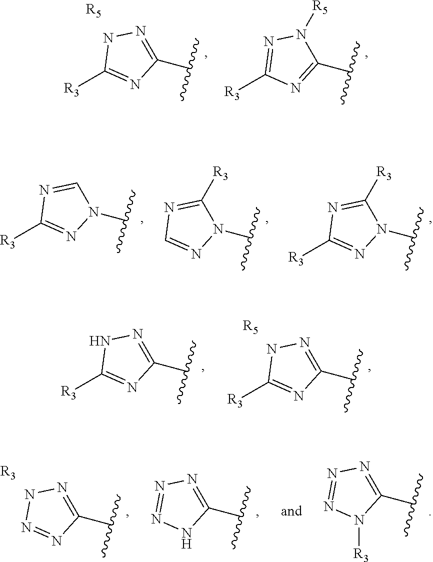

In one embodiment, A is selected from

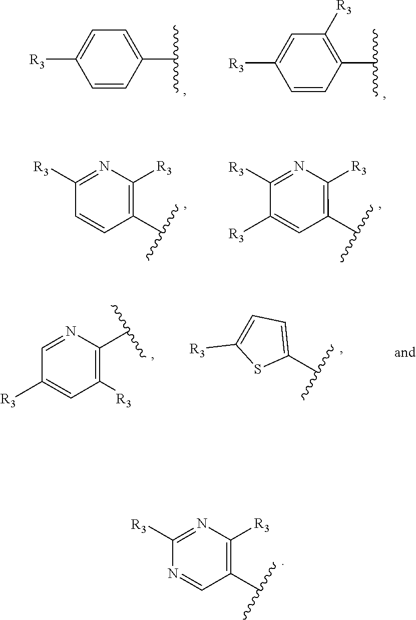

In one embodiment, R₂ is halo. In one embodiment, R₂ is Cl or F.

In one embodiment, R₆ is H.

In one aspect, the disclosure provides a compound of formula (10), formula (11), formula (12), formula (13), formula (14), formula (15), formula (16), or formula (17) or a pharmaceutically acceptable salt, solvate, hydrate, cocrystal, or prodrug thereof:

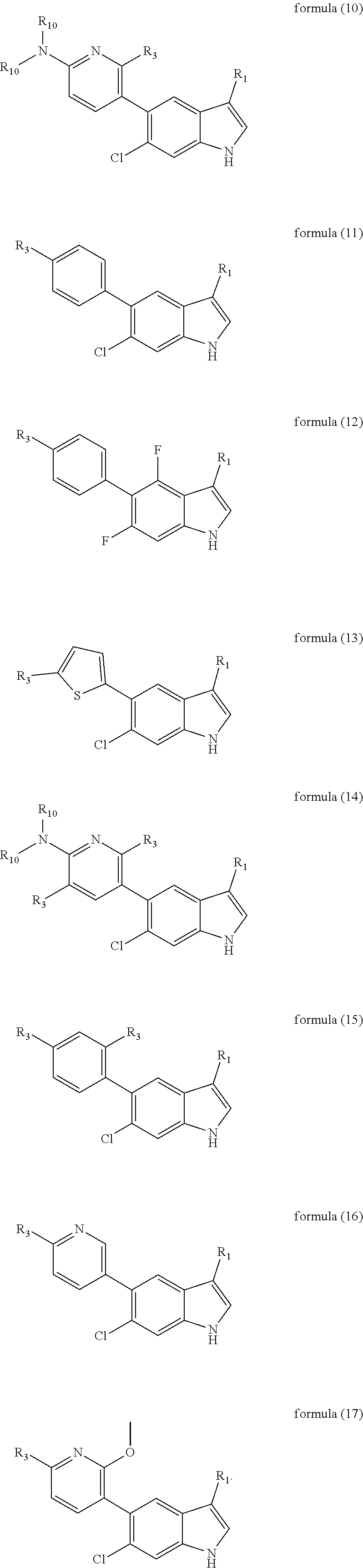

In some embodiments, R₁ is

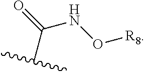

In one aspect, the disclosure provides a compound of formula (100), formula (110), formula (120), formula (130), formula (140), formula (150), formula (160), or formula (170) or a pharmaceutically acceptable salt, solvate, hydrate, cocrystal, or prodrug thereof:

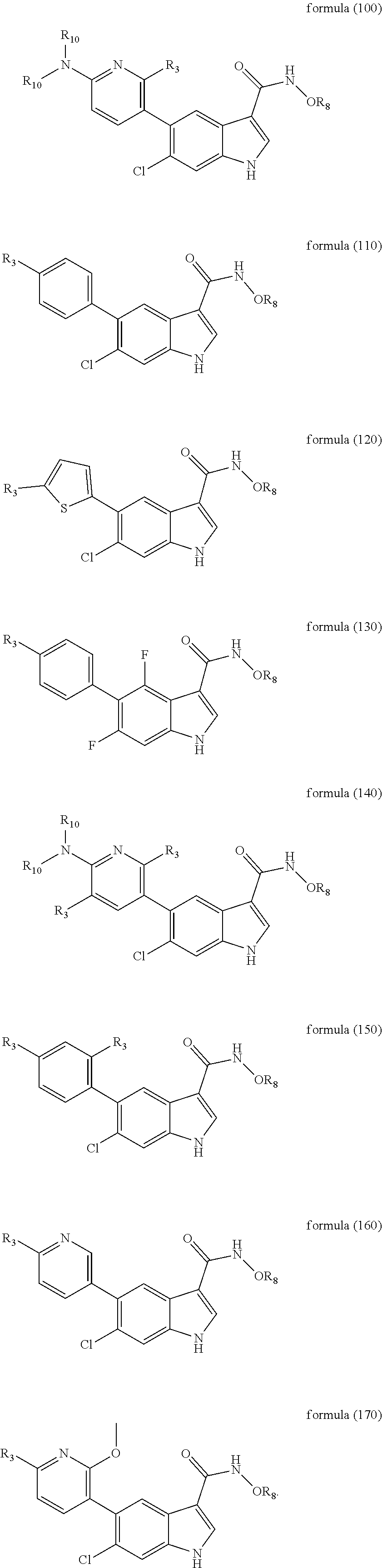

In some embodiments, R₁ is

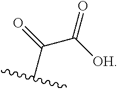 [SMILES: *C(=O)C(=O)O]

In one embodiment, the compound of formula (I) is a compound of formula (200), formula (210), formula (220), formula (230), formula (240), formula (250), formula (260), or formula (270) or a pharmaceutically acceptable salt, solvate, hydrate, cocrystal, or prodrug thereof:

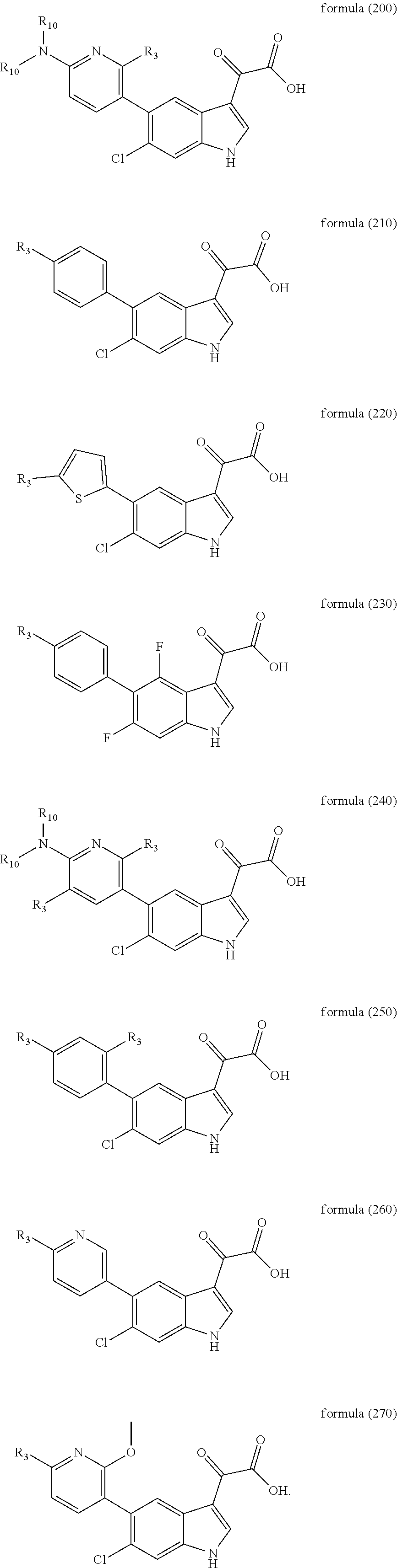

In one embodiment, R₁ is

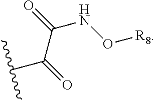

In one embodiment, the compound of formula (I) is a compound of formula (300), formula (310), formula (320), formula (330), formula (340), formula (350), formula (360), or formula (370) or a pharmaceutically acceptable salt, solvate, hydrate, cocrystal, or prodrug thereof:

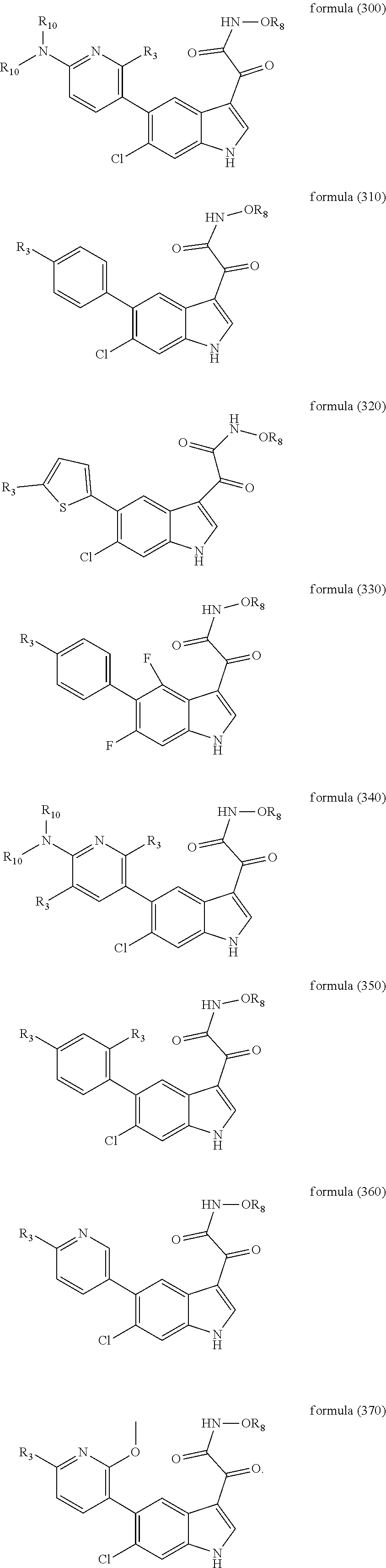

In one embodiment, the compound of any one of formulas (300)-(370) is selected from:

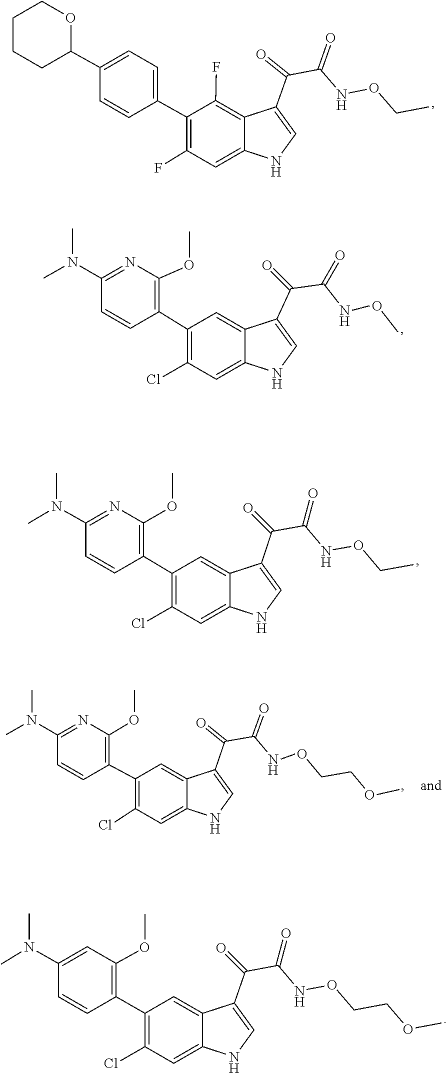

In one embodiment, R₁ is

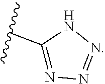 [SMILES: *c1nnn[nH]1]

In one embodiment, the compound of formula (I) is a compound of formula (400), formula (410), formula (420), formula (430), formula (440), formula (450), formula (460), or formula (470) or a pharmaceutically acceptable salt, solvate, hydrate, cocrystal, or prodrug thereof:

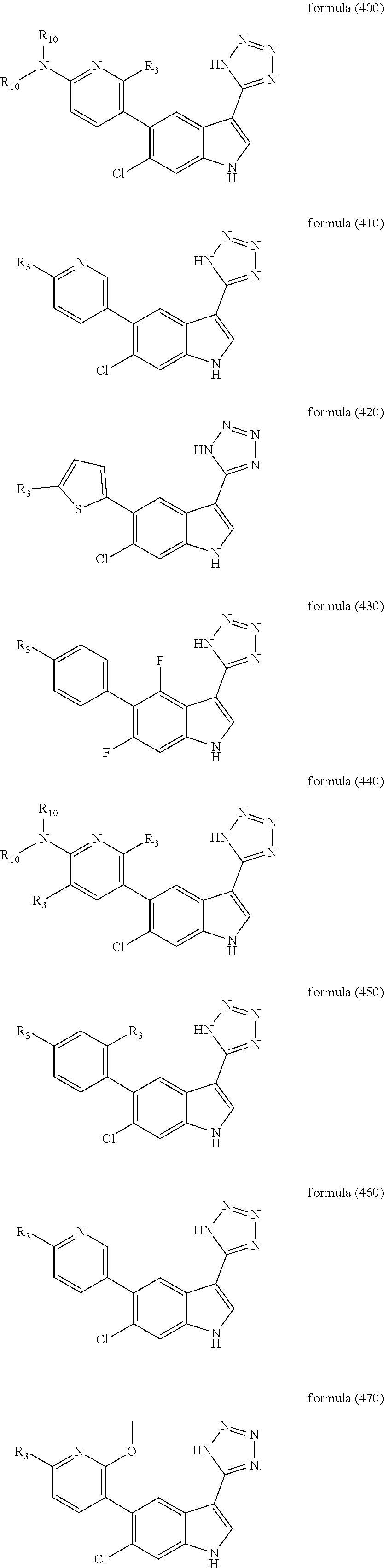

In one embodiment, R₁ is

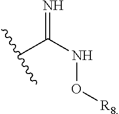

In one embodiment, the compound of formula (I) is a compound of formula (500), formula (510), formula (520), formula (530), formula (540), formula (550), formula (560), or formula (570) or a pharmaceutically acceptable salt, solvate, hydrate, cocrystal, or prodrug thereof:

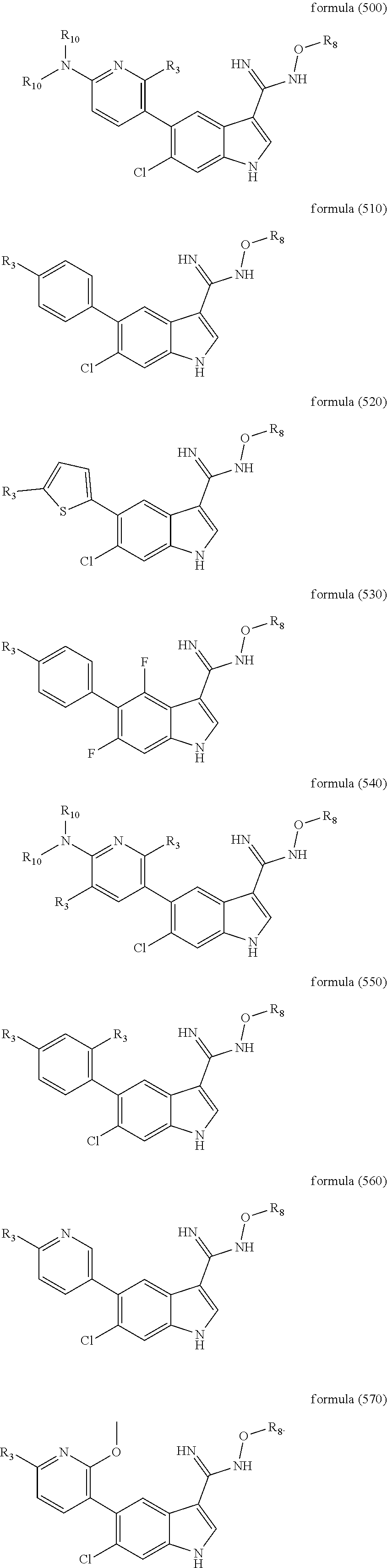

In one embodiment, R₁ is

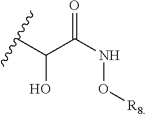

In one embodiment, the compound of formula (I) is a compound of formula (600), formula (610), formula (620), formula (630), formula (640), formula (650), formula (660), or formula (670) or a pharmaceutically acceptable salt, solvate, hydrate, cocrystal, or prodrug thereof:

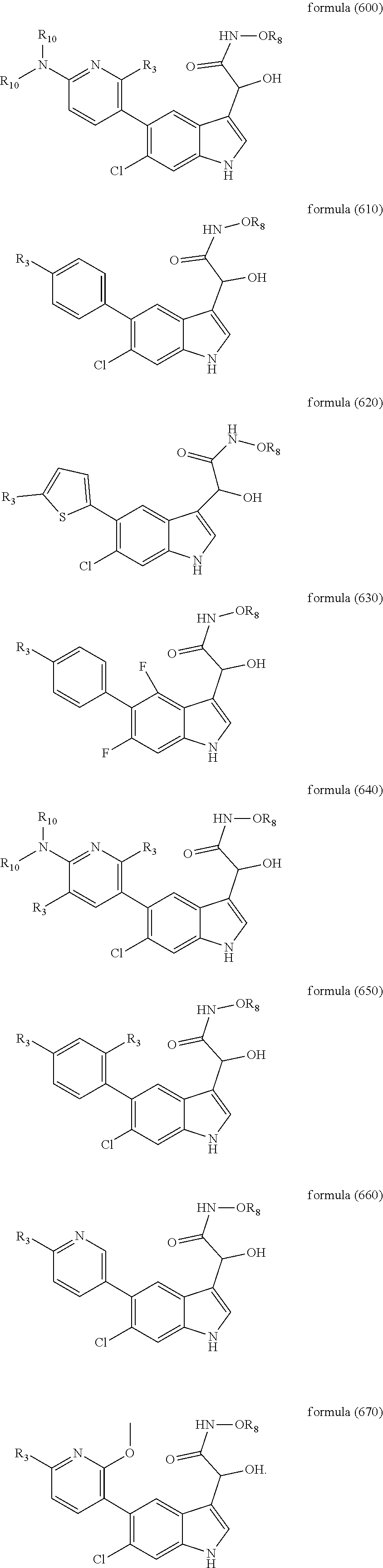

In one embodiment, R₁ is

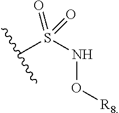

In one embodiment, the compound of formula (I) is a compound of formula (700), formula (710), formula (720), formula (730), formula (740), formula (750), formula (760), or formula (770) or a pharmaceutically acceptable salt, solvate, hydrate, cocrystal, or prodrug thereof:

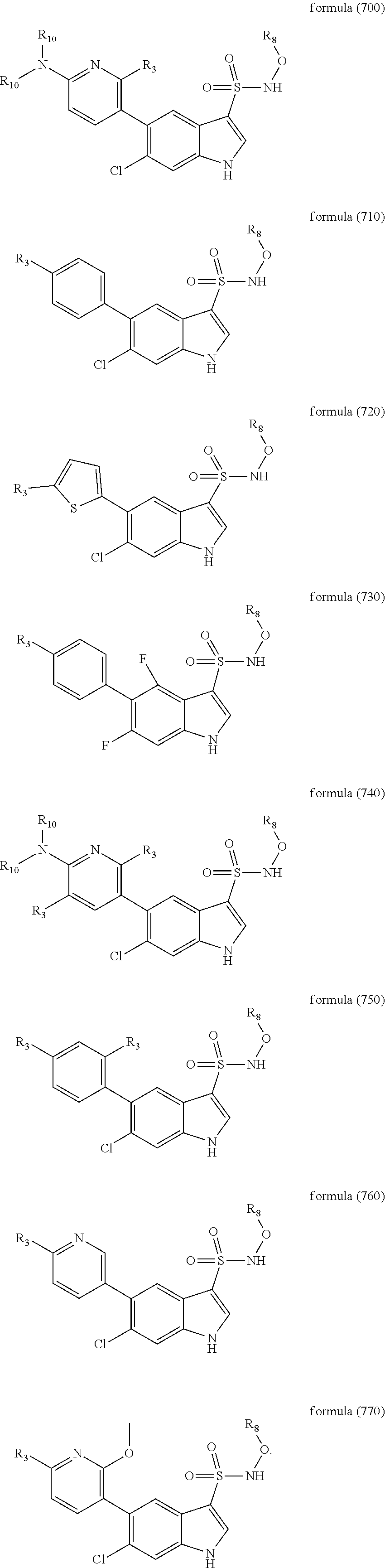

In one embodiment, R₁ is

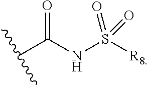

In one embodiment, the compound of formula (I) is a compound of formula (800), formula (810), formula (820), formula (830), formula (840), formula (850), formula (860), or formula (870) or a pharmaceutically acceptable salt, solvate, hydrate, cocrystal, or prodrug thereof:

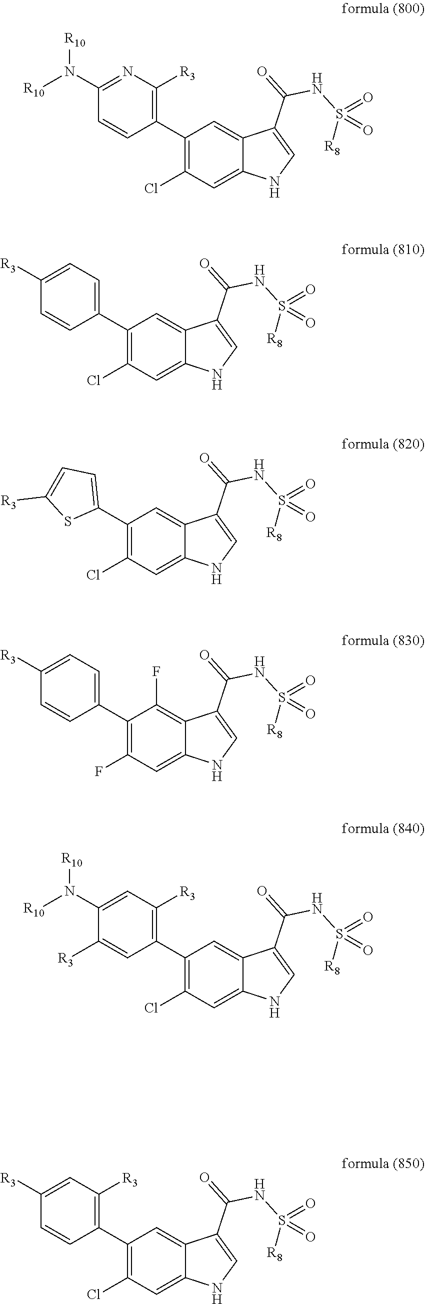

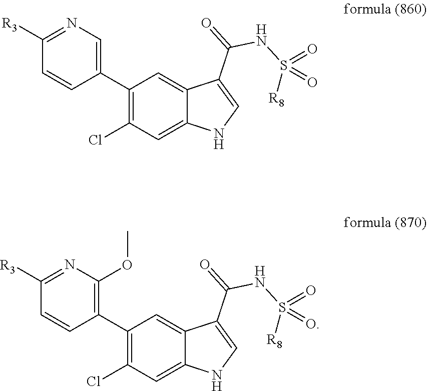

In one embodiment, R₁ is

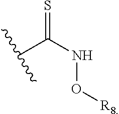

In one embodiment, the compound of formula (I) is a compound of formula (900), formula (910), formula (920), formula (930), formula (940), formula (950), formula (960), or formula (970) or a pharmaceutically acceptable salt, solvate, hydrate, cocrystal, or prodrug thereof.

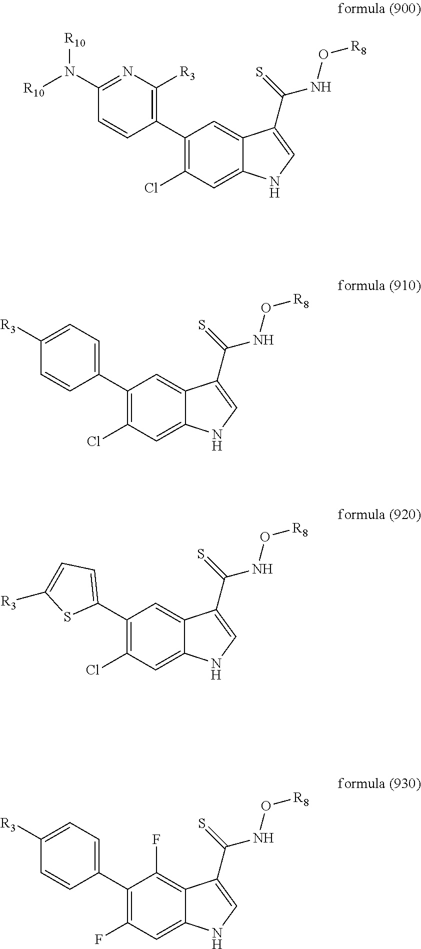

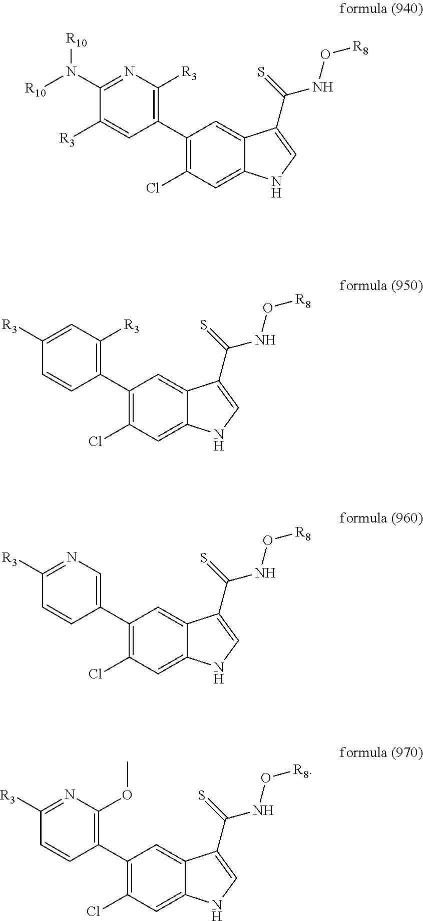

In some embodiments, R₁₀ is selected from H, methyl, ethyl, —CD₃ n-butyl,

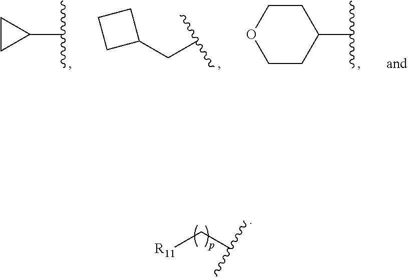

In some embodiments, R₁₁ is selected from

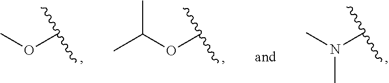

In some embodiments, p is 2 or 3.

In some embodiments, R₃ is selected from —F, —CN, ethyl,

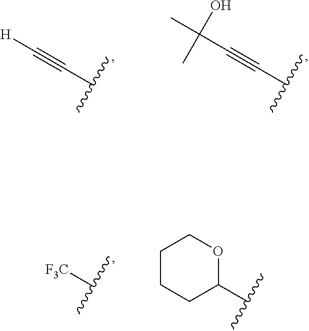

optionally,

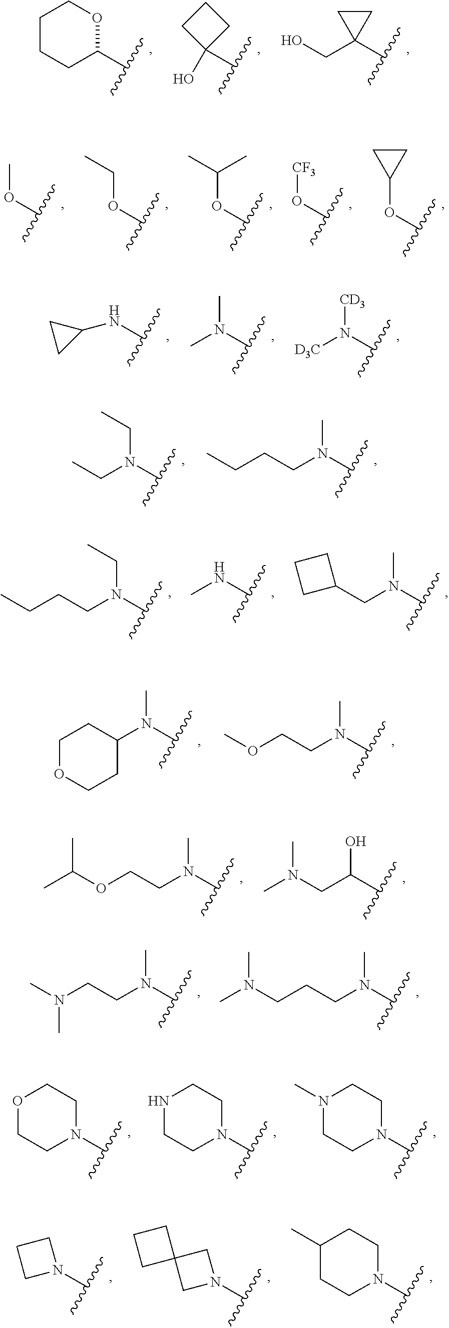

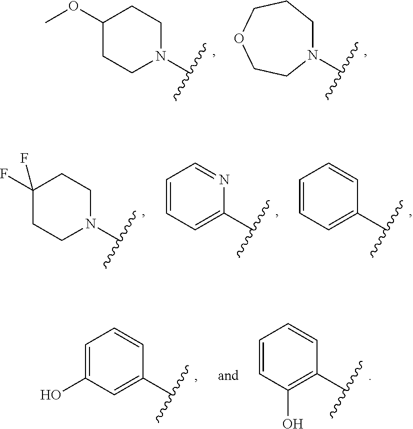

In some embodiment, R₃ is selected from

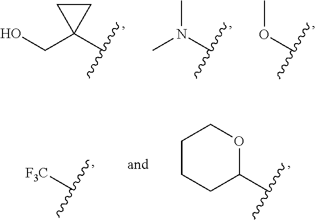

optionally

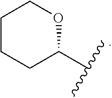 [SMILES: *[C@@H]1CCCCO1]

In some embodiments,

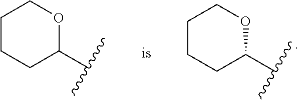 [SMILES: *C1CCCCO1 ; *[C@@H]1CCCCO1]

In some embodiments,

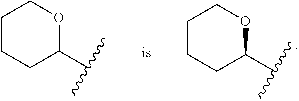 [SMILES: *C1CCCCO1 ; *[C@H]1CCCCO1]

In some embodiments, q is 0 or 1.

In some embodiments, r is 2 or 3.

In some embodiments, s is 1.

In some embodiments, R₁₂ is selected from methyl, ethyl, —OCF₃, and

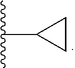 [SMILES: *C1CC1]

n some embodiments, R₈ comprises one or more —OCF₃ groups. In some embodiments, R₈ is selected from H and methyl.

In some embodiments, R₈ is selected from H, methyl, ethyl, isopropyl, n-butyl t-butyl,

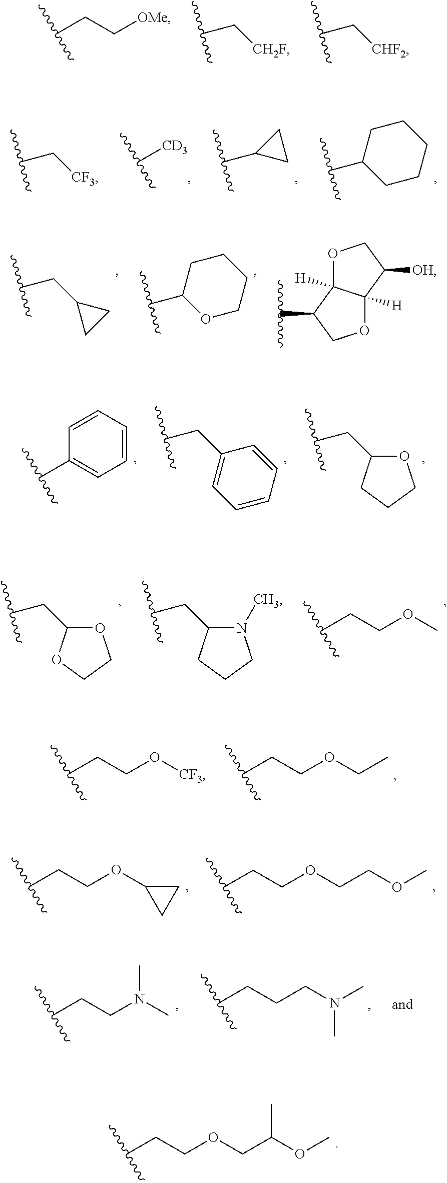

In one embodiment, R₈ is selected from H, methyl, and ethyl.

In some embodiments, in formula (10), formula (100) formula (200), formula (300), formula (500), formula (600), formula (700), or formula (800) R₃ is selected from C₁₋₁₀ alkyl, C₁₋₁₀ alkoxy, —CF₃, —OCF₃,

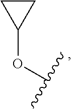 [SMILES: *OC1CC1]

and —OH. In one embodiment, in formula (100), R₃ is selected from C₁₋₁₀ alkyl, C₁₋₁₀ alkoxy, —CF₃, —OCF₃,

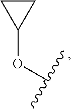 [SMILES: *OC1CC1]

and —OH. In one embodiment, in formula (200), R₃ is C₁₋₁₀ alkoxy. In one embodiment, in formula (300), R₃ is C₁₋₁₀ alkoxy. In one embodiment, in formula (500), R₃ is C₁₋₁₀ alkoxy. In one embodiment, in formula (600), R₃ is C₁₋₁₀ alkoxy. In one embodiment, in formula (700), R₃ is C₁₋₁₀ alkoxy. In one embodiment, in formula (800), R₃ is C₁₋₁₀ alkoxy. In one embodiment, in formula (900), R₃ is C₁₋₁₀ alkoxy.

In some embodiments, in formula (11), formula (110), formula (710), or formula (810), R₃ is selected from

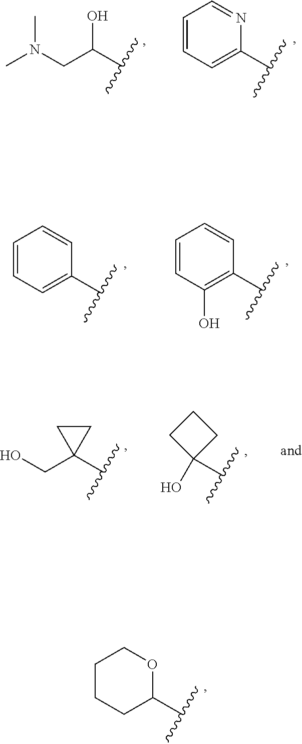

optionally

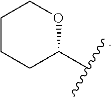 [SMILES: *[C@@H]1CCCCO1]

In one embodiment, in formula (110), R₃ is selected from

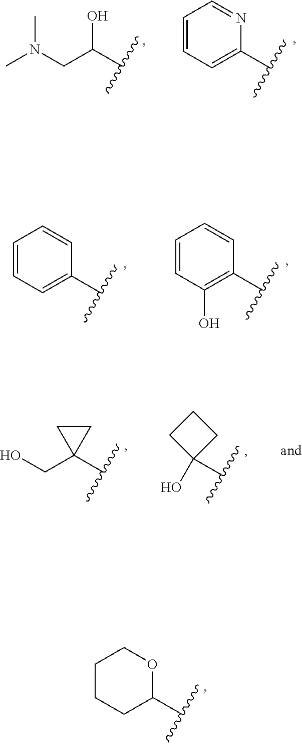

optionally

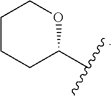 [SMILES: *[C@@H]1CCCCO1]

In one embodiment, in formula (710), R₃ is selected from

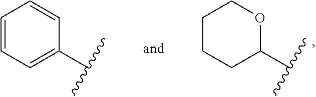 [SMILES: *c1ccccc1 ; *C1CCCCO1]

optionally

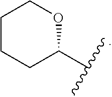 [SMILES: *[C@@H]1CCCCO1]

In one embodiment, in formula (810), R₃ is

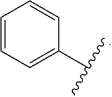 [SMILES: *c1ccccc1]

In some embodiments, in formula (12), formula (130), formula (230), or formula (330), R₃ is

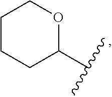 [SMILES: *C1CCCCO1]

optionally

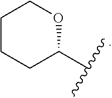 [SMILES: *[C@@H]1CCCCO1]

In some embodiments, in formula (13), formula (120), or formula (420), R₃ is

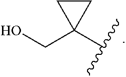 [SMILES: *C1(CO)CC1]

In some embodiments, in formula (14) or formula (140), each R₃ is independently selected from —F and —OCH₃ and each R₁₀ is independently C₁₋₁₀ alkyl. In one embodiment, in formula (140), one R₃ is —F, one R₃ is —OCH₃, and each R₁₀ is —CH₃.

In some embodiments, in formula (15), formula (150), or formula (350), each R₃ is independently selected from —OCH₃, —CN, —N(CH₃)₂, —Cl, and ethyl. In one embodiment, in formula (150), one R₃ is —N(CH₃)₂ and one R₃ is selected from —OCH₃, —Cl, and ethyl. In another embodiment, in formula (150), one R₃ is —CN and one R₃ is —OCH₃. In one embodiment, in formula (350), one R₃ is —N(CH₃)₂ and one R₃ is —OCH₃.

In some embodiments, in formula (16) or formula (160), R₃ is selected from

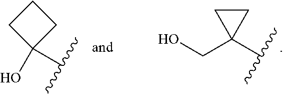 [SMILES: *C1(O)CCC1 ; *C1(CO)CC1]

In some embodiments, in in formula (17) or formula (170), R₃ is selected from

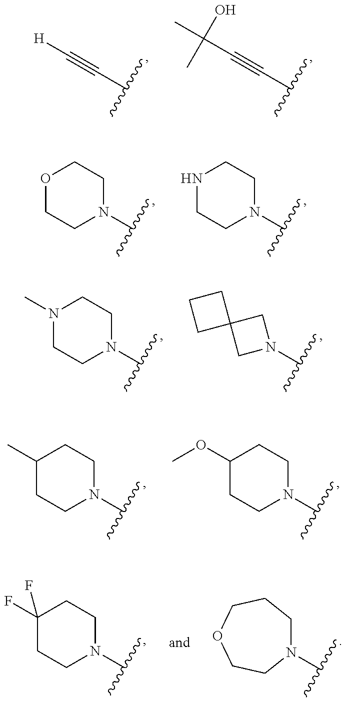

In one embodiment, X₁ is N and the compound of formula (I) is a compound of formula (20), formula (21), formula (22), formula (23), formula (24), formula (25), or formula (26), or a pharmaceutically acceptable salt, solvate, hydrate, cocrystal, or prodrug thereof:

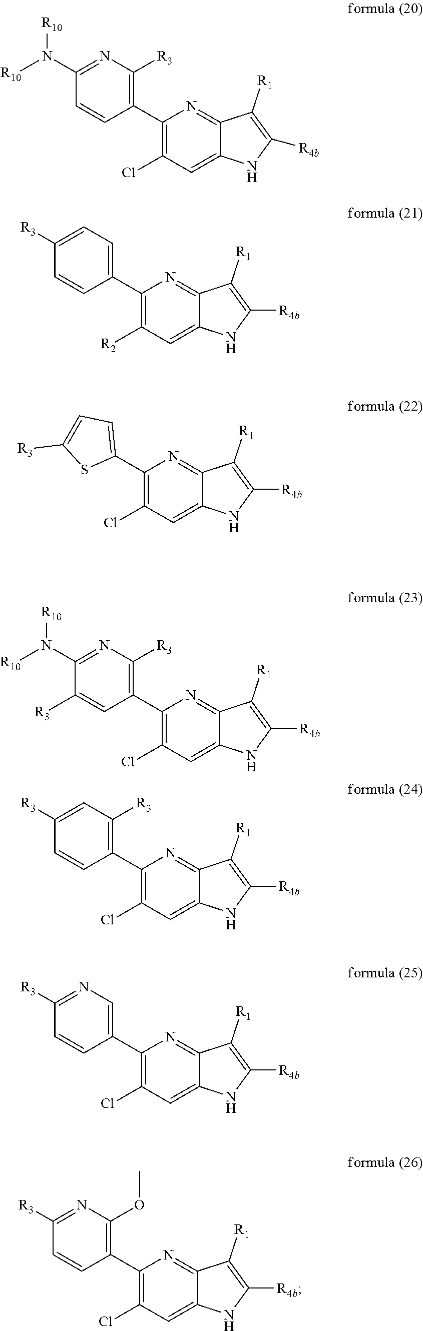

wherein R₂ is selected from H and —Cl and each R_{4b} is independently selected from H and C₁₋₁₀ alkyl.

In one embodiment, the present disclosure provides a compound of formula (20) wherein R₁ is

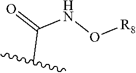

and R_{4b} is H. In one embodiment, the present disclosure provides a compound of formula (20) wherein R₁ is

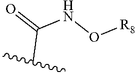

and R₈ is C₁₋₁₀ alkyl. In one embodiment, the present disclosure provides a compound of formula (20) wherein R₁ is

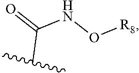

R₃ is C₁₋₁₀ alkoxy, and each R₁₀ is independently C₁₋₁₀ alkyl. In one embodiment, the present disclosure provides a compound of formula (20) wherein R₁ is

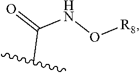

R_{4b} is H, R₃ is C₁₋₁₀ alkoxy, R₈ is C₁₋₁₀ alkyl, and each R₁₀ is independently C₁₋₁₀ alkyl. In one embodiment, In one embodiment, the present disclosure provides a compound of formula (20) wherein R₁ is

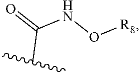

R_{4b} is H, R₃ is —OCH₃, R₈ is selected from methyl and ethyl, and each R₁₀ is independently methyl.

In one embodiment, the present disclosure provides a compound of formula (21) wherein R₁ is

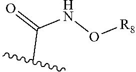

and R_{4b} is H. In one embodiment, the present disclosure provides a compound of formula (21) wherein R₁ is

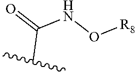

and R₂ is H. In one embodiment, the present disclosure provides a compound of formula (21) wherein R₁ is

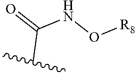

### and R₃ is

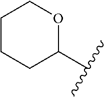 [SMILES: *C1CCCCO1]

optionally

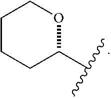 [SMILES: *[C@@H]1CCCCO1]

In one embodiment, the present disclosure provides a compound of formula (21) wherein R₁ is

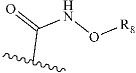

wherein R₈ is C₁₋₁₀ alkyl. In one embodiment, the present disclosure provides a compound of formula (21) wherein R₁ is

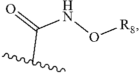

R₂ is H, R₃ is

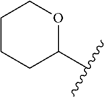 [SMILES: *C1CCCCO1]

optionally

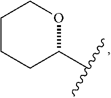 [SMILES: *[C@@H]1CCCCO1]

R_{4b} is H, and R₈ is methyl.

In one embodiment, A is

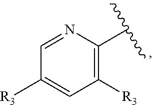

### R₁ is

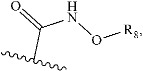

and the compound of formula (I) is a compound of formula (180):

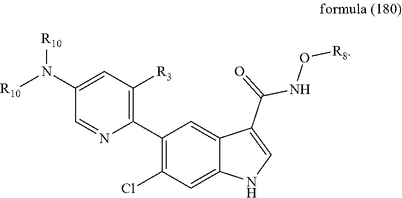

In one embodiment, the compound of formula (I) is a compound of formula (180) wherein R₈ is C₁₋₁₀ alkyl. In one embodiment, the compound of formula (I) is a compound of formula (180) wherein R₃ is C₁₋₁₀ alkoxy and each R₁₀ is independently C₁₋₁₀ alkyl. In one embodiment, the compound of formula (I) is a compound of formula (180) wherein R₃ is —OCH₃, R₈ is methyl, and each R₁₀ is methyl.

In one embodiment, A is

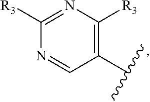

### R₁ is

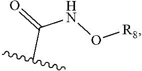

and the compound of formula (I) is a compound of formula (190):

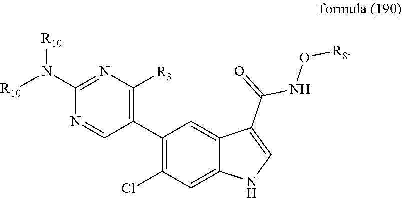

In one embodiment, the compound of formula (I) is a compound of formula (190) wherein R₈ is C₁₋₁₀ alkyl. In one embodiment, the compound of formula (I) is a compound of formula (190) wherein R₃ is C₁₋₁₀ alkoxy and each R₁₀ is independently C₁₋₁₀ alkyl. In one embodiment, the compound of formula (I) is a compound of formula (190) wherein R₃ is —OCH₃, R₈ is methyl, and each R₁₀ is methyl.

In some embodiments, the compound of formula (I) is selected from a compound having a formula selected from formula 1001-1114, or a pharmaceutically acceptable salt, solvate, hydrate, cocrystal, or prodrug thereof:

| Cpd. # | |
|---|---|
| 1001 | 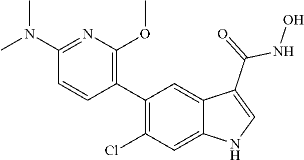 [SMILES: COc1nc(N(C)C)ccc1-c1cc2c(C(=O)NO)c[nH]c2cc1Cl] |
| 1002 | 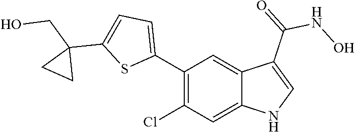 [SMILES: O=C(NO)c1c[nH]c2cc(Cl)c(-c3ccc(C4(CO)CC4)s3)cc12] |
| 1003 | 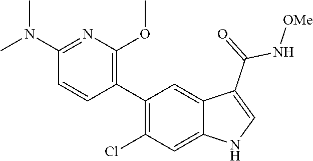 [SMILES: CONC(=O)c1c[nH]c2cc(Cl)c(-c3ccc(N(C)C)nc3OC)cc12] |
| 1004 | 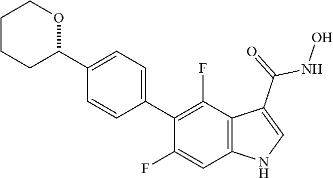 [SMILES: O=C(NO)c1c[nH]c2cc(F)c(-c3ccc([C@@H]4CCCCO4)cc3)c(F)c12] |
| 1005 | 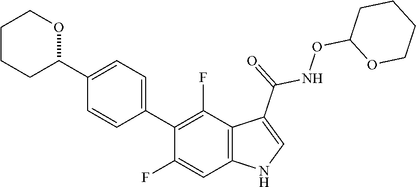 [SMILES: O=C(NOC1CCCCO1)c1c[nH]c2cc(F)c(-c3ccc([C@@H]4CCCCO4)cc3)c(F)c12] |
| 1006 | 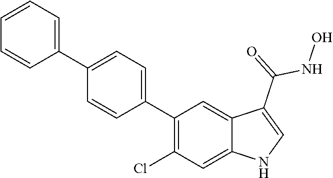 [SMILES: O=C(NO)c1c[nH]c2cc(Cl)c(-c3ccc(-c4ccccc4)cc3)cc12] |
| 1007 | 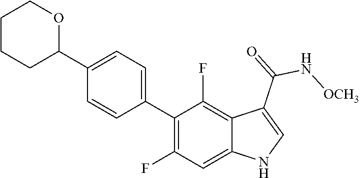 [SMILES: CONC(=O)c1c[nH]c2cc(F)c(-c3ccc(C4CCCCO4)cc3)c(F)c12] |
| 1008 | 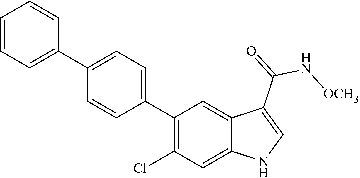 [SMILES: CONC(=O)c1c[nH]c2cc(Cl)c(-c3ccc(-c4ccccc4)cc3)cc12] |
| 1009 | 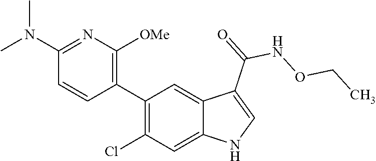 [SMILES: CCONC(=O)c1c[nH]c2cc(Cl)c(-c3ccc(N(C)C)nc3OC)cc12] |
| 1010 | 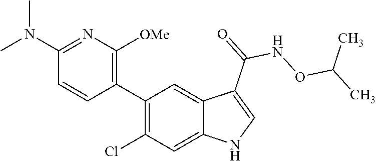 [SMILES: COc1nc(N(C)C)ccc1-c1cc2c(C(=O)NOC(C)C)c[nH]c2cc1Cl] |
| 1011 | 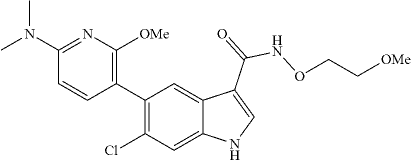 [SMILES: COCCONC(=O)c1c[nH]c2cc(Cl)c(-c3ccc(N(C)C)nc3OC)cc12] |
| 1012 | 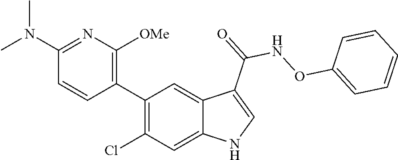 [SMILES: COc1nc(N(C)C)ccc1-c1cc2c(C(=O)NOc3ccccc3)c[nH]c2cc1Cl] |
| 1013 | 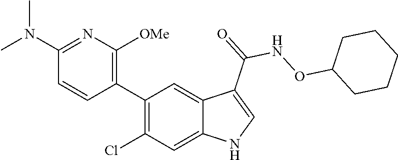 [SMILES: COc1nc(N(C)C)ccc1-c1cc2c(C(=O)NOC3CCCCC3)c[nH]c2cc1Cl] |
| 1014 | 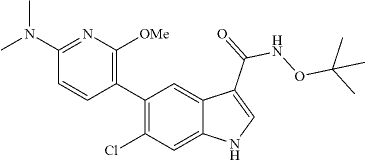 [SMILES: COc1nc(N(C)C)ccc1-c1cc2c(C(=O)NOC(C)(C)C)c[nH]c2cc1Cl] |
| 1015 | 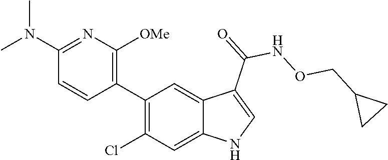 [SMILES: COc1nc(N(C)C)ccc1-c1cc2c(C(=O)NOCC3CC3)c[nH]c2cc1Cl] |
| 1016 | 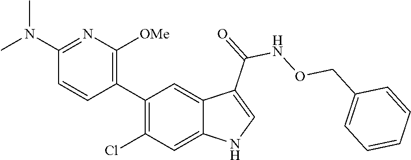 [SMILES: COc1nc(N(C)C)ccc1-c1cc2c(C(=O)NOCc3ccccc3)c[nH]c2cc1Cl] |
| 1017 | 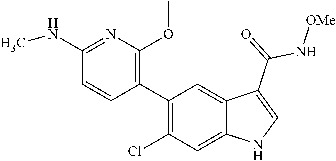 [SMILES: CNc1ccc(-c2cc3c(C(=O)NOC)c[nH]c3cc2Cl)c(OC)n1] |
| 1018 | 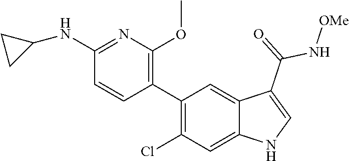 [SMILES: CONC(=O)c1c[nH]c2cc(Cl)c(-c3ccc(NC4CC4)nc3OC)cc12] |
| 1019 | 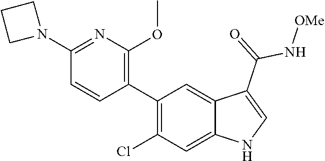 [SMILES: CONC(=O)c1c[nH]c2cc(Cl)c(-c3ccc(N4CCC4)nc3OC)cc12] |
| 1020 | 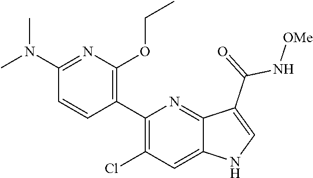 [SMILES: CCOc1nc(N(C)C)ccc1-c1nc2c(C(=O)NOC)c[nH]c2cc1Cl] |
| 1021 | 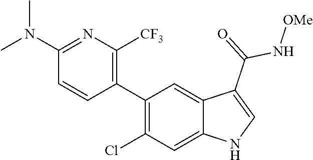 [SMILES: CONC(=O)c1c[nH]c2cc(Cl)c(-c3ccc(N(C)C)nc3C(F)(F)F)cc12] |
| 1022 | 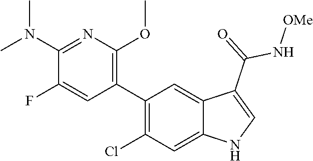 [SMILES: CONC(=O)c1c[nH]c2cc(Cl)c(-c3cc(F)c(N(C)C)nc3OC)cc12] |
| 1023 | 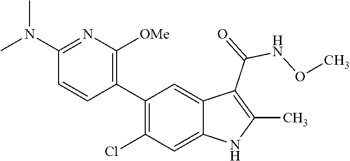 |
| 1024 | 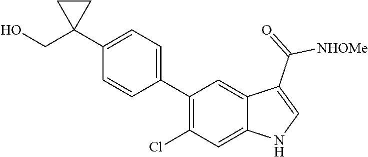 |
| 1025 | 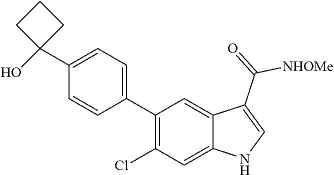 |
| 1026 | 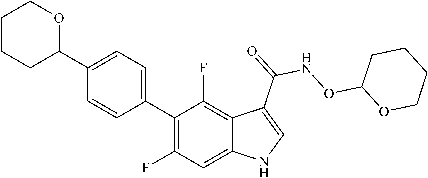 [SMILES: O=C(NOC1CCCCO1)c1c[nH]c2cc(F)c(-c3ccc(C4CCCCO4)cc3)c(F)c12] |
| 1027 | 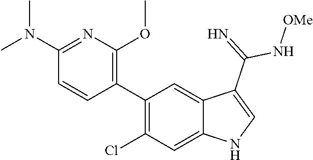 [SMILES: CONC(=N)c1c[nH]c2cc(Cl)c(-c3ccc(N(C)C)nc3OC)cc12] |
| 1028 | 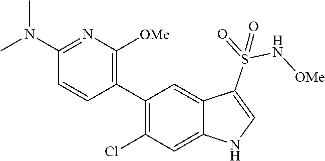 [SMILES: CONS(=O)(=O)c1c[nH]c2cc(Cl)c(-c3ccc(N(C)C)nc3OC)cc12] |
| 1029 | 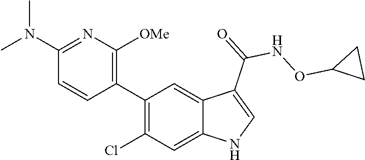 [SMILES: COc1nc(N(C)C)ccc1-c1cc2c(C(=O)NOC3CC3)c[nH]c2cc1Cl] |
| 1030 | 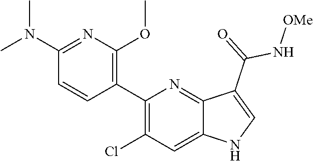 [SMILES: CONC(=O)c1c[nH]c2cc(Cl)c(-c3ccc(N(C)C)nc3OC)nc12] |
| 1031 | 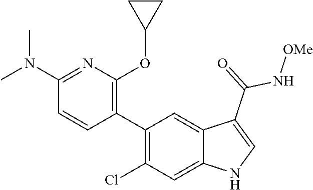 [SMILES: CONC(=O)c1c[nH]c2cc(Cl)c(-c3ccc(N(C)C)nc3OC3CC3)cc12] |
| 1032 | 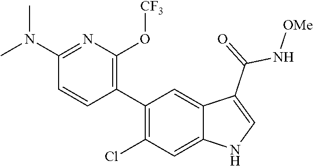 [SMILES: CONC(=O)c1c[nH]c2cc(Cl)c(-c3ccc(N(C)C)nc3OC(F)(F)F)cc12] |
| 1033 | 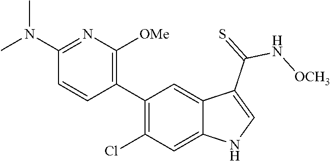 [SMILES: CONC(=S)c1c[nH]c2cc(Cl)c(-c3ccc(N(C)C)nc3OC)cc12] |
| 1034 | 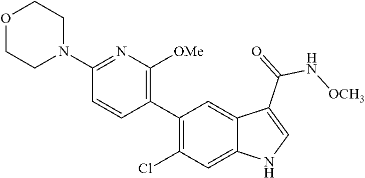 [SMILES: CONC(=O)c1c[nH]c2cc(Cl)c(-c3ccc(N4CCOCC4)nc3OC)cc12] |
| 1035 | 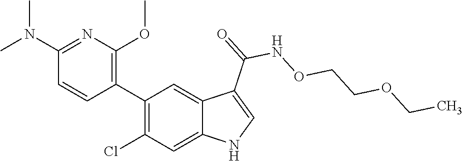 [SMILES: CCOCCONC(=O)c1c[nH]c2cc(Cl)c(-c3ccc(N(C)C)nc3OC)cc12] |
| 1036 | 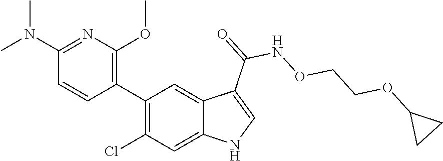 [SMILES: COc1nc(N(C)C)ccc1-c1cc2c(C(=O)NOCCOC3CC3)c[nH]c2cc1Cl] |
| 1037 | 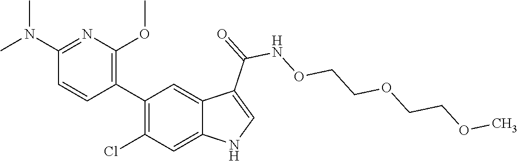 |
| 1038 | 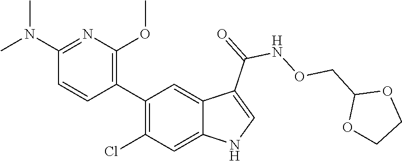 [SMILES: COc1nc(N(C)C)ccc1-c1cc2c(C(=O)NOCC3OCCO3)c[nH]c2cc1Cl] |
| 1039 | 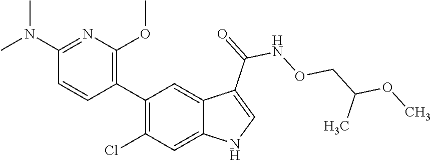 |
| 1040 | 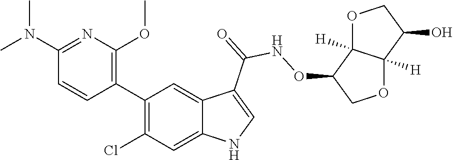 [SMILES: COc1nc(N(C)C)ccc1-c1cc2c(C(=O)NO[C@@H]3CO[C@H]4[C@@H]3OC[C@H]4O)c[nH]c2cc1Cl] |
| 1041 | 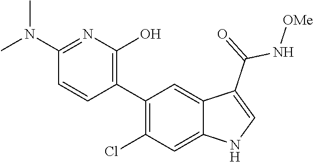 [SMILES: CONC(=O)c1c[nH]c2cc(Cl)c(-c3ccc(N(C)C)nc3O)cc12] |
| 1042 | 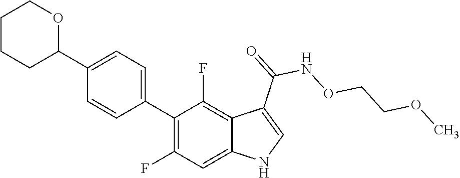 [SMILES: COCCONC(=O)c1c[nH]c2cc(F)c(-c3ccc(C4CCCCO4)cc3)c(F)c12] |
| 1043 | 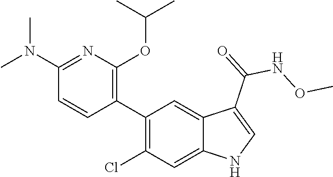 |
| 1044 | 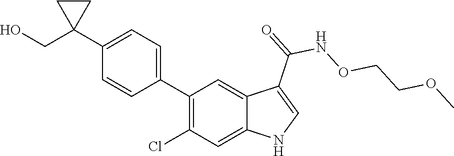 [SMILES: COCCONC(=O)c1c[nH]c2cc(Cl)c(-c3ccc(C4(CO)CC4)cc3)cc12] |
| 1045 | 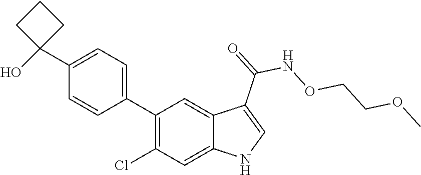 [SMILES: COCCONC(=O)c1c[nH]c2cc(Cl)c(-c3ccc(C4(O)CCC4)cc3)cc12] |
| 1046 | 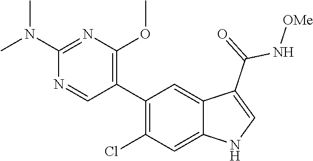 [SMILES: CONC(=O)c1c[nH]c2cc(Cl)c(-c3cnc(N(C)C)nc3OC)cc12] |
| 1047 | 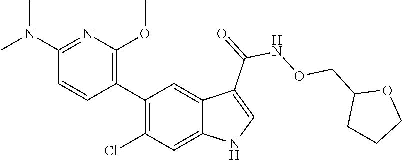 [SMILES: COc1nc(N(C)C)ccc1-c1cc2c(C(=O)NOCC3CCCO3)c[nH]c2cc1Cl] |
| 1048 | 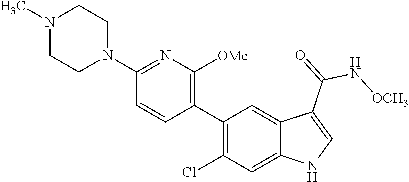 [SMILES: CONC(=O)c1c[nH]c2cc(Cl)c(-c3ccc(N4CCN(C)CC4)nc3OC)cc12] |
| 1049 | 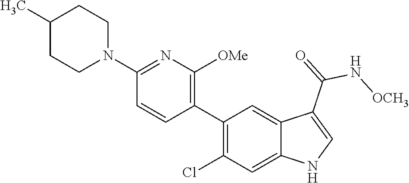 [SMILES: CONC(=O)c1c[nH]c2cc(Cl)c(-c3ccc(N4CCC(C)CC4)nc3OC)cc12] |
| 1050 | 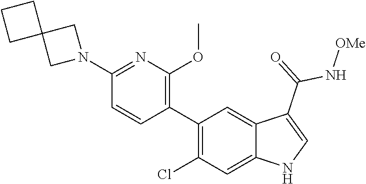 [SMILES: CONC(=O)c1c[nH]c2cc(Cl)c(-c3ccc(N4CC5(CCC5)C4)nc3OC)cc12] |
| 1051 | 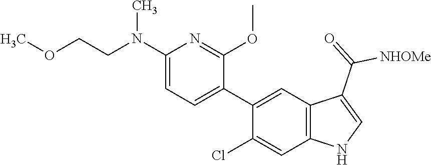 |
| 1052 | 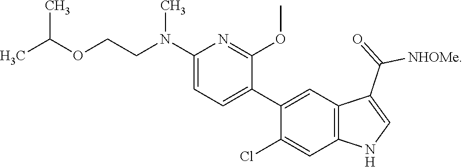 |
| 1053 | 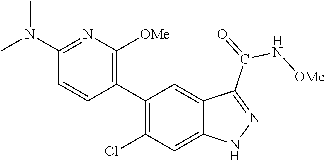 [SMILES: CONC(=O)c1n[nH]c2cc(Cl)c(-c3ccc(N(C)C)nc3OC)cc12] |
| 1054 | 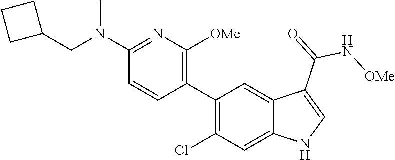 [SMILES: CONC(=O)c1c[nH]c2cc(Cl)c(-c3ccc(N(C)CC4CCC4)nc3OC)cc12] |
| 1055 | 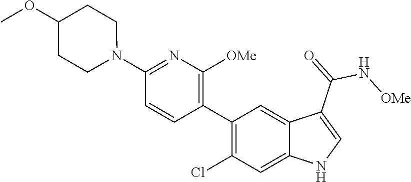 [SMILES: CONC(=O)c1c[nH]c2cc(Cl)c(-c3ccc(N4CCC(OC)CC4)nc3OC)cc12] |
| 1056 | 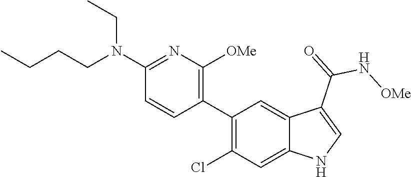 [SMILES: CCCCN(CC)c1ccc(-c2cc3c(C(=O)NOC)c[nH]c3cc2Cl)c(OC)n1] |
| 1057 | 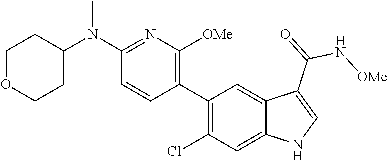 [SMILES: CONC(=O)c1c[nH]c2cc(Cl)c(-c3ccc(N(C)C4CCOCC4)nc3OC)cc12] |
| 1058 | 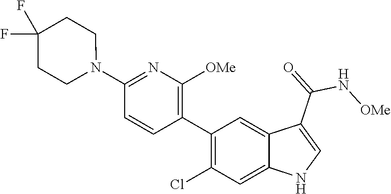 [SMILES: CONC(=O)c1c[nH]c2cc(Cl)c(-c3ccc(N4CCC(F)(F)CC4)nc3OC)cc12] |
| 1059 | 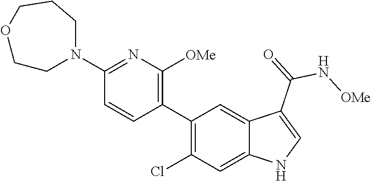 [SMILES: CONC(=O)c1c[nH]c2cc(Cl)c(-c3ccc(N4CCCOCC4)nc3OC)cc12] |
| 1060 | 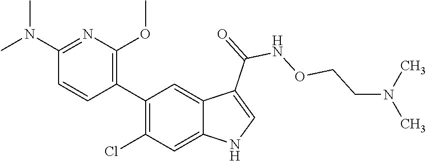 |
| 1061 | 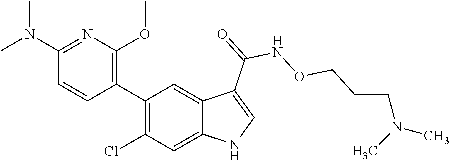 |
| 1062 | 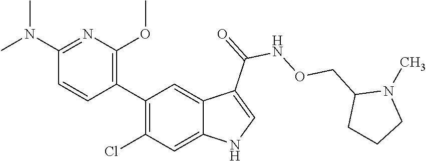 |
| 1063 | 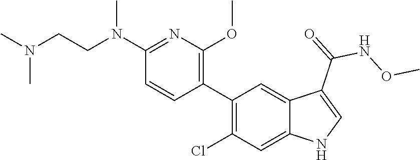 |
| 1064 | 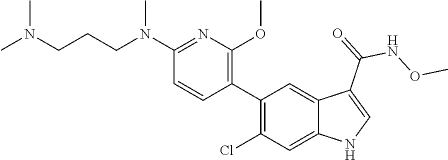 |
| 1065 | 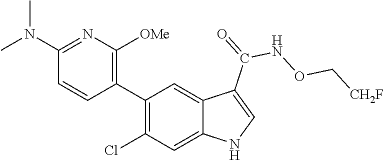 |
| 1066 | 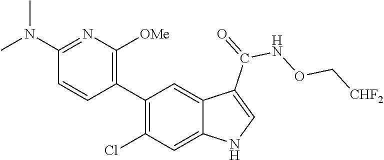 [SMILES: COc1nc(N(C)C)ccc1-c1cc2c(C(=O)NOCC(F)F)c[nH]c2cc1Cl] |
| 1067 | 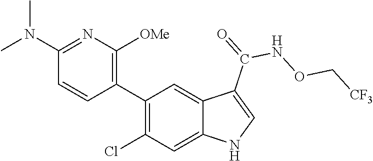 [SMILES: COc1nc(N(C)C)ccc1-c1cc2c(C(=O)NOCC(F)(F)F)c[nH]c2cc1Cl] |
| 1068 | 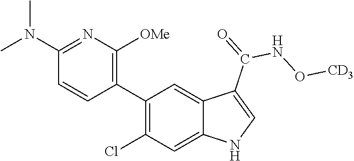 [SMILES: [2H]C([2H])([2H])ONC(=O)c1c[nH]c2cc(Cl)c(-c3ccc(N(C)C)nc3OC)cc12] |
| 1069 | 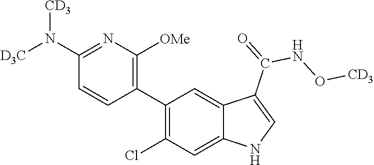 [SMILES: [2H]C([2H])([2H])ONC(=O)c1c[nH]c2cc(Cl)c(-c3ccc(N(C([2H])([2H])[2H])C([2H])([2H])[2H])nc3OC)cc12] |
| 1070 | 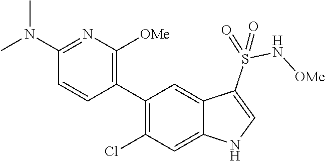 [SMILES: CONS(=O)(=O)c1c[nH]c2cc(Cl)c(-c3ccc(N(C)C)nc3OC)cc12] |
| 1071 | 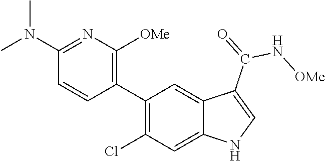 [SMILES: CONC(=O)c1c[nH]c2cc(Cl)c(-c3ccc(N(C)C)nc3OC)cc12] |
| 1072 | 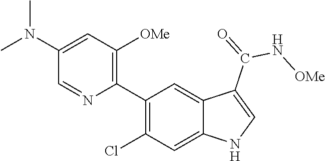 [SMILES: CONC(=O)c1c[nH]c2cc(Cl)c(-c3ncc(N(C)C)cc3OC)cc12] |
| 1073 | 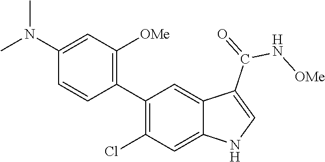 [SMILES: CONC(=O)c1c[nH]c2cc(Cl)c(-c3ccc(N(C)C)cc3OC)cc12] |
| 1074 | 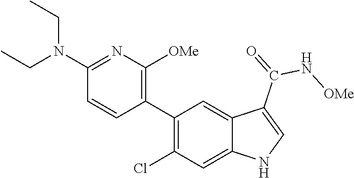 [SMILES: CCN(CC)c1ccc(-c2cc3c(C(=O)NOC)c[nH]c3cc2Cl)c(OC)n1] |
| 1075 | 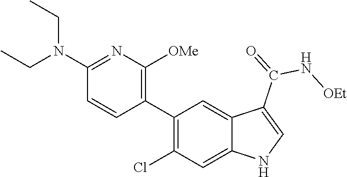 [SMILES: CCONC(=O)c1c[nH]c2cc(Cl)c(-c3ccc(N(CC)CC)nc3OC)cc12] |
| 1076 | 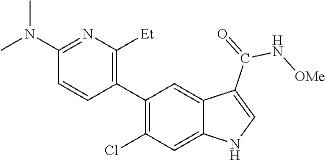 [SMILES: CCc1nc(N(C)C)ccc1-c1cc2c(C(=O)NOC)c[nH]c2cc1Cl] |
| 1077 | 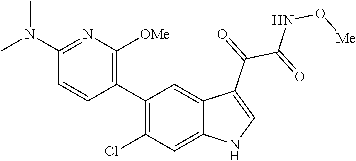 [SMILES: CONC(=O)C(=O)c1c[nH]c2cc(Cl)c(-c3ccc(N(C)C)nc3OC)cc12] |
| 1078 | 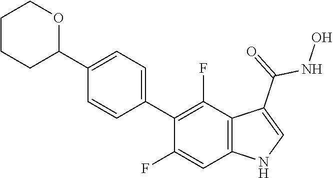 [SMILES: O=C(NO)c1c[nH]c2cc(F)c(-c3ccc(C4CCCCO4)cc3)c(F)c12] |
| 1080 | 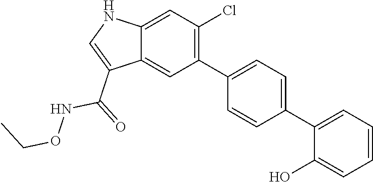 [SMILES: CCONC(=O)c1c[nH]c2cc(Cl)c(-c3ccc(-c4ccccc4O)cc3)cc12] |
| 1081 | 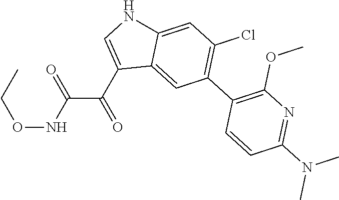 [SMILES: CCONC(=O)C(=O)c1c[nH]c2cc(Cl)c(-c3ccc(N(C)C)nc3OC)cc12] |
| 1082 | 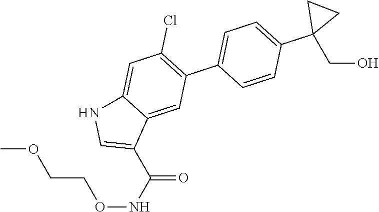 [SMILES: COCCONC(=O)c1c[nH]c2cc(Cl)c(-c3ccc(C4(CO)CC4)cc3)cc12] |
| 1084 | 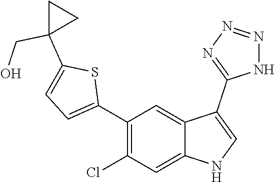 [SMILES: OCC1(c2ccc(-c3cc4c(-c5nnn[nH]5)c[nH]c4cc3Cl)s2)CC1] |
| 1088 | 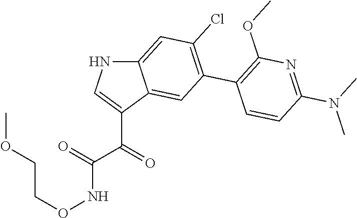 [SMILES: COCCONC(=O)C(=O)c1c[nH]c2cc(Cl)c(-c3ccc(N(C)C)nc3OC)cc12] |
| 1089 | 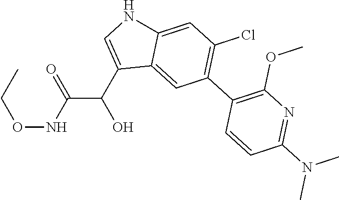 [SMILES: CCONC(=O)C(O)c1c[nH]c2cc(Cl)c(-c3ccc(N(C)C)nc3OC)cc12] |
| 1090 | 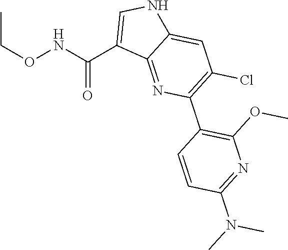 [SMILES: CCONC(=O)c1c[nH]c2cc(Cl)c(-c3ccc(N(C)C)nc3OC)nc12] |
| 1091 | 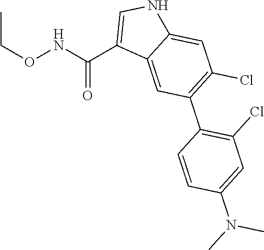 [SMILES: CCONC(=O)c1c[nH]c2cc(Cl)c(-c3ccc(N(C)C)cc3Cl)cc12] |
| 1092 | 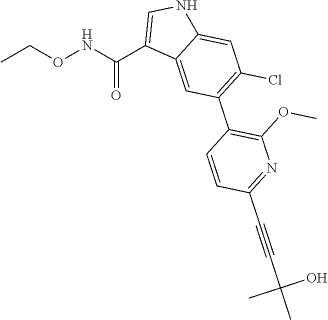 [SMILES: CCONC(=O)c1c[nH]c2cc(Cl)c(-c3ccc(C#CC(C)(C)O)nc3OC)cc12] |
| 1093 | 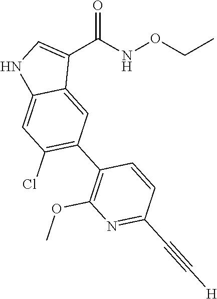 [SMILES: C#Cc1ccc(-c2cc3c(C(=O)NOCC)c[nH]c3cc2Cl)c(OC)n1] |
| 1094 | 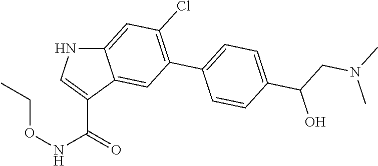 [SMILES: CCONC(=O)c1c[nH]c2cc(Cl)c(-c3ccc(C(O)CN(C)C)cc3)cc12] |
| 1095 | 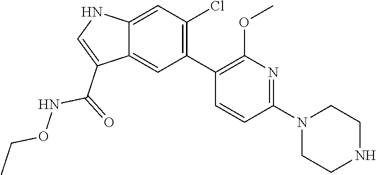 [SMILES: CCONC(=O)c1c[nH]c2cc(Cl)c(-c3ccc(N4CCNCC4)nc3OC)cc12] |
| 1096 | 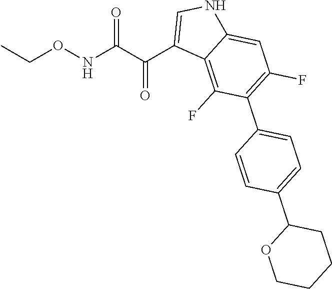 [SMILES: CCONC(=O)C(=O)c1c[nH]c2cc(F)c(-c3ccc(C4CCCCO4)cc3)c(F)c12] |
| 1097 | 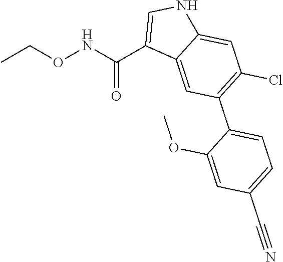 [SMILES: CCONC(=O)c1c[nH]c2cc(Cl)c(-c3ccc(C#N)cc3OC)cc12] |
| 1098 | 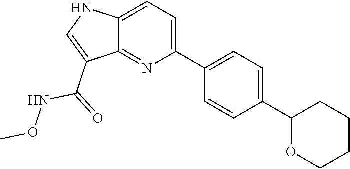 [SMILES: CONC(=O)c1c[nH]c2ccc(-c3ccc(C4CCCCO4)cc3)nc12] |
| 1099 | 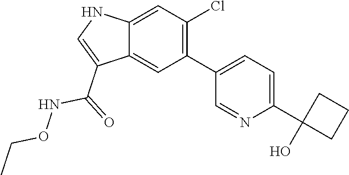 [SMILES: CCONC(=O)c1c[nH]c2cc(Cl)c(-c3ccc(C4(O)CCC4)nc3)cc12] |
| 1100 | 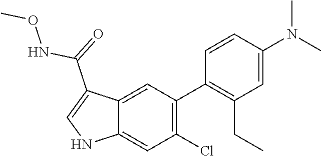 |
| 1101 | 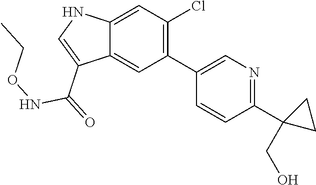 [SMILES: CCONC(=O)c1c[nH]c2cc(Cl)c(-c3ccc(C4(CO)CC4)nc3)cc12] |
| 1102 | 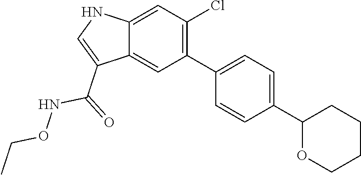 [SMILES: CCONC(=O)c1c[nH]c2cc(Cl)c(-c3ccc(C4CCCCO4)cc3)cc12] |
| 1103 | 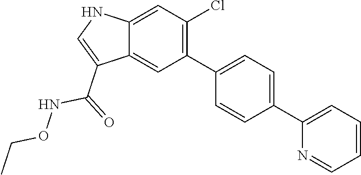 [SMILES: CCONC(=O)c1c[nH]c2cc(Cl)c(-c3ccc(-c4ccccn4)cc3)cc12] |
| 1104 | 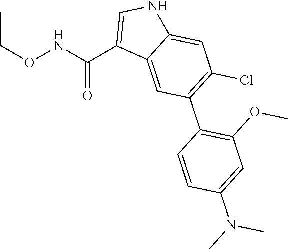 [SMILES: CCONC(=O)c1c[nH]c2cc(Cl)c(-c3ccc(N(C)C)cc3OC)cc12] |
| 1105 | 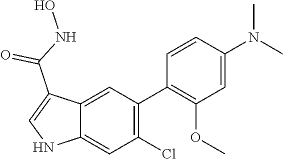 [SMILES: COc1cc(N(C)C)ccc1-c1cc2c(C(=O)NO)c[nH]c2cc1Cl] |
| 1106 | 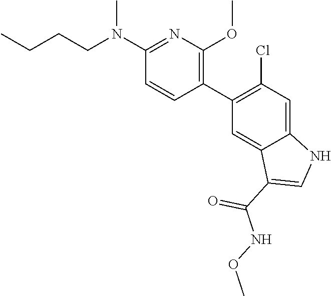 [SMILES: CCCCN(C)c1ccc(-c2cc3c(C(=O)NOC)c[nH]c3cc2Cl)c(OC)n1] |
| 1107 | 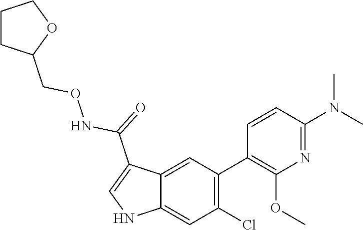 [SMILES: COc1nc(N(C)C)ccc1-c1cc2c(C(=O)NOCC3CCCO3)c[nH]c2cc1Cl] |
| 1108 | 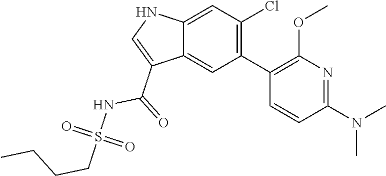 [SMILES: CCCCS(=O)(=O)NC(=O)c1c[nH]c2cc(Cl)c(-c3ccc(N(C)C)nc3OC)cc12] |
| 1109 | 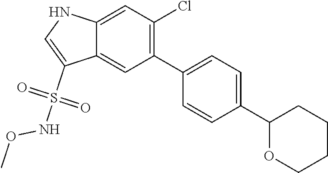 [SMILES: CONS(=O)(=O)c1c[nH]c2cc(Cl)c(-c3ccc(C4CCCCO4)cc3)cc12] |
| 1110 | 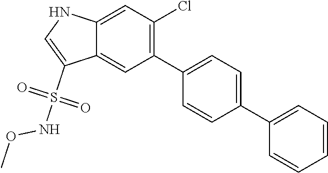 [SMILES: CONS(=O)(=O)c1c[nH]c2cc(Cl)c(-c3ccc(-c4ccccc4)cc3)cc12] |
| 1111 | 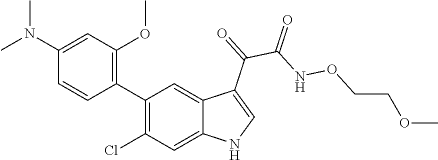 |
| 1112 | 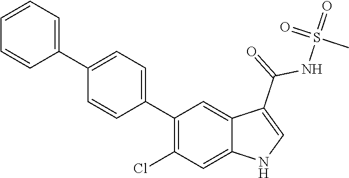 [SMILES: CS(=O)(=O)NC(=O)c1c[nH]c2cc(Cl)c(-c3ccc(-c4ccccc4)cc3)cc12] |
| 1113 | 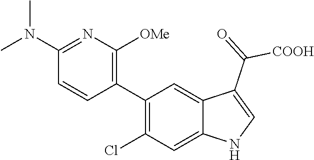 [SMILES: COc1nc(N(C)C)ccc1-c1cc2c(C(=O)C(=O)O)c[nH]c2cc1Cl] |
| 1114 | 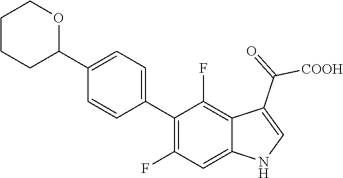 [SMILES: O=C(O)C(=O)c1c[nH]c2cc(F)c(-c3ccc(C4CCCCO4)cc3)c(F)c12] |

or a pharmaceutically acceptable salt, solvate, hydrate, cocrystal, or prodrug thereof.

In some embodiments, the compound of formula (I) is selected from:

| | |
|---|---|
| 1003 | 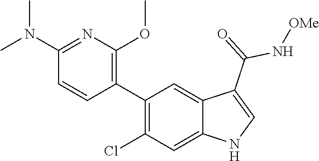 [SMILES: CONC(=O)c1c[nH]c2cc(Cl)c(-c3ccc(N(C)C)nc3OC)cc12] |
| 1009 | 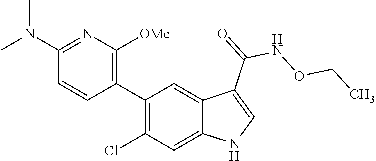 [SMILES: CCONC(=O)c1c[nH]c2cc(Cl)c(-c3ccc(N(C)C)nc3OC)cc12] |
| 1011 | 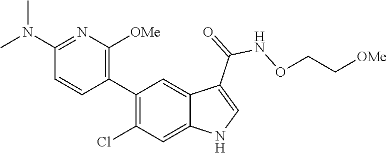 [SMILES: COCCONC(=O)c1c[nH]c2cc(Cl)c(-c3ccc(N(C)C)nc3OC)cc12] |
| 1015 | 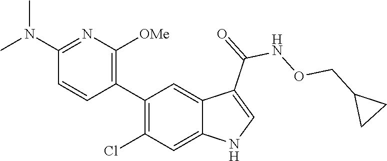 [SMILES: COc1nc(N(C)C)ccc1-c1cc2c(C(=O)NOCC3CC3)c[nH]c2cc1Cl] |

In some embodiments, the compound of formula (I) is selected from:

| | |
|---|---|
| 1003 | 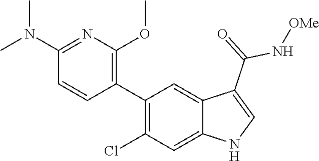 [SMILES: CONC(=O)c1c[nH]c2cc(Cl)c(-c3ccc(N(C)C)nc3OC)cc12] |
| 1007 | 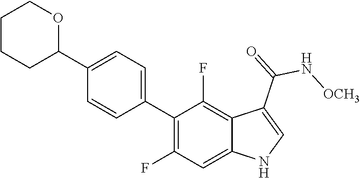 [SMILES: CONC(=O)c1c[nH]c2cc(F)c(-c3ccc(C4CCCCO4)cc3)c(F)c12] |
| 1009 | 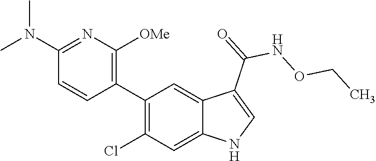 [SMILES: CCONC(=O)c1c[nH]c2cc(Cl)c(-c3ccc(N(C)C)nc3OC)cc12] |
| 1011 | 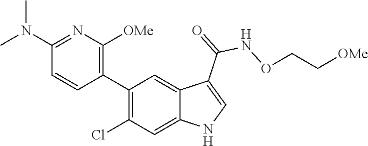 [SMILES: COCCONC(=O)c1c[nH]c2cc(Cl)c(-c3ccc(N(C)C)nc3OC)cc12] |
| 1015 | 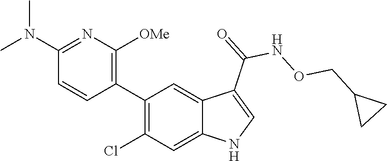 [SMILES: COc1nc(N(C)C)ccc1-c1cc2c(C(=O)NOCC3CC3)c[nH]c2cc1Cl] |
| 1019 | 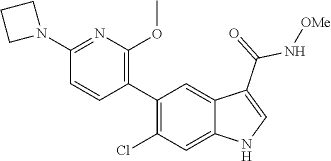 [SMILES: CONC(=O)c1c[nH]c2cc(Cl)c(-c3ccc(N4CCC4)nc3OC)cc12] |
| 1023 | 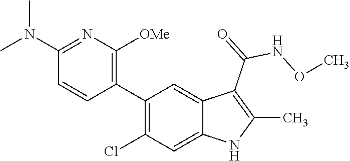 |
| 1024 | 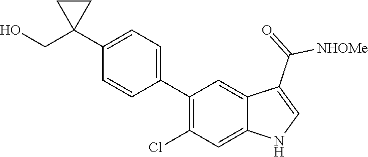 |
| 1025 | 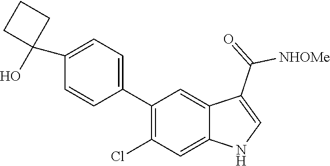 |
| 1026 | 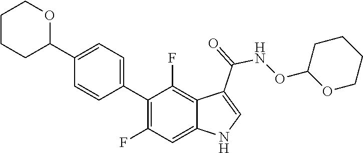 [SMILES: O=C(NOC1CCCCO1)c1c[nH]c2cc(F)c(-c3ccc(C4CCCCO4)cc3)c(F)c12] |
| 1078 | 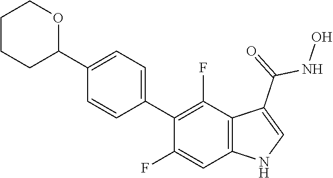 [SMILES: O=C(NO)c1c[nH]c2cc(F)c(-c3ccc(C4CCCCO4)cc3)c(F)c12] |
| 1084 | 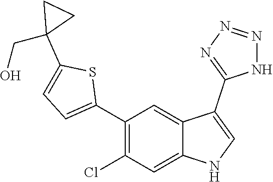 [SMILES: OCC1(c2ccc(-c3cc4c(-c5nnn[nH]5)c[nH]c4cc3Cl)s2)CC1] |

In some embodiments, the compound of formula (I) is selected from:

| Cpd. | |
|---|---|
| # | |
| 1003 | 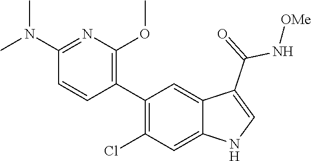 [SMILES: CONC(=O)c1c[nH]c2cc(Cl)c(-c3ccc(N(C)C)nc3OC)cc12] |
| 1007 | 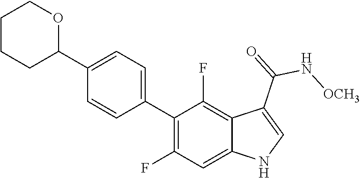 [SMILES: CONC(=O)c1c[nH]c2cc(F)c(-c3ccc(C4CCCCO4)cc3)c(F)c12] |
| 1024 | 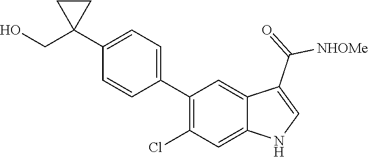 |
| 1044 | 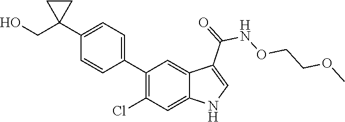 [SMILES: COCCONC(=O)c1c[nH]c2cc(Cl)c(-c3ccc(C4(CO)CC4)cc3)cc12] |
| 1077 | 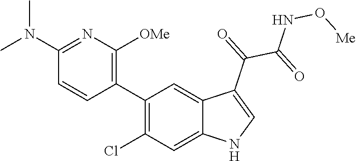 [SMILES: CONC(=O)C(=O)c1c[nH]c2cc(Cl)c(-c3ccc(N(C)C)nc3OC)cc12] |
| 1078 | 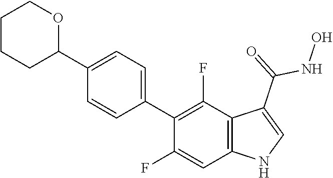 [SMILES: O=C(NO)c1c[nH]c2cc(F)c(-c3ccc(C4CCCCO4)cc3)c(F)c12] |
| 1081 | 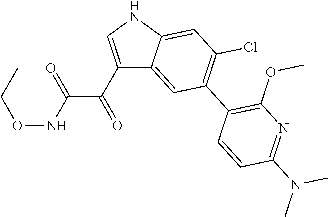 [SMILES: CCONC(=O)C(=O)c1c[nH]c2cc(Cl)c(-c3ccc(N(C)C)nc3OC)cc12] |
| 1084 | 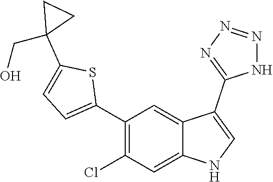 [SMILES: OCC1(c2ccc(-c3cc4c(-c5nnn[nH]5)c[nH]c4cc3Cl)s2)CC1] |
| 1085 | 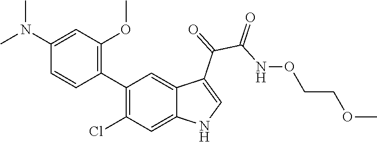 |
| 1088 | 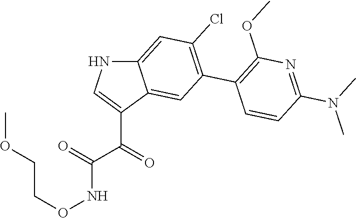 [SMILES: COCCONC(=O)C(=O)c1c[nH]c2cc(Cl)c(-c3ccc(N(C)C)nc3OC)cc12] |
| 1112 | 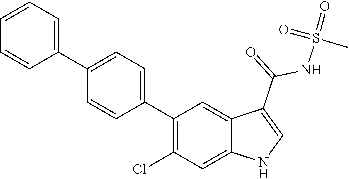 [SMILES: CS(=O)(=O)NC(=O)c1c[nH]c2cc(Cl)c(-c3ccc(-c4ccccc4)cc3)cc12] |
| 1113 | 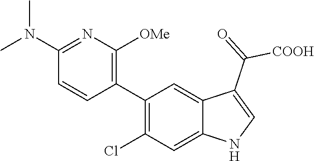 [SMILES: COc1nc(N(C)C)ccc1-c1cc2c(C(=O)C(=O)O)c[nH]c2cc1Cl] |

or a pharmaceutically acceptable salt, solvate, hydrate, cocrystal, or prodrug thereof.

In some embodiments, the disclosure provides a compound having any one of formula (I), formula (10), formula (11), formula (12), formula (13), formula (14), formula (15), formula (16), formula (17), formula (100), formula (110), formula (120), formula (130), formula (140), formula (150), formula (160), (formula 170), formulas 1001 to 1114, but excluding

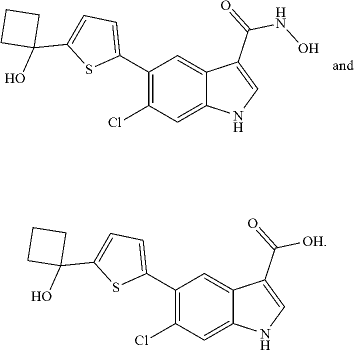 [SMILES: O=C(NO)c1c[nH]c2cc(Cl)c(-c3ccc(C4(O)CCC4)s3)cc12 ; O=C(O)c1c[nH]c2cc(Cl)c(-c3ccc(C4(O)CCC4)s3)cc12]

In some embodiments, the disclosure provides a compound of formula (I), wherein when A is a 5-membered ring heterocycle and R₁ is

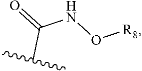

then R₈ is not H.

### Methods of Treatment

The compounds and compositions described herein can be used in methods for treating diseases, disorders, dysfunctions, and/or conditions, including but not limited to: a method of treating a condition by activating AMPK activity in a patient in need of said treatment, the method comprising administering to the patient a therapeutically effective amount of a compound having a formula of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salts, solvates, hydrates, cocrystals, or prodrugs thereof; a method of treating a patient with a mitochondrial disorder and/or dysfunction, the method comprising a therapeutically effective amount of a compound having a formula of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salts, solvates, hydrates, cocrystals, or prodrugs thereof; and a method of treating a patient with a mitochondrial disorder and/or dysfunction the method comprising identifying a mitochondrial dysfunction in an individual, and administering a compound having a formula of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salts, solvates, hydrates, cocrystals, or prodrugs thereof.

The compounds and compositions described herein can be used in methods for treating a disease, disorder, condition, and/or dysfunction, including but not limited to: a method of treating a disease or disorder associated with AMPK activity in the patient, the method comprising modulating AMPK activity in the patient; and a method of treating a mitochondrial disorder and/or dysfunction, the method comprising identifying a mitochondrial disorder and/or dysfunction in an patient, and modulating AMPK activity in the patient. In some embodiments, modulating AMPK activity comprises comprising administering to the patient a therapeutically effective amount of a compound having a formula of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salts, solvates, hydrates, cocrystals, or prodrugs thereof. In some embodiments, the disease or disorder associated with AMPK activity is a mitochondrial disorder and/or dysfunction. In some embodiments, the modulating step comprises activating AMPK in the patient. In some embodiments, the activating step comprises phosphorylating AMPK or providing an agonist to AMPK. In some embodiments, the modulating step comprises inhibiting AMPK in the subject. In some embodiments, the mitochondrial disorder and/or dysfunction is a primary mitochondrial disorder and/or dysfunction. In some embodiments, the mitochondrial disorder and/or dysfunction is a secondary mitochondrial disorder and/or dysfunction. In some embodiments, the method further comprising assessing the efficacy of the compound in the individual.

In some embodiments, the mitochondrial disorder and/or dysfunction is a primary mitochondrial disorder and/or dysfunction. Non-limiting examples of primary mitochondrial dysfunction include Autosomal Dominant Optic Atrophy (ADOA), Alpers-Huttenlocher syndrome (nDNA defect), Ataxia neuropathy syndrome, (nDNA defect), Barth syndrome/Lethal Infantile Cardiomyopathy (LIC), Co-enzyme Q deficiency, Complex I, complex II, complex III, complex IV and complex V deficiencies (either single deficiencies or any combination of deficiency), Chronic progressive external ophthalmoplegia (CPEO), Diabetes mellitus and deafness, Kearns-Sayre syndrome (mtDNA defect), Leukoencephalopathy with Brainstem and Spinal Cord Involvement and Lactate Elevation (LBSL-leukodystrophy), Leigh syndrome (mtDNA and nDNA defects), Leber's hereditary optic neuropathy (LHON), Luft Disease, Mitochondrial myopathy, encephalopathy, lactic acidosis, and stroke syndrome (MELAS) (mtDNA defect), Mitochondrial Enoyl CoA Reductase Protein-Associated Neurodegeneration (MEPAN), Myoclonic epilepsy with ragged red fibers (MERRF), mitochondrial recessive ataxia syndrome (MIRAS), mtDNA deletion syndrome, mtDNA Depletion syndrome, mtDNA maintenance disorders, mtDNA/RNA translation defects, Mitochondrial tRNA synthetase deficiencies, Mitochondrial Myopathy, Mitochondrial neurogastrointestinal encephalopathy syndrome (MNGIE), Neurogenic muscle weakness, ataxia, and retinitis pigmentosa (NARP), Pearson syndrome, Pyruvate dehydrogenase complex deficiency (PDCD/PDH), DNA polymerase gamma deficiency (POLG), Pyruvate carboxylase deficiency, and Thymidine kinase 2 deficiency (TK2).

In some embodiments, the mitochondrial disorder and/or dysfunction is a secondary mitochondrial disorder and/or dysfunction. Non-limiting examples of secondary mitochondrial dysfunction include age-related macular degeneration (AMD), Amyotrophic Lateral Sclerosis (ALS), Alzheimer's disease (AD) and other dementias, Friedreich's ataxia (FA), Huntington's disease (HD), Motor neuron diseases (MND), N-glycanase deficiency (NGLY1), Organic acidemias, Parkinson's disease (PD) and PD-related disorders, Prion disease, Spinal muscular atrophy (SMA), Spinocerebellar ataxia (SCA), Becker muscular dystrophy, Congenital muscular dystrophies, Duchenne muscular dystrophy, Emery-Dreifuss muscular dystrophy, Facioscapulohumeral muscular dystrophy, Myotonic dystrophy, Oculopharyngeal muscular dystrophy, Charcot-Marie-Tooth disease, Congenital myopathies, Distal myopathies, Endocrine myopathies (hyperthyroid myopathy, hypothyroid myopathy), Giant axonal neuropathy, Hereditary spastic paraplegia, Inflammatory myopathies (dermatomyositis, inclusion-body myositis, polymyositis), Metabolic myopathies, Neuromuscular junction diseases, Autism, Cancer, Diabetes, Metabolic syndrome, Chronic fatigue syndrome, an inflammatory disorder, arthritis, aging, and mitochondrial epilepsy (epilepsy secondary to primary mitochondrial disease).

In some embodiments, the compounds and compositions described herein can be used in methods for treating a disease, disorder, condition, and/or dysfunction selected from the treatment of N-glycanase (NGLY1) deficiency, age-related macular degeneration (AMD), ischemic stroke, muscular dystrophies (e.g., Duchenne and Becker), Friedreich ataxia (FA), autoimmune disorders with muscle involvement (e.g., inclusion body myositis, Polymyositis, and Dermatomyositis), and/or neurodegenerative disorders (e.g., Amyotrophic Lateral Sclerosis (ALS), Parkinson's Disease, and Alzheimer's Disease), diabetes, metabolic disorder, and/or obesity. In other embodiments, mitochondrial dysfunction will be identified based on molecular signatures of disease or dysfunction, such that protein blots (western blots), polymerase chain reaction (PCR), genotyping using genetic markers (e.g., single nucleotide polymorphisms (SNPs), expressed sequence tags (ESTs), simple sequence repeats (SSRs), etc.) will identify a particular disease or dysfunction present in the individual. In some embodiments, AMPK activation in cardiac tissue can result in reversible cardiac hypertrophy, thus in some embodiments, the compounds and compositions described herein can be used in methods for treating a disease, disorder, condition, and/or dysfunction associated with dilated cardiomyopathy. (See, e.g., Arad et al, Circ Res. 2007 Mar. 2; 100(4):474-88; Myers et al, Science. 2017 Aug. 4; 357(6350):507-511; the disclosures of which are incorporated herein by reference in their entireties).

In some embodiments, the compounds and compositions described herein can be used in methods for treating a disease, disorder, condition, and/or dysfunction including, but not limited to ophthalmic diseases associated with mitochondrial dysfunction.

In some embodiments, the compounds and compositions described herein provide neuroprotection in individuals with ischemic stroke, improve motor performance in individuals with mitochondrial dysfunction as well as muscle wasting diseases, such as muscular dystrophies and autoimmune myositis disorders, enhance strength, endurance, and overall locomotor function in muscle-degenerative disorders associated with mitochondrial dysfunction, increasing mitochondrial function and/or glycogen storage in skeletal muscle, normalizing energy levels within skeletal muscle, thereby preventing degeneration and weakness, and/or activating AMPK in skeletal muscle.

In some embodiments, the compound having a formula of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salts, solvates, hydrates, cocrystals, or prodrugs thereof is administered in a pharmaceutical formulation. In some embodiments, the pharmaceutical formulation comprises the compound and at least one selected from a binding agent, a lubricating agent, a buffer, and a coating. In some embodiments, the compound and/or pharmaceutical formulation is administered orally. In some embodiments, the compound and/or pharmaceutical formulation is administered daily for at least one week. In some embodiments, the compound and/or pharmaceutical formulation is administered by oral administration, subcutaneous administration, intravenous administration, intraperitoneal administration, intranasal administration, dermal administration, intravitreal injection, or inhalation.

Efficacy of the methods, compounds, and combinations of compounds described herein in treating, preventing and/or managing the indicated diseases or disorders can be tested using various animal models known in the art. For example, methods for determining efficacy of compounds of the disclosure include, but are not limited to, measuring pACC in a sample, as in indicator of increasing AMPK activity. In some embodiments, methods for assessing the disease or disorder symptoms, include, but are not limited to, looking at molecular profiles, such as genotyping, gene expression, and other methods as would be understood by one of ordinary skill in the art. In some embodiments, the diseases and disorders are identified based on symptoms exhibited by an individual. In some embodiments, diseases and disorders are identified based on non-limiting methods including molecular signatures of disease or dysfunction, such that protein blots (western blots), polymerase chain reaction (PCR), genotyping using genetic markers (e.g., single nucleotide polymorphisms (SNPs), expressed sequence tags (ESTs), simple sequence repeats (SSRs), etc.).

### Pharmaceutical Compositions

In an embodiment, the disclosure provides a pharmaceutical composition for use in the treatment of the diseases and conditions described herein.

The pharmaceutical compositions are typically formulated to provide a therapeutically effective amount of a compound of any of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salts, solvates, hydrates, cocrystals, or prodrugs thereof, as described herein, as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

The pharmaceutical compositions described above are for use in the treatment of, without limitation, a mitochondrial dysfunction, the pharmaceutical composition comprising one or more compounds, or pharmaceutically acceptable salts, solvates, hydrates, cocrystals, or prodrugs thereof, having any one of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, and a pharmaceutically acceptable carrier. In some embodiments, the mitochondrial dysfunction is a primary mitochondrial dysfunction. In some embodiments, selected from the group consisting of Autosomal Dominant Optic Atrophy (ADOA), Alpers-Huttenlocher syndrome (nDNA defect), Ataxia neuropathy syndrome, (nDNA defect), Barth syndrome/Lethal Infantile Cardiomyopathy (LIC), Co-enzyme Q deficiency, Complex I, complex II, complex III, complex IV and complex V deficiencies (either single deficiencies or any combination of deficiency), Chronic progressive external ophthalmoplegia (CPEO), Diabetes mellitus and deafness, Kearns-Sayre syndrome (mtDNA defect), Leukoencephalopathy with Brainstem and Spinal Cord Involvement and Lactate Elevation (LBSL-leukodystrophy), Leigh syndrome (mtDNA and nDNA defects), Leber's hereditary optic neuropathy (LHON), Luft Disease, Mitochondrial myopathy, encephalopathy, lactic acidosis, and stroke syndrome (MELAS) (mtDNA defect), Mitochondrial Enoyl CoA Reductase Protein-Associated Neurodegeneration (MEPAN), Myoclonic epilepsy with ragged red fibers (MERRF), mitochondrial recessive ataxia syndrome (MIRAS), mtDNA deletion syndrome, mtDNA Depletion syndrome, mtDNA maintenance disorders, mtDNA/RNA translation defects, Mitochondrial tRNA synthetase deficiencies, Mitochondrial Myopathy, Mitochondrial neurogastrointestinal encephalopathy syndrome (MNGIE), Neurogenic muscle weakness, ataxia, and retinitis pigmentosa (NARP), Pearson syndrome, Pyruvate dehydrogenase complex deficiency (PDCD/PDH), DNA polymerase gamma deficiency (POLG), Pyruvate carboxylase deficiency, and Thymidine kinase 2 deficiency (TK2). In some embodiments, the mitochondrial dysfunction is a secondary mitochondrial dysfunction. In some embodiments, the secondary mitochondrial dysfunction is selected from the group consisting of age-related macular degeneration (AMD), Amyotrophic Lateral Sclerosis (ALS), Alzheimer's disease (AD) and other dementias, Friedreich's ataxia (FA), Huntington's disease (HD), Motor neuron diseases (MND), N-glycanase deficiency (NGLY1), Organic acidemias, Parkinson's disease (PD) and PD-related disorders, Prion disease, Spinal muscular atrophy (SMA), Spinocerebellar ataxia (SCA), Becker muscular dystrophy, Congenital muscular dystrophies, Duchenne muscular dystrophy, Emery-Dreifuss muscular dystrophy, Facioscapulohumeral muscular dystrophy, Myotonic dystrophy, Oculopharyngeal muscular dystrophy, Charcot-Marie-Tooth disease, Congenital myopathies, Distal myopathies, Endocrine myopathies (hyperthyroid myopathy, hypothyroid myopathy), Giant axonal neuropathy, Hereditary spastic paraplegia, Inflammatory myopathies (dermatomyositis, inclusion-body myositis, polymyositis), Metabolic myopathies, Neuromuscular junction diseases, Autism, Cancer, Diabetes, Metabolic syndrome, Chronic fatigue syndrome, an inflammatory disorder, arthritis, aging, and mitochondrial epilepsy (epilepsy secondary to primary mitochondrial disease).

In some embodiments, the concentration of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, provided in the pharmaceutical compositions of the disclosure is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, provided in the pharmaceutical compositions of the disclosure is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, provided in the pharmaceutical compositions of the disclosure is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, provided in the pharmaceutical compositions of the disclosure is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of a compound formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, provided in the pharmaceutical compositions of the disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, provided in the pharmaceutical compositions of the disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the compounds provided according to the disclosure is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Oral Administration

In preferred embodiments, the disclosure provides a pharmaceutical composition for oral administration containing: a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, and a pharmaceutical excipient suitable for administration.

In preferred embodiments, the disclosure provides a solid pharmaceutical composition for oral administration containing: (i) an effective amount of: a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, and (ii) a pharmaceutical excipient suitable for administration. In some embodiments, the composition further contains (iii) an effective amount of an additional active pharmaceutical ingredient. For example, additional active pharmaceutical ingredients, as used herein, may include one or more compounds that modulate AMPK activity. In some embodiments, the one or more compounds activate AMPK activity. In some embodiments, the one or more compounds phosphorylate AMPK and/or are AMPK agonists. In some embodiments, the one or more compounds inhibit AMPK activity. In some embodiments, the one or more compounds are competitive inhibitors and/or an allosteric inhibitors, which prevent AMPK from catalyzing a reaction.

In some embodiments, the pharmaceutical composition may be a liquid pharmaceutical composition suitable for oral consumption.

Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, 6-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the disclosure provides a pharmaceutical composition for injection containing: a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, and a pharmaceutical excipient suitable for injection. Components and amounts of compounds in the compositions are as described herein.

The forms in which the compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating a compound formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In preferred embodiments, the disclosure provides a pharmaceutical composition for transdermal delivery containing: a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of: a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, or a pharmaceutical composition of these compounds can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, can also be administered intraadiposally or intrathecally.

The compositions of the disclosure may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the disclosure may slow or inhibit the migration and proliferation of smooth muscle cells in the arterial wall which contribute to restenosis. A compound of the disclosure may be administered, for example, by local delivery from the struts of a stent, from a stent graft, from grafts, or from the cover or sheath of a stent. In some embodiments, a compound of the disclosure is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. A compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the disclosure in a suitable solvent, followed by evaporation of the solvent. Excess drug on the surface of the stent may be removed via an additional brief solvent wash. In yet other embodiments, compounds of the disclosure may be covalently linked to a stent or graft. A covalent linker may be used which degrades in vivo, leading to the release of the compound of the disclosure. Any bio-labile linkage may be used for such a purpose, such as ester, amide or anhydride linkages. A compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, may additionally be administered intravascularly from a balloon used during angioplasty. Extravascular administration of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, via the pericard or via advential application of formulations of the disclosure may also be performed to decrease restenosis.

Exemplary parenteral administration forms include solutions or suspensions of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, in sterile aqueous solutions, for example, aqueous propylene glycol or dextrose solutions. Such dosage forms can be suitably buffered, if desired.

The disclosure also provides kits. The kits include a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In some embodiments, the compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, and another active pharmaceutical ingredient are provided as separate compositions in separate containers within the kit. In some embodiments, the compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The kits described above are preferably for use in the treatment of the diseases and conditions described herein. In some embodiments, the kits described herein are for use in the treatment of a mitochondrial dysfunction. In some embodiments, the mitochondrial dysfunction is a primary mitochondrial dysfunction. In some embodiments, selected from the group consisting of Autosomal Dominant Optic Atrophy (ADOA), Alpers-Huttenlocher syndrome (nDNA defect), Ataxia neuropathy syndrome, (nDNA defect), Barth syndrome/Lethal Infantile Cardiomyopathy (LIC), Co-enzyme Q deficiency, Complex I, complex II, complex III, complex IV and complex V deficiencies (either single deficiencies or any combination of deficiency), Chronic progressive external ophthalmoplegia (CPEO), Diabetes mellitus and deafness, Kearns-Sayre syndrome (mtDNA defect), Leukoencephalopathy with Brainstem and Spinal Cord Involvement and Lactate Elevation (LBSL-leukodystrophy), Leigh syndrome (mtDNA and nDNA defects), Leber's hereditary optic neuropathy (LHON), Luft Disease, Mitochondrial myopathy, encephalopathy, lactic acidosis, and stroke syndrome (MELAS) (mtDNA defect), Mitochondrial Enoyl CoA Reductase Protein-Associated Neurodegeneration (MEPAN), Myoclonic epilepsy with ragged red fibers (MERRF), mitochondrial recessive ataxia syndrome (MIRAS), mtDNA deletion syndrome, mtDNA Depletion syndrome, mtDNA maintenance disorders, mtDNA/RNA translation defects, Mitochondrial tRNA synthetase deficiencies, Mitochondrial Myopathy, Mitochondrial neurogastrointestinal encephalopathy syndrome (MNGIE), Neurogenic muscle weakness, ataxia, and retinitis pigmentosa (NARP), Pearson syndrome, Pyruvate dehydrogenase complex deficiency (PDCD/PDH), DNA polymerase gamma deficiency (POLG), Pyruvate carboxylase deficiency, and Thymidine kinase 2 deficiency (TK2). In some embodiments, the mitochondrial dysfunction is a secondary mitochondrial dysfunction. In some embodiments, the secondary mitochondrial dysfunction is selected from the group consisting of age-related macular degeneration (AMD), Amyotrophic Lateral Sclerosis (ALS), Alzheimer's disease (AD) and other dementias, Friedreich's ataxia (FA), Huntington's disease (HD), Motor neuron diseases (MND), N-glycanase deficiency (NGLY1), Organic acidemias, Parkinson's disease (PD) and PD-related disorders, Prion disease, Spinal muscular atrophy (SMA), Spinocerebellar ataxia (SCA), Becker muscular dystrophy, Congenital muscular dystrophies, Duchenne muscular dystrophy, Emery-Dreifuss muscular dystrophy, Facioscapulohumeral muscular dystrophy, Myotonic dystrophy, Oculopharyngeal muscular dystrophy, Charcot-Marie-Tooth disease, Congenital myopathies, Distal myopathies, Endocrine myopathies (hyperthyroid myopathy, hypothyroid myopathy), Giant axonal neuropathy, Hereditary spastic paraplegia, Inflammatory myopathies (dermatomyositis, inclusion-body myositis, polymyositis), Metabolic myopathies, Neuromuscular junction diseases, Autism, Cancer, Diabetes, Metabolic syndrome, Chronic fatigue syndrome, an inflammatory disorder, arthritis, aging, and mitochondrial epilepsy (epilepsy secondary to primary mitochondrial disease).

### Dosages and Dosing Regimens

The amounts of: a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, administered will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, may be provided in units of mg/kg of body mass or in mg/m² of body surface area.

In some embodiments, a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein is administered in multiple doses. In a preferred embodiment, a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein is administered in multiple doses. Dosing may be once, twice, three times, four times, five times, six times, or more than six times per day. Dosing may be once a month, once every two weeks, once a week, or once every other day. In other embodiments, a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, is administered about once per day to about 6 times per day. In some embodiments, a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, is administered once daily, while in other embodiments, a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein is administered twice daily, and in other embodiments a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, is administered three times daily.

Administration a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, may continue as long as necessary. In some embodiments, a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, is administered for more than 1, 2, 3, 4, 5, 6, 7, 14, or 28 days. In some embodiments, a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein is administered for less than 28, 14, 7, 6, 5, 4, 3, 2, or 1 day. In some embodiments, a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein is administered chronically on an ongoing basis—e.g., for the treatment of chronic effects. In another embodiment, the administration of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, continues for less than about 7 days. In yet another embodiment, the administration continues for more than about 6, 10, 14, 28 days, two months, six months, or one year. In some cases, continuous dosing is achieved and maintained as long as necessary.

In some embodiments, an effective dosage of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg.

In some embodiments, an effective dosage of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of a compound of formula (I), formulas (10) to (17), formulas (20) to (26), formulas (100) to (190), formulas (200) to (270), formulas (300) to (370), formulas (400) to (470), formulas (500) to (570), formulas (600) to (670), formulas (700) to (770), formulas (800) to (870), formulas (900) to (970), formulas 1001 to 1114, or pharmaceutically acceptable salt thereof, described herein, may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Synthesis of Compounds of the Disclosure

### General Synthesis Route R-1

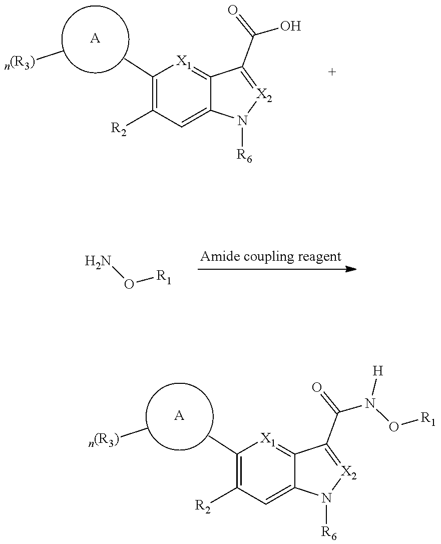

Any amide coupling reagent known in the art is of use in effecting the coupling of the indole to the hydroxylamine. In an exemplary embodiment, the reagent is selected from BOP—Cl, TBTU, BOP, PyBop, HATU, EDCI/HOBT, DIC/HOBT; and DCC/HOBT.

### A. Synthesis of 6-chloro-5-(6-(dimethylamino)-2-methoxypyridin-3-yl)-N-methoxy-1H-indole-3-carboxamide (1003)

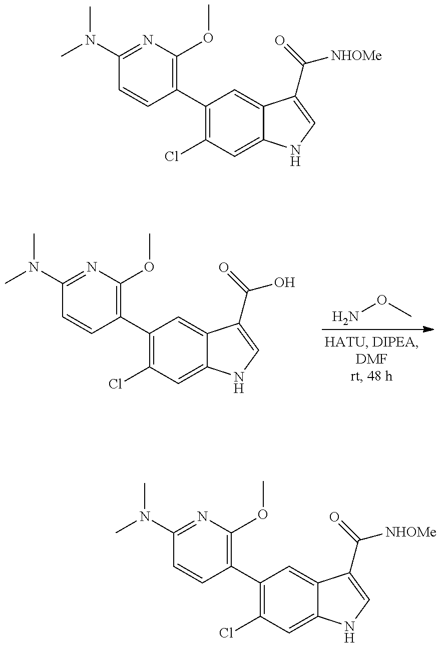

Compound 1003 was prepared according to General Synthesis Route R-1. DIPEA (0.4 mL, 1.734 mmol) and HATU (0.34 g, 0.867 mmol) were added to a solution of 6-chloro-5-(6-(dimethylamino)-2-methoxypyridin-3-yl)-1H-indole-3-carboxylic acid (0.20 g, 0.578 mmol) in DMF (10 mL) at RT. O-methylhydroxylamine (0.06 g, 0.693 mmol) was then added and the reaction mass was stirred for 48 h. The reaction was diluted with ethyl acetate, washed with water and the organic layer was dried over anhydrous sodium sulfate, concentrated under reduced pressure to a crude product which was purified by Prep. HPLC to give 6-chloro-5-(6-(dimethylamino)-2-methoxypyridin-3-yl)-N-methoxy-1H-indole-3-carboxamide as an off-white solid (14 mg, 11%).

LCMS: 99.81% (M/Z 373.15, [M−H]⁺)

¹H NMR; 400 MHz, DMSO-d6: δ 11.42 (br, 2H), 7.93 (s, 1H), 7.92 (s, 1H), 7.55 (s, 1H), 7.31 (d, J=8.40 Hz, 1H), 6.22 (d, J=8.00 Hz, 1H), 3.76 (s, 3H), 3.69 (s, 3H), 3.07 (s, 6H).

### B. Synthesis of 6-chloro-5-(6-(dimethylamino)-2-methoxypyridin-3-yl)-N-ethoxy-1H-indole-3-carboxamide (1009)

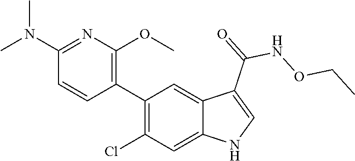 [SMILES: CCONC(=O)c1c[nH]c2cc(Cl)c(-c3ccc(N(C)C)nc3OC)cc12]

Compound 1009 was prepared according to General Synthesis Route R-1 using a similar experimental procedure as used in the preparation of 6-chloro-5-(6-(dimethylamino)-2-methoxypyridin-3-yl)-N-methoxy-1H-indole-3-carboxamide (Compound 1003).

LCMS: 99.12% (M/Z 387.26, [M−H])

¹H NMR 400 MHz, DMSO-d6: δ 11.65-11.15 (br s, 2H), 7.95 (s, 1H), 7.92 (s, 1H), 7.56 (s, 1H), 7.31 (d, J=8.00 Hz, 1H), 6.22 (d, J=8.40 Hz, 1H), 3.93-3.88 (q, J=6.8 Hz, 2H), 3.76 (s, 3H), 3.07 (s, 6H), 1.20 (t, J=7.2 Hz, 3H).

### C. Synthesis of 6-chloro-5-(6-(dimethylamino)-2-methoxypyridin-3-yl)-N-isopropoxy-1H-indole-3-carboxamide (1010)

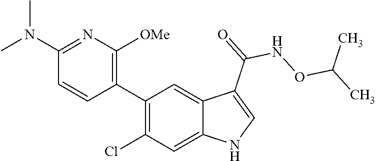 [SMILES: COc1nc(N(C)C)ccc1-c1cc2c(C(=O)NOC(C)C)c[nH]c2cc1Cl]

Compound 1010 was prepared according to General Synthesis Route R-1 using a similar experimental procedure as used in the preparation of 6-chloro-5-(6-(dimethylamino)-2-methoxypyridin-3-yl)-N-methoxy-1H-indole-3-carboxamide (Compound 1003).

LCMS: 99.16% (M/Z 403.34, [M+H]+)

¹H NMR: 400 MHz, DMSO-d6: δ 11.66 (s, 1H), 10.86 (s, 1H), 7.97 (d, J=2.80 Hz, 1H), 7.93 (s, 1H), 7.54 (s, 1H), 7.31 (d, J=8.00 Hz, 1H), 6.22 (d, J=8.00 Hz, 1H), 4.11-4.04 (m, 1H), 3.76 (s, 3H), 3.07 (s, 6H), 1.19 (d, J=6.40 Hz, 6H).

### D. Synthesis of 6-chloro-5-(6-(dimethylamino)-2-methoxypyridin-3-yl)-N-(2-methoxyethoxy)-1H-indole-3-carboxamide (1011)

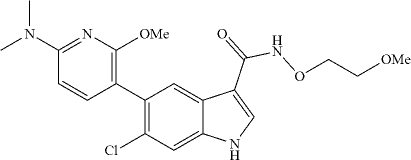 [SMILES: COCCONC(=O)c1c[nH]c2cc(Cl)c(-c3ccc(N(C)C)nc3OC)cc12]

Compound 1011 was prepared according to General Synthesis Route R-1 using a similar experimental procedure as used in the preparation of 6-chloro-5-(6-(dimethylamino)-2-methoxypyridin-3-yl)-N-methoxy-1H-indole-3-carboxamide (Compound 1003).

LCMS: 99.61% (M/Z 419.39, [M+H]+)

¹H NMR: 400 MHz, DMSO-d6: δ 11.68 (s, 1H), 11.12 (s, 1H), 7.98 (s, 1H), 7.93 (s, 1H), 7.54 (s, 1H), 7.31 (d, J=8.00 Hz, 1H), 6.22 (d, J=8.00 Hz, 1H), 3.99 (t, J=4.80 Hz, 2H), 3.76 (s, 3H), 3.57 (t, J=4.40 Hz, 2H), 3.32 (s, 3H), 3.07 (s, 6H).

### E. Synthesis of 6-chloro-5-(6-(dimethylamino)-2-methoxypyridin-3-yl)-N-phenoxy-1H-indole-3-carboxamide (1012)

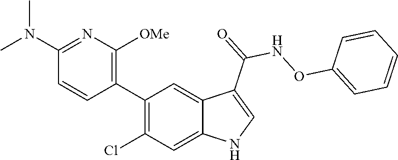 [SMILES: COc1nc(N(C)C)ccc1-c1cc2c(C(=O)NOc3ccccc3)c[nH]c2cc1Cl]

Compound 1012 was prepared according to General Synthesis Route R-1 using a similar experimental procedure as used in the preparation of 6-chloro-5-(6-(dimethylamino)-2-methoxypyridin-3-yl)-N-methoxy-1H-indole-3-carboxamide (Compound 1003).

LCMS: 97.14% (M/Z 437.36, [M+H]+)

¹H NMR: 400 MHz, DMSO-d6: δ 11.83 (s, 2H), 8.10 (s, 1H), 7.92 (s, 1H), 7.59 (s, 1H), 7.35-7.30 (m, 3H), 7.09 (d, J=8.00 Hz, 2H), 7.01 (t, J=7.2 Hz, 1H), 6.21 (d, J=8.40 Hz, 1H), 3.76 (s, 3H), 3.06 (s, 6H).

### F. Synthesis of 6-chloro-N-(cyclohexyloxy)-5-(6-(dimethylamino)-2-methoxypyridin-3-yl)-1H-indole-3-carboxamide (1013)

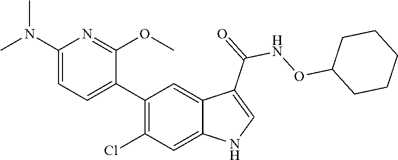 [SMILES: COc1nc(N(C)C)ccc1-c1cc2c(C(=O)NOC3CCCCC3)c[nH]c2cc1Cl]

Compound 1013 was prepared according to General Synthesis Route R-1 using a similar experimental procedure as used in the preparation of 6-chloro-5-(6-(dimethylamino)-2-methoxypyridin-3-yl)-N-methoxy-1H-indole-3-carboxamide (Compound 1003).

LCMS: 96.97% (443.29, M+H);

¹H NMR 400 MHz, DMSO-d6: δ 11.68 (s, 1H), 10.89 (s, 1H), 7.97 (s, 1H), 7.93 (s, 1H), 7.54 (s, 1H), 7.31 (d, J=8.00 Hz, 1H), 6.22 (d, J=8.00 Hz, 1H), 3.79-3.76 (m, 4H), 3.07 (s, 6H), 1.91-1.89 (m, 2H), 1.73-1.71 (m, 2H), 1.39-1.21 (m, 6H).

### G. Synthesis of N-(tert-butoxy)-6-chloro-5-(6-(dimethylamino)-2-methoxypyridin-3-yl)-1H-indole-3-carboxamide (1014)

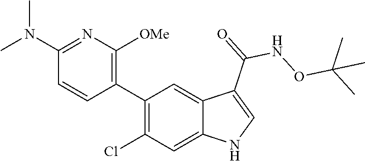 [SMILES: COc1nc(N(C)C)ccc1-c1cc2c(C(=O)NOC(C)(C)C)c[nH]c2cc1Cl]

Compound 1014 was prepared according to General Synthesis Route R-1 using a similar experimental procedure as used in the preparation of 6-chloro-5-(6-(dimethylamino)-2-methoxypyridin-3-yl)-N-methoxy-1H-indole-3-carboxamide (Compound 1003).

LCMS: 99.43% (M/Z 417.41, [M+H]⁺)

¹H NMR 400 MHz, DMSO-d6: δ 11.66 (s, 1H), 10.40 (s, 1H), 8.02 (s, 1H), 7.92 (s, 1H), 7.54 (s, 1H), 7.31 (d, J=8.40 Hz, 1H), 6.22 (d, J=8.40 Hz, 1H), 3.76 (s, 3H), 3.07 (s, 6H), 1.22 (s, 9H).

### H. Synthesis of 6-chloro-N-(cyclopropylmethoxy)-5-(6-(dimethylamino)-2-methoxypyridin-3-yl)-1H-indole-3-carboxamide (1015)

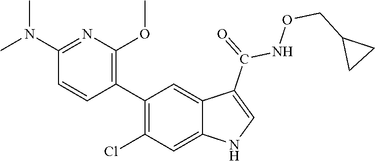 [SMILES: COc1nc(N(C)C)ccc1-c1cc2c(C(=O)NOCC3CC3)c[nH]c2cc1Cl]

Compound 1015 was prepared according to General Synthesis Route R-1 using a similar experimental procedure as used in the preparation of 6-chloro-5-(6-(dimethylamino)-2-methoxypyridin-3-yl)-N-methoxy-1H-indole-3-carboxamide (Compound 1003).

LCMS: 98.86% (M+H=415.69)

¹H NMR (400 MHz, DMSO-d6): δ 11.66 (d, J=2.00 Hz, 1H), 11.05 (s, 1H), 7.94 (t, J=2.40 Hz, 1H), 7.54 (s, 1H), 7.31 (d, J=8.00 Hz, 1H), 6.22 (d, J=8.00 Hz, 1H), 3.76 (s, 3H), 3.69 (d, J=7.20 Hz, 2H), 3.07 (s, 6H), 1.14-1.07 (m, 1H), 0.56-0.54 (m, 2H), 0.34-0.32 (m, 2H).

### I. Synthesis of N-(benzyloxy)-6-chloro-5-(6-(dimethylamino)-2-methoxypyridin-3-yl)-1H-indole-3-carboxamide (1016)

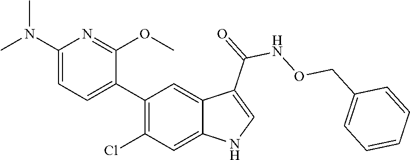 [SMILES: COc1nc(N(C)C)ccc1-c1cc2c(C(=O)NOCc3ccccc3)c[nH]c2cc1Cl]

Compound 1016 was prepared according to General Synthesis Route R-1 using a similar experimental procedure as used in the preparation of 6-chloro-5-(6-(dimethylamino)-2-methoxypyridin-3-yl)-N-methoxy-1H-indole-3-carboxamide (Compound 1003).

LCMS: 95.56% (451.43, M+H);

¹H NMR (400 MHz, DMSO-d6): δ 11.41 (br s, 2H), 7.93 (d, J=3.20 Hz, 2H), 7.55 (s, 1H), 7.45-7.73 (m, 2H), 7.38-7.30 (m, 4H), 6.23 (d, J=8.40 Hz, 1H), 4.91 (s, 2H), 3.77 (s, 3H), 3.07 (s, 6H).

### J. Synthesis of 4,6-difluoro-N-((tetrahydro-2H-pyran-2-yl)oxy)-5-(4-(tetrahydro-2H-pyran-2-yl)phenyl)-1H-indole-3-carboxamide (1026)

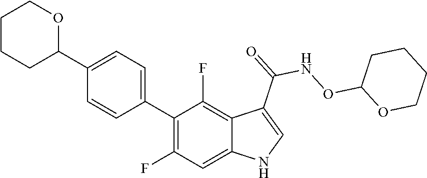 [SMILES: O=C(NOC1CCCCO1)c1c[nH]c2cc(F)c(-c3ccc(C4CCCCO4)cc3)c(F)c12]

Compound 1026 was prepared according to General Synthesis Route R-1 using a similar experimental procedure as used in the preparation of 6-chloro-5-(6-(dimethylamino)-2-methoxypyridin-3-yl)-N-methoxy-1H-indole-3-carboxamide (Compound 1003).

LCMS: 98.78% (M−H=455.38)

¹H NMR (400 MHz, DMSO-d6): δ 11.99 (br, 1H), 7.85 (s, 1H), 7.43-7.37 (m, 4H), 7.23 (d, J=10.40 Hz, 1H), 4.96 (s, 1H), 4.39 (d, J=2.00 Hz, 1H), 4.01-3.98 (m, 2H), 3.59-3.48 (m, 2H), 1.87-1.85 (m, 2H), 1.69-1.60 (m, 4H), 1.57-1.50 (m, 6H).

### General Synthesis Route R-2

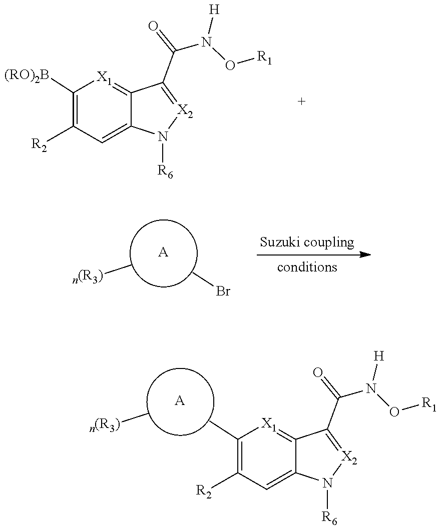

Any Suzuki coupling conditions known in the art is of use in effecting the coupling of the boronic acid/ester to the aryl/heteroaryl halide. In an exemplary embodiment, the Suzuki coupling can be carried out in the presence of a palladium catalyst such as bis(tri-t-butylphosphine)palladium, tetrakis(triphenyl-phosphine)-palladium or a palladacycle catalyst (e.g. the palladacycle catalyst described in Bedford, R. B. and Cazin, C. S. J. (2001) Chem. Commun., 1540-1541) and a base (e.g. a carbonate such as potassium carbonate).

### K. Synthesis of 6-chloro-N-methoxy-5-(2-methoxy-6-(methylamino)pyridin-3-yl)-1H-indole-3-carboxamide (1017)

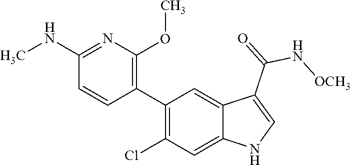 [SMILES: CNc1ccc(-c2cc3c(C(=O)NOC)c[nH]c3cc2Cl)c(OC)n1]

### Step 1: Synthesis of 5-bromo-6-chloro-N-methoxy-1H-indole-3-carboxamide

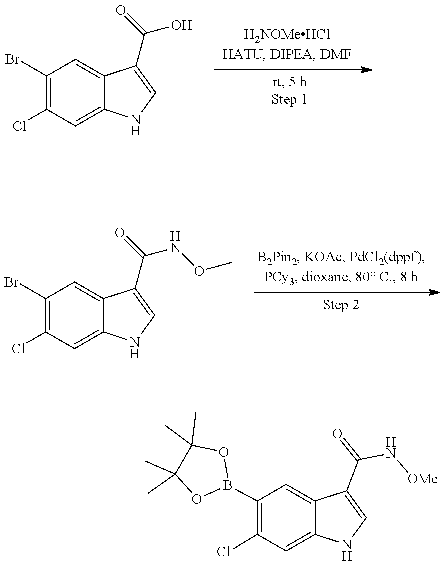 [SMILES: O=C(O)c1c[nH]c2cc(Cl)c(Br)cc12 ; CONC(=O)c1c[nH]c2cc(Cl)c(Br)cc12 ; CONC(=O)c1c[nH]c2cc(Cl)c(B3OC(C)(C)C(C)(C)O3)cc12]

To solution of 5-bromo-6-chloro-1H-indole-3-carboxylic acid (2.0 g, 7.29 mmol) in DMF (20 mL) was added DIPEA (3.82 mL, 21.87 mmol), HATU (4.16 g, 10.94 mmol) and the reaction mixture was stirred for 15 min. Methoxyamine hydrochloride (1.22 g, 14.57 mmol) was added portion wise to the reaction over a period of 15 min and stirred at RT for 5 h. The reaction was diluted with EtOAc (200 mL), washed with water, brine, dried over Na₂SO₄ and concentrated under reduced pressure to afford 5-bromo-6-chloro-N-methoxy-1H-indole-3-carboxamide as off-white solid (1.5 g; 67% yield). LCMS: 84.63% ([M+H]=303.19) which was used directly in the next step.

### Step 2: Synthesis of 6-chloro-N-methoxy-5-(4,4,5,5-tetramethyl-1,3,2-dioxaborolan-2-yl)-1H-indole-3-carboxamide

A stirred solution of the product of Step 1 (1.5 g, 4.96 mmol), Bis-Pin (1.90 g, 7.45 mmol) and KOAc (0.97 g, 9.92 mmol) in dioxane (25 mL) was purged with nitrogen for 10 min. Pd(OAc)₂ (56 mg, 0.248 mmol) and PCy₃ (70 mg, 0.248 mmol) were added and, after again purging with nitrogen the reaction mixture was stirred at 80° C. for 8 h. The reaction mixture was then cooled to RT, diluted with EtOAc and filtered through a bed of celite. The filtrate were concentrated under reduced pressure to afford crude 6-chloro-N-methoxy-5-(4,4,5,5-tetramethyl-1,3,2-dioxaborolan-2-yl)-1H-indole-3-carboxamide which was used directly in the next step.

### Step 3: Synthesis of 6-chloro-N-methoxy-5-(2-methoxy-6-(methylamino)pyridin-3-yl)-1H-indole-3-carboxamide (1017)

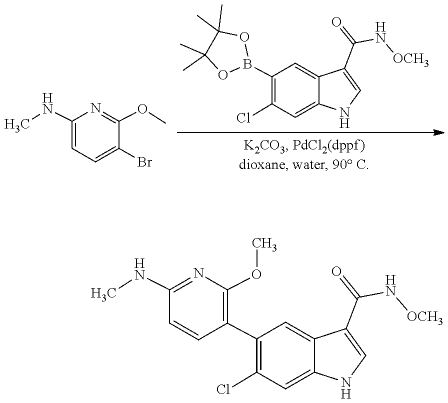

Compound 1017 was prepared according to General Synthesis Route R-2. To a stirred solution of 5-bromo-6-methoxy-N-methylpyridin-2-amine (0.25 g, 1.152 mmol) in 1,4-dioxane (5 mL) and water (1 mL) was added 6-chloro-N-methoxy-5-(4,4,5,5-tetramethyl-1,3,2-dioxaborolan-2-yl)-1H-indole-3-carboxamide (0.5 g, 1.382 mmol) and K₂CO₃ (0.33 g, 2.304 mmol). The reaction mixture was purged with N₂ and PdCl₂(dPPf)₂ and DCM (0.049 g, 0.058 mmol) was added. After stirring, for 5 h at 90° C. the reaction mixture was filtered through a bed of celite and washed through with ethyl acetate. The combined organic layers were concentrated under reduced pressure to obtain a crude product which was purified by preparative HPLC to give 6-chloro-N-methoxy-5-(2-methoxy-6-(methylamino)pyridin-3-yl)-1H-indole-3-carboxamide as an off-white solid (21 mg, 5.06%).

LCMS: 95.87% (361.30, M+H);

¹H NMR 400 MHz, DMSO-d6: δ 11.64 (br s, 1H), 11.17 (br s, 1H), 7.92 (s, 1H), 7.91 (s, 1H), 7.54 (s, 1H), 7.21 (d, J=8.00 Hz, 1H), 6.49-6.45 (m, 1H), 6.06 (d, J=8.00 Hz, 1H), 3.74 (s, 3H), 3.69 (s, 3H), 2.82 (d, J=4.80 Hz, 3H).

### L. Synthesis of 6-chloro-5-(6-(cyclopropylamino)-2-methoxypyridin-3-yl)-N-methoxy-1H-indole-3-carboxamide (1018)

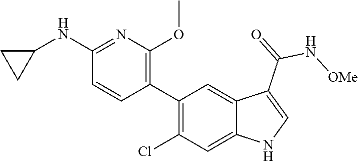 [SMILES: CONC(=O)c1c[nH]c2cc(Cl)c(-c3ccc(NC4CC4)nc3OC)cc12]

Compound 1018 was prepared according to General Synthesis Route R-2 using a similar experimental procedure as used in the preparation of 6-chloro-N-methoxy-5-(2-methoxy-6-(methylamino)pyridin-3-yl)-1H-indole-3-carboxamide (1017).

LCMS: 93.26% (387.34, M+H);

¹H NMR (400 MHz, DMSO-d6): δ 11.83 (br s, 1H), 11.13 (br s, 1H), 7.92 (m, 2H), 7.55 (s, 1H), 7.29 (d, J=7.60 Hz, 1H), 6.74 (s, 1H), 6.26 (d, J=8.00 Hz, 1H), 3.72 (s, 3H), 3.69 (s, 3H), 2.55 (br s, 1H), 0.71-0.70 (m, 2H), 0.48 (br s, 2H).

### M. Synthesis of 5-(6-(azetidin-1-yl)-2-methoxypyridin-3-yl)-6-chloro-N-methoxy-1H-indole-3-carboxamide (1019)

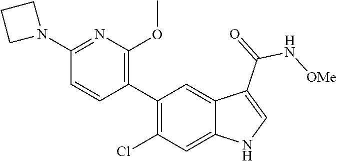 [SMILES: CONC(=O)c1c[nH]c2cc(Cl)c(-c3ccc(N4CCC4)nc3OC)cc12]

Compound 1019 was prepared according to General Synthesis Route R-2 using a similar experimental procedure as used in the preparation of 6-chloro-N-methoxy-5-(2-methoxy-6-(methylamino)pyridin-3-yl)-1H-indole-3-carboxamide (1017).

LCMS: 97.06% (387.34, M+H);

¹H NMR 400 MHz, DMSO-d6: δ 11.35 (br s, 2H), 7.93-7.90 (m, 2H), 7.57 (s, 1H), 7.31 (d, J=8.00 Hz, 1H), 5.96 (d, J=7.60 Hz, 1H), 3.98 (t, J=7.20 Hz, 4H), 3.73 (s, 3H), 3.68 (s, 3H), 2.36-2.32 (m, 2H).

### N. Synthesis of 6-chloro-5-(6-(dimethylamino)-2-ethoxypyridin-3-yl)-N-methoxy-1H-indole-3-carboxamide (1020)

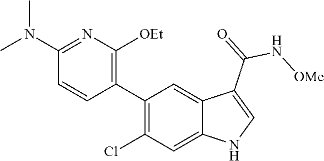 [SMILES: CCOc1nc(N(C)C)ccc1-c1cc2c(C(=O)NOC)c[nH]c2cc1Cl]

Compound 1020 was prepared according to General Synthesis Route R-2 using a similar experimental procedure as used in the preparation of 6-chloro-N-methoxy-5-(2-methoxy-6-(methylamino)pyridin-3-yl)-1H-indole-3-carboxamide (1017).

LCMS: 99.09% (389.36, M+H);

¹H NMR 400 MHz, DMSO-d6: δ 11.29 (br s, 2H), 7.93 (s, 2H), 7.56 (s, 1H), 7.31 (d, J=8.00 Hz, 1H), 6.20 (d, J=8.00 Hz, 1H), 4.28 (q, J=6.80 Hz, 2H), 3.69 (s, 3H), 3.05 (s, 6H), 1.22 (t, J=6.80 Hz, 3H).

### O. Synthesis of 6-chloro-5-(4-(1-(hydroxymethyl)cyclopropyl)phenyl)-N-methoxy-1H-indole-3-carboxamide (1024)

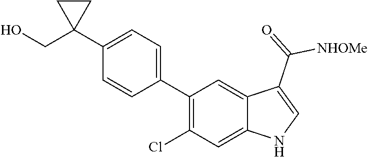

Compound 1024 was prepared according to General Synthesis Route R-2 using a similar experimental procedure as used in the preparation of 6-chloro-N-methoxy-5-(2-methoxy-6-(methylamino)pyridin-3-yl)-1H-indole-3-carboxamide (1017).LCMS: 99.39% ([M+H]=371.34)

¹H NMR (400 MHz, DMSO): δ 11.46 (br s, 2H), 8.01 (s, 1H), 7.96 (s, 1H), 7.62 (s, 1H), 7.38-7.32 (m, 4H), 4.71 (s, 1H), 3.69 (s, 3H), 3.59 (s, 2H), 0.89-0.86 (m, 2H), 0.81-0.78 (m, 2H).

### P. Synthesis of 6-chloro-5-(4-(1-hydroxycyclobutyl)phenyl)-N-methoxy-1H-indole-3-carboxamide (1025)

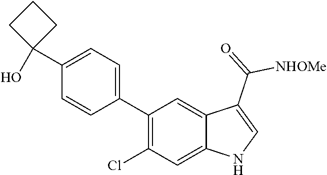

Compound 1025 was prepared according to General Synthesis Route R-2 using a similar experimental procedure as used in the preparation of 6-chloro-N-methoxy-5-(2-methoxy-6-(methylamino)pyridin-3-yl)-1H-indole-3-carboxamide (1017).

LCMS: 97.62% ([M−H]=369.24)

1H NMR (400 MHz, DMSO): δ 11.41 (br s, 2H), 8.04 (s, 1H), 7.97 (s, 1H), 7.64 (s, 1H), 7.56 (d, J=8.40 Hz, 2H), 7.41 (d, J=8.00 Hz, 2H), 5.53 (s, 1H), 3.69 (s, 3H), 2.51-2.42 (m, 2H), 2.34-2.27 (m, 2H), 2.00-1.92 (m, 1H), 1.70-1.60 (m, 1H).

### Q. Synthesis of 6-chloro-5-(6-(dimethylamino)-5-fluoro-2-methoxypyridin-3-yl)-N-methoxy-1H-indole-3-carboxamide (1022)

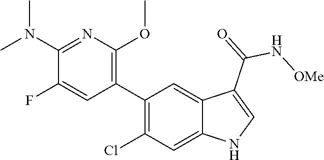 [SMILES: CONC(=O)c1c[nH]c2cc(Cl)c(-c3cc(F)c(N(C)C)nc3OC)cc12]

Compound 1022 was prepared according to General Synthesis Route R-2.

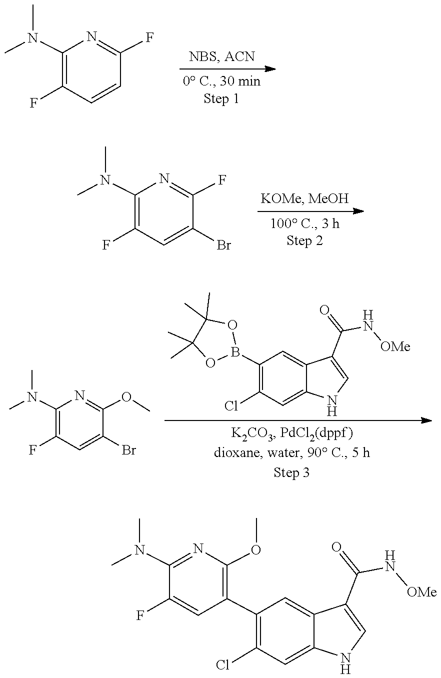

### Step 1: 5-bromo-3,6-difluoro-N,N-dimethylpyridin-2-amine

To a stirred solution of 3,6-difluoro-N,N-dimethylpyridin-2-amine (1 g, 6.323 mmol) in ACN (10 mL) was added NBS (1.125 g, 6.323 mmol) portionwise at RT under nitrogen atmosphere at 0° C. and stirred at 0° C. for 30 min. The reaction mixture was diluted with ethyl acetate (100 mL), washed with brine (100 mL) and concentrated under reduced pressure to give 5-bromo-3,6-difluoro-N,N-dimethylpyridin-2-amine as pale yellow liquid (1.2 g; 80% yield).

### Step 2: 5-bromo-3-fluoro-6-methoxy-N,N-dimethylpyridin-2-amine

To a stirred solution of the product of Step 1 (1.2 g, 0.0051 mol) in DMF (5 mL) was added NaOMe solution (25% in MeOH, 20 mL) and heated at 100° C. for 3 h. The reaction mixture was concentrated under reduced pressure, quenched with chilled water and the precipitated solid was filtered off and dried under vacuum to provide crude 5-bromo-3-fluoro-6-methoxy-N,N-dimethylpyridin-2-amine (1 g; 79%).

Step 3: Coupling of the product of Step 2 with 6-chloro-N-methoxy-5-(4,4,5,5-tetramethyl-1,3,2-dioxaborolan-2-yl)-1H-indole-3-carboxamide as in Example K gave 6-chloro-5-(6-(dimethylamino)-5-fluoro-2-methoxypyridin-3-yl)-N-methoxy-1H-indole-3-carboxamide as a white solid (35 mg; 9% yield).

LCMS: 97.14% (M+H=393.35)

¹H NMR (400 MHz, DMSO-d6): δ 11.73 (s, 1H), 11.14 (s, 1H), 7.95 (s, 2H), 7.57 (s, 1H), 7.35 (d, J=12.80 Hz, 1H), 3.76 (s, 3H), 3.69 (s, 3H), 3.09 (d, J=2.00 Hz, 6H).

### General Synthesis Route R-3

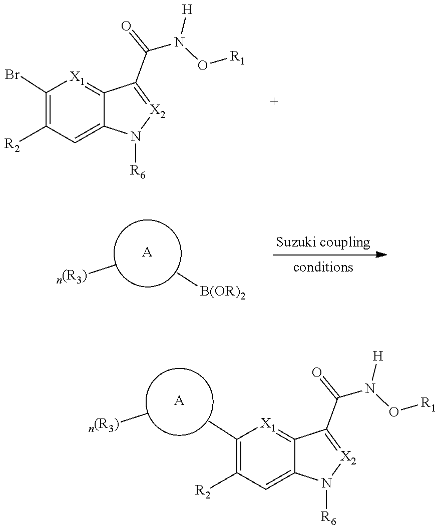

Any Suzuki coupling conditions known in the art is of use in effecting the coupling of the boronic acid/ester to the aryl/heteroaryl halide. In an exemplary embodiment, the Suzuki coupling can be carried out in the presence of a palladium catalyst such as bis(tri-t-butylphosphine)palladium, tetrakis(triphenyl-phosphine)-palladium or a palladacycle catalyst (e.g. the palladacycle catalyst described in Bedford, R. B. and Cazin, C. S. J. (2001) Chem. Commun., 1540-1541) and a base (e.g. a carbonate such as potassium carbonate).

### R. Synthesis of 6-chloro-5-(6-(dimethylamino)-2-methoxypyridin-3-yl)-N-methoxy-2-methyl-1H-indole-3-carboxamide (1023)

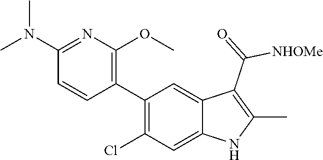

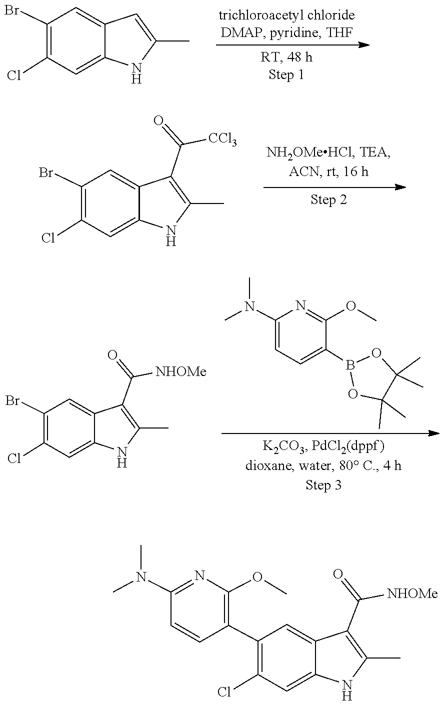

Compound 1023 was prepared according to General synthesis route R-3.

### Step 1: 1-(5-bromo-6-chloro-2-methyl-1H-indol-3-yl)-2,2,2-trichloroethan-1-one

To a stirred solution of 5-bromo-6-chloro-2-methyl-1H-indole (0.5 g, 2.04 mmol) in THE (10 mL) were added DMAP (25 mg, 0.20 mmol) and pyridine (0.6 mL, 7.16 mmol) at RT under nitrogen atmosphere and stirred for 10 min; trichloroacetyl chloride (1.2 mL, 10.22 mmol) was added dropwise and the reaction mixture was stirred at RT for 48 h. The reaction mixture was diluted with water and extracted with EtOAc. The organic layer was washed with water, brine dried over anhydrous Na₂SO₄, filtered and concentrated under reduced pressure to obtain a crude product which was purified by flash column chromatography to give 1-(5-bromo-6-chloro-2-methyl-1H-indol-3-yl)-2,2,2-trichloroethan-1-one as a light red solid (0.55 g; 69%).

### Step 2: 5-bromo-6-chloro-N-methoxy-2-methyl-1H-indole-3-carboxamide

To a stirred solution of the product of Step 1 (0.55 g, 1.41 mmol) in ACN (10 mL) were added O-methylhydroxylamine hydrochloride (0.59 g, 7.05 mmol) and TEA under nitrogen atmosphere and stirred at RT for 16 h. The reaction mixture was diluted with water and extracted with EtOAc. The organic layer was washed with water, brine, dried over Na₂SO₄, filtered and concentrated under reduced pressure to obtain a crude product which was purified by flash column chromatography to give 5-bromo-6-chloro-N-methoxy-2-methyl-1H-indole-3-carboxamide as a brown solid (0.15 g; 33%).

### Step 3: 6-chloro-5-(6-(dimethylamino)-2-methoxypyridin-3-yl)-N-methoxy-2-methyl-1H-indole-3-carboxamide

A stirred solution of the product of Step 2 (150 mg, 0.47 mmol), 6-methoxy-N,N-dimethyl-5-(4,4,5,5-tetramethyl-1,3,2-dioxaborolan-2-yl)pyridin-2-amine (263 mg, 0.96 mmol) and K₂CO₃ (131 mg, 0.96 mmol) in 1,4-dioxane (4 mL) and water (1 mL) was purged with N₂ for 10 min. PdCl₂(dppf).DCM (20 mg, 0.024 mmol) was added and the reaction mixture was stirred at 80° C. for 4 h. The reaction mixture was cooled to rt, filtered through a celite bed, washed through with EtOAc and the combined organic material was concentrated under reduced pressure to obtain a crude product which was purified by prep-HPLC to give 6-methoxy-N,N-dimethyl-5-(4,4,5,5-tetramethyl-1,3,2-dioxaborolan-2-yl)pyridin-2-amine (10 mg, 5%).

LCMS: 99.06% ([M+H]=389.36)

¹H NMR (400 MHz, DMSO): δ 11.63 (s, 1H), 10.70 (s, 1H), 7.57 (s, 1H), 7.40 (s, 1H), 7.32 (d, J=8.00 Hz, 1H), 6.22 (d, J=8.00 Hz, 1H), 3.76 (s, 3H), 3.67 (s, 3H), 3.06 (s, 6H), 2.57 (s, 3H).

### S. Synthesis of 6-chloro-5-(6-(dimethylamino)-2-(trifluoromethyl)pyridin-3-yl)-N-methoxy-1H-indole-3-carboxamide (1021)

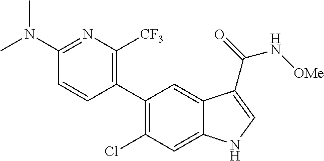 [SMILES: CONC(=O)c1c[nH]c2cc(Cl)c(-c3ccc(N(C)C)nc3C(F)(F)F)cc12]

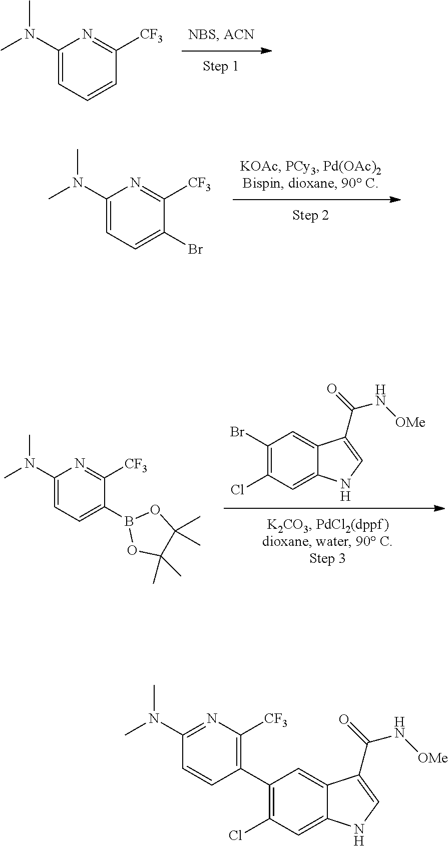

Compound 1021 was prepared according to General Synthesis Route R-3.

### Step 1: 5-bromo-N,N-dimethyl-6-(trifluoromethyl)pyridin-2-amine

To a stirred solution of N,N-dimethyl-6-(trifluoromethyl)pyridin-2-amine (1.9 g, 10.0 mmol) in ACN (20 mL) was added NBS (1.7 g, 10.0 mmol) at 0° C. under nitrogen atmosphere and stirred at RT for 1 h. The solvent was evaporated under reduced pressure to obtain a crude product which was purified by flash column chromatography using neutral alumina to afford 5-bromo-N,N-dimethyl-6-(trifluoromethyl)pyridin-2-amine as a yellowish oil (2.3 g, 83%).

### Step 2: N,N-dimethyl-5-(4,4,5,5-tetramethyl-1,3,2-dioxaborolan-2-yl)-6-(trifluoromethyl)pyridin-2-amine

A solution of the product of Step 1 (250 mg, 268 mmol), Bis-Pin (290 mg, 1.219 mmol) and KOAc (184 mg, 1.9 mmol) in dioxane (10 mL) was purged with nitrogen for 10 min. Pd(OAc)₂ (13 mg, 0.0466 mmol) and PCy₃ (13 mg, 0.0466 mmol) were added and the reaction mixture was stirred at 90° C. for 5 h. The reaction mixture was diluted with EtOAc, filtered through celite and the filtrate concentrated under reduced pressure to obtain crude N,N-dimethyl-5-(4,4,5,5-tetramethyl-1,3,2-dioxaborolan-2-yl)-6-(trifluoromethyl)pyridin-2-amine as pale yellow gum (470 mg). This material was used in next step without purification.

### Step 3: 6-chloro-5-(6-(dimethylamino)-2-(trifluoromethyl)pyridin-3-yl)-N-methoxy-1H-indole-3-carboxamide

A solution of the product of Step 2 (0.300 g, 0.99 mmol), 5-bromo-6-chloro-N-methoxy-1H-indole-3-carboxamide (0.470 g, 1.48 mmol) and K₂CO₃ (0.275 g, 1.98 mmol) in dioxane (10 mL) and water (2 mL) was purged with nitrogen for 10 min. PdCl₂(dppf)·DCM (0.40 g, 0.05 mmol) was added and the reaction mixture was stirred at 90° C. for 5 h after which, it was diluted with EtOAc, filtered through celite and concentrated under reduced pressure to obtain a crude product. Purification by prep-HPLC gave 6-chloro-5-(6-(dimethylamino)-2-(trifluoromethyl)pyridin-3-yl)-N-methoxy-1H-indole-3-carboxamide as a white solid (11 mg, 12%).

LCMS: 98.09% (413.34 [M+H]+)

¹H NMR 400 MHz, DMSO-d6: δ 11.74 (br s, 1H), 11.18 (br s, 1H), 7.96 (m, 2H), 7.61 (s, 1H), 7.48 (d, J=8.80 Hz, 1H), 6.97 (d, J=8.80 Hz, 1H), 3.69 (s, 3H), 3.12 (s, 6H).

### T. Synthesis of 4,6-difluoro-N-methoxy-5-(4-(tetrahydro-2H-pyran-2-yl)phenyl)-1H-indole-3-carboxamide (1007)

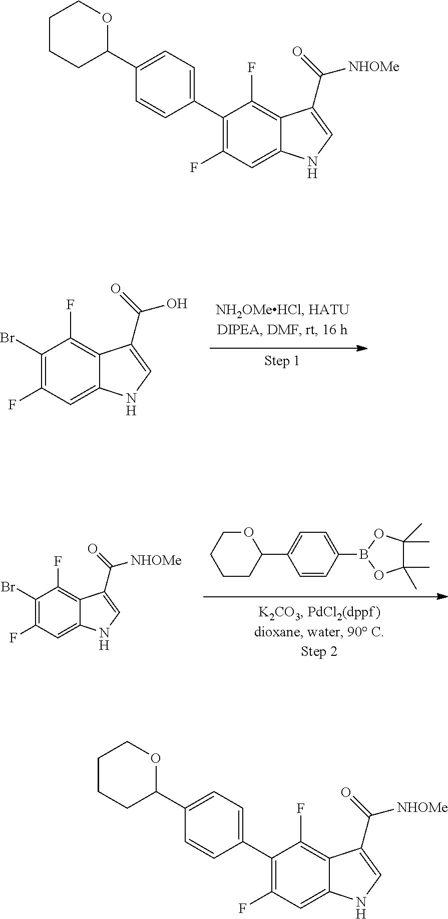

Compound 1007 was prepared according to General Synthesis Route R-3.

### Step 1: 5-bromo-4,6-difluoro-N-methoxy-1H-indole-3-carboxamide

To a solution of 5-bromo-4,6-difluoro-1H-indole-3-carboxylic acid (100 mg, 0.36 mmol) in DMF (5 mL) was added HATU (207 mg, 0.54 mmol) and DIPEA (0.2 ml, 1.1 mmol). After stirring at RT for 5 min, NH₂OMe·HCl (45 mg, 0.55 mmol) was added and the whole was stirred at RT for 16 h. The reaction was diluted with water and the product was extracted into EtOAc. After drying the organic layer was concentrated under reduced pressure to obtain a crude product which was purified by flash column chromatography to give 5-bromo-4,6-difluoro-N-methoxy-1H-indole-3-carboxamide as white solid (70 mg).

### Step 2: 4,6-difluoro-N-methoxy-5-(4-(tetrahydro-2H-pyran-2-yl)phenyl)-1H-indole-3-carboxamide

To a solution of the product of Step 1 (70 mg, 0.23 mmol), 4,4,5,5-tetramethyl-2-(4-(tetrahydro-2H-pyran-2-yl)phenyl)-1,3,2-dioxaborolane (80 mg, 0.27 mmol) and K₂CO₃ (80 mg, 0.5765 mmol) in dioxane (5 mL) and water (1 mL) was purged with nitrogen for 10 min. PdCl₂(dppf)·DCM (9.4 mg, 0.012 mmol) was added and stirred at 90° C. for 4 h. The reaction mixture was diluted with EtOAc filtered through celite and concentrated under reduced pressure to obtain a crude product which was purified by prep-HPLC, to give 4,6-difluoro-N-methoxy-5-(4-(tetrahydro-2H-pyran-2-yl)phenyl)-1H-indole-3-carboxamide as white solid (8 mg).

LCMS: 97.78% (M−H=385.44)

¹H NMR (400 MHz, DMSO-d6): δ 11.10 (s, 1H), 7.85 (s, 1H), 7.44-7.26 (m, 5H), 4.38 (d, J=10.40 Hz, 1H), 4.05 (d, J=10.40 Hz, 1H), 3.66.3.56 (m, 4H), 1.88-1.86 (m, 2H), 1.65-1.47 (m, 4H).

### U. Synthesis of 5-([1,1′-biphenyl]-4-yl)-6-chloro-N-methoxy-1H-indole-3-carboxamide (1008)

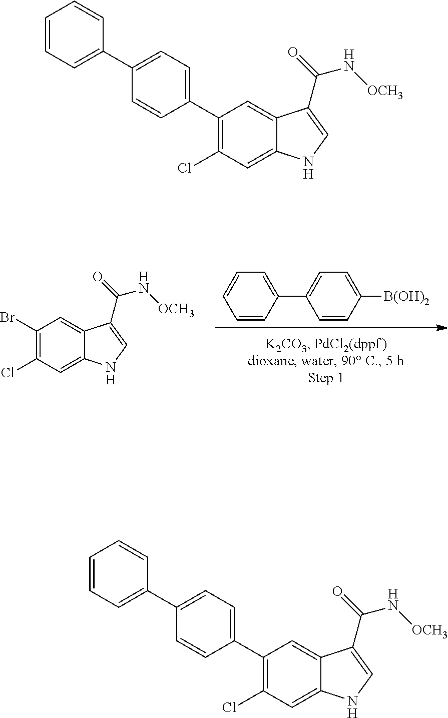 [SMILES: CONC(=O)c1c[nH]c2cc(Cl)c(-c3ccc(-c4ccccc4)cc3)cc12 ; CONC(=O)c1c[nH]c2cc(Cl)c(Br)cc12 ; OB(O)c1ccc(-c2ccccc2)cc1 ; CONC(=O)c1c[nH]c2cc(Cl)c(-c3ccc(-c4ccccc4)cc3)cc12]

Compound 1008 was prepared according to General Synthesis Route R-3.

To a stirred solution of 5-bromo-6-chloro-N-methoxy-1H-indole-3-carboxamide (200 mg, 0.65 mmol) in 1,4-dioxane (10 mL) and water (2 mL), was added [1,1′-biphenyl]-4-ylboronic acid (0.16 g, 0.79 mmol) and K₂CO₃ (0.27 g, 1.98 mmol). The reaction mixture was purged with N₂, then PdCl₂(dPPf)₂·DCM (0.4 g, 0.652 mmol) was added and the reaction mixture was heated at 90° C. for 5 h. The reaction mixture was cooled to 25° C., filtered through a celite bed and concentrated under reduced pressure to obtain a crude product which was purified by preparative HPLC purification to give 5-([1,1′-biphenyl]-4-yl)-6-chloro-N-methoxy-1H-indole-3-carboxamide as a white solid (15 mg, 6.1%).

LCMS: 97.20% (377.14, M+H);

¹H NMR 400 MHz, DMSO-d6: δ 11.48 (br s, 2H), 8.10 (s, 1H), 7.99 (s, 1H), 7.78-7.74 (m, 4H), 7.66 (s, 1H), 7.55-7.48 (m, 4H), 7.42-7.37 (m, 1H), 3.71 (s, 3H).

### V. Synthesis of 4,6-difluoro-N-hydroxy-5-(4-(tetrahydro-2H-pyran-2-yl)phenyl)-1H-indole-3-carboxamide (1078)

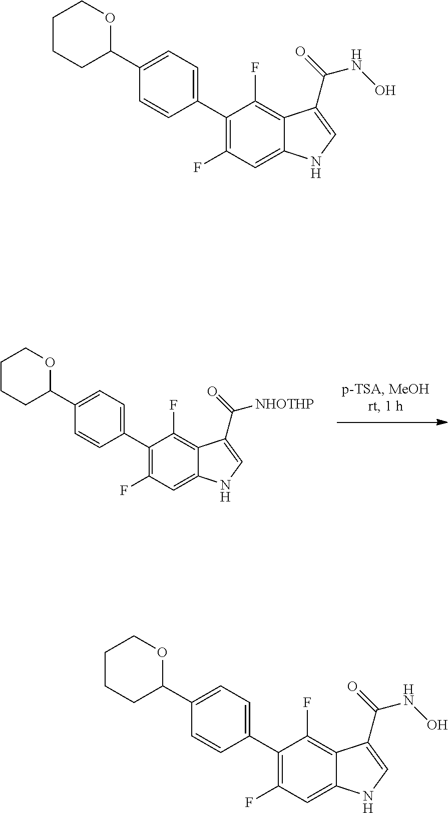

Compound 1078 was prepared according to General Synthesis Route R-3.

To a solution of 4,6-difluoro-N-((tetrahydro-2H-pyran-2-yl)oxy)-5-(4-(tetrahydro-2H-pyran-2-yl)phenyl)-1H-indole-3-carboxamide (see Example J) (70 mg, 0.15 mmol) in MeOH (5 mL) was added pTSA (5.2 mg, 0.03 mmol) and stirred at RT for 1 h. The reaction mixture was concentrated under reduced pressure to obtain a crude product which was purified by preparative HPLC to give 4,6-difluoro-N-hydroxy-5-(4-(tetrahydro-2H-pyran-2-yl)phenyl)-1H-indole-3-carboxamide as white solid (8 mg, 13% yield).

LCMS: 98.53% (M+H=373.31)

¹H NMR (400 MHz, DMSO-d6): δ 11.12 (br, 1H), 8.85 (br s, 1H), 7.76 (s, 1H), 7.44-7.38 (m, 4H), 7.23 (d, J=10.00 Hz, 1H), 4.39 (d, J=1.60 Hz, 1H), 4.05 (d, J=10.80 Hz, 1H), 3.59-3.54 (m, 1H), 1.87-1.84 (m, 2H), 1.59-1.53 (m, 4H).

### W. Synthesis of 6-chloro-5-(6-(dimethylamino)-2-methoxypyridin-3-yl)-N-hydroxy-1H-indole-3-carboxamide (1001)

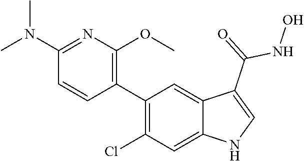 [SMILES: COc1nc(N(C)C)ccc1-c1cc2c(C(=O)NO)c[nH]c2cc1Cl]

Compound 1001 was prepared according to General Synthesis Route R-3 using a similar experimental procedure as used in the preparation of 4,6-difluoro-N-hydroxy-5-(4-(tetrahydro-2H-pyran-2-yl)phenyl)-1H-indole-3-carboxamide (1078).

LCMS: 95.20% (361.22 [M+H]+)

¹H NMR; 400 MHz, DMSO-d6: δ 11.55-10.71 (br, 2H), 8.75 (s, 1H), 7.94 (s, 1H), 7.89 (s, 1H), 7.53 (s, 1H), 7.31 (d, J=8.4 Hz, 1H), 6.21 (d, J=8.00 Hz, 1H), 3.76 (s, 3H), 3.07 (s, 6H).

### X. Synthesis of 5-([1,1′-biphenyl]-4-yl)-6-chloro-N-hydroxy-1H-indole-3-carboxamide (1006)

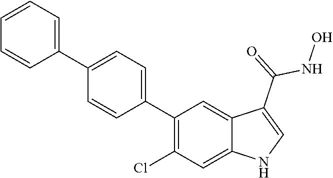 [SMILES: O=C(NO)c1c[nH]c2cc(Cl)c(-c3ccc(-c4ccccc4)cc3)cc12]

Compound 1006 was prepared according to General Synthesis Route R-3 using a similar experimental procedure as used in the preparation of 4,6-difluoro-N-hydroxy-5-(4-(tetrahydro-2H-pyran-2-yl)phenyl)-1H-indole-3-carboxamide (1078).

LCMS: 99.04% (361.10 [M−H]−)

¹H NMR 400 MHz, DMSO-d6: δ 11.69 (br s, 1H), 10.69 (br s, 1H), 8.79 (s, 1H), 8.11 (s, 1H), 7.97 (s, 1H), 7.77-7.73 (m, 4H), 7.65 (s, 1H), 7.55-7.48 (m, 4H), 7.39 (t, J=7.2 Hz, 1H).

### Building Blocks

### Example A1. Synthesis of 3-bromo-6-(4,4-difluoropiperidin-1-yl)-2-methoxypyridine (for 1058)

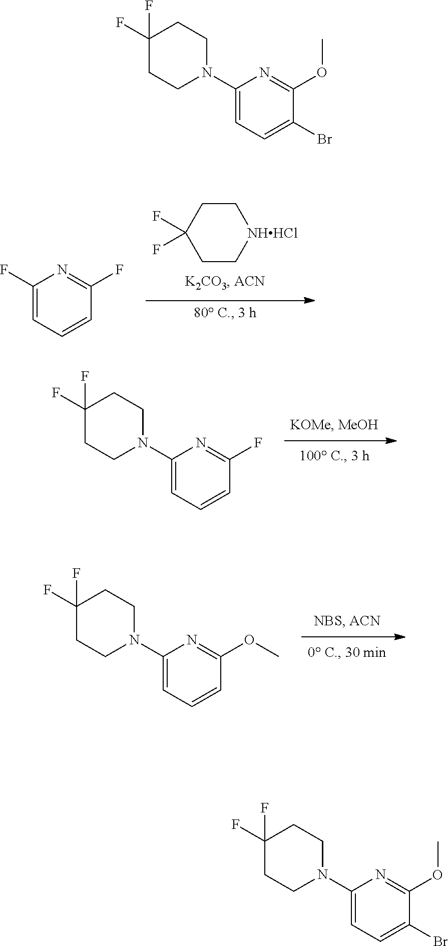

A mixture of 2,6-difluoropyridine (1.0 g, 0.87 mmol), K₂CO₃ (3.59 g, 0.0260 mol) and 4,4-difluoropiperidine hydrochloride in acetonitrile (25 mL) was heated to 80° C. for 3 h. The reaction was cooled to RT, the solid material was filtered-off and the filtrate concentrated under reduced pressure to give 3 as a pale yellow, thick gum (1 g, 53%). This material was dissolved in DMF (2.5 mL) and reacted with NaOMe (25% in MeOH) (10 mL) at 100° C. for 3 h. The product was isolated by a water/diethyl ether extraction procedure to give 4 as a pale-yellow liquid (1 g). This material was dissolved in acetonitrile (50 mL) and reacted with NBS (313 mg, 0.0018 mol) under a nitrogen atmosphere at −10° C. for 30 min. The reaction mixture was then concentrated under reduced pressure and the product was isolated by a water/diethyl ether extraction procedure followed by column chromatography to give 3-bromo-6-(4,4-difluoropiperidin-1-yl)-2-methoxypyridine (5) as a pale-yellow liquid (0.5 g). LCMS (M+H=307.18).

The following intermediates were prepared in a similar fashion:
5-bromo-N-(cyclobutylmethyl)-6-methoxy-N-methylpyridin-2-amine for 1054

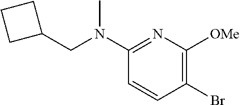 [SMILES: COc1nc(N(C)CC2CCC2)ccc1Br]

4-(5-bromo-6-methoxypyridin-2-yl)-1,4-oxazepane, LCMS: (M+H=287.24) for 1059

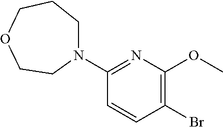 [SMILES: COc1nc(N2CCCOCC2)ccc1Br]

N1-(5-bromo-6-methoxypyridin-2-yl)-N1,N2,N2-trimethylethane-1,2-diamine for

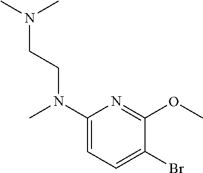

N1-(5-bromo-6-methoxypyridin-2-yl)-N1,N3,N3-trimethylpropane-1,3-diamine for 1064; LCMS: 302.27 (M+H)

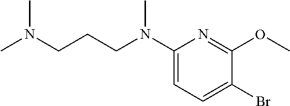

tert-butyl 4-(5-bromo-6-methoxypyridin-2-yl)piperazine-1-carboxylate for 1095; LCMS: 88% (M+H=372.15)

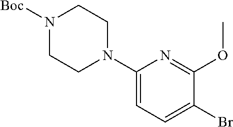 [SMILES: COc1nc(N2CCN(C(=O)OC(C)(C)C)CC2)ccc1Br]

5-bromo-4-methoxy-N,N-dimethylpyrimidin-2-amine for 1046; LCMS: ([M+H]=232.01)

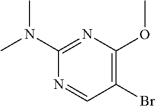

5-bromo-6-methoxy-N-methyl-N-(tetrahydro-2H-pyran-4-yl)pyridin-2-amine for 1057; LCMS: ([M+H]=301.17)

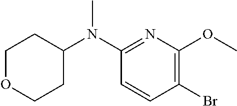 [SMILES: COc1nc(N(C)C2CCOCC2)ccc1Br]

### Example A2. Synthesis of 4-(5-bromo-6-methoxypyridin-2-yl)morpholine (for synthesis of 1034)

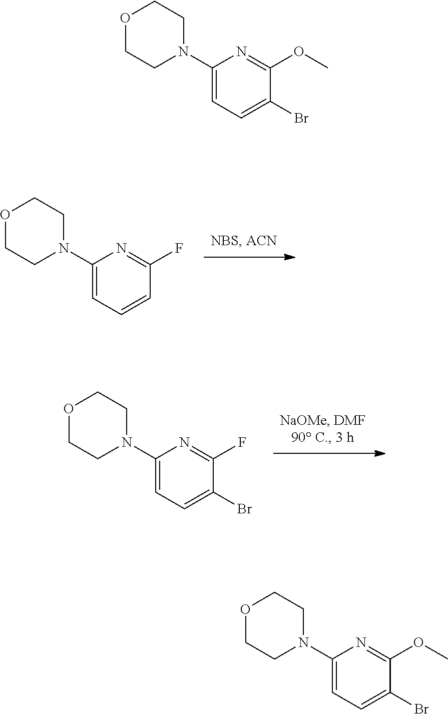

A solution of 4-(6-fluoropyridin-2-yl)morpholine (2.0 gm, 10.98 mmol), prepared as in Example A1 above using morpholine, in ACN was added NBS (1.95 gm, 10.98 mmol) at 0° C. under nitrogen. After 1 hr at RT the product was isolated by aqueous organic work up and purified by flash column chromatography to give 4-(5-bromo-6-fluoropyridin-2-yl)morpholine (1.7 g, 83% yield) as a yellow liquid, LCMS: (269.40 (M+H)). This material in DMF (5 mL) was reacted with NaOMe at 90° C. for 3 h to give 4-(5-bromo-6-methoxypyridin-2-yl)morpholine (0.8 g) as a pale yellow gum, LCMS: (316.28) (M+H).

The following intermediates were prepared in a similar fashion:
1-(5-bromo-6-methoxypyridin-2-yl)-4-methylpiperazine for 1048; LCMS: (286.05 (M+H))

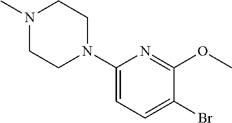

3-bromo-2-methoxy-6-(4-methylpiperidin-1-yl)pyridine for 1049; LCMS: (285.13 (M+H))

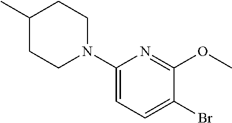

2-(5-bromo-6-methoxypyridin-2-yl)-2-azaspiro[3.3]heptane for 1050; LCMS: (285.13, M+H)

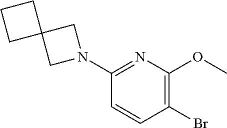 [SMILES: COc1nc(N2CC3(CCC3)C2)ccc1Br]

5-bromo-6-methoxy-N-(2-methoxyethyl)-N-methylpyridin-2-amine for 1051; LCMS: (275.13 [M+H]⁺)

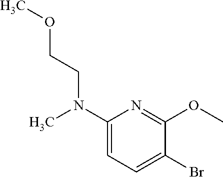

5-bromo-N-(2-isopropoxyethyl)-6-methoxy-N-methylpyridin-2-amine for 1052; LCMS: (301.13, M+H)

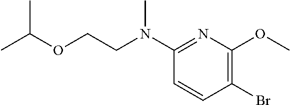 [SMILES: COc1nc(N(C)CCOC(C)C)ccc1Br]

5-bromo-N-butyl-N-ethyl-6-methoxypyridin-2-amine for 1056

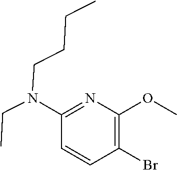 [SMILES: CCCCN(CC)c1ccc(Br)c(OC)n1]

3-bromo-2-methoxy-6-(4-methoxypiperidin-1-yl)pyridine for 1055; LCMS: (301.13, M+H)

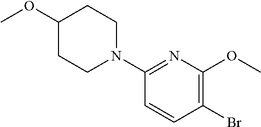

### Example B. 1-(5-bromopyridin-2-yl)cyclopropyl)methanol for the synthesis of 1101

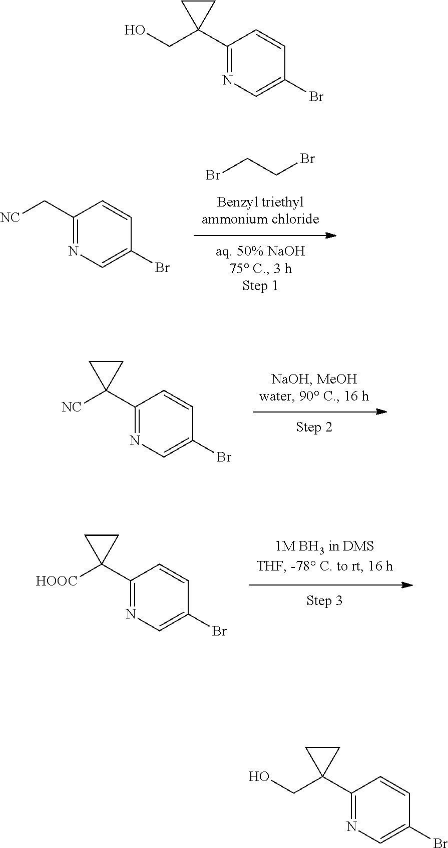

A mixture 2-(5-bromopyridin-2-yl)acetonitrile (2 g, 10.2 mmol), 1,2-dibromoethane (1.74 g, 20.3 mmol), benzyl triethylammonium chloride (BTEAC, 254 mg, 1.116 mmol) in aq. 50% NaOH (20 mL) was heated at 75° C. for 3 h. After an aqueous organic work, purification by flash chromatography gave 1-(5-bromopyridin-2-yl)cyclopropane-1-carbonitrile (1.8 g, 79.6% yield) as a pale brown solid. LCMS: (M+2=225.12). Treatment of this product with NaOH (1.93 g, 48.415 mmol) in MeOH:H₂O (18 mL: 18 mL) at 90° C. for 16 h gave 1-(5-bromopyridin-2-yl)cyclopropane-1-carboxylic acid (1.6 g, 82.05%) as off-white solid; LCMS: 95.93% (M+2=244.06). Reduction of this product with BH₃ (23 mL, 23.54 mmol, 1 M in DMS) in THE (16 mL) at −78° C. and then 16 h at RT gave (1-(5-bromopyridin-2-yl)cyclopropyl)methanol (0.9 g; 60% yield) as a white solid.

LCMS: 79.97% (M+H=228.15)

¹H NMR 400 MHz, DMSO-d6: δ 8.52 (s, 1H), 7.92 (dd, J=2.40, 8.40 Hz, 1H), 7.49 (d, J=8.80 Hz, 1H), 4.80 (t, J=5.60 Hz, 1H), 3.72 (d, J=5.20 Hz, 2H), 1.08 (d, J=2.40 Hz, 2H), 0.92 (t, J=2.40 Hz, 2H).

### Example C. Synthesis of 1-(5-bromopyridin-2-yl)cyclobutan-1-ol (for Synthesis of 1099)

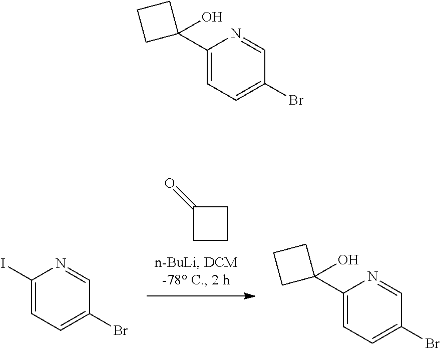 [SMILES: OC1(c2ccc(Br)cn2)CCC1 ; O=C1CCC1 ; Brc1ccc(I)nc1 ; OC1(c2ccc(Br)cn2)CCC1]

n-BuLi (4 mL, 0.0247) was added slowly to a stirred solution of 5-bromo-2-iodopyridine (3.0 g, 0.026 mmol) in DCM (30 mL) at −78° C. After 45 min, cyclobutanone (2.99 g, 0.052 mmol) was added and the reaction stirred for 2 h at −78° C. The reaction was quenched with NH₄Cl solution (50 mL) and, after an aqueous organic work and purification by flash chromatography gave 1-(5-bromopyridin-2-yl)cyclobutan-1-ol (0.9 g) as a brown gum.

LCMS: 83% (228, M+H)

¹H NMR 400 MHz, CDCl3: δ 8.66 (d, J=2.40 Hz, 1H), 7.99 (dd, J=2.40, 8.40 Hz, 1H), 7.52 (d, J=8.40 Hz, 1H), 5.82 (s, 1H), 2.50-0.00 (m, 2H), 2.18-2.25 (m, 2H), 1.82-1.87 (m, 1H), 1.89-1.92 (m, 1H).

### Example D. Synthesis of 3-bromo-2-methoxy-6-((trimethylsilyl)ethynyl)pyridine (for Synthesis of 1093)

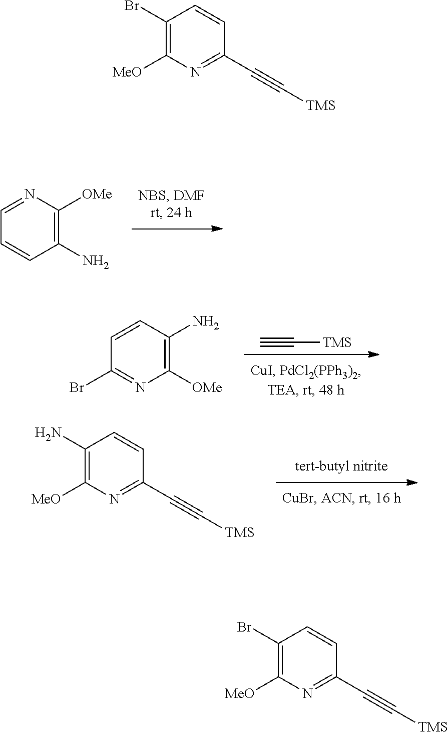

NBS (14.27 g, 80.64 mmol) in DMF (20 mL) was added dropwise at 0° C. to a solution of a solution of 2-methoxypyridin-3-amine (10.0 g, 80.64 mmol) in DMF (60 mL) and then stirred for 16 h at RT. After an aqueous organic work and purification by flash column chromatography 2-methoxypyridin-3-amine (8.5 g, 52% yield) was obtained as a brown solid (LCMS: ([M+H]=205). A solution of this product (8 g, 39.40 mmol) and TMS acetylene (11.5 g, 118.2 mmol) in TEA (70 mL) was purged with nitrogen. CuI (748 mg, 0.003 mmol) and PdCl₂(PPh₃) (1.3 g, 1.97 mmol) were added and the reaction mixture was stirred for 48 h at RT. After an aqueous organic work and purification by flash column chromatography 2-methoxy-6-((trimethylsilyl)ethynyl)pyridin-3-amine (7.5 g, 86% yield) was obtained as a brown solid LCMS: ([M+H]=221). A solution of this product (2 g, 43.10 mmol) in acetonitrile (20 mL) at 0° C. was treated dropwise with tert-butyl nitrite (4 g) and then CuBr₂ (4 g). The reaction mixture was stirred at RT for 16 h, evaporated under reduced pressure and the crude product purified by flash column chromatography to give 3-bromo-2-methoxy-6-((trimethylsilyl)ethynyl)pyridine (1.8 g, 70% yield) as a orange liquid.

LCMS: (M+H]=284).

### Example E. Synthesis of 4-(5-bromo-6-methoxypyridin-2-yl)-2-methylbut-3-yn-2-ol (for Synthesis of 1093)

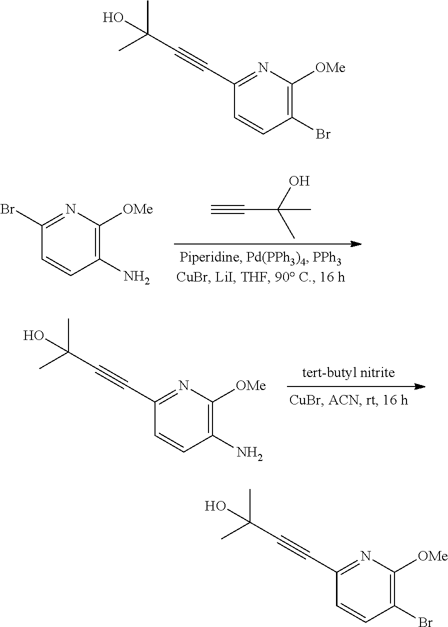

A solution of 6-bromo-2-methoxypyridin-3-amine (2.5 g, 12.31 mmol), 2-methylbut-3-yn-2-ol (2.07 g, 24.62 mmol) and piperidine (3.71 g, 43.7 mmol) in THF (10 mL) was purged with nitrogen for 5 min. Pd(PPh₃)₄ (32 mg, 0.027 mmol) and PPh₃ (12 mg, 0.046 mmol) were added. Purging was continued followed by the addition of CuBr (12 mg, 0.086 mmol) and LiI (66 mg, 0.492 mmol). After 16 h at 90° C. the reaction mixture was dried under reduced pressure and the crude product purified by flash column to give 4-(5-amino-6-methoxypyridin-2-yl)-2-methylbut-3-yn-2-ol (1.8 g, 72% yield) as an orange solid LCMS: ([M+H]=207.32). Reaction of this product with tert-butyl nitrite (3.6 g) and CuBr₂ (3.6 g) in acetonitrile (18 mL) for 18 h at RT as described in Example D gave 4-(5-bromo-6-methoxypyridin-2-yl)-2-methylbut-3-yn-2-ol (0.8 g, 34% yield) as an off-white solid.

LCMS: ([M+H]=270.09).

### Synthesis of Compounds of the Disclosure

### Example 1. Synthesis of 2-(4,6-difluoro-5-(4-(tetrahydro-2H-pyran-2-yl)phenyl)-1H-indol-3-yl)-2-oxoacetic acid (1096)

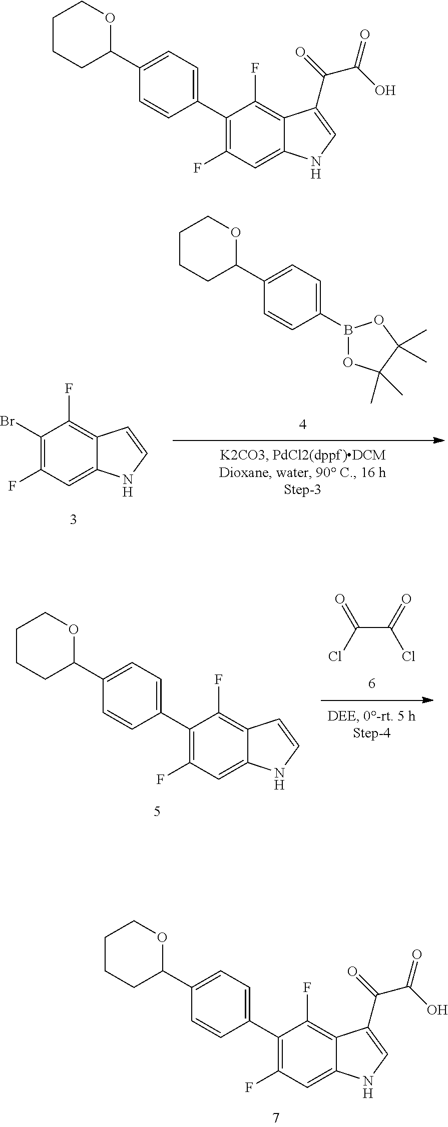 [SMILES: O=C(O)C(=O)c1c[nH]c2cc(F)c(-c3ccc(C4CCCCO4)cc3)c(F)c12 ; Fc1cc2[nH]ccc2c(F)c1Br ; CC1(C)OB(c2ccc(C3CCCCO3)cc2)OC1(C)C ; Fc1cc2[nH]ccc2c(F)c1-c1ccc(C2CCCCO2)cc1 ; O=C(Cl)C(=O)Cl ; O=C(O)C(=O)c1c[nH]c2cc(F)c(-c3ccc(C4CCCCO4)cc3)c(F)c12]

A solution of 5-bromo-4,6-difluoro-1H-indole (100 mg, 0.43 mmol), 4,4,5,5-tetramethyl-2-(4-(tetrahydro-2H-pyran-2-yl)phenyl)-1,3,2-dioxaborolane (148 mg, 0.51 mmol) and K₂CO₃ (118 mg, 0.86 mmol) in 1,4-dioxane (10 mL) and water (2 mL) was purged with nitrogen for 10 min. PdCl₂(dppf)·DCM (17.5 mg, 0.021 mmol) was added and the reaction mixture was stirred at 90° C. After 16 h the reaction mixture was filtered through celite bed, concentrated under reduced pressure and purified by flash column chromatography to give 4,6-difluoro-5-(4-(tetrahydro-2H-pyran-2-yl)phenyl)-1H-indole (60 mg. 44% yield) as an off white solid; LCMS: 67.50% ([M−H]=312.24). This product was dissolved in diethyl ether (10 mL) at 0° C., oxalyl chloride (0.2 mL, 0.9 mmol) was added and the mixture was stirred for 5 h at RT. Evaporation to dryness gave 2-(4,6-difluoro-5-(4-(tetrahydro-2H-pyran-2-yl)phenyl)-1H-indol-3-yl)-2-oxoacetic acid (50 mg) as a yellow solid.

LCMS: ([M+H]386.29).

### Example 2. Synthesis of N-(butylsulfonyl)-6-chloro-5-(6-(dimethylamino)-2-methoxypyridin-3-yl)-1H-indole-3-carboxamide (1108)

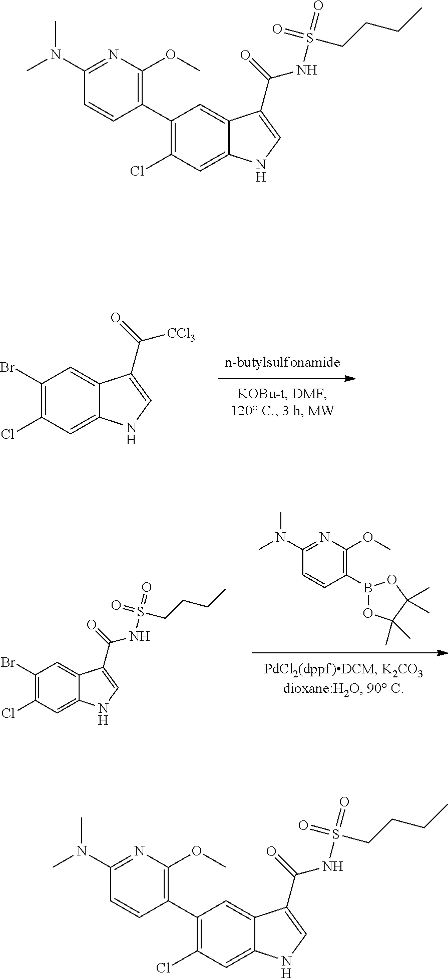

Potassium tert-butoxide (268 mg, 2.4 mmol) was slowly added to a stirred a solution of 1-(5-bromo-6-chloro-1H-indol-3-yl)-2,2,2-trichloroethan-1-one (300 mg, 0.6 mmol) in DMF (5 mL) at 0° C. N-butyl sulfonamide (219 mg, 1.6 mmol) was added and the reaction was heated at 120° C. for 3 h in a microwave reactor. The reaction mixture was cooled, filtered through celite, evaporated to dryness and purified by reverse phase chromatography to give 5-bromo-N-(butylsulfonyl)-6-chloro-1H-indole-3-carboxamide (100 mg, 32% yield) as a pale yellow liquid (LCMS: (M+H=393.26)). This product was mixed with 6-methoxy-N,N-dimethyl-5-(4,4,5,5-tetramethyl-1,3,2-dioxaborolan-2-yl)pyridin-2-amine (283 mg, 1 mmol; 30% pure) and K₂CO₃ (247 mg, 1.7895 mmol) in dioxane (10 mL) and water (1 mL) and purged with nitrogen for 10 min. PdCl₂(dppf)·DCM (28 mg, 0.03479 mmol) was added and the reaction mixture was heated to 90° C. for 5 h. The reaction mixture was cooled, filtered through celite, evaporated to dryness and purified by reverse phase chromatography to give N-(butylsulfonyl)-6-chloro-5-(6-(dimethylamino)-2-methoxypyridin-3-yl)-1H-indole-3-carboxamide (5 mg; 4% yield) as a white solid.

LCMS: (M+H=465.34)

¹H NMR 400 MHz, CDCl3: δ 11.19 (s, 1H), 8.12 (s, 1H), 7.66 (s, 1H), 7.40 (s, 1H), 7.28 (d, J=8.00 Hz, 1H), 6.20 (d, J=8.40 Hz, 1H), 3.76 (s, 3H), 3.06-3.01 (m, 8H), 1.57-1.53 (m, 2H), 1.33-1.24 (m, 3H), 0.83 (t, J=7.6 Hz, 3H).

### Example 3. Synthesis of 4,6-difluoro-N-(2-methoxyethoxy)-5-(4-(tetrahydro-2H-pyran-2-yl)phenyl)-1H-indole-3-carboxamide (1042)

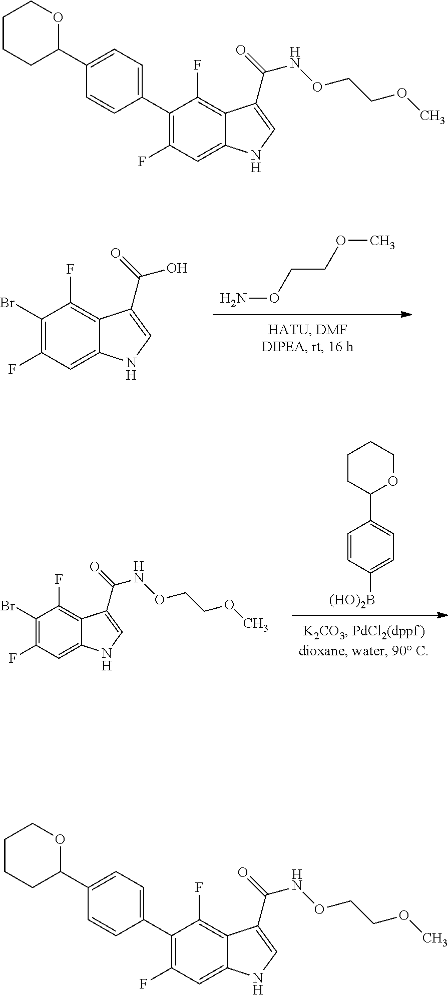

To a solution of 5-bromo-4,6-difluoro-1H-indole-3-carboxylic acid (250 mg, 0.90 mmol)) in DMF (5 mL) was added HATU (516 mg, 1.35 mmol), DIPEA (0.5 ml, 2.1 mmol) and stirred for 5 min; O-(2-methoxyethyl)hydroxylamine (124 mg, 1.35 mmol) was added and stirred at rt for 2 h. The product was isolated via an aqueous organic work up followed by flash column chromatography to give 5-bromo-4,6-difluoro-N-(2-methoxyethoxy)-1H-indole-3-carboxamide (80 mg, 25% yield) as white solid, LCMS: (349.26 [M+H]⁺). A solution of this product with (4-(tetrahydro-2H-pyran-2-yl)phenyl)boronic acid (66.2 mg, 0.2298 mmol) and K₂CO₃ (80 mg, 0.57 mmol) in dioxane:water (4 mL:1 mL) was purged nitrogen for 10 min; PdCl₂(dppf)·DCM (9.38 mg, 0.0114 mmol) was added and the reaction was heated at 90° C. for 4 h. Filtration through celite, evaporation and purification by preparative HPLC gave 4,6-difluoro-N-(2-methoxyethoxy)-5-(4-(tetrahydro-2H-pyran-2-yl)phenyl)-1H-indole-3-carboxamide (12 mg) as an off-white solid.

¹H NMR (400 MHz, DMSO-d6): δ 12.12 (s, 1H), 11.01 (s, 1H), 7.84 (s, 1H), 7.44-7.38 (m, 4H), 7.24 (d, J=10.00 Hz, 1H), 4.39 (d, J=1.60 Hz, 1H), 4.05 (d, J=11.60 Hz, 1H), 3.99 (t, J=4.40 Hz, 2H), 3.59-3.53 (m, 3H), 3.28 (s, 3H), 1.88-1.85 (m, 2H), 1.70-1.49 (m, 4H).

LCMS: 99.66% (429.53 [M−H]).

### Example 4. Synthesis of 6-chloro-5-(6-(dimethylamino)-2-methoxypyridin-3-yl)-N-methoxy-1H-indole-3-sulfonamide (1070)

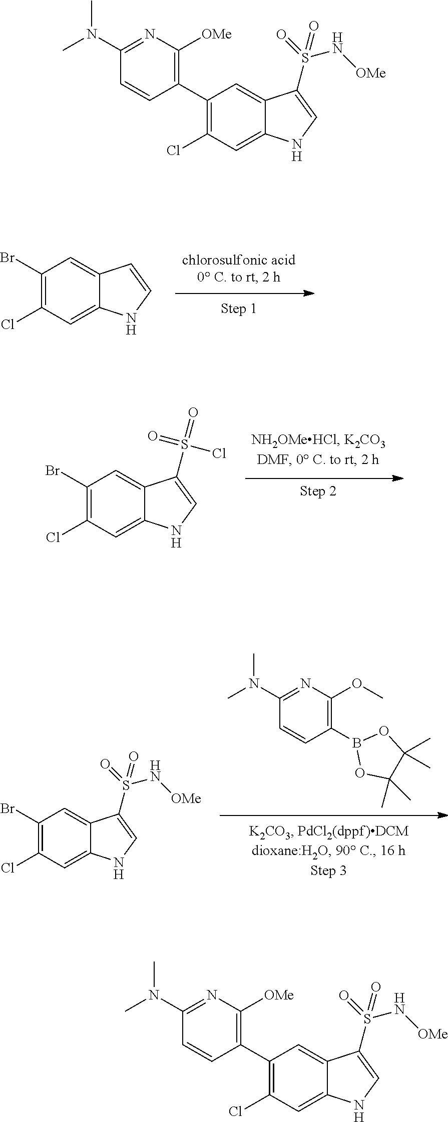

### Step 1: 5-bromo-6-chloro-1H-indole-3-sulfonyl chloride

Chlorosulfonic acid (4 mL) was added drop wise to a solution of 5-bromo-6-chloro-1H-indole (2.0 g, 8.68 mmol) in acetonitrile at 0° C. After 2 h at RT the reaction mixture was diluted with ice cold water (and the precipitated solid was filtered, washed with water, and dried under vacuum to give 5-bromo-6-chloro-1H-indole-3-sulfonyl chloride as an off-white solid (1.7 g, 59% yield. LCMS: ([M−H]=325.91).

### Step 2: 6-chloro-5-(6-(dimethylamino)-2-methoxypyridin-3-yl)-N-methoxy-1H-indole-3-sulfonamide

A solution of the product of Step 1 (1.5 g, 4.56 mmol) in DMF (15 mL) was cooled to 0° C. K₂CO₃ (2.52 g, 18.24 mmol) and methoxyamine hydrochloride (0.762 g, 9.12 mmol) were added at 0° C. and the reaction mixture was stirred at RT. After 2 h it was diluted with ice cold water (200 mL) and the precipitated solid was filtered, washed with water, and dried under vacuum to give 6-chloro-5-(6-(dimethylamino)-2-methoxypyridin-3-yl)-N-methoxy-1H-indole-3-sulfonamide (0.5 g, 32% yield) as an off-white solid.

¹H NMR (400 MHz, DMSO-d6): δ 12.41 (s, 1H), 10.24 (s, 1H), 8.10 (d, J=4.00 Hz, 2H), 7.80 (s, 1H), 3.66 (s, 3H). LCMS: ([M−H]=336.99).

### Step 3: 6-chloro-5-(6-(dimethylamino)-2-methoxypyridin-3-yl)-N-methoxy-1H-indole-3-sulfonamide

A stirred solution of the product of Step 2 (100 mg, 0.3 mmol), 6-methoxy-N,N-dimethyl-5-(4,4,5,5-tetramethyl-1,3,2-dioxaborolan-2-yl)pyridin-2-amine (126 mg, 0.45 mmol) and K₂CO₃ (83 mg, 0.6 mmol) in 1,4-dioxane (1.8 mL) and water (0.2 mL) was purged with nitrogen for 10 min. PdCl₂(dppf)·DCM (13 mg, 0.015 mmol) was added and the reaction mixture was stirred at 100° C. After 16 h it was cooled, filtered through celite, concentrated under reduced pressure and purified by flash column chromatography to give 6-chloro-5-(6-(dimethylamino)-2-methoxypyridin-3-yl)-N-methoxy-1H-indole-3-sulfonamide (25 mg, 20% yield).

¹H NMR (400 MHz, DMSO-d6): δ 12.20 (s, 1H), 10.14 (s, 1H), 8.03 (s, 1H), 7.65 (s, 1H), 7.61 (s, 1H), 7.32 (d, J=8.00 Hz, 1H), 6.23 (d, J=8.40 Hz, 1H), 3.77 (s, 3H), 3.63 (s, 3H), 3.07 (s, 6H); LCMS: ([M+H]=411.25).

The following compounds were prepared in a similar fashion:
5-([1,1′-biphenyl]-4-yl)-6-chloro-N-methoxy-1H-indole-3-sulfonamide (1110)

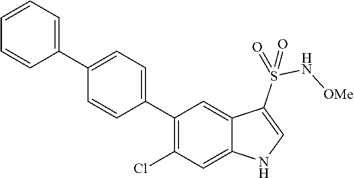 [SMILES: CONS(=O)(=O)c1c[nH]c2cc(Cl)c(-c3ccc(-c4ccccc4)cc3)cc12]

¹H NMR (400 MHz, DMSO-d6): δ 12.32 (s, 1H), 10.20 (s, 1H), 8.10 (s, 1H), 7.73-7.82 (m, 6H), 7.49-7.57 (m, 4H), 7.38-7.42 (m, 1H), 3.65 (s, 3H); LCMS: ([M−H]=411.18).
6-chloro-N-methoxy-5-(4-(tetrahydro-2H-pyran-2-yl)phenyl)-1H-indole-3-sulfonamide (1109)

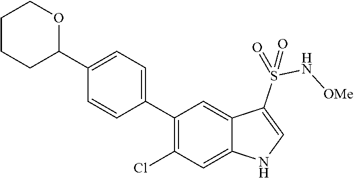 [SMILES: CONS(=O)(=O)c1c[nH]c2cc(Cl)c(-c3ccc(C4CCCCO4)cc3)cc12]

¹H NMR (400 MHz, DMSO-d6): δ 12.32 (s, 1H), 10.20 (s, 1H), 8.00 (s, 1H), 7.71 (s, 1H), 7.64 (s, 1H), 7.38-7.42 (m, 4H), 4.37 (dd, J=1.60, 11.00 Hz, 1H), 4.05 (d, J=10.80 Hz, 1H), 3.53-3.61 (m, 4H), 1.84-1.90 (m, 2H), 1.53-1.70 (m, 4H); LCMS: ([M−H]=419.20).

### Example 5. Synthesis of 2-(6-chloro-5-(6-(dimethylamino)-2-methoxypyridin-3-yl)-1H-indol-3-yl)-2-oxoacetic acid (1113)

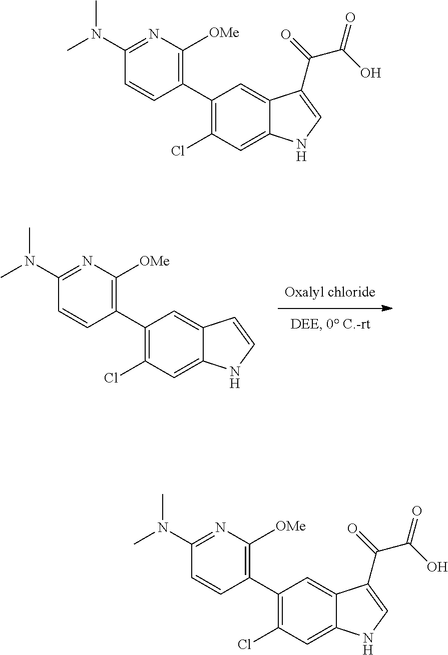 [SMILES: COc1nc(N(C)C)ccc1-c1cc2c(C(=O)C(=O)O)c[nH]c2cc1Cl ; COc1nc(N(C)C)ccc1-c1cc2cc[nH]c2cc1Cl ; COc1nc(N(C)C)ccc1-c1cc2c(C(=O)C(=O)O)c[nH]c2cc1Cl]

Oxalyl chloride (0.28 ml, 3.31 mmol) was added dropwise to a solution of 5-(6-chloro-1H-indol-5-yl)-6-methoxy-N,N-dimethylpyridin-2-amine (200 mg, 0.66 mmol) in diethyl ether (2 mL) at 0° C. After stirring at RT for 0.5 h the reaction mixture was filtered and dried under vacuum and the crude product was purified by prep-HPLC to give 2-(6-chloro-5-(6-(dimethylamino)-2-methoxypyridin-3-yl)-1H-indol-3-yl)-2-oxoacetic acid (15 mg, 6% yield) as a pale yellow solid.

LCMS: 97.23% (374.20 [M+H]⁻).

¹H NMR (400 MHz, DMSO-d6): δ 13.30 (br s, 1H), 11.78 (s, 1H), 8.13 (s, 1H), 8.00 (s, 1H), 7.54 (s, 1H), 7.30 (d, J=8.00 Hz, 1H), 6.22 (d, J=8.00 Hz, 1H), 3.76 (s, 3H), 3.07 (s, 6H).

### Example 6. Synthesis of 2-(6-chloro-5-(6-(dimethylamino)-2-methoxypyridin-3-yl)-1H-indol-3-yl)-N-methoxy-2-oxoacetamide (1083)

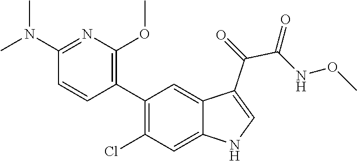

To a solution of 2-(6-chloro-5-(6-(dimethylamino)-2-methoxypyridin-3-yl)-1H-indol-3-yl)-2-oxoacetic acid (400 mg, 1.0701 mmol)) in DMF (5 mL) at rt was added HATU (610 mg, 1.6 mmol) and DIPEA (0.6 mL, 3.2 mmol) and Reaction was stirred for 15 min. Then HCl·NH₂OMe (134 mg, 1.6 mmol) was added and the reaction was stirred at rt for 16 h. After aqueous organic work up, the crude product was purified by prep-HPLC to give 2-(6-chloro-5-(6-(dimethylamino)-2-methoxypyridin-3-yl)-1H-indol-3-yl)-N-methoxy-2-oxoacetamide (20 mg, 5% yield) as a yellow solid.

LCMS: 92.53% (403.32 [M+H]⁻).

HPLC: 91.95%

¹H NMR (400 MHz, DMSO-d6): δ 12.33 (s, 1H), 12.03 (s, 1H), 8.70 (s, 1H), 8.03 (s, 1H), 7.65 (s, 1H), 7.33 (d, J=8.40 Hz, 1H), 6.23 (d, J=8.40 Hz, 1H), 3.76 (s, 3H), 3.70 (s, 3H), 3.07 (s, 6H).

### Example 2: EC₅₀ Data

EC₅₀ data of compounds of the disclosure was determined and the values are shown below in Table A.

TABLE A
EC₅₀ data (compound numbers are shown)

| <100 nM | 100 nM-10 μM | >10 μM |
|---|---|---|
| 1113 | 1022 | 1021 |
| 1084 | 1018 | 1020 |
| 1024 | 1017 | 1012 |
| 1007 | 1013 | 1049 |
| 1003 | 1016 | 1050 |
| 1044 | 1014 | 1051 |
| 1077 | 1010 | 1064 |
| 1081 | 1008 | 1075 |
| 1088 | 1006 | 1074 |
| 1112 | 1001 | 1041 |
| | 1085 | 1117 |
| | 1025 | 1060 |
| | 1023 | 1058 |
| | 1022 | 1052 |
| | 1019 | 1054 |
| | 1018 | 1090 |
| | 1017 | 1101 |
| | 1004 | 1099 |
| | 1013 | 1098 |
| | 1016 | 1097 |
| | 1015 | 1100 |
| | 1014 | 1028 |
| | 1005 | 1110 |
| | 1010 | 1109 |
| | 1009 | |
| | 1011 | |
| | 1008 | |
| | 1006 | |
| | 1001 | |
| | 1078 | |
| | 1027 | |
| | 1108 | |
| | 1026 | |
| | 1034 | |
| | 1046 | |
| | 1045 | |
| | 1047 | |
| | 1067 | |
| | 1042 | |
| | 1048 | |
| | 1035 | |
| | 1039 | |
| | 1064 | |
| | 1061 | |
| | 1062 | |
| | 1059 | |
| | 1076 | |
| | 1073 | |
| | 1055 | |
| | 1104 | |
| | 1103 | |
| | 1080 | |
| | 1102 | |
| | 1096 | |
| | 1093 | |
| | 1092 | |
| | 1095 | |
| | 1091 | |
| | 1094 | |

### Example 3: Blood-Brain Barrier Data

Blood Brain Barrier (BBB) data was evaluated for compounds of the disclosure and the values are shown in Table B. Brain/plasma ratio was calculated at a 3 hr time point).

TABLE B
BBB Data (compound numbers are shown)

| <0.1 | 0.1-1 | >1 |
|---|---|---|
| 1001 | 1015 | 1003 |
| 1084 | 1009 | |
| 1045 | 1011 | |
| 1077 | | |
| 1081 | | |
| 1112 | | |

### Example 4: Spectral Data for Compounds of the Disclosure

NMR and LCMS data are shown below in Table C for compounds of the disclosure:

TABLE C
Spectral data

| | Cmpd. | | | Synthesis |
|---|---|---|---|---|
| Structure | # | NMR Data | MS Data | Scheme |
| 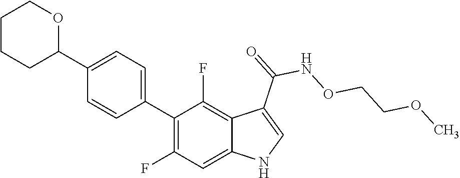 [SMILES: COCCONC(=O)c1c[nH]c2cc(F)c(-c3ccc(C4CCCCO4)cc3)c(F)c12] | 1042 | ¹H NMR (400 MHz, DMSO- d6): δ 12.12 (s, 1H), 11.01 (s, 1H), 7.84 (s, 1H), 7.44-7.38 (m, 4H), 7.24 (d, J = 10.00 Hz, 1H), 4.39 (d, J = 1.60 Hz, 1H), 4.05 (d, J = 11.60 Hz, 1H), 3.99 (t, J = 4.40 Hz, 2H), 3.59- 3.53 (m, 3H), 3.28 (s, 3H), 1.88-1.85 (m, 2H), 1.70-1.49 (m, 4H). | LCMS: 99.66% (429.53 [M − H]⁺). | R3 |
| 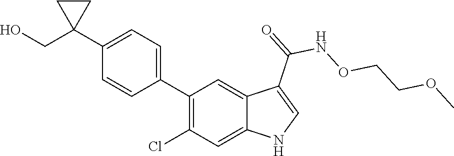 [SMILES: COCCONC(=O)c1c[nH]c2cc(Cl)c(-c3ccc(C4(CO)CC4)cc3)cc12] | 1044 | ¹H NMR (400 MHz, DMSO): δ 11.43 (br s, 2H), 8.02 (s, 1H), 8.01 (s, 1H), 7.62 (s, 1H), 7.38-7.33 (m, 4H), 4.71 (br s, 1H), 3.99 (t, J = 4.40 Hz, 2H), 3.58-3.55 (m, 4H), 3.29 (s, 3H), 0.88-0.86 (m, 2H), 0.80- 078 (m, 2H). | LCMS: 99.87% ([M + H] = 415.42) | R1 |
| 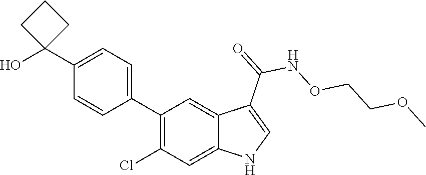 [SMILES: COCCONC(=O)c1c[nH]c2cc(Cl)c(-c3ccc(C4(O)CCC4)cc3)cc12] | 1045 | ¹H NMR (400 MHz, DMSO): δ 11.25 (br s, 2H), 8.03 (s, 1H), 7.95 (s, 1H), 7.61 (s, 1H), 7.55 (d, J = 8.40 Hz, 2H), 7.40 (d, J = 8.40 Hz, 2H), 5.51 (s, 1H), 3.96 (t, J = 4.80 Hz, 2H), 3.56 (t, J = 4.40 Hz, 2H), 3.28 (s, 3H), 2.47-2.42 (m, 2H), 2.34-2.26 (m, 2H), 1.98-1.91 (m, 1H), 1.75-1.65 (m, 1H). | LCMS: 97.32% ([M + H] = 413.30) | R1 |
| 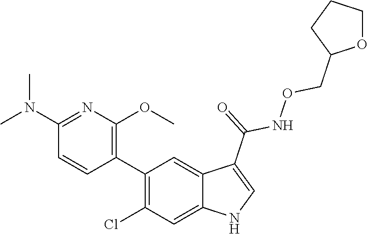 [SMILES: COc1nc(N(C)C)ccc1-c1cc2c(C(=O)NOCC3CCCO3)c[nH]c2cc1Cl] | 1047 | ¹H NMR (400 MHz, DMSO- d6): δ 11.7 (s, 1H), 11.2 (s, 1H), 7.95 (d, J = 16.8 Hz, 2H), 7.54 (s, 1H), 7.31-7.29 (m, 1H), 6.21 (d, J = 8.0 Hz, 1H), 4.09-4.07 (m, 1H), 3.88-3.79 (m, 2H), 3.76-3.62 (m, 5H), 3.06 (s, 6H), 1.96-1.93 (m, 1H), 1.83-1.79 (m, 2H), 1.65- 1.61 (m, 1H). | LCMS: 91.97% (M − H = 443.51) | R1 |
| 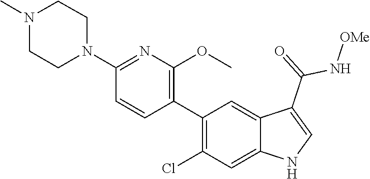 [SMILES: CONC(=O)c1c[nH]c2cc(Cl)c(-c3ccc(N4CCN(C)CC4)nc3OC)cc12] | 1048 | ¹H NMR: 400 MHz, DMSO- d6: δ 11.72 (s, 1H), 11.15 (s, 1H), 7.94-7.93 (m, 2H), 7.56 (s, 1H), 7.42 (d, J = 8.00 Hz, 1H), 6.50 (d, J = 8.00 Hz, 1H), 4.22-3.68 (m, 10H), 3.31 (br, 7H). | LCMS: 96.15% (430.45 [M + H]) | R3 |
| 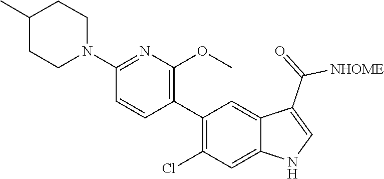 | 1049 | ¹H NMR: 400 MHz, DMSO- d6: δ 11.36 (br s, 2H), 7.93 (d, J = 8.00 Hz, 2H), 7.57 (s, 1H), 7.31 (d, J = 8.40 Hz, 1H), 6.39 (d, J = 8.00 Hz, 1H), 4.29 (d, J = 12.80 Hz, 2H), 3.73 (s, 3H), 3.68 (s, 3H), 2.82 (t, J = 22.80 Hz, 2H), 1.71-1.59 (m, 3H), 1.20-1.10 (m, 2H), 0.95 (d, J = 6.40 Hz, 3H). | LCMS: 95.83% (M + H = 429.47) | R3 |
| 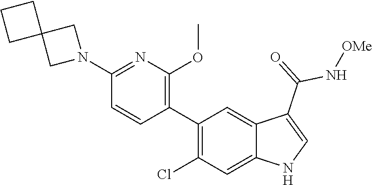 [SMILES: CONC(=O)c1c[nH]c2cc(Cl)c(-c3ccc(N4CC5(CCC5)C4)nc3OC)cc12] | 1050 | ¹H NMR: 400 MHz, DMSO- d6: δ 11.70 (br s, 1H), 11.13 (br s, 1H), 7.91 (s, 2H), 7.54 (s, 1H), 7.30 (d, J = 8.00 Hz, 1H), 5.96 (d, J = 8.00 Hz, 1H), 3.95 (s, 4H), 3.71 (s, 6H). | LCMS: 97.90% (427.46, M + H); | R2 |
| 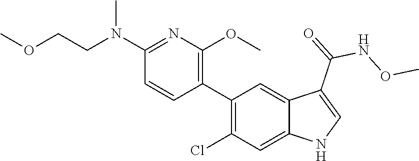 | 1051 | ¹H NMR: 400 MHz, DMSO- d6: δ 11.35 (br s, 2H), 7.94 (s, 1H), 7.91 (s, 1H), 7.59 (s, 1H), 7.31 (d, J = 8.00 Hz, 1H), 6.20 (d, J = 8.40 Hz, 1H), 3.74-3.71 (m, 5H), 3.68 (s, 3H), 3.58- 3.55 (m, 2H), 3.29 (s, 3H), 3.06 (s, 3H). | LCMS: 99.26% (419.50 [M + H]⁺). | R2 |
| 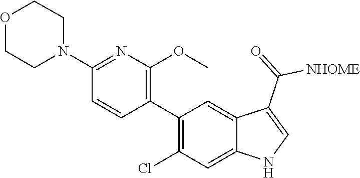 | 1034 | ¹H NMR; 400 MHz, DMSO- d6: δ 11.31 (br s, 2H), 7.96 (s, 1H), 7.92 (s, 1H), 7.64 (br s, 1H), 7.37 (d, J = 8.00 Hz, 1H), 6.41 (d, J = 8.40 Hz, 1H), 3.75-3.72 (m, 7H), 3.67 (s, 3H), 3.49-3.47 (m, 4H). | LCMS: 98.09% (413.34 [M + H]) | R2 |
| 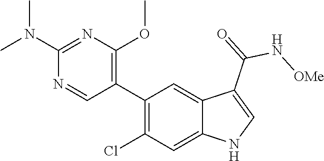 [SMILES: CONC(=O)c1c[nH]c2cc(Cl)c(-c3cnc(N(C)C)nc3OC)cc12] | 1046 | ¹H NMR (400 MHz, DMSO): δ 11.34 (br s, 2H), 7.96 (s, 2H), 7.92 (s, 1H), 7.67 (s, 1H), 3.83 (s, 3H), 3.67 (s, 3H), 3.17 (s, 6H). | LCMS: 97.20% ([M + H] = 376.39) | R2 |
| 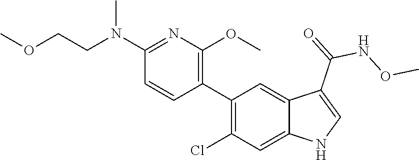 | 1051 | ¹H NMR: 400 MHz, DMSO- d6: δ 11.35 (br s, 2H), 7.94 (s, 1H), 7.91 (s, 1H), 7.59 (s, 1H), 7.31 (d, J = 8.00 Hz, 1H), 6.20 (d, J = 8.40 Hz, 1H), 3.74-3.71 (m, 5H), 3.68 (s, 3H), 3.58- 3.55 (m, 2H), 3.29 (s, 3H), 3.06 (s, 3H) | LCMS: 99.26% (419.50 [M + H]⁺). | R2 |
| 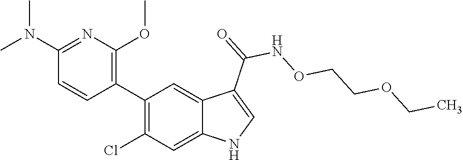 [SMILES: CCOCCONC(=O)c1c[nH]c2cc(Cl)c(-c3ccc(N(C)C)nc3OC)cc12] | 1035 | ¹H NMR (400 MHz, DMSO- d6): δ 11.37 (s, 2H), 7.99 (s, 1H), 7.92 (s, 1H), 7.59 (s, 1H), 7.31 (d, J = 8.00 Hz, 1H), 6.21 (d, J = 8.00 Hz, 1H), 3.98 (t, J = 4.80 Hz, 2H), 3.76 (s, 3H), 3.60 (t, J = 4.80 Hz, 2H), 3.47 (q, J = 7.20 Hz, 2H), 3.07 (s, 6H), 1.12 (t, J = 6.80 Hz, 3H). | LCMS: 95.31% (433.38 [M + H]⁺). | R1 |
| 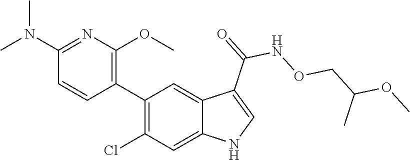 | 1039 | ¹H NMR 400 MHz, DMSO- d6: δ 11.70 (s, 1H), 11.11 (s, 1H), 7.98 (d, J = 2.40 Hz, 1H), 7.94 (s, 1H), 7.55 (s, 1H), 7.32 (d, J = 8.40 Hz, 1H), 6.22 (d, J = 8.40 Hz, 1H), 3.85-3.83 (m, 2H), 3.77 (s, 3H), 3.61-3.59 (m, 1H), 3.31 (s, 3H), 3.08 (s, 6H), 1.12 (d, J = 6.40 Hz, 3H). | LCMS: 98.86% (M + H = 433.51) | R1 |
| 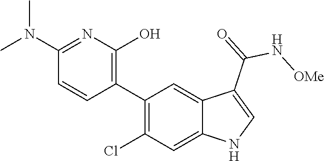 [SMILES: CONC(=O)c1c[nH]c2cc(Cl)c(-c3ccc(N(C)C)nc3O)cc12] | 1041 | ¹H NMR 400 MHz, DMSO- d6: δ 12.5-10.5 (br s, 2H), 7.93 (d, J = 9.60 Hz, 2H), 7.55 (s, 1H), 7.24 (d, J = 8.00 Hz, 1H), 5.84 (s, 1H), 3.69 (s, 3H), 3.01 (s, 6H). | LCMS: 97.84% (M + H = 361.25) | R1 |
| 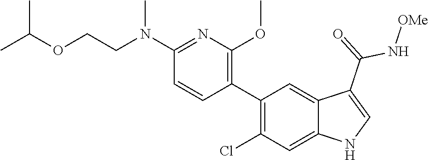 [SMILES: CONC(=O)c1c[nH]c2cc(Cl)c(-c3ccc(N(C)CCOC(C)C)nc3OC)cc12] | 1052 | ¹H NMR, 400 MHz, DMSO- d6: δ 11.74 (s, 1H), 11.15 (s, 1H), 7.93 (d, J = 3.60 Hz, 2H), 7.55 (s, 1H), 7.31 (d, J = 8.00 Hz, 1H), 6.19 (d, J = 8.40 Hz, 1H), 3.74 (s, 3H), 3.68 (s, 5H), 3.57-3.58 (m, 3H), 3.07 (s, 3H), 1.07 (d, J = 13.20 Hz, 6H). | LCMS: 93.01% (445.55 M − H); | R2 |
| 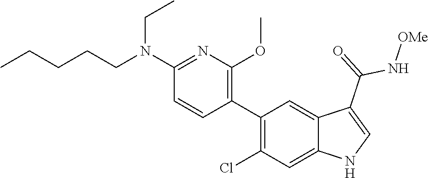 | 1117 | ¹H NMR; 400 MHz, DMSO- d6: δ 11.68 (br s, 1H), 11.11 (br s, 1H), 7.92 (s, 2H), 7.54 (s, 1H), 7.28 (d, J = 8.00 Hz, 1H), 6.16 (d, J = 8.00 Hz, 1H), 3.73 (s, 3H), 3.71 (s, 3H), 3.48-3.46 (m, 4H), 1.60-1.58 (m, 2H), 1.37-1.35 (m, 2H), 1.16 (t, J = 7.20 Hz, 3H), 0.95 (t, J = 7.20 Hz, 3H). | LCMS: 99.15% (431.36 M + H); | R2 |
| 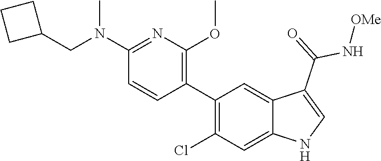 [SMILES: CONC(=O)c1c[nH]c2cc(Cl)c(-c3ccc(N(C)CC4CCC4)nc3OC)cc12] | 1054 | ¹H NMR, 400 MHz, DMSO- d6: δ 11.69 (s, 1H), 11.12 (s, 1H), 7.92 (s, 2H), 7.54 (s, 1H), 7.29 (d, J = 8.00 Hz, 1H), 6.17 (d, J = 8.00 Hz, 1H), 3.75 (s, 3H), 3.69 (s, 3H), 3.61 (d, J = 6.80 Hz, 2H), 3.02 (s, 3H), 2.73-2.67 (m, 1H), 2.01-1.91 (m, 2H), 1.88-1.76 (m, 4H). | LCMS: 95.38% (M − H = 427.33) | R3 |
| 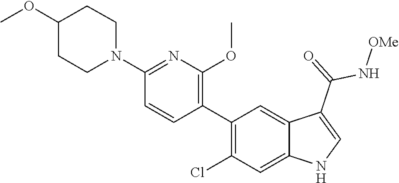 [SMILES: CONC(=O)c1c[nH]c2cc(Cl)c(-c3ccc(N4CCC(OC)CC4)nc3OC)cc12] | 1055 | ¹H NMR, 400 MHz, DMSO- d6: δ 11.12 (b, s, 2H), 7.93 (s, 2H), 7.55 (s, 1H), 7.33 (d, J = 8.40 Hz, 1H), 6.42 (d, J = 8.00 Hz, 1H), 3.97-3.98 (m, 2H), 3.63 (s, 6H), 3.42-3.40 (m, 1H), 3.32 (s, 3H), 3.24-3.22 (m, 2H), 1.95-1.93 (m, 2H), 1.49-1.47 (m, 2H). | LCMS: 97.25% (445.40 M + H); | R2 |
| 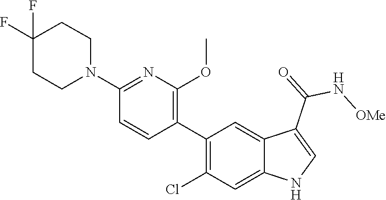 [SMILES: CONC(=O)c1c[nH]c2cc(Cl)c(-c3ccc(N4CCC(F)(F)CC4)nc3OC)cc12] | 1058 | ¹H NMR (400 MHz, DMSO- d6: δ 11.76 (s, 1H), 11.16 (s, 1H), 7.94 (s, 1H), 7.93 (s, 1H), 7.56 (s, 1H), 7.39 (d, J = 8.00 Hz, 1H), 6.53 (d, J = 8.00 Hz, 1H), 3.76-3.69 (m, 10H), 2.07- 2.01 (m, 4H). | LCMS: 97.01% (M − H = 449.27) | R1 |
| 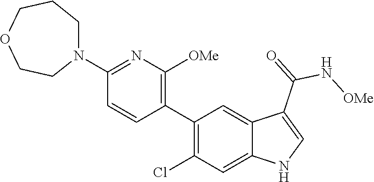 [SMILES: CONC(=O)c1c[nH]c2cc(Cl)c(-c3ccc(N4CCCOCC4)nc3OC)cc12] | 1059 | ¹H NMR 400 MHz, DMSO- d6: δ 11.40 (s, 2H), 7.93 (d, J = 3.20 Hz, 2H), 7.57 (s, 1H), 7.31 (d, J = 8.00 Hz, 1H), 6.28 (d, J = 8.00 Hz, 1H), 3.79 (s, 3H), 3.74 (s, 3H), 3.73 (m, 4H), 3.64 (t, J = 5.60 Hz, 4H), 1.94 (t, J = 5.60 Hz, 2H). | LCMS: 88.49% (M + H = 431.33) | R3 |
| 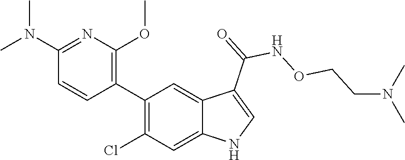 | 1060 | ¹H NMR (400 MHz, DMSO- d6): δ 11.71 (s, 1H), 11.08 (s, 1H), 7.98 (s, 1H), 7.93 (s, 1H), 7.54 (s, 1H), 7.31 (d, J = 8.00 Hz, 1H), 6.22 (d, J = 8.00 Hz, 1H), 3.95 (t, J = 5.60 Hz, 2H), 3.76 (s, 3H), 3.07 (s, 6H), 2.50 (t, J = 2.00 Hz, 2H), 2.19 (s, 6H). | LCMS: 96.61% (430.27 [M − H]) | R1 |
| 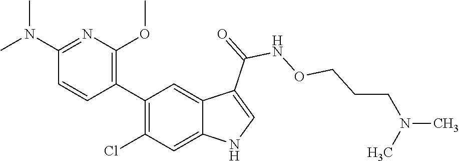 | 1061 | ¹H NMR (400 MHz, DMSO- d6): δ 11.68 (s, 1H), 11.06 (s, 1H), 7.93 (d, J = 8.40 Hz, 2H), 7.54 (s, 1H), 7.31 (d, J = 8.00 Hz, 1H), 6.22 (d, J = 8.00 Hz, 1H), 3.88 (t, J = 6.40 Hz, 2H), 3.76 (s, 3H), 3.07 (s, 6H), 2.31 (t, J = 7.20 Hz, 2H), 2.12 (s, 6H), 1.76-1.69 (m, 2H). | LCMS: 97.82% (444.36 [M − H]⁻). | R1 |
| 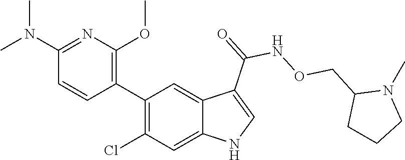 | 1062 | ¹H NMR 400 MHz, DMSO- d6: δ 11.69 (s, 1H), 11.10 (s, 1H), 7.95 (d, J = 2.00 Hz, 1H), 7.92 (s, 1H), 7.54 (s, 1H), 7.31 (d, J = 8.00 Hz, 1H), 6.22 (d, J = 8.00 Hz, 1H), 3.88-3.91 (m, 1H), 3.73-3.76 (m, 4H), 3.07 (s, 6H), 2.90-2.93 (m, 1H), 2.45-2.46 (m, 1H), 2.32 (s, 3H), 2.10-2.17 (m, 1H), 1.88- | LCMS: 95.15% (456.31, M − H) | R1 |
| | | 1.95 (m, 1H), 1.60-1.67 (m, | | |
| | | 3H). | | |
| 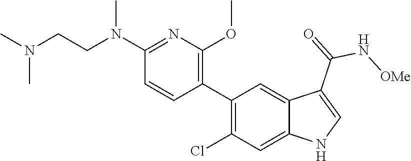 [SMILES: CONC(=O)c1c[nH]c2cc(Cl)c(-c3ccc(N(C)CCN(C)C)nc3OC)cc12] | 1063 | ¹H NMR (400 MHz, DMSO- d6): δ 11.68 (s, 1H), 11.12 (s, 1H), 7.93 (s, 2H), 7.54 (s, 1H), 7.30 (d, J = 8.40 Hz, 1H), 6.17 (d, J = 8.00 Hz, 1H), 3.75 (s, 3H), 3.69 (s, 3H), 3.66 (t, J = 8.80 Hz, 2H), 3.03 (s, 3H), 2.49 (t, J = 14.40 Hz, 2H), 2.21 (s, 6H). | LCMS: 98.29% (M + H = 432.33) | R3 |
| 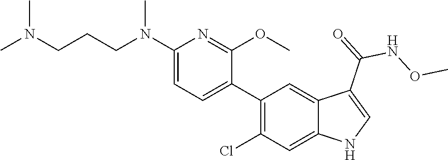 | 1064 | ¹H NMR (DMSO-d6, 400 Hz): δ 11.68 (s, 1H), 11.12 (s, 1H), 7.93 (d, J = 3.2 Hz, 2H), 7.54 (s, 1H), 7.30 (d, J = 8.0 Hz, 1H), 6.19 (d, J = 8.0 Hz, 1H), 3.75 (s, 3H), 3.69 (s, 3H), 3.56 (t, J = 6.8 Hz, 2H), 3.03 (s, 3H), 2.37-2.30 (m, 2H), 2.21 (s, 6H), 1.30-1.18 (m, 2H). | LCMS: 97.00%, 444.43 (M − H) | R3 |
| 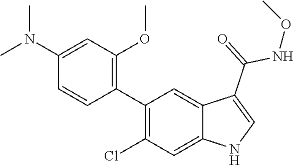 [SMILES: CONC(=O)c1c[nH]c2cc(Cl)c(-c3ccc(N(C)C)cc3OC)cc12] | 1073 | ¹H NMR, 400 MHz, DMSO- d6: δ 11.70 (br s, 1H), 11.13 (bs, 1H), 7.91 (d, J = 9.20 Hz, 2H), 7.52 (s, 1H), 6.93 (d, J = 8.40 Hz, 1H), 6.35 (d, J = 2.00 Hz, 2H), 3.68 (s, 6H), 2.96 (s, 6H). | LCMS: 99.37% (372.19 M − H) | R1 |
| 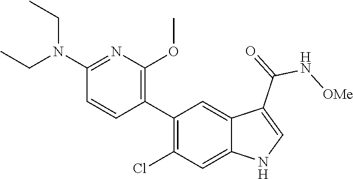 [SMILES: CCN(CC)c1ccc(-c2cc3c(C(=O)NOC)c[nH]c3cc2Cl)c(OC)n1] | 1074 | ¹H NMR (400 MHz, DMSO- d6: δ 11.69 (s, 1H), 11.12 (s, 1H), 7.93 (s, 2H), 7.55 (s, 1H), 7.29 (d, J = 8.40 Hz, 1H), 6.18 (d, J = 8.40 Hz, 1H), 3.75 (s, 3H), 3.70 (s, 3H), 3.52 (q, J = 6.80 Hz, 4H), 1.18 (t, J = 7.2 Hz, 6H). | LCMS: 99.87% (M + H = 403.39) | R1 |
| 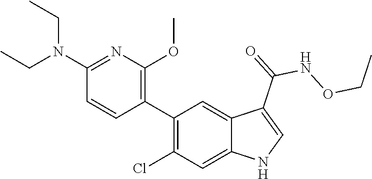 [SMILES: CCONC(=O)c1c[nH]c2cc(Cl)c(-c3ccc(N(CC)CC)nc3OC)cc12] | 1075 | ¹H NMR (400 MHz, DMSO- d6: δ 11.66 (s, 1H), 11.02 (s, 1H), 7.94-7.94 (m, 2H), 7.55 (s, 1H), 7.28 (d, J = 8.40 Hz, 1H), 6.17 (d, J = 8.40 Hz, 1H), 3.91 (q, J = 7.20 Hz, 2H), 3.74 (s, 3H), 3.52 (q, J = 6.80 Hz, 4H), 1.23-1.12 (m, 9H). | LCMS: 99.51% (M − H = 415.32) | R1 |
| 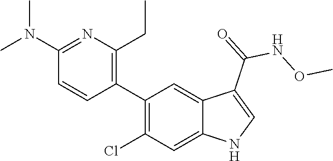 | 1076 | ¹H NMR (400 MHz, DMSO- d6: δ 11.75 (s, 1H), 11.14 (s, 1H), 7.96 (s, 1H), 7.93 (s, 1H), 7.61 (s, 1H), 7.24 (d, J = 8.40 Hz, 1H), 6.52 (d, J = 8.40 Hz, 1H), 3.69 (s, 3H), 3.07 (s, 6H), 2.37 (q, J = 7.60 Hz, 2H), 1.06 (t, J = 7.60 Hz, 3H). | LCMS: 95.8% (M + H = 373.24) | R1 |
| 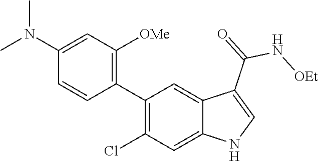 [SMILES: CCONC(=O)c1c[nH]c2cc(Cl)c(-c3ccc(N(C)C)cc3OC)cc12] | 1104 | ¹H NMR 400 MHz, DMSO- d6: δ 11.32 (br s, 2H), 7.94 (s, 1H), 7.89 (s, 1H), 7.54 (s, 1H), 6.93 (d, J = 8.00 Hz, 1H), 6.37-6.33 (m, 2H), 3.90 (q, J = 7.2 Hz, 2H), 3.68 (s, 3H), 2.96 (s, 6H), 1.19 (t, J = 6.8 Hz, 3H). | LCMS: 98.81% (M + H = 386.26) | R1 |
| 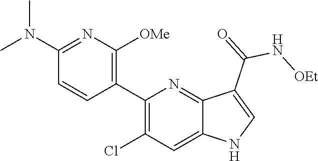 [SMILES: CCONC(=O)c1c[nH]c2cc(Cl)c(-c3ccc(N(C)C)nc3OC)nc12] | 1090 | ¹H NMR (400 MHz, DMSO- d6): δ 12.13 (s, 1H), 10.94 (s, 1H), 8.27 (d, J = 2.40 Hz, 1H), 8.06 (s, 1H), 7.53 (d, J = 8.40 Hz, 1H), 6.28 (d, J = 8.00 Hz, 1H), 3.95 (q, J = 6.80 Hz, 2H), 3.81 (s, 3H), 3.10 (s, 6H), 1.19 (t, J = 7.20 Hz, 3H). | LCMS: 96.55% ([M + H] = 390.24) | R1 |
| 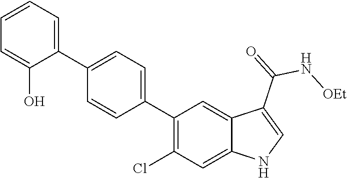 [SMILES: CCONC(=O)c1c[nH]c2cc(Cl)c(-c3ccc(-c4ccccc4O)cc3)cc12] | 1080 | ¹H NMR, 400 MHz, DMSO- d6: δ 11.77 (s, 1H), 11.07 (s, 1H), 9.59 (s, 1H), 8.10 (s, 1H), 8.00 (s, 1H), 7.64 (d, J = 8.40 Hz, 3H), 7.47 (d, J = 8.40 Hz, 2H), 7.34 (d, J = 6.40 Hz, 1H), 7.18 (t, J = 7.20 Hz, 1H), 6.98 (d, J = 8.00 Hz, 1H), 6.91 (t, J = 7.60 Hz, 1H), 3.92 (q, J = 6.80 Hz, 2H), 1.21 (t, J = 6.80 Hz, 3H). | LCMS: 97.38% (M + H = 407.24) | R1 |
| 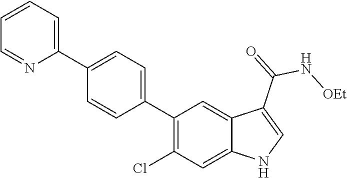 [SMILES: CCONC(=O)c1c[nH]c2cc(Cl)c(-c3ccc(-c4ccccn4)cc3)cc12] | 1103 | ¹H NMR, 400 MHz, DMSO- d6: δ 11.82 (s, 1H), 11.09 (s, 1H), 8.71-8.70 (m, 1H), 8.15 (dd, J = −31.60, Hz, 2H), 8.05 (s, 1H), 8.02 (d, J = 10.00 Hz, 2H), 7.94 (d, J = 1.60 Hz, 1H), 7.67 (s, 1H), 7.57 (d, J = 8.40 Hz, 2H), 7.40-7.37 (m, 1H), 3.92 (q, J = 6.80 Hz, 2H), 1.21 (t, J = 6.80 Hz, 3H). | LCMS: 99.65% (M + H = 392.24) | R3 |
| 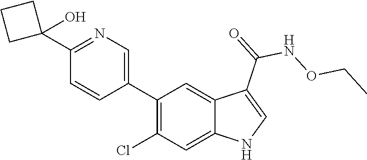 [SMILES: CCONC(=O)c1c[nH]c2cc(Cl)c(-c3ccc(C4(O)CCC4)nc3)cc12] | 1099 | ¹H NMR (400 MHz, DMSO- d6: δ 11.80 (br s, 1H), 11.09 (br s, 1H), 8.60 (d, J = 1.60 Hz, 1H), 8.08 (s, 1H), 8.01 (s, 1H), 7.85 (dd, J = 2.0, 8.40, Hz, 1H), 7.68-7.65 (m, 2H), 5.78 (s, 1H), 3.92 (q, J = 6.8 Hz, 2H), 2.67-2.59 (m, 2H), 2.33-2.22 (m, 2H), 1.98-1.86 (m, 2H), 1.21 (t, J = 7.2 Hz, 3H). | LCMS: 99.37% (M + H = 386.26) | R1 |
| 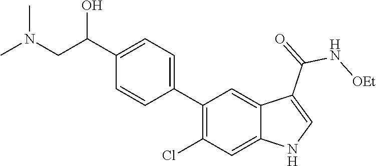 [SMILES: CCONC(=O)c1c[nH]c2cc(Cl)c(-c3ccc(C(O)CN(C)C)cc3)cc12] | 1094 | ¹H NMR (400 MHz, DMSO- d6): δ 11.76 (s, 1H), 11.06 (s, 1H), 8.03 (s, 1H), 7.98 (s, 1H), 7.62 (s, 1H), 7.43-7.36 (m, 4H), 5.04 (br s, 1H), 4.71 (br s, 1H), 3.92 (q, J = 7.2 Hz, 2H), 3.50-3.40 (m, 2H), 2.23 (s, 6H), 1.20 (t, J = 7.2 Hz, 3H). | LCMS: 90.92% (M + H = 402.29) | |
| 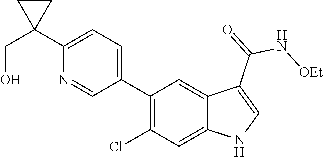 [SMILES: CCONC(=O)c1c[nH]c2cc(Cl)c(-c3ccc(C4(CO)CC4)nc3)cc12] | 1101 | ¹H NMR 400 MHz, DMSO- d6: δ 11.95 (br s, 1H), 11.09 (br s, 1H), 8.48 (d, J = 1.60 Hz, 1H), 8.05 (s, 1H), 8.01 (s, 1H), 7.77 (dd, J = 2.40, 8.00 Hz, 1H), 7.67 (s, 1H), 7.59 (d, J = 8.40 Hz, 1H), 4.82 (br, s, 1H), 3.92 (q, J = 7.20 Hz, 2H), 3.81 (d, J = 4.40 Hz, 2H), 1.17-1.17 (m, 5H), 0.94 (d, J = 2.00 Hz, 2H). | LCMS: 97.86% (M+ = 386.26) | R1 |
| 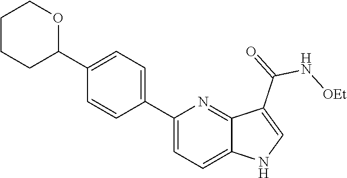 [SMILES: CCONC(=O)c1c[nH]c2ccc(-c3ccc(C4CCCCO4)cc3)nc12] | 1098 | ¹H NMR, 400 MHz, DMSO- d6: δ 12.18 (s, 1H), 11.36 (s, 1H), 8.26 (s, 1H), 8.06 (d, J = 8.40 Hz, 2H), 8.00 (d, J = 8.80 Hz, 1H), 7.81 (d, J = 8.80 Hz, 1H), 7.48 (d, J = 8.00 Hz, 2H), 4.40 (d, J = 9.60 Hz, 1H), 4.04 (q, J = 7.20 Hz, 3H), 3.57 (q, J = 11.20 Hz, 1H), 1.88 (t, J = 11.60 Hz, 2H), 1.70-1.58 (m, 4H), 1.31 (t, J = 7.20 Hz, 3H). | LCMS: 98.04% (M − H = 366.30) | R3 |
| 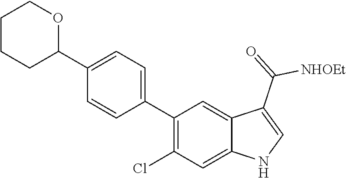 | 1102 | ¹H NMR, 400 MHz, DMSO- d6: δ 11.82 (s, 1H), 11.12 (s, 1H), 8.03 (s, 1H), 7.98 (s, 1H), 7.62 (s, 1H), 7.42-7.37 (m, 4H), 4.38 (d, J = 9.20 Hz, 1H), 4.07 (d, J = 6.40 Hz, 1H), 3.94-3.89 (m, 2H), 3.60-3.53 (m, 1H), 1.91-1.68 (m, 2H), 1.66-1.48 (m, 4H), 1.20 (t, J = 7.20 Hz, 3H). | LCMS: 99.87% (M + H = 399.32) | R3 |
| 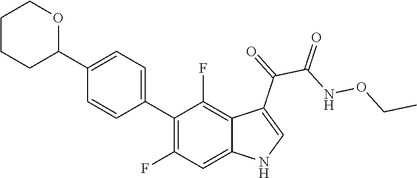 [SMILES: CCONC(=O)C(=O)c1c[nH]c2cc(F)c(-c3ccc(C4CCCCO4)cc3)c(F)c12] | 1096 | ¹H NMR (400 MHz, DMSO- d6): δ 12.70-11.00 (br s, 2H), 8.59 (s, 1H), 7.45-7.38 (m, 4H), 7.33 (d, J = 9.6 Hz, 1H), 4.38 (d, J = 10.00 Hz, 1H), 4.05 (d, J = 11.20 Hz, 1H), 3.93 (q, J = 7.20 Hz, 2H), 3.53-3.60 (m, 1H), 1.91-1.86 (m, 2H), 1.70-1.40 (m, 4H), 1.22 (t, J = 7.20 Hz, 3H). | LCMS: 93.20% ([M − H] = 427.26) | R1 |
| 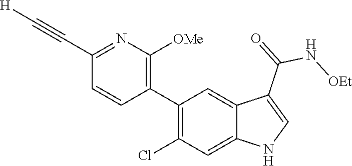 [SMILES: C#Cc1ccc(-c2cc3c(C(=O)NOCC)c[nH]c3cc2Cl)c(OC)n1] | 1093 | ¹H NMR (400 MHz, DMSO- d6):: δ 11.79 (s, 1H), 11.07 (s, 1H), 7.98 (d, J = 9.6 Hz, 2H), 7.65-7.61 (m, 2H), 7.29 (d, J = 7.6 Hz, 1H), 4.36 (s, 1H), 3.90 (q, J = 7.6 Hz, 2H), 3.80 (s, 3H), 1.20 (t, J = 6.8 Hz, 3H). | LCMS: 99.08% ([M + H] = 370.18) | R3 |
| 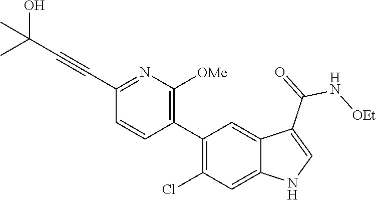 [SMILES: CCONC(=O)c1c[nH]c2cc(Cl)c(-c3ccc(C#CC(C)(C)O)nc3OC)cc12] | 1092 | ¹H NMR (400 MHz, DMSO- d6): δ 11.79 (s, 1H), 11.08 (s, 1H), 7.99 (s, 1H), 7.97 (s, 1H), 7.66 (s, 1H), 7.61 (d, J = 7.20 Hz, 1H), 7.18 (d, J = 7.20 Hz, 1H), 5.62 (s, 1H), 3.91 (q, J = 7.20 Hz, 2H), 3.80 (s, 3H), 1.50 (s, 6H), 1.20 (t, J = 7.20 Hz, 3H). | LCMS: 99.36% ([M − H] = 426.23) | R2 |
| 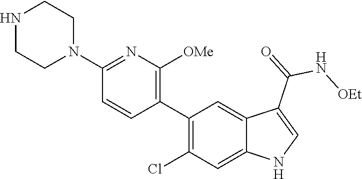 [SMILES: CCONC(=O)c1c[nH]c2cc(Cl)c(-c3ccc(N4CCNCC4)nc3OC)cc12] | 1095 | ¹H NMR, 400 MHz, DMSO- d6: δ 11.70 (s, 1H), 11.03 (s, 1H), 7.94 (d, J = 8.40 Hz, 2H), 7.55 (s, 1H), 7.34 (s, 1H), 6.37 (s, 1H), 3.91 (q, J = 7.20 Hz, 2H), 3.74 (s, 3H), 3.49-3.33 (m, 4H), 2.82 (br, s, 4H), 1.20 (t, J = 6.80 Hz, 3H). | LCMS: 98.38% (M + H = 428.28) | R3 |
| 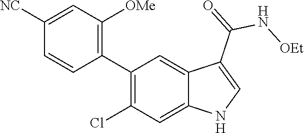 [SMILES: CCONC(=O)c1c[nH]c2cc(Cl)c(-c3ccc(C#N)cc3OC)cc12] | 1097 | ¹H NMR 400 MHz, DMSO- d6: δ 11.74 (br, s, 1H), 11.11 (br, s, 1H), 7.99 (s, 1H), 7.94 (s, 1H), 7.61 (s, 1H), 7.59 (s, 1H), 7.51 (d, J = 7.60 Hz, 1H), 7.38 (d, J = 7.60 Hz, 1H), 3.89 (q, J = 6.80 Hz, 2H), 3.77 (s, 1H), 1.20 (t, J = 7.20 Hz, 3H). | LCMS: 99.00% (M+ = 370.22) | R3 |
| 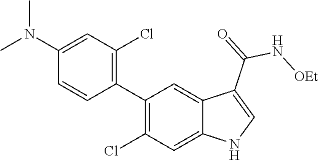 [SMILES: CCONC(=O)c1c[nH]c2cc(Cl)c(-c3ccc(N(C)C)cc3Cl)cc12] | 1091 | ¹H NMR 400 MHz, DMSO- d6: δ 11.73 (br, s, 1H), 11.05 (br, s, 1H), 7.97 (d, J = 2.8 Hz, 1H), 7.93 (s, 1H), 7.58 (s, 1H), 7.12 (d, J = 8.4 Hz, 1H), 6.79 (d, J = 2.4 Hz, 1H), 6.74 (dd, J = 8.4 Hz, 2.4 Hz, 2H), 3.91 (q, J = 7.2 Hz, 2H), 2.96 (s, 6H), 1.19 (t, J = 14 Hz, 7.2 Hz, 3H). | LCMS: 95.10% (M + H = 392.26) | R1 |
| 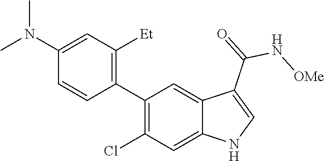 [SMILES: CCc1cc(N(C)C)ccc1-c1cc2c(C(=O)NOC)c[nH]c2cc1Cl] | 1100 | ¹H NMR, 400 MHz, DMSO- d6: δ 11.72 (s, 1H), 11.13 (s, 1H), 7.94 (s, 1H), 7.92 (s, 1H), 7.58 (s, 1H), 6.90 (d, J = 8.40 Hz, 1H), 6.66-6.60 (m, 2H), 3.69 (s, 3H), 2.94 (s, 6H), 2.31 (q, J = 7.60 Hz, 2H), 0.96 (t, J = 7.60 Hz, 3H). | LCMS: 97.81% (M + H = 372.22) | R1 |
| 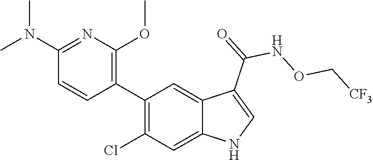 [SMILES: COc1nc(N(C)C)ccc1-c1cc2c(C(=O)NOCC(F)(F)F)c[nH]c2cc1Cl] | 1067 | ¹H NMR (400 MHz, DMSO- d6): δ 11.68 (d, J = 56.40 Hz, 2H), 7.97 (s, 1H), 7.91 (s, 1H), 7.56 (s, 1H), 7.31 (d, J = 8.40 Hz, 1H), 6.22 (d, J = 8.00 Hz, 1H), 4.55-4.48 (m, 2H), 3.76 (s, 3H), 3.07 (s, 6H). | LCMS: 99.03% (443.29 [M + H]⁺). | R1 |
| 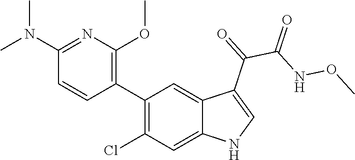 | 1077 | ¹H NMR (400 MHz, DMSO- d6): δ 12.33 (s, 1H), 12.03 (s, 1H), 8.70 (s, 1H), 8.03 (s, 1H), 7.65 (s, 1H), 7.33 (d, J = 8.40 Hz, 1H), 6.23 (d, J = 8.40 Hz, 1H), 3.76 (s, 3H), 3.70 (s, 3H), 3.07 (s, 6H). | LCMS: 92.53% (403.32 [M + H]⁻) | R1 |
| 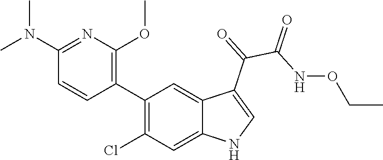 [SMILES: CCONC(=O)C(=O)c1c[nH]c2cc(Cl)c(-c3ccc(N(C)C)nc3OC)cc12] | 1081 | ¹H NMR (400 MHz, DMSO- d6): δ 12.30 (s, 1H), 11.90 (s, 1H), 8.67 (s, 1H), 8.03 (s, 1H), 7.65 (s, 1H), 7.33 (d, J = 8.40 Hz, 1H), 6.23 (d, J = 8.40 Hz, 1H), 3.93 (q, J = 7.20 Hz, 2H), 3.76 (s, 3H), 3.07 (s, 6H), 1.20 (t, J = 7.20 Hz, 3H). | LCMS: 99.49% (417.36 [M + H]⁻) | R1 |
| 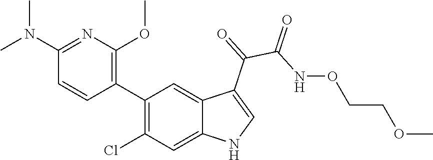 | 1088 | ¹H NMR (400 MHz, DMSO- d6): δ 12.13 (br s, 2H), 8.65 (s, 1H), 8.02 (s, 1H), 7.64 (s, 1H), 7.33 (d, J = 8.0 Hz, 1H), 6.23 (d, J = 8.4 Hz, 1H), 4.01 (t, J = 4.4 Hz, 2H), 3.76 (s, 3H), 3.57 (t, J = 4.4 Hz, 2H), 3.29 (s, 3H), 3.07 (s, 6H) | LCMS: 98.91% (M + H = 447.29) | R1 |
| 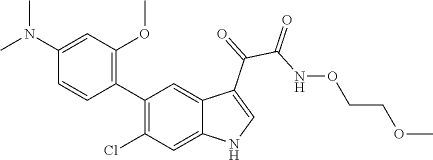 | 1111 | ¹H NMR (400 MHz, DMSO- d6): δ 12.28 (br s, 1H), 11.99 (br s, 1H), 8.65 (s, 1H), 7.99 (s, 1H), 7.62 (s, 1H), 6.94 (d, J = 8.4 Hz, 1H), 6.40-6.34 (m, 2H), 4.01 (s, 2H), 3.69 (s, 3H), 3.57 (t, J = 4.4 Hz, 2H), 3.29 (s, 3H), 2.97 (s, 6H) | LCMS: 98.53% (M + H = 446.27) | R1 |
| 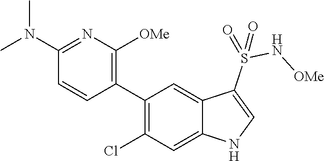 [SMILES: CONS(=O)(=O)c1c[nH]c2cc(Cl)c(-c3ccc(N(C)C)nc3OC)cc12] | 1028 | ¹H NMR (400 MHz, DMSO- d6): δ 12.20 (s, 1H), 10.14 (s, 1H), 8.03 (s, 1H), 7.65 (s, 1H), 7.61 (s, 1H), 7.32 (d, J = 8.00 Hz, 1H), 6.23 (d, J = 8.40 Hz, 1H), 3.77 (s, 3H), 3.63 (s, 3H), 3.07 (s, 6H). | LCMS: 97.28% ([M + H] = 411.25) | Synthesis in Example 4 |
| 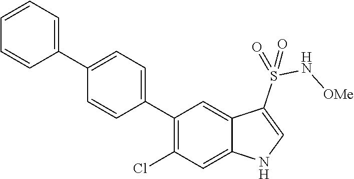 [SMILES: CONS(=O)(=O)c1c[nH]c2cc(Cl)c(-c3ccc(-c4ccccc4)cc3)cc12] | 1110 | ¹H NMR (400 MHz, DMSO- d6): δ 12.32 (s, 1H), 10.20 (s, 1H), 8.10 (s, 1H), 7.73-7.82 (m, 6H), 7.49-7.57 (m, 4H), 7.38-7.42 (m, 1H), 3.65 (s, 3H). | LCMS: 99.11% ([M − H] = 411.18) | Synthesis in Example 4 |
| 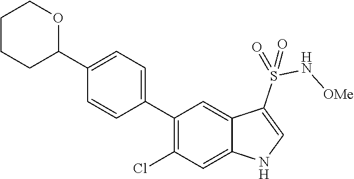 [SMILES: CONS(=O)(=O)c1c[nH]c2cc(Cl)c(-c3ccc(C4CCCCO4)cc3)cc12] | 1109 | ¹H NMR (400 MHz, DMSO- d6): δ 12.32 (s, 1H), 10.20 (s, 1H), 8.00 (s, 1H), 7.71 (s, 1H), 7.64 (s, 1H), 7.38-7.42 (m, 4H), 4.37 (dd, J = 1.60, 11.00 Hz, 1H), 4.05 (d, J = 10.80 Hz, 1H), 3.53-3.61 (m, 4H), 1.84- 1.90 (m, 2H), 1.53-1.70 (m, 4H). | LCMS: 97.90% ([M − H] = 419.20) | Synthesis in Example 4 |
| 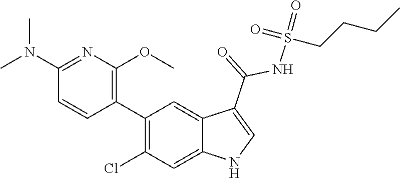 [SMILES: CCCCS(=O)(=O)NC(=O)c1c[nH]c2cc(Cl)c(-c3ccc(N(C)C)nc3OC)cc12] | 1108 | ¹H NMR 400 MHz, CDCl3: δ 11.19 (s, 1H), 8.12 (s, 1H), 7.66 (s, 1H), 7.40 (s, 1H), 7.28 (d, J = 8.00 Hz, 1H), 6.20 (d, J = 8.40 Hz, 1H), 3.76 (s, 3H), 3.06-3.01 (m, 8H), 1.57-1.53 (m, 2H), 1.33-1.24 (m, 3H), 0.83 (t, J = 7.6 Hz, 3H). | LCMS: 97.28% (M + H = 465.34) | Synthesis in Example 2 |
| 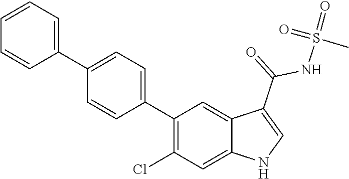 [SMILES: CS(=O)(=O)NC(=O)c1c[nH]c2cc(Cl)c(-c3ccc(-c4ccccc4)cc3)cc12] | 1112 | ¹HNMR 400 MHz, DMSO-d6: δ 12.06 (s, 1H), 11.66 (s, 1H), 8.41 (s, 1H), 8.16 (s, 1H), 7.80- 7.60 (m, 5H), 7.70 (s, 1H), 7.60-7.50 (m, 4H), 7.40 (t, J = 7.20 Hz, 1H), 3.34 (s, 3H). | LCMS: 96.02% (M/Z 423.21, [M + H]+) | R1 |
| 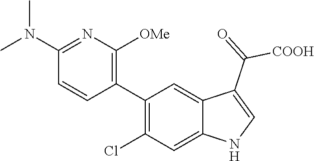 [SMILES: COc1nc(N(C)C)ccc1-c1cc2c(C(=O)C(=O)O)c[nH]c2cc1Cl] | 1113 | ¹H NMR (400 MHz, DMSO- d6): δ 13.30 (br s, 1H), 11.78 (s, 1H), 8.13 (s, 1H), 8.00 (s, 1H), 7.54 (s, 1H), 7.30 (d, J = 8.00 Hz, 1H), 6.22 (d, J = 8.00 Hz, 1H), 3.76 (s, 3H), 3.07 (s, 6H). | LCMS: 97.23% (374.20 [M + H]⁻). | Synthesis in Example 5 |
| 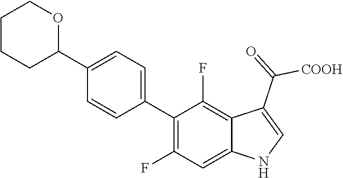 [SMILES: O=C(O)C(=O)c1c[nH]c2cc(F)c(-c3ccc(C4CCCCO4)cc3)c(F)c12] | 1114 | | LCMS: ([M + H] = 386.29) | Synthesis in Example 1 |

A number of patent and non-patent publications are cited herein in order to describe the state of the art to which this disclosure pertains. The entire disclosure of each of these publications is incorporated by reference herein.

While certain embodiments of the present disclosure have been described and/or exemplified above, various other embodiments will be apparent to those skilled in the art from the foregoing disclosure. The present disclosure is, therefore, not limited to the particular embodiments described and/or exemplified, but is capable of considerable variation and modification without departure from the scope and spirit of the appended claims.

1. A compound of formula (I), or a pharmaceutically acceptable salt, solvate, hydrate, cocrystal, or prodrug thereof: 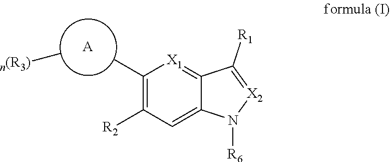
wherein in formula (I):
X₁ is CR₄ₐ or N;
X₂ is CR_{4b} or N;
R₁ is selected from 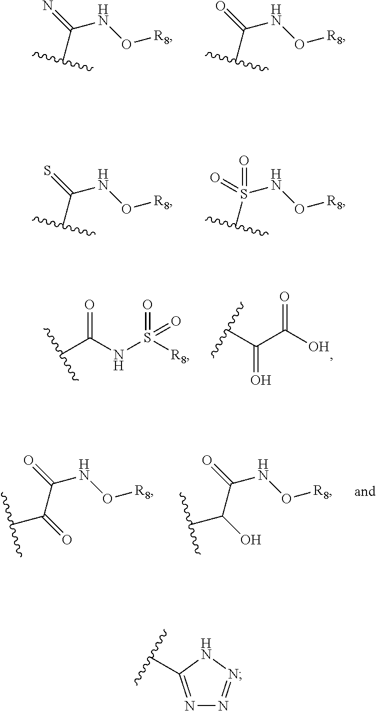
R₂ is selected from H, C₁₋₃ alkyl, —CF₃, and halo;
R₃ is at each occurrence independently selected from halo, —CN, 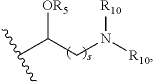
—N(R₁₀)₂, C₁₋₁₀ alkyl, C₂₋₁₀ alkynyl, C₁₋₁₀ alkoxy, C₃₋₇ cycloalkyl, C₃₋₇ cycloalkyloxy, C₃₋₁₀ heterocyclyl, C₄₋₁₂ alkylcycloalkyl, C₄₋₁₀ cycloalkylalkyl, C₃₋₇ heterocycloalkenyl, C₄₋₁₂ alkylheterocycloalkenyl, C₄₋₁₀ heterocycloalkenylalkyl, aryl and heteroaryl, wherein the C₁₋₁₀ alkyl, C₂₋₁₀ alkynyl, C₁₋₁₀ alkoxy, C₃₋₇ cycloalkyl, C₃₋₁₀ heterocyclyl, C₄₋₁₂ alkylcycloalkyl, C₄₋₁₀ cycloalkylalkyl, C₃₋₇ heterocycloalkenyl, C₄₋₁₂ alkylheterocycloalkenyl, C₄₋₁₀ heterocycloalkenylalkyl, aryl, or heteroaryl is unsubstituted or substituted with one to three substituents selected from deuterium, halo, C₁₋₆ alkyl, C₃₋₆ cycloalkyl, —OR₅ and —OCOR₇;
R₄ₐ is at each occurrence independently selected from H, halo, C₁₋₁₀ alkyl, C₃₋₇ cycloalkyl, C₃₋₁₀ heterocyclyl, C₄₋₁₂ alkylcycloalkyl, and C₄₋₁₀ cycloalkylalkyl, wherein the C₁₋₁₀ alkyl, C₃₋₇ cycloalkyl, C₃₋₁₀ heterocyclyl, C₄₋₁₂ alkylcycloalkyl, or C₄₋₁₀ cycloalkylalkyl is unsubstituted or substituted with one to three halogen substituents;
R_{4b} is at each occurrence independently selected from H, C₁₋₁₀ alkyl, C₃₋₇ cycloalkyl, C₃₋₁₀ heterocyclyl, C₄₋₁₂ alkylcycloalkyl, and C₄₋₁₀ cycloalkylalkyl, wherein the C₁₋₁₀ alkyl, C₃₋₇ cycloalkyl, C₃₋₁₀ heterocyclyl, C₄₋₁₂ alkylcycloalkyl, or C₄₋₁₀ cycloalkylalkyl is unsubstituted or substituted with one to three halogen substituents;
R₅ is at each occurrence independently selected from H, C₁₋₁₀ alkyl, C₁₋₁₀ alkoxy, C₃₋₇ cycloalkyl, C₄₋₁₂ alkylcycloalkyl, and C₄₋₁₀ cycloalkylalkyl, wherein the C₁₋₁₀ alkyl, C₁₋₁₀ alkoxy, C₃₋₇ cycloalkyl, C₄₋₁₂ alkylcycloalkyl, or C₄₋₁₀ cycloalkylalkyl is unsubstituted or substituted with one to three halogen substituents;
R₆ is at each occurrence independently selected from H, C₁₋₆ alkyl, and C₃₋₆ cycloalkyl;
R₇ is at each occurrence independently selected from C₁₋₁₀ alkyl, C₃₋₇ cycloalkyl, and C₄₋₁₂ alkylcycloalkyl, wherein the C₁₋₁₀ alkyl, C₃₋₇ cycloalkyl, or C₄₋₁₂ alkylcycloalkyl is unsubstituted or substituted with one to three substituents selected from halo, C₁₋₁₀ alkyl, and —NR₁₂,R₁₂;
R₈ is selected from H, C₁₋₁₀ alkyl, C₁₋₁₀ alkoxy, C₃₋₇ cycloalkyl, C₄₋₁₂ alkylcycloalkyl, C₄₋₁₀ cycloalkylalkyl, C₃₋₁₀ heterocyclyl, C₄₋₁₂ alkylheterocyclyl, C₃₋₇ heterocycloalkenyl, C₄₋₁₂ alkylheterocycloalkenyl, C₄₋₁₀ heterocycloalkenylalkyl, aryl, heteroaryl, 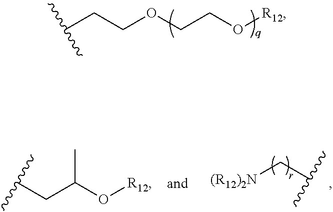
wherein the C₁₋₁₀ alkyl, C₁₋₁₀ alkoxy, C₃₋₇ cycloalkyl, C₄₋₁₂ alkylcycloalkyl, C₄₋₁₀ cycloalkylalkyl, C₃₋₁₀ heterocyclyl, C₄₋₁₂ alkylheterocyclyl, C₃₋₇ heterocycloalkenyl, C₄₋₁₂ alkylheterocycloalkenyl, C₄₋₁₀ heterocycloalkenylalkyl, aryl, or heteroaryl is unsubstituted or substituted with one to three substituents selected from deuterium, halo and —OR₅;
R₁₀ is at each occurrence independently selected from H, C₁₋₆ alkyl, C₃₋₆ cycloalkyl, C₄₋₁₂ alkylcycloalkyl, 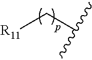
and C₃₋₁₀ heterocyclyl, wherein the C₁₋₆ alkyl, C₃₋₆ cycloalkyl, C₄₋₁₂ alkylcycloalkyl, or C₃₋₁₀ heterocyclyl is unsubstituted or substituted with one to three substituents selected from deuterium, halo, C₁₋₆ alkyl, and C₃₋₆ cycloalkyl substituents;
R₁₁ is at each occurrence independently selected from —OR₁₂ and —N(R₁₂)₂;
R₁₂ is at each occurrence independently selected from H, C₁₋₆ alkyl, C₁₋₁₀ haloalkoxy, and C₃₋₆ cycloalkyl;
n is an integer from 0-3;
p is an integer from 1-5;
q is an integer from 0-5;
r is an integer from 1-5;
s is an integer from 1-5;
A is selected from phenyl, pyridyl, pyrimidyl, pyridazyl and the following 5-membered ring heterocycles: 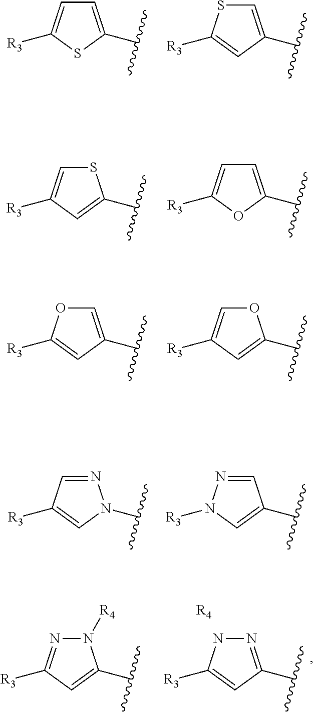
provided that the compound of formula (I) is not

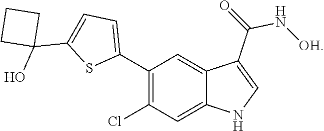 [SMILES: O=C(NO)c1c[nH]c2cc(Cl)c(-c3ccc(C4(O)CCC4)s3)cc12]

2. The compound of claim 1, wherein X₁ is CR₄ₐ.

3. The compound of claim 2, wherein R₄ₐ is H, Cl, or F.

4. The compound of any one of claims 1-3, wherein X₂ is N or CR₄, wherein R_{4b} is H.

5. The compound of any one of claims 1-4, wherein R₂ is halo.

6. The compound of claim 5, wherein R₂ is Cl or F.

7. The compound of any one of claims 1-6, wherein R₆ is H.

8. The compound of any one of claims 1-7, wherein A is selected from:

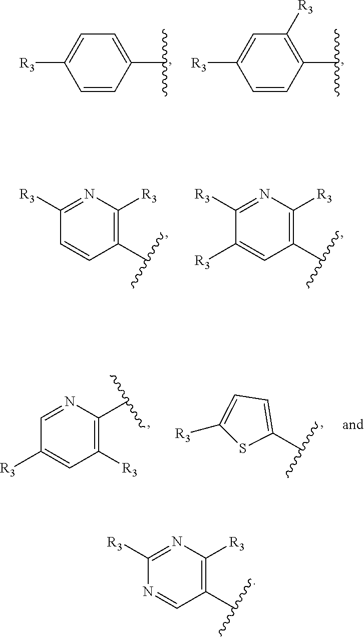

9. The compound of any one of claims 1-8, wherein the compound of formula (I) is a compound of formula (10), formula (11), formula (12), formula (13), formula (14), formula (15), formula (16), or formula (17) or a pharmaceutically acceptable salt, solvate, hydrate, cocrystal, or prodrug thereof:

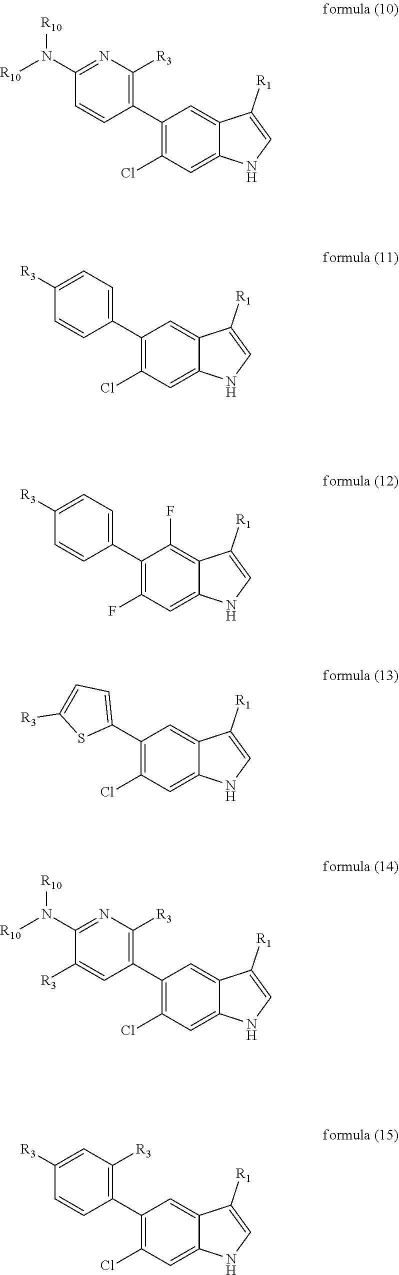

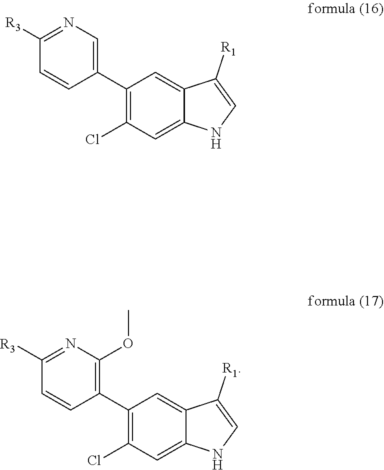

10. The compound of any one of claims 1-9, wherein R₁ is

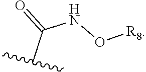

11. The compound of claim 10, wherein the compound of formula (I) is a compound of formula (100), formula (110), formula (120), formula (130), formula (140), formula (150), formula (160), or formula (170) or a pharmaceutically acceptable salt, solvate, hydrate, cocrystal, or prodrug thereof:

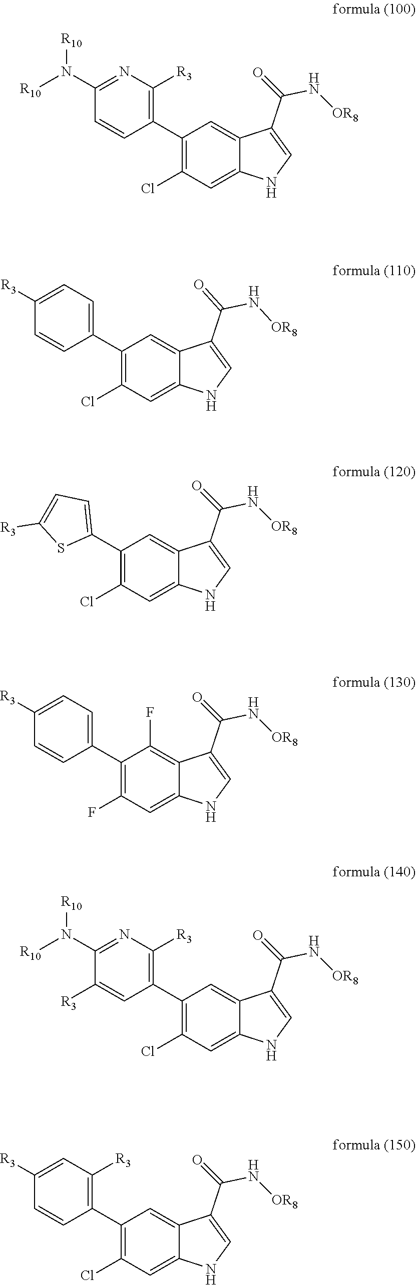

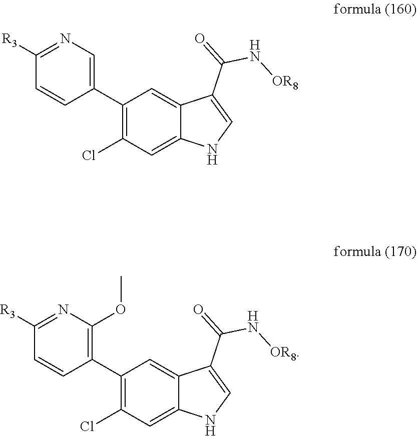

12. The compound of any one of claims 1-9, wherein R₁ is

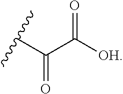 [SMILES: *C(=O)C(=O)O]

13. The compound of claim 12, wherein the compound of formula (I) is a compound of formula (200), formula (210), formula (220), formula (230), formula (240), formula (250), formula (260), or formula (270) or a pharmaceutically acceptable salt, solvate, hydrate, cocrystal, or prodrug thereof:

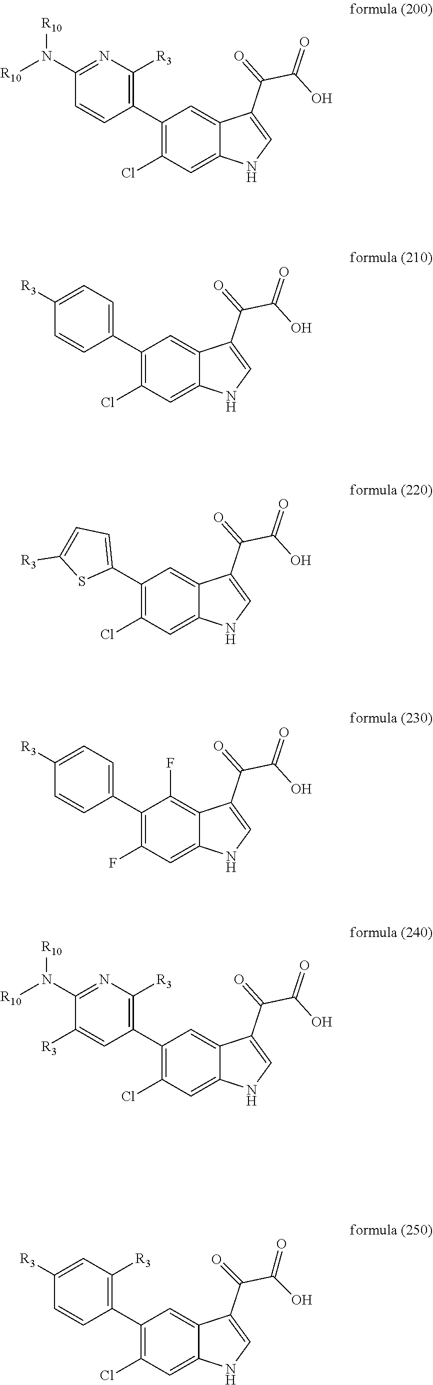

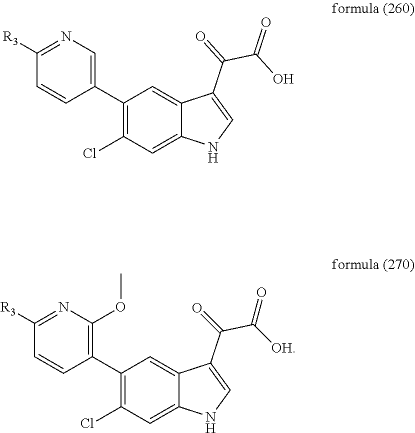

14. The compound of any one of claims 1-9, wherein R₁ is

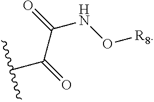

15. The compound of claim 14, wherein the compound of formula (I) is a compound of formula (300), formula (310), formula (320), formula (330), formula (340), formula (350), formula (360), or formula (370) or a pharmaceutically acceptable salt, solvate, hydrate, cocrystal, or prodrug thereof:

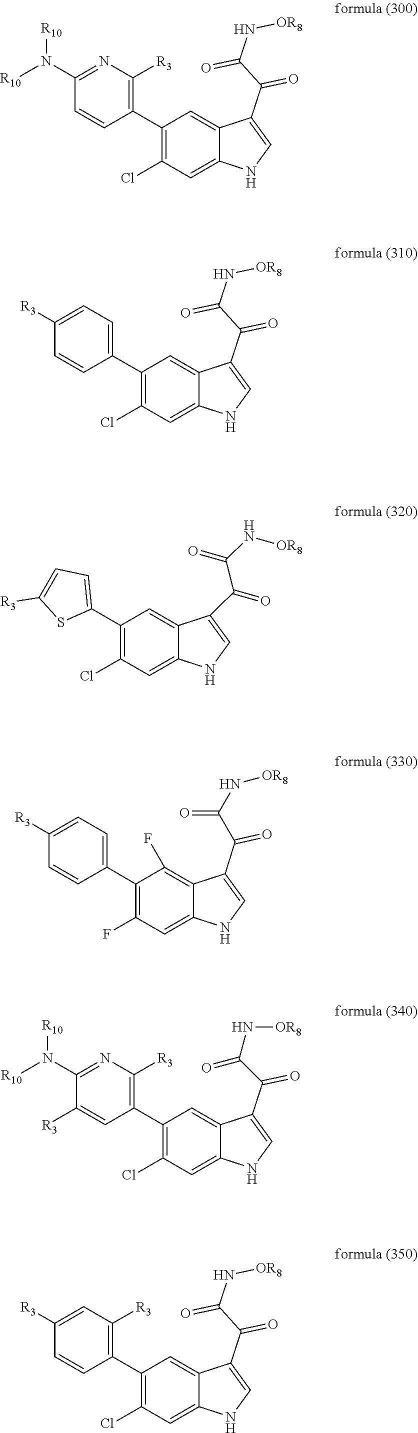

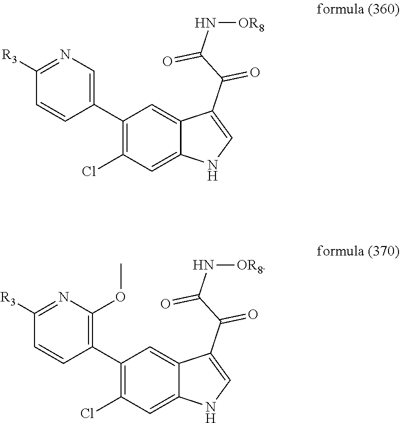

16. The compound of claim 14 or 15, wherein the compound is selected from:

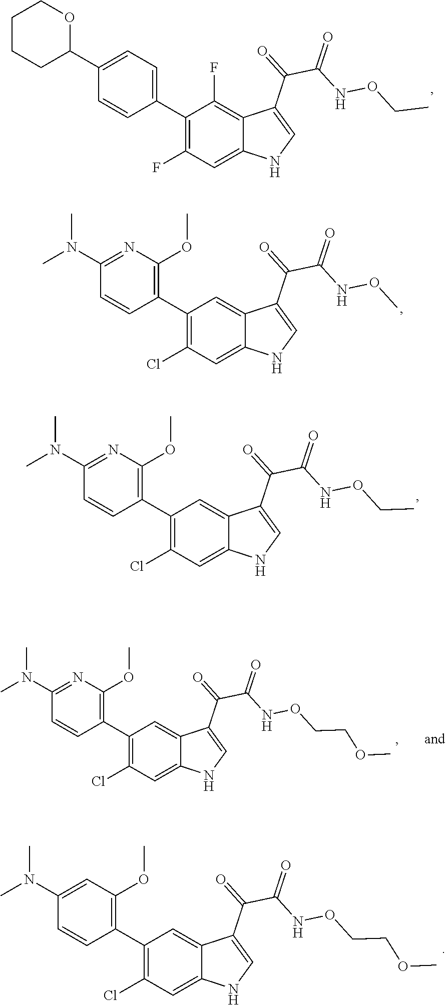

17. The compound of any one of claims 1-9, wherein R₁ is

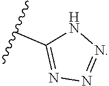 [SMILES: *c1nnn[nH]1]

18. The compound of claim 17, wherein the compound of formula (I) is a compound of formula (400), formula (410), formula (420), formula (430), formula (440), formula (450), formula (460), or formula (470) or a pharmaceutically acceptable salt, solvate, hydrate, cocrystal, or prodrug thereof:

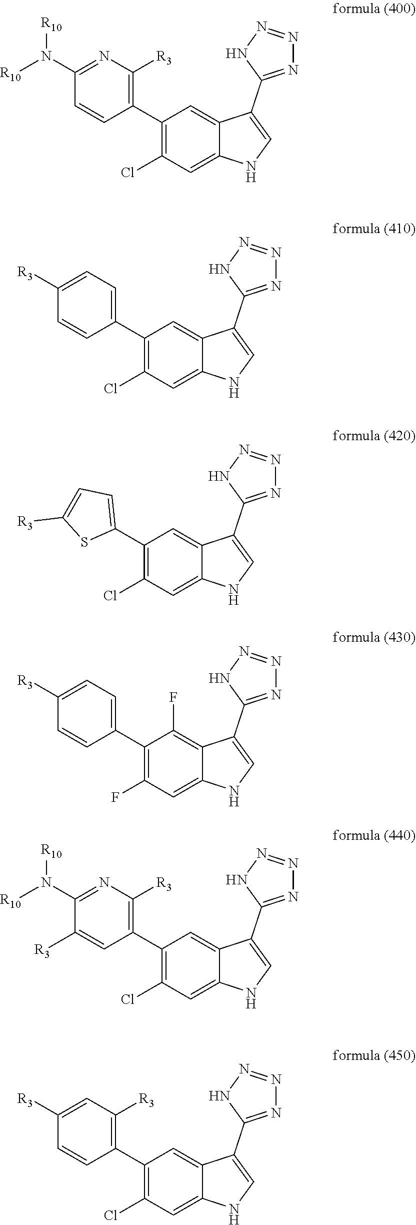

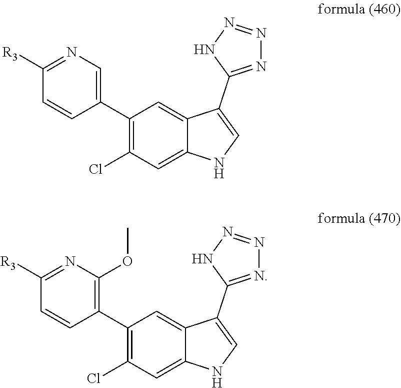

19. The compound of any one of claims 1-9, wherein R₁ is

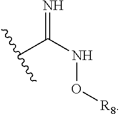

20. The compound of claim 19, wherein the compound of formula (I) is a compound of formula (500), formula (510), formula (520), formula (530), formula (540), formula (550), formula (560), or formula (570) or a pharmaceutically acceptable salt, solvate, hydrate, cocrystal, or prodrug thereof:

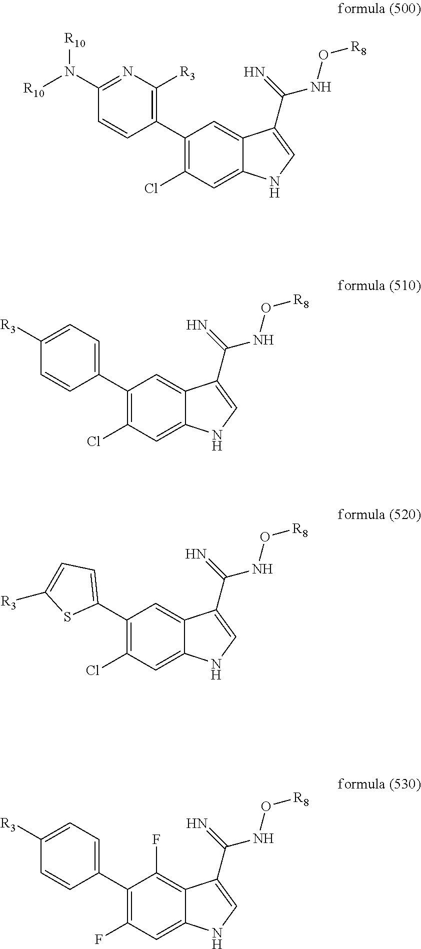

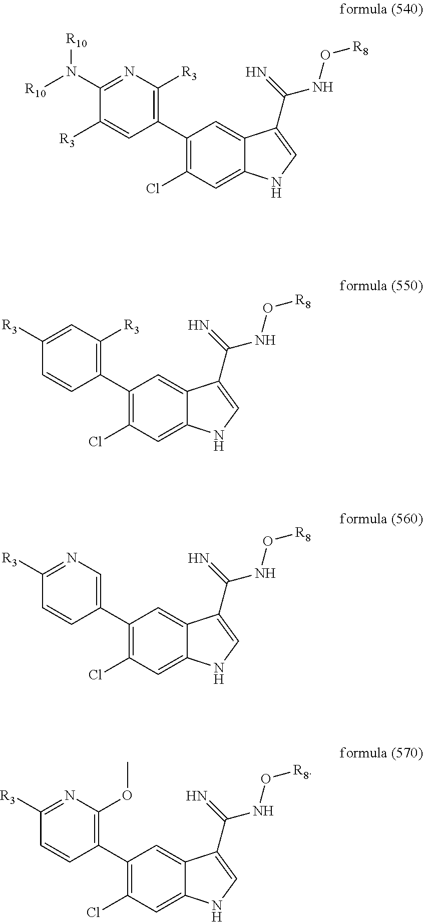

21. The compound of any one of claims 1-9, wherein R₁ is

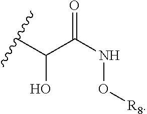

22. The compound of claim 21, wherein the compound of formula (I) is a compound of formula (600), formula (610), formula (620), formula (630), formula (640), formula (650), formula (660), or formula (670) or a pharmaceutically acceptable salt, solvate, hydrate, cocrystal, or prodrug thereof:

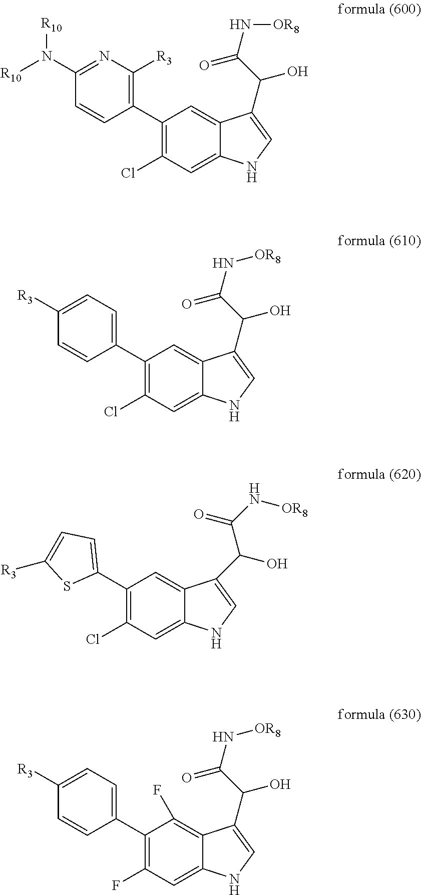

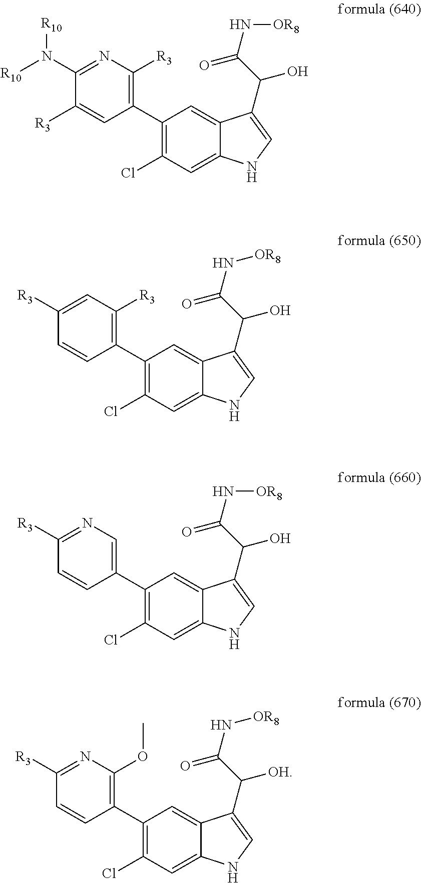

23. The compound of any one of claims 1-9, wherein R₁ is

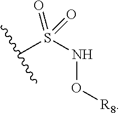

24. The compound of claim 23, wherein the compound of formula (I) is a compound of formula (700), formula (710), formula (720), formula (730), formula (740), formula (750), formula (760), or formula (770) or a pharmaceutically acceptable salt, solvate, hydrate, cocrystal, or prodrug thereof:

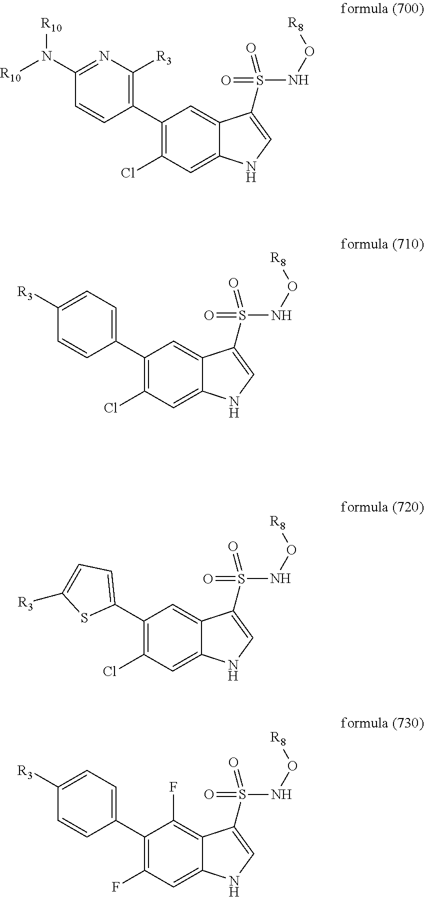

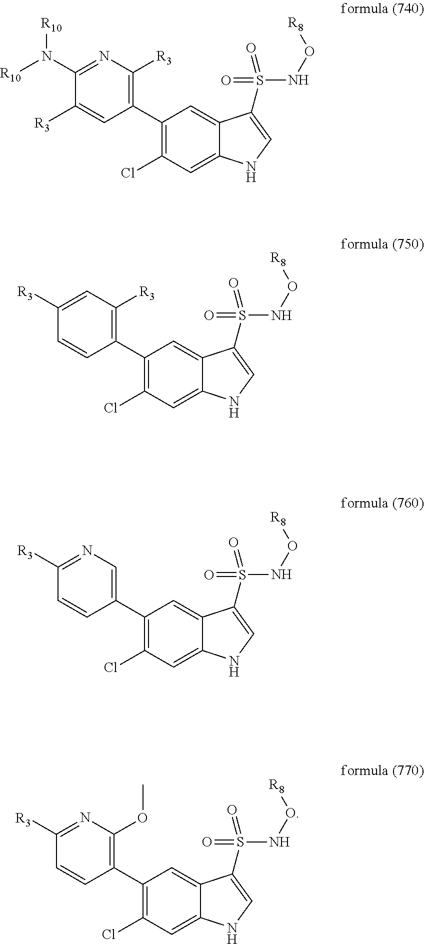

25. The compound of any one of claims 1-9, wherein R₁ is

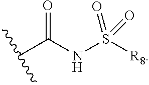

26. The compound of claim 25, wherein the compound of formula (I) is a compound of formula (800), formula (810), formula (820), formula (830), formula (840), formula (850), formula (860), or formula (870) or a pharmaceutically acceptable salt, solvate, hydrate, cocrystal, or prodrug thereof:

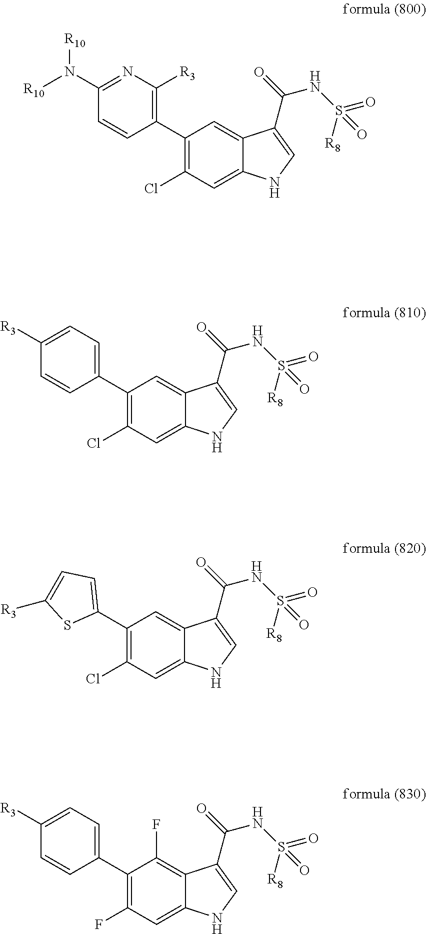

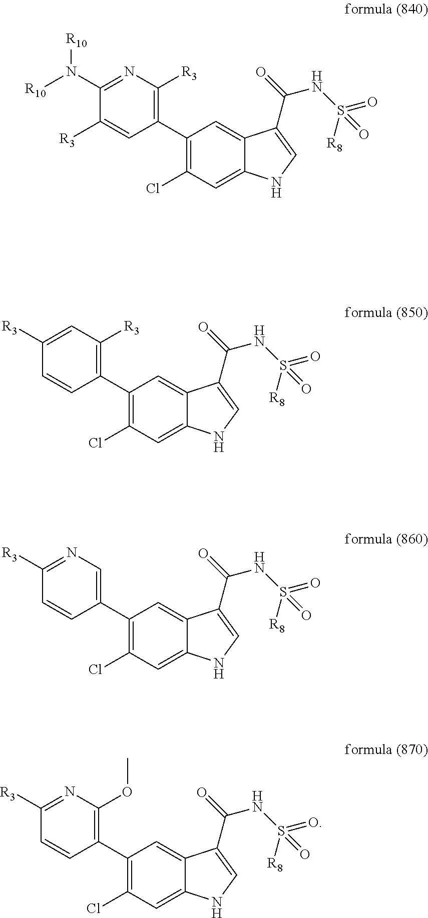

27. The compound of any one of claims 1-9, wherein R₁ is

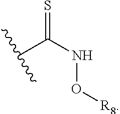

28. The compound of claim 27, wherein the compound of formula (I) is a compound of formula (900), formula (910), formula (920), formula (930), formula (940), formula (950), formula (960), or formula (970) or a pharmaceutically acceptable salt, solvate, hydrate, cocrystal, or prodrug thereof:

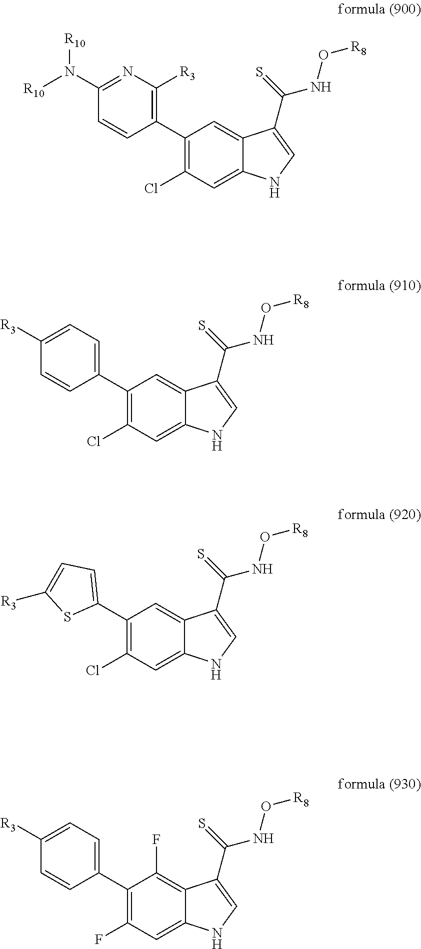

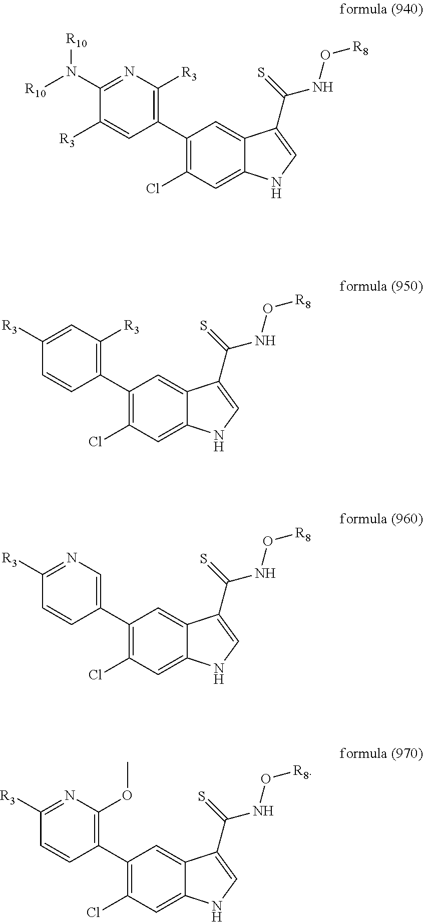

29. The compound of any one of claims 1-28, wherein R₁₀ is selected from H, methyl, ethyl, —CD₃, n-butyl,

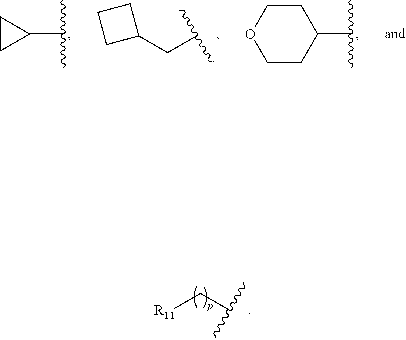

30. The compound of any one of claims 1-29, wherein R₁ is selected from

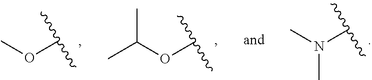

31. The compound of any one of claims 1-30, wherein p is 2 or 3.

32. The compound of any one of claims 1-31, wherein R₃ is selected from —F, —CN, ethyl, 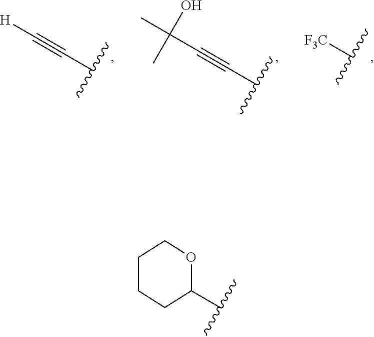
optionally

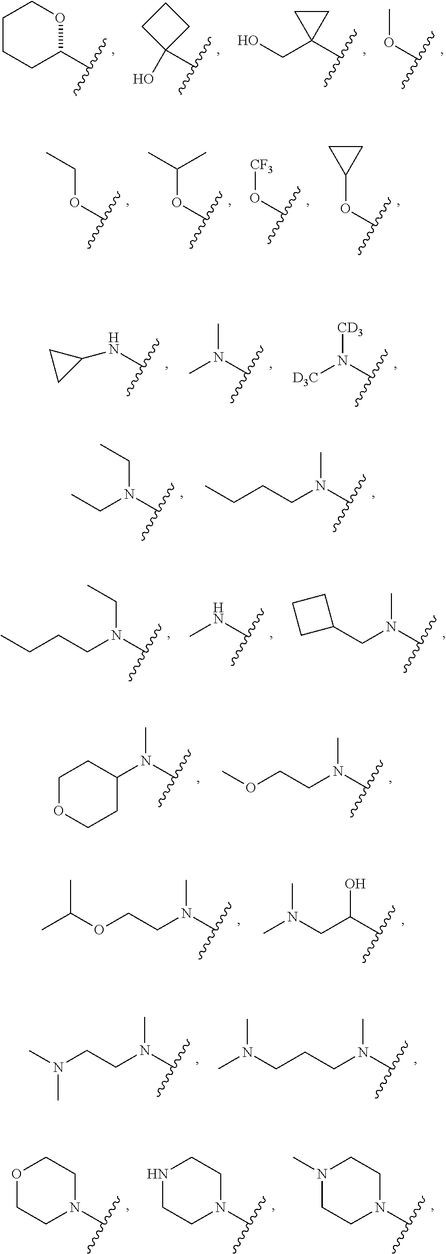

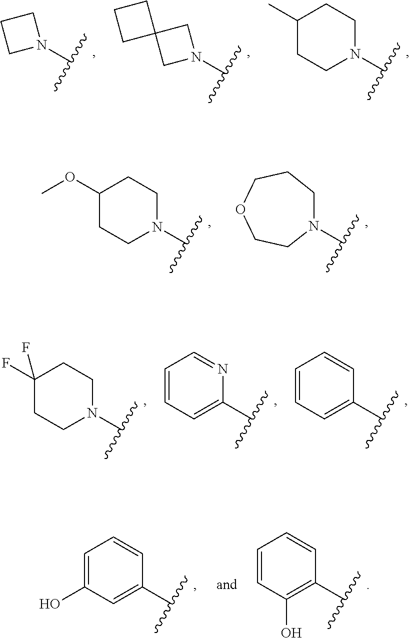

33. The compound of any one of claims 1-32, wherein R₃ is selected from 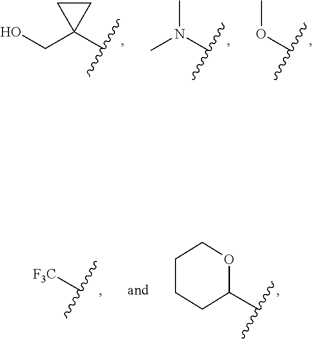
optionally

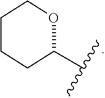 [SMILES: *[C@@H]1CCCCO1]

34. The compound of any one of claims 1-33, wherein q is 0 or 1.

35. The compound of any one of claims 1-33, wherein r is 2 or 3.

36. The compound of any one of claims 1-33, wherein s is 1.

37. The compound of any one of claims 1-36, wherein R₁₂ is selected from methyl, ethyl, —OCF₃, and

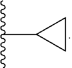 [SMILES: *C1CC1]

38. The compound of any one of claims 1-37, wherein R₈ is selected from H, methyl, ethyl, isopropyl, n-butyl, t-butyl,

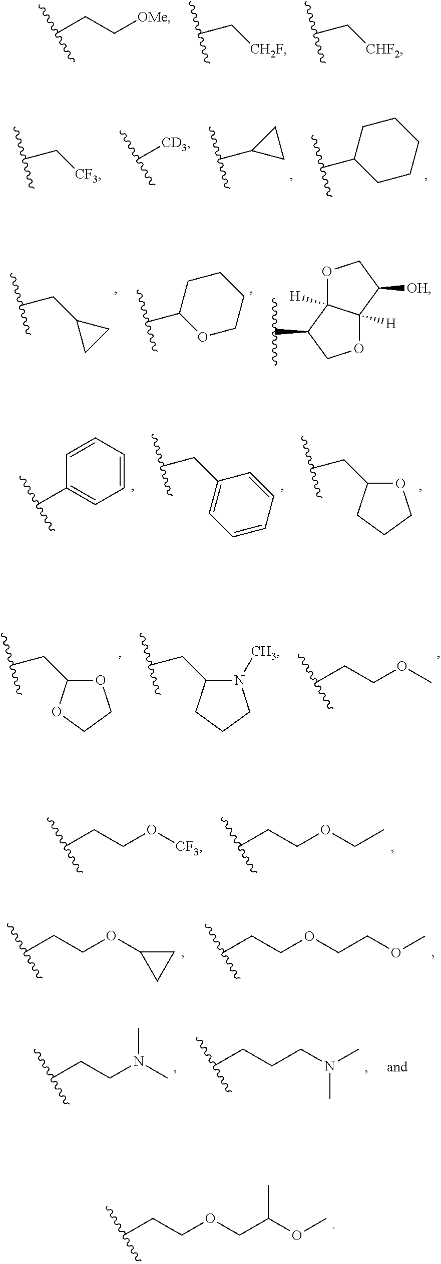

39. The compound of any one of claims 1-38, wherein R₈ is selected from H, methyl, ethyl, n-butyl,

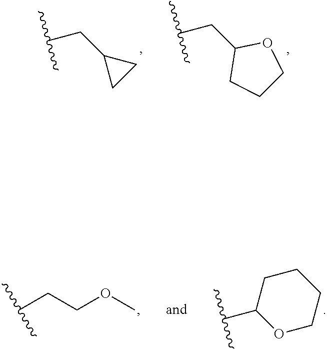

40. The compound of any one of claims 1-39, wherein R₈ is selected from H, methyl, and ethyl.

41. The compound of claim 1, wherein the compound of formula (I) is selected from a compound having a formula selected from formula 1001-1114, or a pharmaceutically acceptable salt, solvate, hydrate, cocrystal, or prodrug thereof:

| Cpd. | |
|---|---|
| # | |
| 1001 | 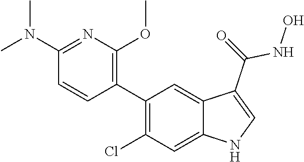 [SMILES: COc1nc(N(C)C)ccc1-c1cc2c(C(=O)NO)c[nH]c2cc1Cl] |
| 1002 | 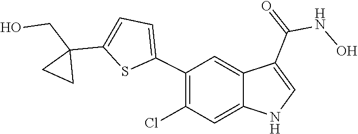 [SMILES: O=C(NO)c1c[nH]c2cc(Cl)c(-c3ccc(C4(CO)CC4)s3)cc12] |
| 1003 | 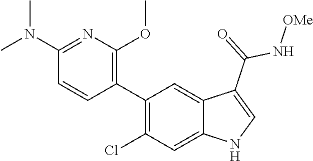 [SMILES: CONC(=O)c1c[nH]c2cc(Cl)c(-c3ccc(N(C)C)nc3OC)cc12] |
| 1004 | 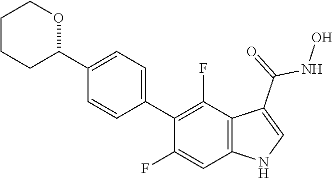 [SMILES: O=C(NO)c1c[nH]c2cc(F)c(-c3ccc([C@@H]4CCCCO4)cc3)c(F)c12] |
| 1005 | 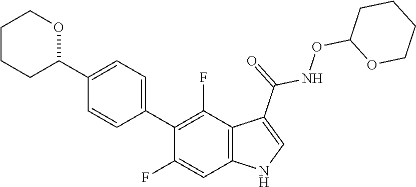 [SMILES: O=C(NOC1CCCCO1)c1c[nH]c2cc(F)c(-c3ccc([C@@H]4CCCCO4)cc3)c(F)c12] |
| 1006 | 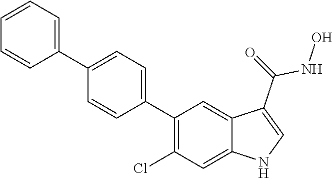 [SMILES: O=C(NO)c1c[nH]c2cc(Cl)c(-c3ccc(-c4ccccc4)cc3)cc12] |
| 1007 | 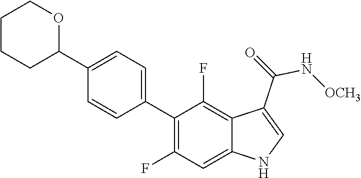 [SMILES: CONC(=O)c1c[nH]c2cc(F)c(-c3ccc(C4CCCCO4)cc3)c(F)c12] |
| 1008 | 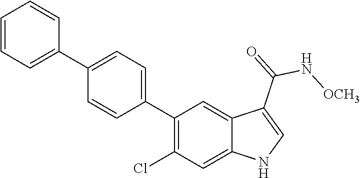 [SMILES: CONC(=O)c1c[nH]c2cc(Cl)c(-c3ccc(-c4ccccc4)cc3)cc12] |
| 1009 | 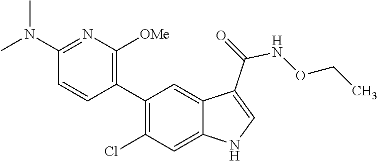 [SMILES: CCONC(=O)c1c[nH]c2cc(Cl)c(-c3ccc(N(C)C)nc3OC)cc12] |
| 1010 | 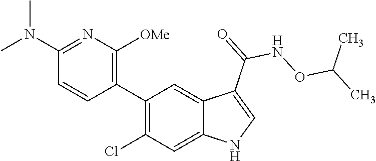 [SMILES: COc1nc(N(C)C)ccc1-c1cc2c(C(=O)NOC(C)C)c[nH]c2cc1Cl] |
| 1011 | 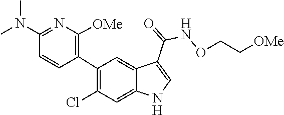 [SMILES: COCCONC(=O)c1c[nH]c2cc(Cl)c(-c3ccc(N(C)C)nc3OC)cc12] |
| 1012 | 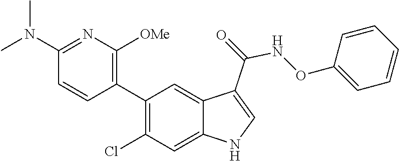 [SMILES: COc1nc(N(C)C)ccc1-c1cc2c(C(=O)NOc3ccccc3)c[nH]c2cc1Cl] |
| 1013 | 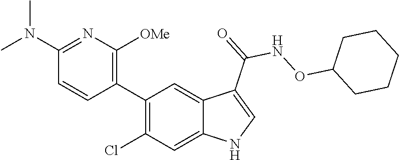 [SMILES: COc1nc(N(C)C)ccc1-c1cc2c(C(=O)NOC3CCCCC3)c[nH]c2cc1Cl] |
| 1014 | 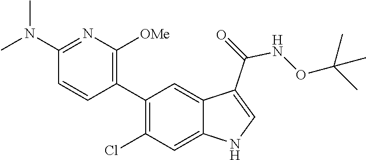 [SMILES: COc1nc(N(C)C)ccc1-c1cc2c(C(=O)NOC(C)(C)C)c[nH]c2cc1Cl] |
| 1015 | 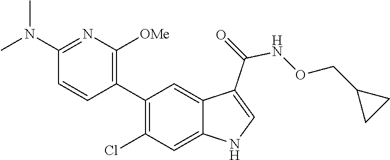 [SMILES: COc1nc(N(C)C)ccc1-c1cc2c(C(=O)NOCC3CC3)c[nH]c2cc1Cl] |
| 1016 | 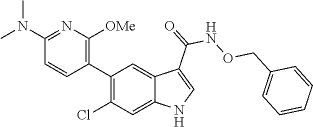 [SMILES: COc1nc(N(C)C)ccc1-c1cc2c(C(=O)NOCc3ccccc3)c[nH]c2cc1Cl] |
| 1017 | 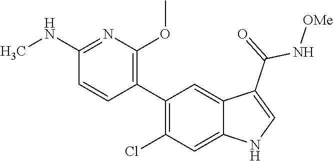 [SMILES: CNc1ccc(-c2cc3c(C(=O)NOC)c[nH]c3cc2Cl)c(OC)n1] |
| 1018 | 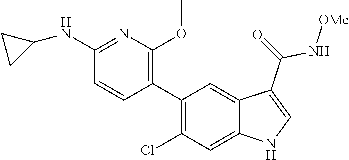 [SMILES: CONC(=O)c1c[nH]c2cc(Cl)c(-c3ccc(NC4CC4)nc3OC)cc12] |
| 1019 | 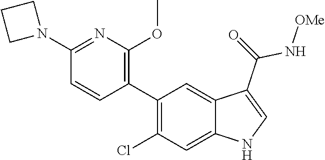 [SMILES: CONC(=O)c1c[nH]c2cc(Cl)c(-c3ccc(N4CCC4)nc3OC)cc12] |
| 1020 | 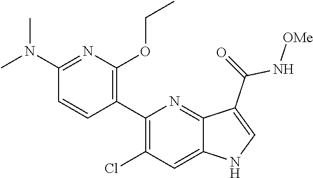 [SMILES: CCOc1nc(N(C)C)ccc1-c1nc2c(C(=O)NOC)c[nH]c2cc1Cl] |
| 1021 | 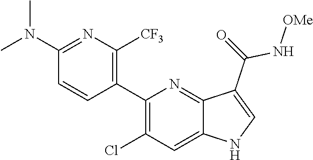 [SMILES: CONC(=O)c1c[nH]c2cc(Cl)c(-c3ccc(N(C)C)nc3C(F)(F)F)nc12] |
| 1022 | 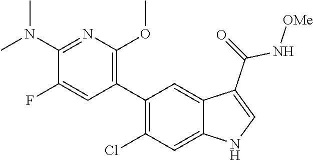 [SMILES: CONC(=O)c1c[nH]c2cc(Cl)c(-c3cc(F)c(N(C)C)nc3OC)cc12] |
| 1023 | 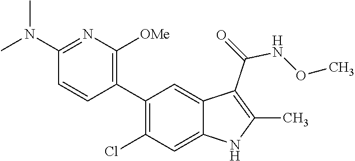 |
| 1024 | 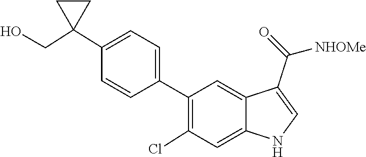 |
| 1025 | 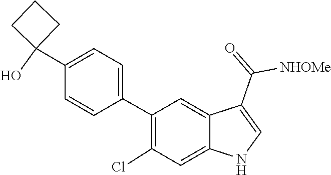 |
| 1026 | 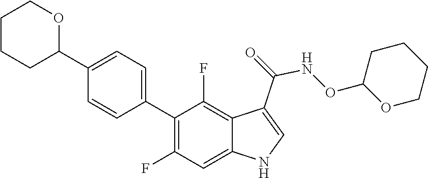 [SMILES: O=C(NOC1CCCCO1)c1c[nH]c2cc(F)c(-c3ccc(C4CCCCO4)cc3)c(F)c12] |
| 1027 | 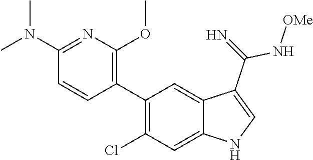 [SMILES: CONC(=N)c1c[nH]c2cc(Cl)c(-c3ccc(N(C)C)nc3OC)cc12] |
| 1028 | 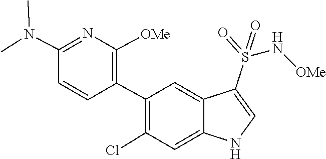 [SMILES: CONS(=O)(=O)c1c[nH]c2cc(Cl)c(-c3ccc(N(C)C)nc3OC)cc12] |
| 1029 | 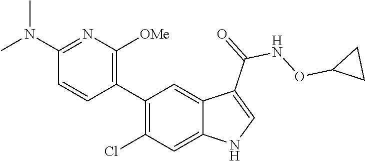 [SMILES: COc1nc(N(C)C)ccc1-c1cc2c(C(=O)NOC3CC3)c[nH]c2cc1Cl] |
| 1030 | 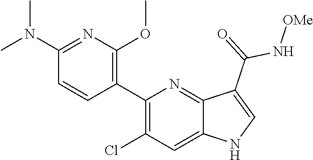 [SMILES: CONC(=O)c1c[nH]c2cc(Cl)c(-c3ccc(N(C)C)nc3OC)nc12] |
| 1031 | 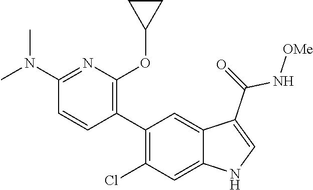 [SMILES: CONC(=O)c1c[nH]c2cc(Cl)c(-c3ccc(N(C)C)nc3OC3CC3)cc12] |
| 1032 | 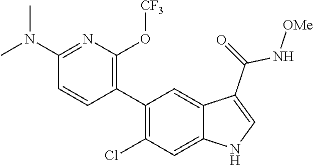 [SMILES: CONC(=O)c1c[nH]c2cc(Cl)c(-c3ccc(N(C)C)nc3OC(F)(F)F)cc12] |
| 1033 | 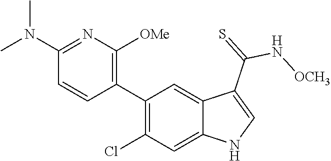 [SMILES: CONC(=S)c1c[nH]c2cc(Cl)c(-c3ccc(N(C)C)nc3OC)cc12] |
| 1034 | 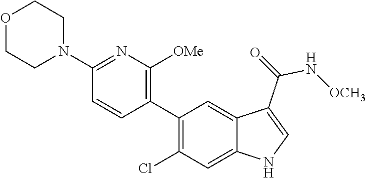 [SMILES: CONC(=O)c1c[nH]c2cc(Cl)c(-c3ccc(N4CCOCC4)nc3OC)cc12] |
| 1035 | 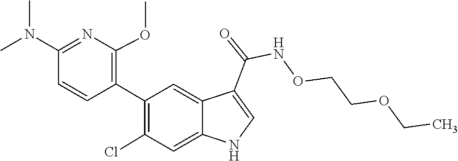 [SMILES: CCOCCONC(=O)c1c[nH]c2cc(Cl)c(-c3ccc(N(C)C)nc3OC)cc12] |
| 1036 | 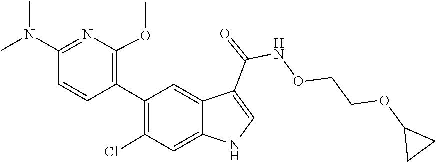 [SMILES: COc1nc(N(C)C)ccc1-c1cc2c(C(=O)NOCCOC3CC3)c[nH]c2cc1Cl] |
| 1037 | 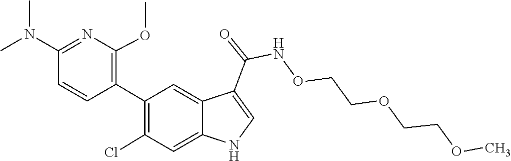 |
| 1038 | 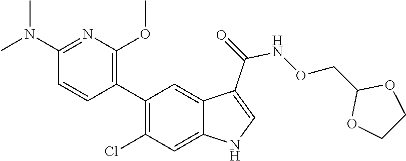 [SMILES: COc1nc(N(C)C)ccc1-c1cc2c(C(=O)NOCC3OCCO3)c[nH]c2cc1Cl] |
| 1039 | 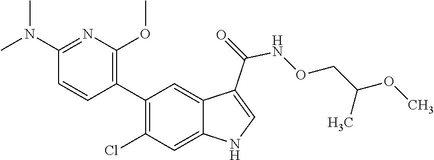 |
| 1040 | 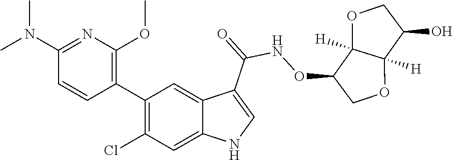 [SMILES: COc1nc(N(C)C)ccc1-c1cc2c(C(=O)NO[C@@H]3CO[C@H]4[C@@H]3OC[C@H]4O)c[nH]c2cc1Cl] |
| 1041 | 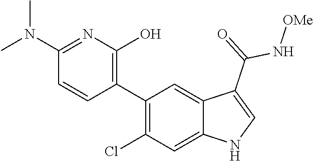 [SMILES: CONC(=O)c1c[nH]c2cc(Cl)c(-c3ccc(N(C)C)nc3O)cc12] |
| 1042 | 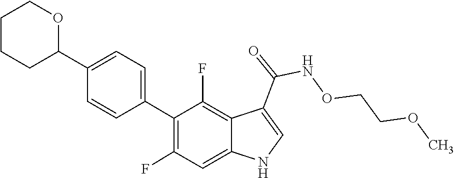 [SMILES: COCCONC(=O)c1c[nH]c2cc(F)c(-c3ccc(C4CCCCO4)cc3)c(F)c12] |
| 1043 | 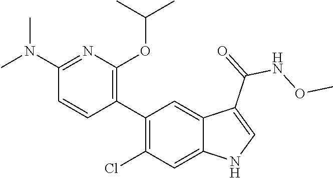 |
| 1044 | 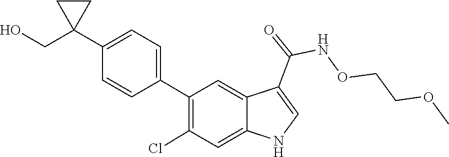 [SMILES: COCCONC(=O)c1c[nH]c2cc(Cl)c(-c3ccc(C4(CO)CC4)cc3)cc12] |
| 1045 | 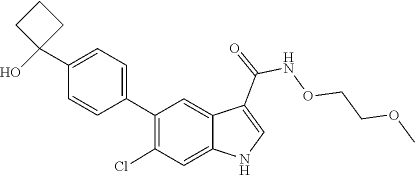 [SMILES: COCCONC(=O)c1c[nH]c2cc(Cl)c(-c3ccc(C4(O)CCC4)cc3)cc12] |
| 1046 | 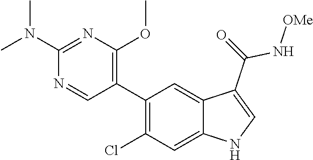 [SMILES: CONC(=O)c1c[nH]c2cc(Cl)c(-c3cnc(N(C)C)nc3OC)cc12] |
| 1047 | 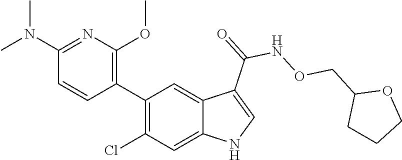 [SMILES: COc1nc(N(C)C)ccc1-c1cc2c(C(=O)NOCC3CCCO3)c[nH]c2cc1Cl] |
| 1048 | 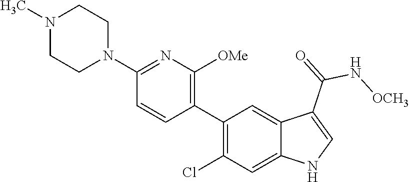 [SMILES: CONC(=O)c1c[nH]c2cc(Cl)c(-c3ccc(N4CCN(C)CC4)nc3OC)cc12] |
| 1049 | 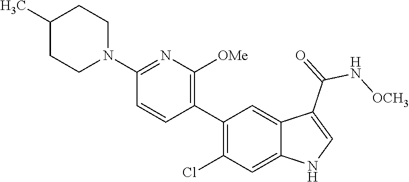 [SMILES: CONC(=O)c1c[nH]c2cc(Cl)c(-c3ccc(N4CCC(C)CC4)nc3OC)cc12] |
| 1050 | 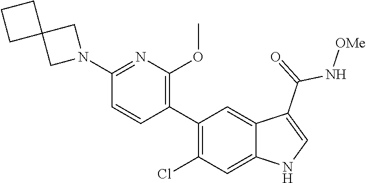 [SMILES: CONC(=O)c1c[nH]c2cc(Cl)c(-c3ccc(N4CC5(CCC5)C4)nc3OC)cc12] |
| 1051 | 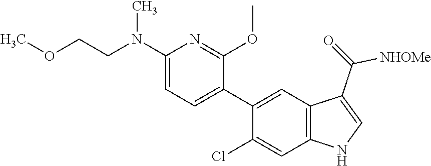 |
| 1052 | 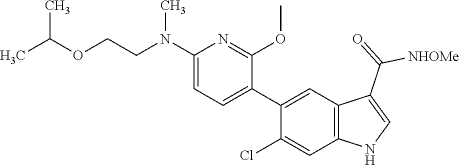 |
| 1053 | 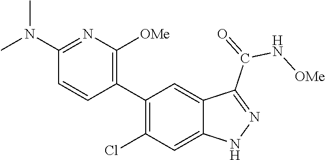 [SMILES: CONC(=O)c1n[nH]c2cc(Cl)c(-c3ccc(N(C)C)nc3OC)cc12] |
| 1054 | 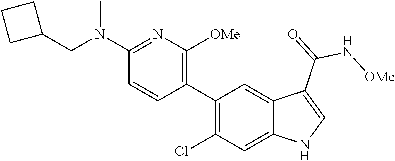 [SMILES: CONC(=O)c1c[nH]c2cc(Cl)c(-c3ccc(N(C)CC4CCC4)nc3OC)cc12] |
| 1055 | 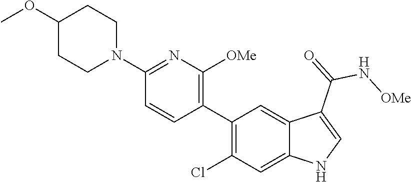 [SMILES: CONC(=O)c1c[nH]c2cc(Cl)c(-c3ccc(N4CCC(OC)CC4)nc3OC)cc12] |
| 1056 | 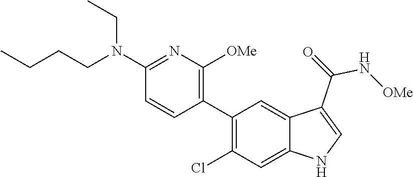 [SMILES: CCCCN(CC)c1ccc(-c2cc3c(C(=O)NOC)c[nH]c3cc2Cl)c(OC)n1] |
| 1057 | 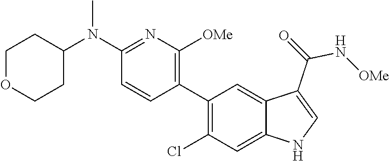 [SMILES: CONC(=O)c1c[nH]c2cc(Cl)c(-c3ccc(N(C)C4CCOCC4)nc3OC)cc12] |
| 1058 | 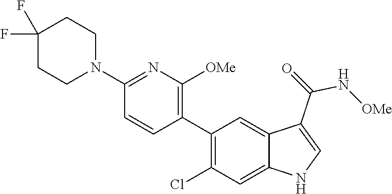 [SMILES: CONC(=O)c1c[nH]c2cc(Cl)c(-c3ccc(N4CCC(F)(F)CC4)nc3OC)cc12] |
| 1059 | 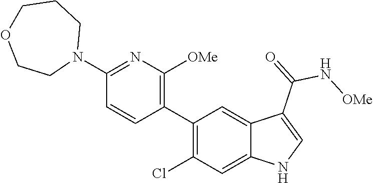 [SMILES: CONC(=O)c1c[nH]c2cc(Cl)c(-c3ccc(N4CCCOCC4)nc3OC)cc12] |
| 1060 | 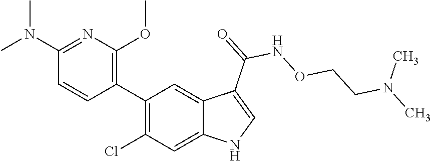 |
| 1061 | 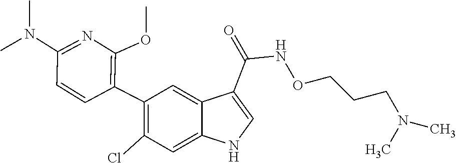 |
| 1062 | 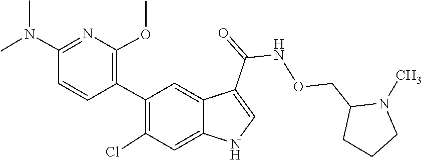 |
| 1063 | 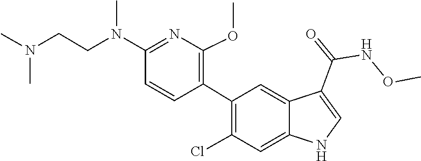 |
| 1064 | 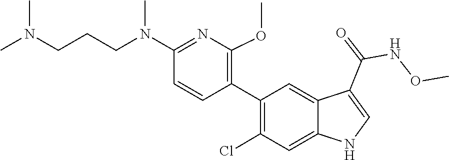 |
| 1065 | 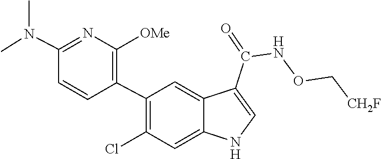 |
| 1066 | 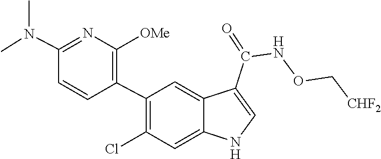 [SMILES: COc1nc(N(C)C)ccc1-c1cc2c(C(=O)NOCC(F)F)c[nH]c2cc1Cl] |
| 1067 | 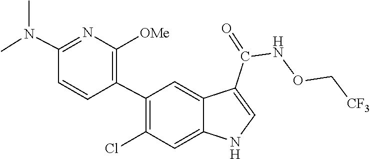 [SMILES: COc1nc(N(C)C)ccc1-c1cc2c(C(=O)NOCC(F)(F)F)c[nH]c2cc1Cl] |
| 1068 | 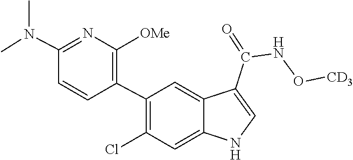 [SMILES: [2H]C([2H])([2H])ONC(=O)c1c[nH]c2cc(Cl)c(-c3ccc(N(C)C)nc3OC)cc12] |
| 1069 | 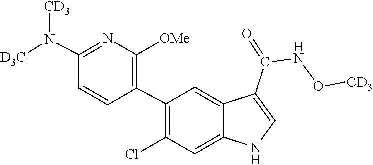 [SMILES: [2H]C([2H])([2H])ONC(=O)c1c[nH]c2cc(Cl)c(-c3ccc(N(C([2H])([2H])[2H])C([2H])([2H])[2H])nc3OC)cc12] |
| 1070 | 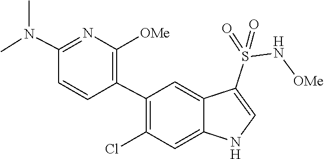 [SMILES: CONS(=O)(=O)c1c[nH]c2cc(Cl)c(-c3ccc(N(C)C)nc3OC)cc12] |
| 1071 | 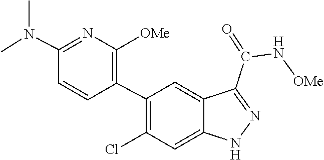 [SMILES: CONC(=O)c1n[nH]c2cc(Cl)c(-c3ccc(N(C)C)nc3OC)cc12] |
| 1072 | 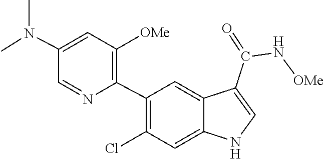 [SMILES: CONC(=O)c1c[nH]c2cc(Cl)c(-c3ncc(N(C)C)cc3OC)cc12] |
| 1073 | 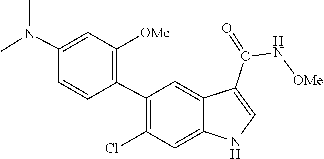 [SMILES: CONC(=O)c1c[nH]c2cc(Cl)c(-c3ccc(N(C)C)cc3OC)cc12] |
| 1074 | 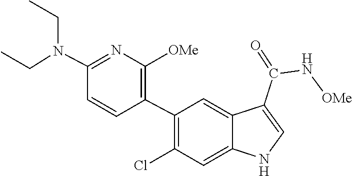 [SMILES: CCN(CC)c1ccc(-c2cc3c(C(=O)NOC)c[nH]c3cc2Cl)c(OC)n1] |
| 1075 | 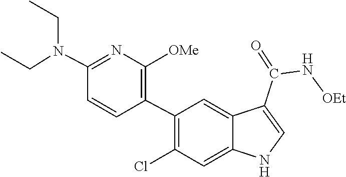 [SMILES: CCONC(=O)c1c[nH]c2cc(Cl)c(-c3ccc(N(CC)CC)nc3OC)cc12] |
| 1076 | 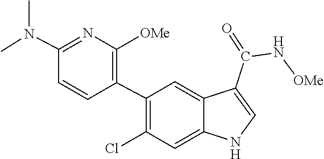 [SMILES: CONC(=O)c1c[nH]c2cc(Cl)c(-c3ccc(N(C)C)nc3OC)cc12] |
| 1077 | 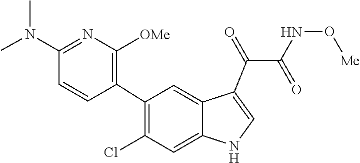 [SMILES: CONC(=O)C(=O)c1c[nH]c2cc(Cl)c(-c3ccc(N(C)C)nc3OC)cc12] |
| 1078 | 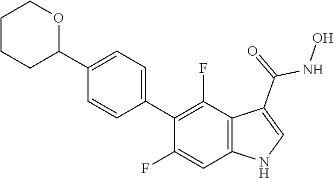 [SMILES: O=C(NO)c1c[nH]c2cc(F)c(-c3ccc(C4CCCCO4)cc3)c(F)c12] |
| 1080 | 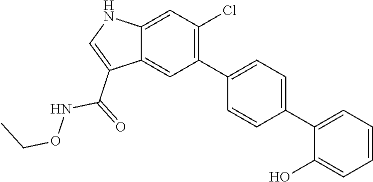 [SMILES: CCONC(=O)c1c[nH]c2cc(Cl)c(-c3ccc(-c4ccccc4O)cc3)cc12] |
| 1081 | 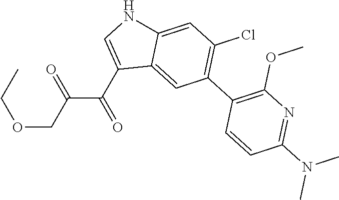 [SMILES: CCOCC(=O)C(=O)c1c[nH]c2cc(Cl)c(-c3ccc(N(C)C)nc3OC)cc12] |
| 1082 | 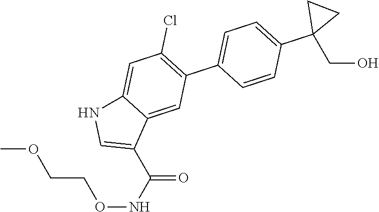 [SMILES: COCCONC(=O)c1c[nH]c2cc(Cl)c(-c3ccc(C4(CO)CC4)cc3)cc12] |
| 1084 | 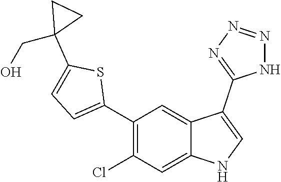 [SMILES: OCC1(c2ccc(-c3cc4c(-c5nnn[nH]5)c[nH]c4cc3Cl)s2)CC1] |
| 1085 | 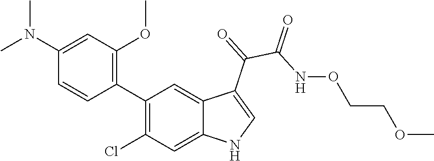 |
| 1088 | 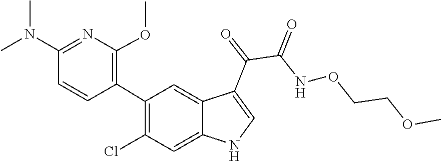 |
| 1089 | 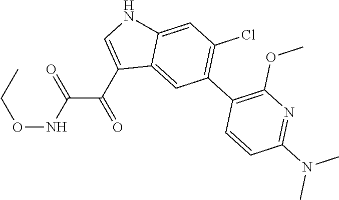 [SMILES: CCONC(=O)C(=O)c1c[nH]c2cc(Cl)c(-c3ccc(N(C)C)nc3OC)cc12] |
| 1090 | 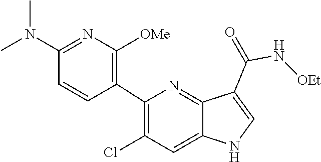 [SMILES: CCONC(=O)c1c[nH]c2cc(Cl)c(-c3ccc(N(C)C)nc3OC)nc12] |
| 1091 | 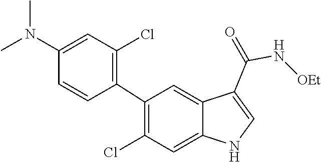 [SMILES: CCONC(=O)c1c[nH]c2cc(Cl)c(-c3ccc(N(C)C)cc3Cl)cc12] |
| 1092 | 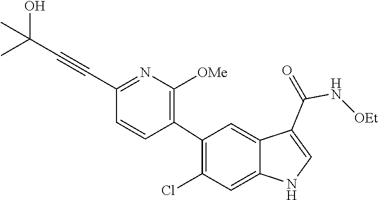 [SMILES: CCONC(=O)c1c[nH]c2cc(Cl)c(-c3ccc(C#CC(C)(C)O)nc3OC)cc12] |
| 1093 | 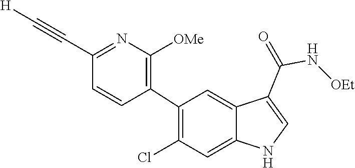 [SMILES: C#Cc1ccc(-c2cc3c(C(=O)NOCC)c[nH]c3cc2Cl)c(OC)n1] |
| 1094 | 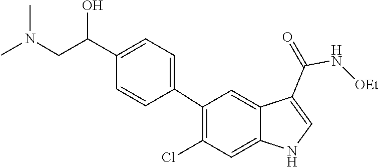 [SMILES: CCONC(=O)c1c[nH]c2cc(Cl)c(-c3ccc(C(O)CN(C)C)cc3)cc12] |
| 1095 | 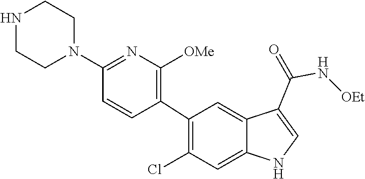 [SMILES: CCONC(=O)c1c[nH]c2cc(Cl)c(-c3ccc(N4CCNCC4)nc3OC)cc12] |
| 1096 | 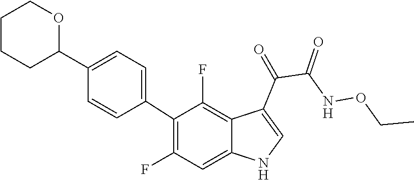 [SMILES: CCONC(=O)C(=O)c1c[nH]c2cc(F)c(-c3ccc(C4CCCCO4)cc3)c(F)c12] |
| 1097 | 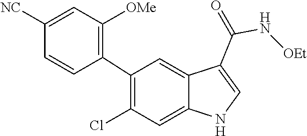 [SMILES: CCONC(=O)c1c[nH]c2cc(Cl)c(-c3ccc(C#N)cc3OC)cc12] |
| 1098 | 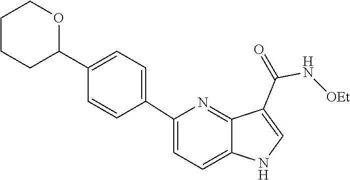 [SMILES: CCONC(=O)c1c[nH]c2ccc(-c3ccc(C4CCCCO4)cc3)nc12] |
| 1099 | 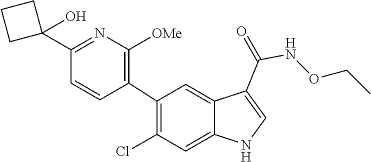 [SMILES: CCONC(=O)c1c[nH]c2cc(Cl)c(-c3ccc(C4(O)CCC4)nc3OC)cc12] |
| 1100 | 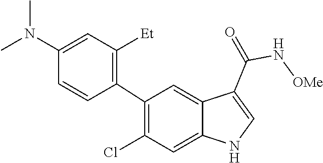 [SMILES: CCc1cc(N(C)C)ccc1-c1cc2c(C(=O)NOC)c[nH]c2cc1Cl] |
| 1101 | 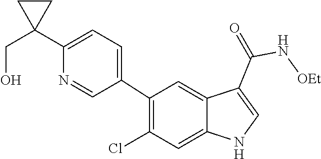 [SMILES: CCONC(=O)c1c[nH]c2cc(Cl)c(-c3ccc(C4(CO)CC4)nc3)cc12] |
| 1102 | 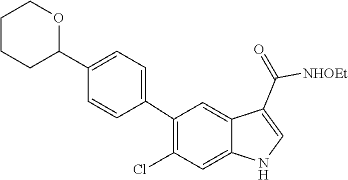 |
| 1103 | 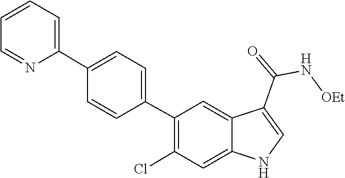 [SMILES: CCONC(=O)c1c[nH]c2cc(Cl)c(-c3ccc(-c4ccccn4)cc3)cc12] |
| 1104 | 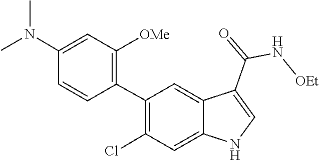 [SMILES: CCONC(=O)c1c[nH]c2cc(Cl)c(-c3ccc(N(C)C)cc3OC)cc12] |
| 1105 | 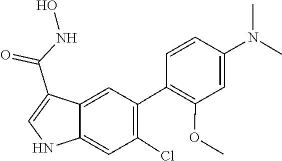 [SMILES: COc1cc(N(C)C)ccc1-c1cc2c(C(=O)NO)c[nH]c2cc1Cl] |
| 1106 | 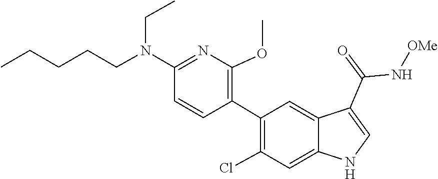 [SMILES: CCCCCN(CC)c1ccc(-c2cc3c(C(=O)NOC)c[nH]c3cc2Cl)c(OC)n1] |
| 1107 | 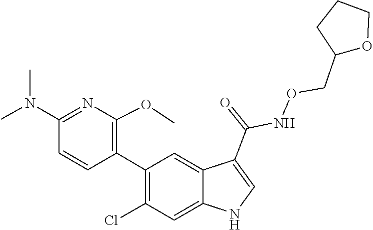 [SMILES: COc1nc(N(C)C)ccc1-c1cc2c(C(=O)NOCC3CCCO3)c[nH]c2cc1Cl] |
| 1108 | 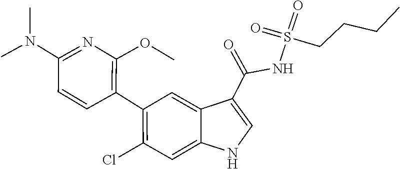 [SMILES: CCCCS(=O)(=O)NC(=O)c1c[nH]c2cc(Cl)c(-c3ccc(N(C)C)nc3OC)cc12] |
| 1109 | 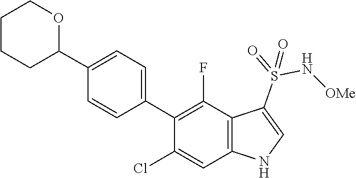 [SMILES: CONS(=O)(=O)c1c[nH]c2cc(Cl)c(-c3ccc(C4CCCCO4)cc3)c(F)c12] |
| 1110 | 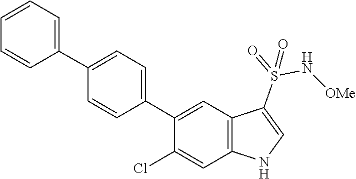 [SMILES: CONS(=O)(=O)c1c[nH]c2cc(Cl)c(-c3ccc(-c4ccccc4)cc3)cc12] |
| 1111 | 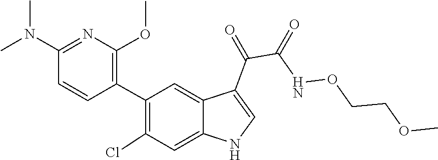 |
| 1112 | 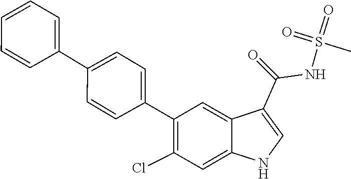 [SMILES: CS(=O)(=O)NC(=O)c1c[nH]c2cc(Cl)c(-c3ccc(-c4ccccc4)cc3)cc12] |
| 1113 | 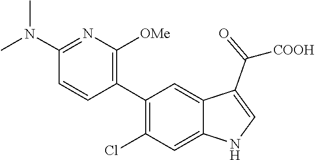 [SMILES: COc1nc(N(C)C)ccc1-c1cc2c(C(=O)C(=O)O)c[nH]c2cc1Cl] |
| 1114 | 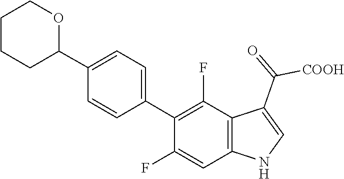 [SMILES: O=C(O)C(=O)c1c[nH]c2cc(F)c(-c3ccc(C4CCCCO4)cc3)c(F)c12] |

42. The compound of claim 1, wherein the compound of formula (I) is selected from:

| Cpd. | |
|---|---|
| # | |
| 1003 | 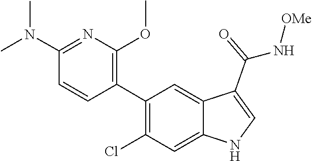 [SMILES: CONC(=O)c1c[nH]c2cc(Cl)c(-c3ccc(N(C)C)nc3OC)cc12] |
| 1007 | 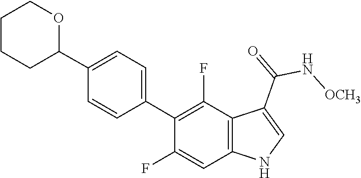 [SMILES: CONC(=O)c1c[nH]c2cc(F)c(-c3ccc(C4CCCCO4)cc3)c(F)c12] |
| 1024 | 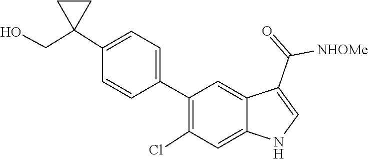 |
| 1044 | 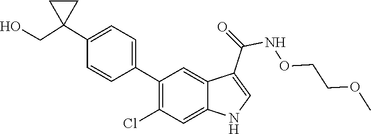 [SMILES: COCCONC(=O)c1c[nH]c2cc(Cl)c(-c3ccc(C4(CO)CC4)cc3)cc12] |
| 1077 | 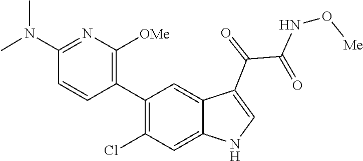 [SMILES: CONC(=O)C(=O)c1c[nH]c2cc(Cl)c(-c3ccc(N(C)C)nc3OC)cc12] |
| 1078 | 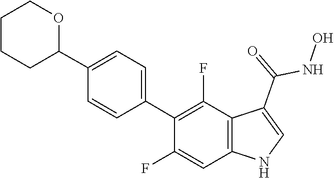 [SMILES: O=C(NO)c1c[nH]c2cc(F)c(-c3ccc(C4CCCCO4)cc3)c(F)c12] |
| 1081 | 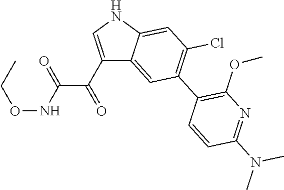 [SMILES: CCONC(=O)C(=O)c1c[nH]c2cc(Cl)c(-c3ccc(N(C)C)nc3OC)cc12] |
| 1084 | 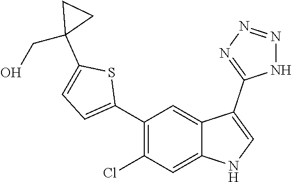 [SMILES: OCC1(c2ccc(-c3cc4c(-c5nnn[nH]5)c[nH]c4cc3Cl)s2)CC1] |
| 1088 | 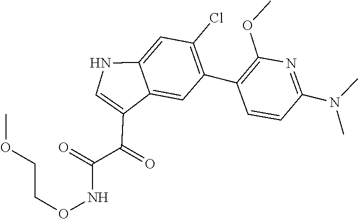 [SMILES: COCCONC(=O)C(=O)c1c[nH]c2cc(Cl)c(-c3ccc(N(C)C)nc3OC)cc12] |
| 1112 | 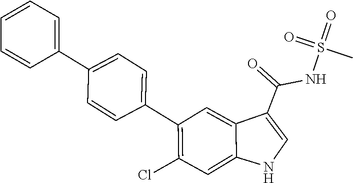 [SMILES: CS(=O)(=O)NC(=O)c1c[nH]c2cc(Cl)c(-c3ccc(-c4ccccc4)cc3)cc12] |
| 1113 | 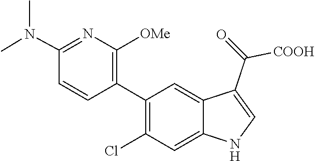 [SMILES: COc1nc(N(C)C)ccc1-c1cc2c(C(=O)C(=O)O)c[nH]c2cc1Cl] |

or a pharmaceutically acceptable salt, solvate, hydrate, cocrystal, or prodrug thereof.

43. The compound of claim 1, wherein the compound of formula (I) is selected from:

| | |
|---|---|
| 1001 | 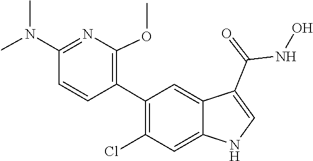 [SMILES: COc1nc(N(C)C)ccc1-c1cc2c(C(=O)NO)c[nH]c2cc1Cl] |
| 1006 | 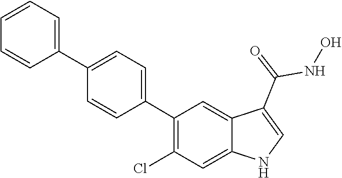 [SMILES: O=C(NO)c1c[nH]c2cc(Cl)c(-c3ccc(-c4ccccc4)cc3)cc12] |
| 1008 | 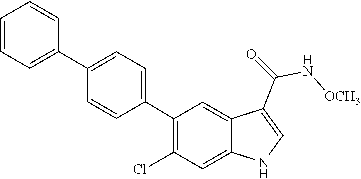 [SMILES: CONC(=O)c1c[nH]c2cc(Cl)c(-c3ccc(-c4ccccc4)cc3)cc12] |
| 1010 | 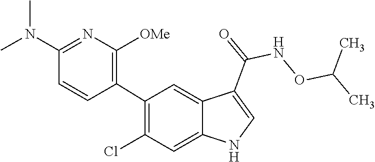 [SMILES: COc1nc(N(C)C)ccc1-c1cc2c(C(=O)NOC(C)C)c[nH]c2cc1Cl] |
| 1013 | 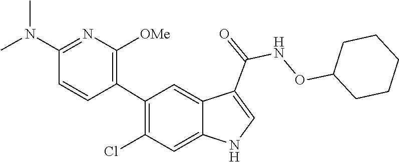 [SMILES: COc1nc(N(C)C)ccc1-c1cc2c(C(=O)NOC3CCCCC3)c[nH]c2cc1Cl] |
| 1014 | 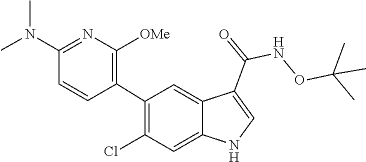 [SMILES: COc1nc(N(C)C)ccc1-c1cc2c(C(=O)NOC(C)(C)C)c[nH]c2cc1Cl] |
| 1016 | 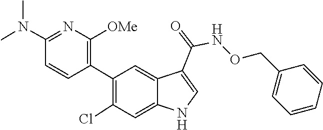 [SMILES: COc1nc(N(C)C)ccc1-c1cc2c(C(=O)NOCc3ccccc3)c[nH]c2cc1Cl] |
| 1017 | 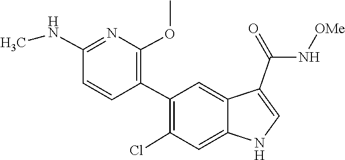 [SMILES: CNc1ccc(-c2cc3c(C(=O)NOC)c[nH]c3cc2Cl)c(OC)n1] |
| 1018 | 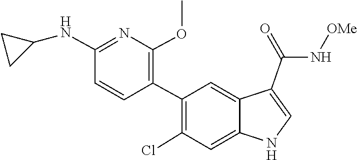 [SMILES: CONC(=O)c1c[nH]c2cc(Cl)c(-c3ccc(NC4CC4)nc3OC)cc12] |
| 1022 | 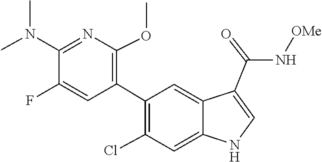 [SMILES: CONC(=O)c1c[nH]c2cc(Cl)c(-c3cc(F)c(N(C)C)nc3OC)cc12] |

or a pharmaceutically acceptable salt, solvate, hydrate, cocrystal, or prodrug thereof.

44. The compound of claim 1, wherein the compound of formula (I) is selected from:

| | |
|---|---|
| 1003 | 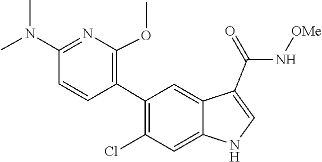 [SMILES: CONC(=O)c1c[nH]c2cc(Cl)c(-c3ccc(N(C)C)nc3OC)cc12] |
| 1009 | 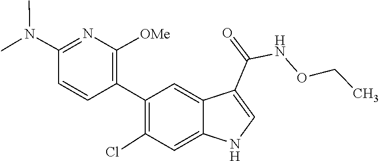 [SMILES: CCONC(=O)c1c[nH]c2cc(Cl)c(-c3ccc(N(C)C)nc3OC)cc12] |
| 1011 | 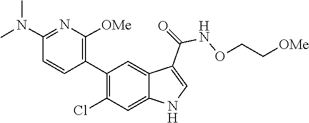 [SMILES: COCCONC(=O)c1c[nH]c2cc(Cl)c(-c3ccc(N(C)C)nc3OC)cc12] |
| 1015 | 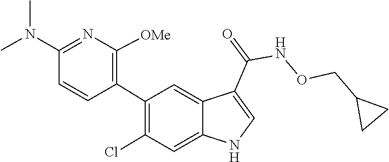 [SMILES: COc1nc(N(C)C)ccc1-c1cc2c(C(=O)NOCC3CC3)c[nH]c2cc1Cl] |

or a pharmaceutically acceptable salt, solvate, hydrate, cocrystal, or prodrug thereof.

45. The compound of claim 1, wherein when A is a 5-membered ring heterocycle and R₁ is 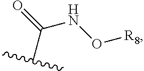
R₈ is not H.

46. A pharmaceutical formulation comprising the compound of any one of claims 1-45.

47. A method of treating a patient with a mitochondrial dysfunction comprising:
identifying a mitochondrial dysfunction in an individual; and
administering a compound of any one of claims 1-44 to the patient.

48. A method of treating a patient with a mitochondrial dysfunction, comprising administering a therapeutically effective amount of a compound of any one of claims 1-45 to the patient.

49. The method of claim 47 or 48, wherein the mitochondrial dysfunction is a primary mitochondrial dysfunction.

50. The method of claim 49, wherein the primary mitochondrial dysfunction is selected from the group consisting of Autosomal Dominant Optic Atrophy (ADOA), Alpers-Huttenlocher syndrome (nDNA defect), Ataxia neuropathy syndrome, (nDNA defect), Barth syndrome/Lethal Infantile Cardiomyopathy (LIC), Co-enzyme Q deficiency, Complex I, complex II, complex III, complex IV and complex V deficiencies (either single deficiencies or any combination of deficiency), Chronic progressive external ophthalmoplegia (CPEO), Diabetes mellitus and deafness, Kearns-Sayre syndrome (mtDNA defect), Leukoencephalopathy with Brainstem and Spinal Cord Involvement and Lactate Elevation (LBSL-leukodystrophy), Leigh syndrome (mtDNA and nDNA defects), Leber's hereditary optic neuropathy (LHON), Luft Disease, Mitochondrial myopathy, encephalopathy, lactic acidosis, and stroke syndrome (MELAS) (mtDNA defect), Mitochondrial Enoyl CoA Reductase Protein-Associated Neurodegeneration (MEPAN), Myoclonic epilepsy with ragged red fibers (MERRF), mitochondrial recessive ataxia syndrome (MIRAS), mtDNA deletion syndrome, mtDNA Depletion syndrome, mtDNA maintenance disorders, mtDNA/RNA translation defects, Mitochondrial tRNA synthetase deficiencies, Mitochondrial Myopathy, Mitochondrial neurogastrointestinal encephalopathy syndrome (MNGIE), Neurogenic muscle weakness, ataxia, and retinitis pigmentosa (NARP), Pearson syndrome, Pyruvate dehydrogenase complex deficiency (PDCD/PDH), DNA polymerase gamma deficiency (POLG), Pyruvate carboxylase deficiency, and Thymidine kinase 2 deficiency (TK2).

51. The method of claim 47 or 48, wherein the mitochondrial dysfunction is a secondary mitochondrial dysfunction.

52. The method of claim 51, wherein the secondary mitochondrial dysfunction is selected from the group consisting of age-related macular degeneration (AMD), Amyotrophic Lateral Sclerosis (ALS), Alzheimer's disease (AD) and other dementias, Friedreich's ataxia (FA), Huntington's disease (HD), Motor neuron diseases (MND), N-glycanase deficiency (NGLY1), Organic acidemias, Parkinson's disease (PD) and PD-related disorders, Prion disease, Spinal muscular atrophy (SMA), Spinocerebellar ataxia (SCA), Becker muscular dystrophy, Congenital muscular dystrophies, Duchenne muscular dystrophy, Emery-Dreifuss muscular dystrophy, Facioscapulohumeral muscular dystrophy, Myotonic dystrophy, Oculopharyngeal muscular dystrophy, Charcot-Marie-Tooth disease, Congenital myopathies, Distal myopathies, Endocrine myopathies (hyperthyroid myopathy, hypothyroid myopathy), Giant axonal neuropathy, Hereditary spastic paraplegia, Inflammatory myopathies (dermatomyositis, inclusion-body myositis, polymyositis), Metabolic myopathies, Neuromuscular junction diseases, Autism, Cancer, Diabetes, Metabolic syndrome, Chronic fatigue syndrome, an inflammatory disorder, arthritis, aging, and mitochondrial epilepsy (epilepsy secondary to primary mitochondrial disease).

53. The method of claim 47 or 48, wherein the compound is administered in a pharmaceutical formulation.

54. The method of claim 53, wherein the pharmaceutical formulation comprises the compound and at least one selected from a binding agent, a lubricating agent, a buffer, and a coating.

55. The method of claim 47 or 48, wherein the compound is administered orally.

56. The method of claim 47 or 48, wherein the compound is administered daily for at least one week.

57. The method of claim 47, further comprising assessing the efficacy of the compound in the individual.

58. The method of claim 47 or 48, wherein the compound is administered by oral administration, subcutaneous administration, intravenous administration, intraperitoneal administration, intranasal administration, dermal administration, intravitreal injection, or inhalation.
